(19) United States
(12) Patent Application Publication
(10) Pub. No.: US 2023/0147657 A1
(43) Pub. Date: May 11, 2023

(54) CHIMERIC ANTIGEN RECEPTOR SPECIFIC FOR HUMAN CD45RC AND USES THEREOF

(71) Applicants: INSERM (INSTITUT NATIONAL DE LA SANTÉ ET DE LA RECHERCHE MÉDICALE), Tolbiac (FR); NANTES UNIVERSITE, Nantes (FR)

(72) Inventors: Carole GUILLONNEAU, La Chevrolière (FR); Ignacio ANEGON, Nantes (FR)

(73) Assignees: INSERM (INSTITUT NATIONAL DE LA SANTÉ ET DE LA RECHERCHE MÉDICALE), Paris Cedex 13 (FR); NANTES UNIVERSITE, Nantes (FR)

(21) Appl. No.: 17/912,726

(22) PCT Filed: Mar. 19, 2021
(86) PCT No.: PCT/EP2021/057132

§ 371 Date: Sep. 19, 2022

(30) Foreign Application Priority Data
Mar. 20, 2020 (EP) 20305298.0

(51) Int. Cl. C07K 16/28; A61P 37/06; C12N 15/63

(52) U.S. Cl. CPC C07K 16/289; A61P 37/06; C12N 15/63; C07K 2319/03; C07K 2319/02; C07K 2317/565; C07K 2317/622

(57) ABSTRACT

In the field of immunotherapy, a chimeric antigen receptor (CAR) specific for-human CD45RC. Also, the immune cells expressing this CAR and the use thereof as a medicament. In particular, the use of this CAR for preventing or treating CD45RC^{high}-related diseases (including autoimmune diseases, undesired immune responses, monogenic diseases, lymphoma or cancer), or graft-versus-host disease (GVHD).

### FIELD OF INVENTION

The present invention relates to the field of immunotherapy. In particular, the present invention relates to a chimeric antigen receptor (CAR) specific for-human CD45RC, to T cells expressing said CAR and to the use thereof as a medicament, in particular for preventing or treating CD45RC^{high}-related diseases (including autoimmune diseases, undesired immune responses, monogenic diseases, lymphoma or cancer), or graft-versus-host disease (GVHD).

### BACKGROUND OF INVENTION

CD45 (also known as leukocyte common antigen (LCA), EC3.1.3.48, T200, Ly5, and PTPRC) constitutes the first and prototypic receptor-like protein tyrosine phosphatase (RPTP). Its expression is restricted to all nucleated hematopoietic cells, where it is one of the most abundant cell surface glycoproteins, constituting almost 10 percent of the cell surface, and estimated to be present at approximately 25 μM in the plasma membrane (Trowbridge & Thomas, 1994. Annu Rev Immunol. 12:85-116; Hermiston et al., 2003. Annu Rev Immunol. 21:107-37; Holmes, 2006. Immunology. 117(2):145-55).

CD45 comprises an extracellular domain, a single transmembrane domain and a large cytoplasmic domain. The transmembrane and cytoplasmic domains are highly conserved amongst species. In particular, the cytoplasmic domain of CD45 comprises two tandemly duplicated phosphatase domains, of which only the membrane-proximal domain has enzymatic activity (Desai et al., 1994. EMBO J. 13(17):4002-10). The function of the more C-terminal second phosphatase domain in CD45 remains uncertain although it is suggested that it may contribute to CD45 activity indirectly by stabilizing the first domain. Through this cytoplasmic domain, CD45 functions as a central regulator of phosphotyrosine levels in hematopoietic cells, by modulating the activity of Src family of tyrosine-protein kinases (such as Lck in T cells; or Lyn, Fyn and Lck in B cells) (Palacios & Weiss, 2004. Oncogene. 23(48):7990-8000; Lowell, 2004. Mol Immunol. 41(6-7):631-43).

By contrast with the transmembrane and cytoplasmic domains, the extracellular domain of CD45 shows a higher polymorphism among different leukocyte lineages. Indeed, this extracellular domain is heavily glycosylated and contains three alternatively spliced exons (4, 5, and 6—which encode the A, B and C determinants, respectively) that are both O-linked glycosylated and sialylated (Hermiston et al., 2003. Annu Rev Immunol. 21:107-37; Holmes, 2006. Immunology. 117(2):145-55). CD45 isoforms differing in size, shape, and charge can therefore be generated by a dynamically-controlled alternative splicing in both leukocyte differentiation and cellular activation, leading to changes in the extracellular domain of the molecule (Hall et al., 1988. J Immunol. 141(8):2781-7; Lynch, 2004. Nat Rev Immunol. 4(12):931-40).

The largest CD45 isoform containing all three alternatively spliced exons, CD45RABC, is approximately 235 kDa, while the smallest isoform lacking all three exons, CD45RO, is approximately 180 kDa. In between, isoforms comprising only two (e.g., CD45RAB, CD45RBC) or only one (e.g., CD45RB) of the three exons are possible.

While the function of the different CD45 isoforms is not clear, differential expression of these isoforms has been associated with the level of activation of T cells and allows dissociation of naive vs memory T cells (Birkeland et al., 1989. Proc Natl Acad Sci USA. 86(17):6734-8). For example, CD45RA is present on peripheral naive mature CD4⁺ T cells, while CD45RO is expressed on activated and memory CD4⁺ T cells. CD45RABC is expressed on B cells and their precursors, on a sub-group of dendritic cells and other antigen-presenting cells. Effector memory RA T cells (T_{EMRA}), a subtype of terminally differentiated memory T cells, also re-express the naive T cell marker CD45RA (Koch et al., 2008. Immun Ageing. 5:6). Importantly, this pattern of isoforms expression is highly conserved across species emphasizing its functional role and importance (Hermiston et al., 2003. Annu Rev Immunol. 21:107-37).

The particular expression pattern of the CD45RC isoform on CD4⁺ and CD8⁺T cells allows to differentiate between functionally distinct alloreactive T cell subsets that behave differently in terms of proliferation and cytokine secretion. In rodents for example, it has been shown that both CD4⁺ and CD8⁺ T cells CD45RC^{high} are potent Tₕ1 effector cells capable of promoting transplant rejection and organ inflammation (Spickett et al., 1983. J Exp Med. 158(3):795-810; Xystrakis et al., 2004. Eur J Immunol. 34(2):408-17), while T cells expressing undetectable or low levels of CD45RC are Tₕ2 and regulatory T cells and inhibit allograft rejection, graft-versus-host disease (GVHD) and cell-mediated autoimmune diseases (Xystrakis et al., 2004. Blood. 104(10):3294-30; Guillonneau et al., 2007. J Clin Invest. 117(4):1096-106; Powrie & Mason, 1990. J Exp Med. 172(6):1701-8). In humans, a high proportion of CD45RC⁺ CD8⁺ T cells before transplantation has been correlated with decreased graft survival in kidney transplanted patients (Ordonez et al., 2013. PLoS One. 8(7):e69791).

The elimination of the CD45RC^{high} T cells population represents therefore a promising approach for inducing immune tolerance in human, thus for preventing, reducing and/or treating transplant rejection (in particular GVHD) and autoimmune diseases.

GVHD is a significant cause of morbidity and mortality in stem cell transplant patients. It is a T cell-mediated immunoreactive process in which donor cells react against recipient cells. Presently, immunosuppression with immunomodulating drugs such as corticosteroids are the mainstay of GVHD prevention. Whilst progresses have been made with improvements in survival outcomes over time, corticosteroids do not prevent GVHD in a high proportion of patients (less than 50% of patients with acute GVHD and 40-50% of patients with chronic GVHD depending on initial disease severity—Garnett et al., 2013. Ther Adv Hematol. 4(6):366-378), are associated with significant toxicities, and many of the currently available salvage therapies are associated with increased immunosuppression and infectious complications. Thus, there remains an unmet need for the development of new treatment strategies for GVHD to improve long-term post-transplant outcomes.

The Inventors have previously described that the depletion of CD45RC^{high} T cells may represent a potential new therapy in preventing or reducing transplant rejection by decreasing aggressive effector T cells, while increasing tolerogenic regulatory T cells. Indeed, transient anti-CD45RC mAb treatment triggers rapid CD45RC^{high} T cell apoptosis, while preserving memory immunity. Moreover, the Inventors showed that short term anti-CD45RC antibody treatment results in permanent allograft survival with no signs of chronic rejection (International patent WO2016016442; Picarda et al., 2017. JCI Insight. 2(3):e90088).

Here, the Inventors have developed a chimeric antigen receptor (CAR) comprising an antigen-binding fragment directed against human CD45RC. This antigen-binding fragment competes with the anti-human CD45RC antibodies currently available on the market (such as the MT2 clone), exhibiting a comparable pattern of reactivity, however with a significantly better cytotoxic activity to T cells and at a lowest concentration. Indeed, the anti-hCD45RC antigen-binding fragment according to the present invention shows a better affinity than other antigen-binding fragments currently available, and thereby have better therapeutic effects.

Interestingly, the CAR according to the invention may also be useful in the prevention or treatment of certain monogenic diseases in which immune responses are involved in the pathology. Monogenic diseases are caused by single-gene defects. Over 4000 human diseases are caused by these defects linked to one particular gene. Up to now, most treatment options revolve around treating the symptoms of the disorders, in an attempt to improve patient quality of life. Gene therapy is the main hope for durable treatments of this type of diseases. However, major obstacles have been encountered during the development of techniques for the delivery of genes to the appropriate cells affected by the disorder as well as the fact that immune responses against the transgene product or the vector limit the therapeutic efficacy.

Among monogenic diseases, some are linked to genes involved in the immune system (such as T and/or B cells primary immunodeficiencies and polyendocrinopathy candidiasis-ectodermal dystrophy [APECED]), or to genes not associated with immune functions but whose deficiency is associated with inflammation and/or immune reactions [such as Duchenne muscular dystrophy (DMD)].

APECED, also known as auto-immune polyglandular syndrome type I (APS 1) is a rare multi-organ autosomal recessive auto-immune disease caused by mutations in the AIRE gene, a transcription regulator that allows the expression of tissue-restricted antigens (TRA) in medullary epithelial thymic cells (mTECs) and auto-reactive T cells deletion. In human, more than 100 mutations have been described in the AIRE gene to cause APECED with a prevalence of 1-9:1000000 (Orphanet, http://www.orpha.net). The clinical phenotype of APECED is usually defined by the presence of 2 of the 3 major symptoms: hypoparathyroidism, adrenal insufficiency (Addison's disease) and chronical muco-cutaneous candidiasis (CMC). This disease is also associated with multiple autoimmune and ectodermal features such as type 1 diabetes, enamel hypoplasia, vitiligo, premature ovarian failure, keratitis, pernicious anemia, alopecia, exocrine pancreatitis, interstitial lung disease, nephritis and other disorders.

DMD is a monogenic disease wherein mutations of the DMD gene coding for the protein dystrophin lead to severe X-linked muscular dystrophy, which affects all voluntary muscles as well as the heart and breathing muscles in later stages. Immune responses are involved in the pathophysiology of disease in both DMD patients and mdx mice (for a review, see Rosenberg et al., 2015. Sci Transl Med. 7(299):299rv4). The standard treatments of DMD are corticoids, such as prednisolone. In mdx mice, treatments decreasing effector immune responses or inflammation have also been employed, such as intravenous immunoglobulins, tranilast, heme oxygenase-1 inducers, IL-1 receptor antagonist and IL-2, to amplify regulatory T cells (Tregs) (Villalta et al., 2014. Sci Transl Med. 6(258):258ra142; Rosenberg et al., 2015. Sci Transl Med. 7(299):299rv4). However, despite recent promising new treatments, the average life expectancy of DMD patients is still severely reduced.

Surprisingly, the Inventors have demonstrated that treatment of Dmd^{-/-} rats (Dmd^{mds}) with an anti-CD45RC antibody specifically depleting CD45RC^{high} cells ameliorated muscle strength (Ouisse et al., 2019. Front Immunol. 9; 10:2131). They have also demonstrated that administration to Aire^{-/-} rats of an anti-CD45RC monoclonal antibody results in a strong depletion of CD45RC^{high} T cells, and to the removal of symptoms characteristics of APECED (International patent application WO2019115791).

The CAR according to the present invention represents therefore a promising approach for preventing and/or treating monogenic diseases such as DMD and APECED. The Inventors have also demonstrated that immune cells could be engineered to express a transduced CD45RC-CAR at their cell surface. The inventors have also shown that cells transduced with a CD45RC-CAR induced apoptosis in human T cells, and could be activated after contact with human T cells.

### SUMMARY

The invention relates to a chimeric antigen receptor (CAR) specific for human CD45RC, wherein said CAR comprises:
(a) at least one extracellular binding domain, wherein said binding domain binds to said human CD45RC,
(b) optionally at least one extracellular hinge domain,
(c) at least one transmembrane domain, and
(d) at least one intracellular signaling domain, wherein the intracellular domain comprises at least one T cell primary signaling domain and optionally at least one T cell costimulatory signaling domain.

In one embodiment, the extracellular binding domain comprises at least one antigen-binding fragment that binds to human CD45RC comprising:
(a) a HCVR which comprises the following three CDRs:
   (i) V_{H}-CDR1 of sequence SEQ ID NO: 1;
   (ii) V_{H}-CDR2 with a sequence selected from the group comprising sequences SEQ ID NOs: 4, 5, 6, 8, 100, 116, 117, 118 and 119; and
   (iii) V_{H}-CDR3 of sequence SEQ ID NO: 3; and
(b) a LCVR which comprises the following three CDRs:
   (i) V_{L}-CDR1 with a sequence selected from the group comprising sequences SEQ ID NO: 15 (SASSSVS-X₁₂-YMH) and 18 (RASSSVS-X₁₂-YMH), wherein X₁₂ is absent or is selected from Asn (N), Ser (S) and Gly (G);
   (ii) V_{L}-CDR2 with a sequence selected from the group comprising sequences SEQ ID NO: 16, 111, and 120; and
   (iii) V_{L}-CDR3 of sequence SEQ ID NO: 17.

In one embodiment, the extracellular binding domain comprises at least one antigen-binding fragment that binds to human CD45RC comprising:
(a) a HCVR which comprises the following three CDRs:
   (i) V_{H}-CDR1 of sequence SEQ ID NO: 1;
   (ii) V_{H}-CDR2 with a sequence selected from the group comprising sequences SEQ ID NOs: 4 and 5; and
   (iii) V_{H}-CDR3 of sequence SEQ ID NO: 3; and
(b) a LCVR which comprises the following three CDRs:
   (i) V_{L}-CDR1 of sequence SEQ ID NO: 15, wherein X₁₂ is absent;
   (ii) V_{L}-CDR2 of sequence SEQ ID NO: 16; and
   (iii) V_{L}-CDR3 of sequence SEQ ID NO: 17.

In one embodiment, the extracellular binding domain comprises at least one antigen-binding fragment that binds to human CD45RC comprising:
(a) a HCVR which comprises the following three CDRs:
   (i) V_{H}-CDR1 of sequence SEQ ID NO: 1;
   (ii) V_{H}-CDR2 of sequence 4; and
   (iii) V_{H}-CDR3 of sequence SEQ ID NO: 3; and
(b) a LCVR which comprises the following three CDRs:
   (i) V_{L}-CDR1 of sequence SEQ ID NO: 15, wherein X₁₂ is absent;
   (ii) V_{L}-CDR2 of sequence SEQ ID NO: 16; and
   (iii) V_{L}-CDR3 of sequence SEQ ID NO: 17.

In one embodiment, the extracellular binding domain comprises at least one antigen-binding fragment that binds to human CD45RC comprising:
(a) a HCVR which comprises the following three CDRs:
   (i) V_{H}-CDR1 of sequence SEQ ID NO: 1;
   (ii) V_{H}-CDR2 with a sequence selected from the group comprising sequences SEQ ID NOs: 4, 6, and 100; and
   (iii) V_{H}-CDR3 of sequence SEQ ID NO: 3; and
(b) a LCVR which comprises the following three CDRs:
   (i) V_{L}-CDR1 with a sequence selected from the group comprising sequences SEQ ID NOs: 15 and 18, wherein X₁₂ is absent;
   (ii) V_{L}-CDR2 with a sequence selected from the group comprising sequences SEQ ID NO: 16, 111, and 120; and
   (iii) V_{L}-CDR3 of sequence SEQ ID NO: 17.

In one embodiment, the extracellular binding domain comprises at least one antigen-binding fragment that binds to human CD45RC comprising:
1) a HCVR of sequence SEQ ID NO: 61 and a LCVR of sequence SEQ ID NO: 81;
2) a HCVR of sequence SEQ ID NO: 62 and a LCVR of sequence SEQ ID NO: 82;
3) a HCVR of sequence SEQ ID NO: 62 and a LCVR of sequence SEQ ID NO: 83;
4) a HCVR of sequence SEQ ID NO: 62 and a LCVR of sequence SEQ ID NO: 84;
5) a HCVR of sequence SEQ ID NO: 63 and a LCVR of sequence SEQ ID NO: 82;
6) a HCVR of sequence SEQ ID NO: 63 and a LCVR of sequence SEQ ID NO: 83;
7) a HCVR of sequence SEQ ID NO: 63 and a LCVR of sequence SEQ ID NO: 84;
8) a HCVR of sequence SEQ ID NO: 64 and a LCVR of sequence SEQ ID NO: 82;
9) a HCVR of sequence SEQ ID NO: 64 and a LCVR of sequence SEQ ID NO: 83;
10) a HCVR of sequence SEQ ID NO: 64 and a LCVR of sequence SEQ ID NO: 84;
11) a HCVR of sequence SEQ ID NO: 101 and a LCVR of sequence SEQ ID NO: 85;
12) a HCVR of sequence SEQ ID NO: 101 and a LCVR of sequence SEQ ID NO: 103;
13) a HCVR of sequence SEQ ID NO: 65 and a LCVR of sequence SEQ ID NO: 85;
14) a HCVR of sequence SEQ ID NO: 65 and a LCVR of sequence SEQ ID NO: 103;
15) a HCVR of sequence SEQ ID NO: 62 and a LCVR of sequence SEQ ID NO: 85;
16) a HCVR of sequence SEQ ID NO: 101 and a LCVR of sequence SEQ ID NO: 82;
17) a HCVR of sequence SEQ ID NO: 121 and a LCVR of sequence SEQ ID NO: 85;
18) a HCVR of sequence SEQ ID NO: 122 and a LCVR of sequence SEQ ID NO: 85;
19) a HCVR of sequence SEQ ID NO: 123 and a LCVR of sequence SEQ ID NO: 85;
20) a HCVR of sequence SEQ ID NO: 124 and a LCVR of sequence SEQ ID NO: 85;
21) a HCVR of sequence SEQ ID NO: 63 and a LCVR of sequence SEQ ID NO: 85;
22) a HCVR of sequence SEQ ID NO: 67 and a LCVR of sequence SEQ ID NO: 85;
23) a HCVR of sequence SEQ ID NO: 67 and a LCVR of sequence SEQ ID NO: 103;
24) a HCVR of sequence SEQ ID NO: 61 and a LCVR of sequence SEQ ID NO: 113;
25) a HCVR of sequence SEQ ID NO: 61 and a LCVR of sequence SEQ ID NO: 126; or
26) a HCVR and a LCVR comprising a sequence of the non-CDR regions sharing at least 70% of identity with the sequence of the non-CDR regions of the HCVR and LCVR according to 1) to 23).

In one embodiment, the extracellular binding domain comprises at least one antigen-binding fragment that binds to human CD45RC comprising:
(a) a HCVR which comprises the following three CDRs:
   (i) V_{H}-CDR1 of sequence SEQ ID NO: 1;
   (ii) V_{H}-CDR2 with a sequence selected from the group comprising sequences SEQ ID NOs: 4, 5, 6, 8, 100, 116, 117, 118 and 119; and
   (iii) V_{H}-CDR3 of sequence SEQ ID NO: 3; and
(b) a LCVR which comprises the following three CDRs:
   (i) V_{L}-CDR1 with a sequence selected from the group comprising sequences SEQ ID NOs: 15 and 18, wherein X₁₂ in SEQ ID NOs: 15 and 18 is selected from Asn (N), Ser (S) and Gly (G);
   (ii) V_{L}-CDR2 of sequence SEQ ID NO: 16; and
   (iii) V_{L}-CDR3 of sequence SEQ ID NO: 17;
preferably wherein the amino acid residue at Kabat position L71 of the LCVR is Phe (F).

In one embodiment, the extracellular binding domain comprises an scFv fragment directed against human CD45RC.

In one embodiment, the hinge domain is a hinge region of human CD8α, preferably having the sequence of SEQ ID NO: 145 or a sequence having at least 70% identity thereto.

In one embodiment, the transmembrane domain is a transmembrane domain derived from the human CD8α, preferably having the sequence of SEQ ID NO: 153 or a sequence having at least 70% identity thereto.

In one embodiment, the primary intracellular signaling domain comprises a T cell primary intracellular signaling domain of human CD3 zeta, preferably having the sequence of SEQ ID NO: 157 or a sequence having at least 70% identity thereto.

In one embodiment, the costimulatory signaling domain is selected from the group consisting of a CD28 cytoplasmic signaling domain, a 4-1BB cytoplasmic signaling domain, a OX40 cytoplasmic signaling domain, a ICOS cytoplasmic signaling domain, a CD27 cytoplasmic signaling domain and a DAP10 cytoplasmic signaling domain.

In one embodiment, the CAR according to the invention comprises:
(i) an anti-human CD45RC scFv, preferably comprising a HCVR having the sequence of SEQ ID NO: 61 and a LCVR having the sequence of SEQ ID NO: 81, preferably linked by a linker having the sequence of SEQ ID NO: 134,
(ii) a hinge domain derived from CD8α, preferably having the sequence of SEQ ID NO: 145,
(iii) a human CD8α transmembrane domain, preferably having the sequence of SEQ ID NO: 153, and
(iv) an intracellular signaling domain comprising a human CD28 signaling domain, preferably having the sequence of SEQ ID NO: 167 and a human CD3 zeta signaling domain, preferably having the sequence of SEQ ID NO: 157.

The invention also relates to a nucleic acid encoding the CAR according to the invention.

The invention also pertains to an expression vector comprising the nucleic acid according to the invention.

The invention further relates to an immune cell population, engineered to express at the cell surface a CAR according to the invention.

In one embodiment, said immune cell population is a CD45RC^{neg} cell population.

In one embodiment, said immune cell population is a regulatory T cell population, an effector T cell population, a memory T cell population, an NKT cell population or a MAIT cell population.

In one embodiment, said immune cell population is a regulatory T cell population, preferably wherein said regulatory T cell population is selected from the group consisting of CD4⁺ CD25⁺ Foxp3⁺ Treg, Tr1 cells, TGF-β secreting Th3 cells, regulatory NKT cells, regulatory γδ T cells, regulatory CD8⁺ T cells, and double negative regulatory T cells.

The invention also relates to a composition comprising at least one immune cell population engineered to express at the cell surface a CAR according to the invention, wherein said composition is preferably a pharmaceutical composition further comprising at least one pharmaceutically acceptable excipient or carrier.

The invention further relates to an immune cell population according to the invention, or the pharmaceutical composition according the invention, for use as a medicament.

The invention also pertains to an immune cell population according to the invention, or a composition according to the invention, for use in inducing immune tolerance, in preventing or reducing transplant rejection, or in preventing or treating graft-versus-host disease (GVHD) in a subject in need thereof.

The invention further relates to an immune cell population according to the invention, or a composition according to the invention for use in preventing, reducing and/or treating a CD45RC^{high}-related condition selected from the group consisting of an autoimmune disease, an undesired immune response, a monogenic disease, lymphoma and cancer.

### Definitions

“Antibody” or “Immunoglobulin” As used herein, the term “immunoglobulin” refers to a protein having a combination of two heavy and two light chains whether or not it possesses any relevant specific immunoreactivity. “Antibodies” refers to such assemblies which have significant known specific immunoreactive activity to an antigen of interest (e.g., human CD45RC). The term “anti-hCD45RC antibodies” is used herein to refer to antibodies which exhibit immunological specificity for human CD45RC protein. As explained elsewhere herein, “specificity” for human CD45RC does not exclude cross-reaction with species homologues of hCD45RC.

Antibodies and immunoglobulins comprise light and heavy chains, with or without an interchain covalent linkage between them. Basic immunoglobulin structures in vertebrate systems are relatively well understood. The generic term “immunoglobulin” comprises five distinct classes of antibody that can be distinguished biochemically. Although the following discussion will generally be directed to the IgG class of immunoglobulin molecules, all five classes of antibodies are within the scope of the present invention. With regard to IgG, immunoglobulins comprise two identical light polypeptide chains of molecular weight of about 23 kDa, and two identical heavy chains of molecular weight of about 53-70 kDa. The four chains are joined by disulfide bonds in a “Y” configuration wherein the light chains bracket the heavy chains starting at the mouth of the “Y” and continuing through the variable region. The light chains of an antibody are classified as either kappa (κ) or lambda (λ). Each heavy chain class may be bonded with either a x or k light chain. In general, the light and heavy chains are covalently bonded to each other, and the “tail” regions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain. Those skilled in the art will appreciate that heavy chains are classified as gamma (γ), mu (μ), alpha (α), delta (δ) or epsilon (ε) with some subclasses among them (e.g., γ1-γ4). It is the nature of this chain that determines the “class” of the antibody as IgG, IgM, IgA IgD or IgE, respectively. The immunoglobulin subclasses or “isotypes” (e.g., IgG1, IgG2, IgG3, IgG4, IgA1, etc.) are well characterized and are known to confer functional specialization. Modified versions of each of these classes and isotypes are readily discernable to the skilled artisan in view of the instant disclosure and, accordingly, are within the scope of the present invention. As indicated above, the variable region of an antibody allows the antibody to selectively recognize and specifically bind epitopes on antigens. That is, the light chain variable domain (V_{L} domain) and heavy chain variable domain (V_{H} domain) of an antibody combine to form the variable region that defines a three-dimensional antigen binding site. This quaternary antibody structure forms the antigen binding site presents at the end of each arm of the “Y”. More specifically, the antigen binding site is defined by three complementarity determining regions (CDRs) on each of the V_{H} and V_{L} chains.

“Antibody Fragment”

As used herein, the term “antibody fragment” refers to at least one portion of an intact antibody, preferably the antigen binding region or variable region of the intact antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)₂, Fv fragments, scFv antibody fragments, disulfide-linked Fvs (sdFv), a Fd fragment consisting of the VH and CHI domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, a v-NAR and a bis-scFv (see, e.g., Hollinger and Hudson, Nature Biotechnology 23:1126-1136, 2005). Antigen binding fragments can also be grafted into scaffolds based on polypeptides such as a fibronectin type III (see U.S. Pat. No. 6,703,199, which describes fibronectin polypeptide minibodies). Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CH1). Each Fab fragment is monovalent with respect to antigen binding, i.e., it has a single antigen-binding site. Pepsin treatment of an antibody yields a single large F(ab′)₂ fragment that roughly corresponds to two disulfide linked Fab fragments having divalent antigen-binding activity and is still capable of crosslinking antigen. Fab′ fragments differ from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments that have hinge cysteines between them. Other chemical couplings of antibody fragments are also known.

“Binding Fragment” or “Antigen-Binding Fragment”

As used herein, the term “binding fragment”, refers to a part or region of the antibody according to the present invention, which comprises fewer amino acid residues than the whole antibody. A “binding fragment” binds antigen and/or competes with the whole antibody from which it was derived for antigen binding (e.g., specific binding to human CD45RC). Antibody binding fragments encompasses, without any limitation, single chain antibodies, Fv, Fab, Fab′, Fab′-SH, F(ab)′₂, Fd, defucosylated antibodies, diabodies, triabodies and tetrabodies.

“Characterized as Having [ . . . ] Amino Acids being Substituted by a Different Amino Acid”

As used herein, the phrase “characterized as having [ . . . ] amino acids being substituted by a different amino acid” in reference to a given sequence, refers to the occurrence, in said sequence, of “conservative amino acid modifications”.

“Conservative Amino Acid Modifications”

As used herein, the term “conservative amino acid modifications” refers to modifications that do not significantly affect or alter the binding characteristics of the antibody or binding fragment thereof containing the amino acid sequence. Such conservative modifications include amino acid substitutions, additions and deletions. Modifications can be introduced into an antibody or binding fragment thereof by standard techniques known in the art, such as site-directed mutagenesis and PCR-mediated mutagenesis.

Conservative amino acid substitutions are typically those in which an amino acid residue is replaced with an amino acid residue having a side chain with similar physicochemical properties. Specified variable region and CDR sequences may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or more amino acid insertions, deletions and/or substitutions. Where substitutions are made, preferred substitutions will be conservative modifications. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), β-branched side chains (e.g., threonine, valine, isoleucine) and aromatic side chains (e.g., tyrosine, phenylalanine, tryptophan, histidine). Thus, one or more amino acid residues within the CDRs and/or variable regions of the antibody or binding fragment thereof according to the present invention can be replaced with other amino acid residues from the same side chain family and the altered antibody can be tested for retained function (i.e., the properties set forth herein, such as, e.g., the binding to hCD45RC) using the assays described herein. In another embodiments, a string of amino acids within the CDRs and/or variable regions of the antibody or binding fragment thereof according to the present invention can be replaced with a structurally similar string that differs in order and/or composition of side chain family members.

“CDR” or “Complementarity Determining Region” As used herein, the term “CDR” or “complementarity determining region” refers to the non-contiguous antigen combining sites found within the variable region of both heavy and light chain polypeptides. CDRs were identified according to the rules of Table 1, as deduced from Kabat et al., 1991. Sequences of proteins of immunological interest (5^{th} ed.). Bethesda, Md.: U.S. Dep. of Health and Human Services; and Chothia and Lesk, 1987. J Mol Biol. 196(4):901-17:

| | Heavy chain variable region (HCVR or V_{H}) | | |
|---|---|---|---|
| | V_{H}-CDR1 | V_{H}-CDR2 | V_{H}-CDR3 |
| Start | Approx. at residue 26 | Always 15 residues after | Always 33 residues after |
| | (always 4 after a Cys) | the end of V_{H}-CDR1 | end of V_{H}-CDR2 |
| | according to | according to Kabat/AbM's | Always 2 residues after a |
| | Chothia/AbM's definition | definition | Cys |
| | Kabat's definition starts 5 | | |
| | residues later | | |
| Residue | Always Cys-Xaa-Xaa-Xaa, | Typically, Leu-Glu-Trp-Ile- | Always Cys-Xaa-Xaa, with |
| before | with Xaa being any amino | Gly, but a number of | Xaa being any amino acid |
| | acid according to | variations | Typically, Cys-Ala-Arg |
| | Chothia/AbM's definition | | |
| Residue | Always Trp | Lys/Arg- | Always Trp-Gly-Xaa-Gly, |
| after | Typically, Trp-Val, but | Leu/Ile/Val/Phe/Thr/Ala- | with Xaa being any amino |
| | also, Trp-Ile or Trp-Ala | Thr/Ser/Ile/Ala | acid |
| Length | 10 to 12 residues according | 16 to 19 residues according | 3 to 25 residues |
| | to AbM's definition | to Kabat's definition | |
| | Chothia's definition | AbM's definition ends 7 | |
| | excludes the last 4 residues | residues earlier | |
| | 5 to 7 residues according to | | |
| | Kabat's definition | | |
| | Light chain variable region (LCVR or V_{L}) | | |
| | V_{L}-CDR1 | V_{L}-CDR2 | V_{L}-CDR3 |
| Start | Approx. at residue 24 | Always 16 residues after | Always 33 residues after |
| | | the end of V_{L}-CDR1 | end of V_{L}-CDR2 (except |
| | | | NEW (PDB ID: 7FAB) |
| | | | which has the deletion at |
| | | | the end of CDR-L2*) |
| Residue | Always Cys | Generally, Ile-Tyr, but also, | Always Cys |
| before | | Val-Tyr, Ile-Lys or Ile-Phe | |
| Residue | Always Trp | | Always Phe-Gly-Xaa-Gly, |
| after | Typically, Trp-Tyr-Gln, but | | with Xaa being any amino |
| | also, Trp-Leu-Gln, Trp- | | acid |
| | Phe-Gln or Trp-Tyr-Leu | | |
| Length | 10 to 17 residues | Always 7 residues (except | 7 to 11 residues |
| | | NEW (PDB ID: 7FAB) | |
| | | which has a deletion in this | |
| | | region*) | |

*Saul & Poljak, 1992. Proteins. 14(3): 363-71

“Engineered”

As used herein, the term “engineered” or “modified” refers to a cell that has been transfected, transformed or transduced

“Epitope”

As used herein, the term “epitope” refers to a specific arrangement of amino acids located on a protein or proteins to which an antibody or binding fragment thereof binds. Epitopes often consist of a chemically active surface grouping of molecules such as amino acids or sugar side chains, and have specific three-dimensional structural characteristics as well as specific charge characteristics. Epitopes can be linear (or sequential) or conformational, i.e., involving two or more sequences of amino acids in various regions of the antigen that may not necessarily be contiguous.

“Fragment”

As used herein, the term “fragment” of an antigen refers to any subset of an antigen, as a shorter peptide. In some embodiments, a fragment of an antigen is a peptide of at least 6 amino acids in length. In some embodiments, a fragment of an antigen is a peptide of 6 to 50 amino acids in length, of 6 to 30 amino acids, or of 6 to 20 amino acids in length.

“Framework region” or “FR” or “non-CDR regions” As used herein, the terms “framework region”, “FR” or “non-CDR regions” include the amino acid residues that are part of the variable region, but are not part of the CDRs (e.g., using the Kabat/Chothia definition of CDRs). Therefore, a variable region framework is between about 100-120 amino acids in length but includes only those amino acids outside of the CDRs.

For the specific example of a HCVR and for the CDRs as defined by Kabat/Chothia:
FR1 may correspond to the domain of the variable region encompassing amino acids 1-25 according to Chothia/AbM's definition, or 5 residues later according to Kabat's definition;
FR2 may correspond to the domain of the variable region encompassing amino acids 36-49;
FR3 may correspond to the domain of the variable region encompassing amino acids 67-98; and
FR4 may correspond to the domain of the variable region from amino acids 104-110 to the end of the variable region.

The framework regions for the light chain are similarly separated by each of the LCVR's CDRs. In naturally occurring antibodies, the six CDRs present on each monomeric antibody are short, non-contiguous sequences of amino acids that are specifically positioned to form the antigen binding site as the antibody assumes its three-dimensional configuration in an aqueous environment. The remainders of the heavy and light variable domains show less inter-molecular variability in amino acid sequence and are termed the framework regions. The framework regions largely adopt a R-sheet conformation and the CDRs form loops which connect, and in some cases form part of, the R-sheet structure. Thus, these framework regions act to form a scaffold that provides for positioning the six CDRs in correct orientation by inter-chain, non-covalent interactions. The antigen binding site formed by the positioned CDRs defines a surface complementary to the epitope on the immunoreactive antigen. This complementary surface promotes the non-covalent binding of the antibody to the immunoreactive antigen epitope. The position of CDRs can be readily identified by one of ordinary skill in the art.

“Heavy Chain Region”

As used herein, the term “heavy chain region” includes amino acid sequences derived from the constant domains of an immunoglobulin heavy chain. A protein comprising a heavy chain region comprises at least one of a C_{H}1 domain, a hinge (e.g., upper, middle, and/or lower hinge region) domain, a C_{H}2 domain, a C_{H}3 domain, or a variant or fragment thereof. In an embodiment, the antibody or binding fragment thereof according to the present invention may comprise the Fc region of an immunoglobulin heavy chain (e.g., a hinge portion, a C_{H}2 domain, and a C_{H}3 domain). In another embodiment, the antibody or binding fragment thereof according to the present invention lacks at least a region of a constant domain (e.g., all or part of a C_{H}2 domain). In certain embodiments, at least one, and preferably all, of the constant domains are derived from a human immunoglobulin heavy chain. For example, in one preferred embodiment, the heavy chain region comprises a fully human hinge domain. In other preferred embodiments, the heavy chain region comprising a fully human Fc region (e.g., hinge, C_{H}2 and C_{H}3 domain sequences from a human immunoglobulin). In certain embodiments, the constituent constant domains of the heavy chain region are from different immunoglobulin molecules. For example, a heavy chain region of a protein may comprise a C_{H}2 domain derived from an IgG1 molecule and a hinge region derived from an IgG3 or IgG4 molecule. In other embodiments, the constant domains are chimeric domains comprising regions of different immunoglobulin molecules. For example, a hinge may comprise a first region from an IgG1 molecule and a second region from an IgG3 or IgG4 molecule. As set forth above, it will be understood by one of ordinary skill in the art that the constant domains of the heavy chain region may be modified such that they vary in amino acid sequence from the naturally occurring (wild-type) immunoglobulin molecule. That is, the antibody or binding fragment thereof according to the present invention may comprise alterations or modifications to one or more of the heavy chain constant domains (C_{H}1, hinge, C_{H}2 or C_{H}3) and/or to the light chain constant domain (C_{L}). Exemplary modifications include additions, deletions or substitutions of one or more amino acids in one or more domains.

“Hinge Region”

Within an antibody, the term “hinge region” includes the region of a heavy chain molecule that joins the C_{H}1 domain to the C_{H}2 domain. This hinge region comprises approximately 25 residues and is flexible, thus allowing the two N-terminal antigen binding regions to move independently. Hinge regions can be subdivided into three distinct domains: upper, middle, and lower hinge domains (Roux et al., 1998. J Immunol. 161(8):4083-90). Within a CAR molecule, the term “hinge region” refers to the region that connects the extracellular binding domain with the transmembrane domain.

“Hypervariable Loop”

The term “hypervariable loop” is not strictly synonymous to complementarity determining region (CDR), since the hypervariable loops (HVs) are defined on the basis of structure, whereas CDRs are defined based on sequence variability (Kabat et al., 1991. Sequences of proteins of immunological interest (5^{th} ed.). Bethesda, Md.: U.S. Dep. of Health and Human Services) and the limits of the HVs and the CDRs may be different in some V_{H} and V_{L} domains. The CDRs of the V_{L} and V_{H} domains can typically be defined by the Kabat/Chothia definition as already explained hereinabove.

“Identity” or “Identical”

As used herein, the term “identity” or “identical”, when used in a relationship between the sequences of two or more amino acid sequences, or of two or more nucleic acid sequences, refers to the degree of sequence relatedness between amino acid sequences or nucleic acid sequences, as determined by the number of matches between strings of two or more amino acid residues or nucleic acid residues. “Identity” measures the percent of identical matches between the smaller of two or more sequences with gap alignments (if any) addressed by a particular mathematical model or computer program (i.e., “algorithms”).

Identity of related amino acid sequences or nucleic acid sequences can be readily calculated by known methods. Such methods include, but are not limited to, those described in Lesk A. M. (1988). Computational molecular biology: Sources and methods for sequence analysis. New York, N.Y.: Oxford University Press; Smith D. W. (1993). Biocomputing: Informatics and genome projects. San Diego, Calif.: Academic Press; Griffin A. M. & Griffin H. G. (1994). Computer analysis of sequence data, Part 1. Totowa, N.J.: Humana Press; von Heijne G. (1987). Sequence analysis in molecular biology: treasure trove or trivial pursuit. San Diego, Calif.: Academic press; Gribskov M. R. & Devereux J. (1991). Sequence analysis primer. New York, N.Y.: Stockton Press; Carillo et al., 1988. SIAM J Appl Math. 48(5):1073-82.

Preferred methods for determining identity are designed to give the largest match between the sequences tested. Methods of determining identity are described in publicly available computer programs. Preferred computer program methods for determining identity between two sequences include the GCG program package, including GAP (Genetics Computer Group, University of Wisconsin, Madison, Wis.; Devereux et al., 1984. Nucleic Acids Res. 12(1 Pt 1):387-95), BLASTP, BLASTN, and FASTA (Altschul et al., 1990. J Mol Biol. 215(3):403-10). The BLASTX program is publicly available from the National Center for Biotechnology Information (NCBI) and other sources (BLAST Manual, Altschul et al. NCB/NLM/NIH Bethesda, Md. 20894). The well-known Smith Waterman algorithm may also be used to determine identity.

“Immune Cells”

As used herein, the term “immune cells” generally includes white blood cells (leukocytes) that are derived from hematopoietic stem cells (HSC) produced in the bone marrow. Examples of immune cells include, but are not limited to, lymphocytes (T cells, B cells, and natural killer (NK) cells) and myeloid-derived cells (neutrophil, eosinophil, basophil, monocyte, macrophage, dendritic cells).

“Immunospecific”, “Specific For” or “Specifically Bind”

As used herein, an antibody or binding fragment thereof is said to be “immunospecific”, “specific for” or to “specifically bind” an antigen if it reacts at a detectable level with said antigen (e.g., hCD45RC), preferably with an affinity constant (K_{A}) of greater than or equal to about 10⁶ M⁻¹, preferably greater than or equal to about 10⁷ M⁻¹, 108 M⁻¹, 5×10⁸ M⁻¹, 10⁹ M⁻¹, 5×10⁹ M⁻¹ or more.

Affinity of an antibody or binding fragment thereof for its cognate antigen is also commonly expressed as an equilibrium dissociation constant (K_{D}). an antibody or binding fragment thereof is said to be “immunospecific”, “specific for” or to “specifically bind” an antigen if it reacts at a detectable level with said antigen (e.g., hCD45RC), preferably with a K_{D} of less than or equal to 10⁻⁶ M, preferably less than or equal to 10⁻⁷ M, 5×10⁻⁸ M, 10⁻⁸ M, 5×10⁻⁹ M, 10⁻⁹ M or less.

Affinities of antibodies or binding fragment thereof can be readily determined using conventional techniques, for example, those described by Scatchard, 1949. Ann NY Acad Sci. 51:660-672. Binding properties of an antibody or binding fragment thereof to antigens, cells or tissues may generally be determined and assessed using immunodetection methods including, for example, ELISA, immunofluorescence-based assays, such as immuno-histochemistry (IHC) and/or fluorescence-activated cell sorting (FACS) or by surface plasmon resonance (SPR, e.g., using BIAcore®).

“Isolated Antibody”

As used herein, the term “isolated antibody” is intended to refer to an antibody that is substantially free of other antibodies having different antigenic specificities (e.g., an isolated antibody that specifically binds hCD45RC is substantially free of antibodies that specifically bind antigens other than hCD45RC). An isolated antibody that specifically binds hCD45RC may, however, have cross-reactivity to other antigens, such as CD45RC molecules from other species. Moreover, an isolated antibody may be substantially free of other cellular material and/or chemicals, in particular those that would interfere with diagnostic or therapeutic uses of the antibody, including without limitation, enzymes, hormones, and other proteinaceous or non-proteinaceous components.

“Isolated Nucleic Acid”

As used herein, the term “isolated nucleic acid” is intended to refer to a nucleic acid that is substantially separated from other genome DNA sequences as well as proteins or complexes such as ribosomes and polymerases, which naturally accompany a native sequence. The term embraces a nucleic acid sequence that has been removed from its naturally occurring environment, and includes recombinant or cloned DNA isolates and chemically synthesized analogues or analogues biologically synthesized by heterologous systems. A substantially pure nucleic acid includes isolated forms of the nucleic acid. Of course, this refers to the nucleic acid as originally isolated and does not exclude genes or sequences later added to the isolated nucleic acid by the hand of man.

“Ligand”

As used herein, the term “ligand” refers to a member of a pair ligand/receptor, and binds to the other member of the pair.

“Monoclonal Antibody”

As used herein, the term “monoclonal antibody” refers to an antibody obtained from a population of substantially homogeneous antibodies, i.e., the individual antibodies comprised in the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies or binding fragment thereof according to the present invention may be prepared by the hybridoma methodology first described by Kohler et al., 1975. Nature. 256(5517):495-7, or may be made using recombinant DNA methods in bacterial, eukaryotic animal or plant cells (Patent U.S. Pat. No. 4,816,567). The “monoclonal antibodies” may also be isolated from phage antibody libraries using the techniques described in Clackson et al., 1991. Nature. 352(6336):624-8 and Marks et al., 1991. J Mol Biol. 222(3):581-97, for example.

“Mait Cell”

As used herein, the term “MAIT cell” refers to mucosal-associated invariant T cell. MAIT cells can be activated via their TCR and TCR-independent signals (e.g. cytokines). MAIT cells are able to sense bacterial or viral infections and to produce effector cytokines and/or degranulate in response to these signals.

“NK Cell”

As used herein, “NK cell” or “natural killer cell” refers to a cytotoxic lymphocyte playing an important role in the innate immunity. NK cells are constantly in contact with other cells. NK cells express activating and inhibitory receptors in their cell surface. This mechanism allows NK cells to recognize if a cell is “normal” (and thus not to be eliminated) or if a cell is “anormal” (and thus to be killed) such as a tumor cell or an infected cell.

“NKT Cell” As used herein, “NKT cell” or “natural killer T cell” refers to a cytotoxic lymphocyte which display T lymphocyte markers as well as NK lymphocyte markers. Natural killer (NK) cells and natural killer T (NKT) cells are two types of important cells in innate immunity. Both NK and NKT cells are cytotoxic cells, which induce cell death of pathogenic cells as well as tumor cells. The main difference between NK cells and NKT cells is that NK cells are large granular lymphocytes while NKT cells are a type of T cells.

“Nucleid Acid” or “Polynucleotide”

As used herein, the term “nucleic acid” or “polynucleotide” refers to a polymer of nucleotides covalently linked by phosphodiester bonds, such as deoxyribonucleic acids (DNA) or ribonucleic acids (RNA), in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol.Chem. 260:2605-2608 (1985); and Rossolini et al., Mol. Cell. Probes 8:91-98 (1994)).

“Prevent” or “Preventing” or “Prevention”

As used herein, the terms “prevent”, “preventing” and “prevention” refer to prophylactic and preventative measures, wherein the object is to reduce the chances that a subject will develop the pathologic condition or disorder over a given period of time. Such a reduction may be reflected, e.g., in a delayed onset of at least one symptom of the pathologic condition or disorder in the subject.

“Promoter”

As used herein, the term “promoter” refers to a DNA sequence recognized by the synthetic machinery of the cell, or introduced synthetic machinery, required to initiate the specific transcription of a polynucleotide sequence.

“Subject”

As used herein, the term “subject” refers to a mammal, preferably a human. In one embodiment, a subject may be a “patient”, i.e., a warm-blooded animal, more preferably a human, who/which is awaiting the receipt of, or is receiving medical care or was/is/will be the object of a medical procedure, or is monitored for the development of a disease. The term “mammal” refers here to any mammal, including humans, domestic and farm animals, and zoo, sports, or pet animals, such as dogs, cats, cattle, horses, sheep, pigs, goats, rabbits, etc. Preferably, the mammal is a primate, more preferably a human.

“Transfected” or “Transformed” or “Transduced”

As used herein, the term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

“Treating” or “Treatment” or “Alleviation”

As used herein, the terms “treating” or “treatment” or “alleviation” refer to therapeutic treatment, excluding prophylactic or preventative measures; wherein the object is to slow down (lessen) the targeted pathologic condition or disorder. Those in need of treatment include those already with the disorder as well those suspected to have the disorder. A subject is successfully “treated” for the targeted pathologic condition or disorder if, after receiving a therapeutic amount of the isolated antibody or binding fragment thereof, nucleic acid, expression vector, composition, pharmaceutical composition or medicament according to the present invention, said subject shows observable and/or measurable reduction in or absence of one or more of the following: reduction in the number of CD45RC^{high} cells; reduction in the percent of total cells that are CD45RC^{high}; relief to some extent, of one or more of the symptoms associated with the specific disease or condition; reduced morbidity and mortality; and/or improvement in quality of life issues. The above parameters for assessing successful treatment and improvement in the disease are readily measurable by routine procedures familiar to a physician.

“Treg cell”

As used herein, the term “Treg cell” refers to a cell capable of suppressing, inhibiting or preventing excessive or unwanted inflammatory responses, such as, for example, autoimmunity or allergic reactions. In one embodiment, the Treg cell population of the invention is capable of suppressive activity. In one embodiment, said suppressive activity is contact independent. In another embodiment, said suppressive activity is contact dependent. In one embodiment, the Treg cell population of the invention presents a suppressive action on effector T cells, preferably said suppressive action is dependent on TCR expression and/or activation.

“Teff Cell”

As used herein, the term “Teff cell” refers to a T effector cell. Teff cells include CD4⁺ T helper cells and CD8⁺ cytotoxic T cells. Teff cells play a central role in cellular-mediated immunity following antigen challenge. Treg cells are a key regulator of the Teff cells.

“Memory T Cell”

As used herein, the term “memory T cell” refers to a subset of T cells that have previously encountered and responded to their cognate antigen. In comparison to naive T cells, which are T cells have not been exposed to antigens yet, memory T cells can mount a faster and stronger immune response.

“Variable Region” or “Variable Domain”

As used herein, the term “variable” refers to the fact that certain regions of the variable domains V_{H} and V_{L} differ extensively in sequence among antibodies and are used in the binding and specificity of each particular antibody for its target antigen. However, the variability is not evenly distributed throughout the variable domains of antibodies. It is concentrated in three segments called “hypervariable loops” in each of the V_{L} domain and the V_{H} domain which form part of the antigen binding site.

The first, second and third hypervariable loops of the Vλ light chain domain are referred to herein as L1 (λ), L2 (λ) and L3 (λ) and may be defined as comprising residues 24-33 (L1(λ), consisting of 9, 10 or 11 amino acid residues), 49-53 L2 (λ), consisting of 3 residues) and 90-96 (L3(λ), consisting of 6 residues) in the V_{L} domain (Morea et al., 2000. Methods. 20(3):267-79).

The first, second and third hypervariable loops of the Vκ light chain domain are referred to herein as L1(κ), L2(κ) and L3(κ) and may be defined as comprising residues 25-33 (L1(κ), consisting of 6, 7, 8, 11, 12 or 13 residues), 49-53 (L2(κ), consisting of 3 residues) and 90-97 (L3(κ), consisting of 6 residues) in the V_{L} domain (Morea et al., 2000. Methods. 20(3):267-79).

The first, second and third hypervariable loops of the V_{H} domain are referred to herein as H1, H2 and H3 and may be defined as comprising residues 25-33 (H1, consisting of 7, 8 or 9 residues), 52-56 (H2, consisting of 3 or 4 residues) and 91-105 (H3, highly variable in length) in the V_{H} domain (Morea et al., 2000. Methods. 20(3):267-79).

Unless otherwise indicated, the terms L1, L2 and L3 respectively refer to the first, second and third hypervariable loops of a V_{L} domain, and encompass hypervariable loops obtained from both Vκ and Vλ isotypes. The terms H1, H2 and H3 respectively refer to the first, second and third hypervariable loops of the V_{H} domain, and encompass hypervariable loops obtained from any of the known heavy chain isotypes, including gamma (γ), mu (μ), alpha (α), delta (δ) or epsilon (ε). The hypervariable loops L1, L2, L3, H1, H2 and H3 may each comprise part of a “complementarity determining region” or “CDR”, as defined hereinabove.

“Variant”

The term “variant” of an antigen refers herein to an antigen that is almost identical to the natural antigen and which shares the same biological activity. The minimal difference between the natural antigen and its variant may lie for example in an amino acid substitution, deletion, and/or addition. Such variants may contain, for example, conservative amino acid substitutions.

In some embodiments, the variant of an antigen presents a sequence identity of at least or of about 70, 75, 80, 85, 90, 91, 92, 93, 94, 95, 96, 97, 98, or 99% with the sequence of the natural antigen.

### DETAILED DESCRIPTION

A first aspect of the present invention relates to a chimeric antigen receptor (CAR) specific for human CD45RC, wherein said CAR comprises at least one extracellular binding domain that binds to said human CD45RC. In one embodiment, the extracellular binding domain is an antigen-binding domain, such as for instance an antibody or a binding fragment thereof as described hereafter.

It is herein disclosed an isolated antibody or a binding fragment thereof, binding to human CD45RC (hCD45RC). The isolated antibody or a binding fragment thereof may be purified.

Preferably, the isolated antibody or a binding fragment thereof is purified to:
(1) greater than 80%, 85%, 90%, 91%, 92%, 93%, 94%, 95% or more by weight of antibody or binding fragment thereof as determined by the Lowry method, and most preferably more than 96%, 97%, 98% or 99% by weight;
(2) a degree sufficient to obtain at least 15 residues of N-terminal or internal amino acid sequence by use of a spinning cup sequenator; or
(3) homogeneity as shown by SDS-PAGE under reducing or non-reducing conditions and using Coomassie blue or, preferably, silver staining.

The antibody or binding fragment thereof described herein binds to human CD45RC (hCD45RC).

As used herein, the term “CD45” (also known as CD45R or PTPRC) refers to a transmembrane glycoprotein existing in different isoforms. These distinct isoforms of CD45 differ in their extracellular domain structures which arise from alternative splicing of 3 variable exons (exons 4, 5 and 6) coding for the A, B and C determinants, respectively, of the CD45 extracellular region. Antibodies reactive with restricted epitope are clustered as “CD45R”. Hence, anti-CD45RA, anti-CD45RB and anti-CD45RC antibodies recognize CD45 isoforms which include the expression of the A, B and C determinants, respectively. The various isoforms of CD45 have different extracellular domains, but have an identical extracellular sequence proximal to the membrane, as well as for the transmembrane domain and a large cytoplasmic tail segments containing two tandemly homologous highly conserved phosphatase domains of approximately 300 residues. CD45 and its isoforms non-covalently associate with lymphocyte phosphatase-associated phosphoprotein (LPAP) on T and B lymphocytes. CD45 has been reported to be associated with several other cell surface antigens, including CD1, CD2, CD3, and CD4. CD45 is involved in signaling lymphocytes activation. When preceded by the letter “h” (e.g., hCD45), it is implied that the CD45 is of human origin.

As used herein, the term “CD45RC” refers to a 200-220 kDa single chain type I membrane glycoprotein well-known from the skilled artisan. CD45RC is an alternative splicing isoform of CD45 comprising exon 6 encoding the C determinant (hence the terminology CD45RC, i.e., CD45 Restricted to the C determinant), but lacking exons 4 and 5, respectively encoding the A and B determinants. An amino acid sequence of human CD45RC is given in SEQ ID NO: 104, corresponding to UniProt Accession P08575-10 (version 10, modified Mar. 28, 2018—Checksum: F92C874C9A114890). This CD45RC isoform is expressed on B cells, and a subset of CD8⁺ T cells and CD4⁺ T cells, but not on CD8⁺ or CD4⁺ Treg, CD14⁺ monocytes or PMN (Picarda et al., 2017. JCI Insight. 2(3):e90088). While some monoclonal antibodies can recognize an epitope in the portion of CD45 common to all the different isoforms (these are termed anti-CD45 antibodies), other monoclonal antibodies have restricted specificity to a given isoform, depending on which determinant they recognize (A, B or C). When preceded by the letter “h” (e.g., hCD45RC), it is implied that the CD45RC is of human origin.

In one embodiment, the antibody or binding fragment thereof binds to the extracellular domain of hCD45RC. In one embodiment, the antibody or binding fragment thereof binds to at least one epitope present on the extracellular domain of hCD45RC.

In one embodiment, the antibody or binding fragment thereof binds to the C determinant encoded by exon 6 of hCD45. In one embodiment, the antibody or binding fragment thereof binds to at least one epitope on the C determinant encoded by exon 6 of hCD45.

In one embodiment, the amino acid sequence of the C determinant encoded by exon 6 of hCD45 comprises or consists of SEQ ID NO: 23. In one embodiment, the nucleic acid sequence of exon 6 encoding the C determinant of hCD45 comprises or consists of SEQ ID NO: 24.

SEQ ID NO: 23
DVPGERSTASTFPTDPVSPLTTTLSLAHHSSAALPARTSNTTITANTS
SEQ ID NO: 24
GATGTCCCAGGAGAGAGGAGTACAGCCAGCACCTTTCCTACAGACCCAGT
TTCCCCATTGACAACCACCCTCAGCCTTGCACACCACAGCTCTGCTGCCT
TACCTGCACGCACCTCCAACACCACCATCACAGCGAACACCTCA

In one embodiment, the antibody or binding fragment thereof binds to at least one epitope comprising or consisting of SEQ ID NO: 23 or a fragment thereof.

In one embodiment, the antibody or binding fragment thereof binds to at least one epitope comprising or consisting of a sequence sharing at least about 70%, preferably at least 75%, 80%, 85%, 90%, 91%, 92%, 93%, 94%, 95%, 96%, 97%, 98%, 99% or more identity with SEQ ID NO: 23 or a fragment thereof.

In one embodiment, the antibody or binding fragment thereof binds to at least one epitope encoded by a nucleic acid sequence comprising or consisting of SEQ ID NO: 24 or a fragment thereof.

In one embodiment, the antibody or binding fragment thereof binds to at least one epitope encoded by a nucleic acid sequence comprising or consisting of a sequence sharing at least about 70%, preferably at least 75%, 80%, 85%, 90%, 91%, 92%, 93%, 94%, 95%, 96%, 97%, 98%, 99% or more identity with SEQ ID NO: 24 or a fragment thereof.

In one embodiment, the antibody or binding fragment thereof binds to at least one epitope comprising or consisting of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 or 47 amino acids of SEQ ID NO: 23 or a fragment thereof; or of a sequence sharing at least about 70%, preferably at least 75%, 80%, 85%, 90%, 91%, 92%, 93%, 94%, 95%, 96%, 97%, 98%, 99% or more identity with SEQ ID NO: 23 or a fragment thereof.

In one embodiment, the antibody or binding fragment thereof binds to at least one epitope comprising or consisting of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 or 47 contiguous amino acids of SEQ ID NO: 23 or a fragment thereof; or of a sequence sharing at least about 70%, preferably at least 75%, 80%, 85%, 90%, 91%, 92%, 93%, 94%, 95%, 96%, 97%, 98%, 99% or more identity with SEQ ID NO: 23 or a fragment thereof.

In one embodiment, a fragment of the at least one epitope comprising or consisting of SEQ ID NO: 23 comprises or consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 or 47 amino acid residues.

In one embodiment, a fragment of the at least one epitope comprising or consisting of SEQ ID NO: 23 comprises or consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 or 47 amino acid residues spread over a span of 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 73, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100 or more contiguous amino acid residues of a sequence comprising or consisting of SEQ ID NO: 23.

In one embodiment, a sequence comprising SEQ ID NO: 23 is the sequence of hCD45 set forth in SEQ ID NO: 99, corresponding to UniProt Accession P08575-3 (version 3, modified Mar. 28, 2018—Checksum: 6E942E2BF6B17AC5).

SEQ ID NO: 99
MTMYLWLKLLAFGFAFLDTEVFVTGQSPTPSPTGLTTAKMPSVPLSSDP
LPTHTTAFSPASTFERENDFSETTTSLSPDNTSTQVSPDSLDNASAFNT
TGVSSVQTPHLPTHADSQTPSAGTDTQTFSGSAANAKLNPTPGSNAISD
VPGERSTASTFPTDPVSPLTTTLSLAHHSSAALPARTSNTTITANTSDA
YLNASETTTLSPSGSAVISTTTIATTPSKPTCDEKYANITVDYLYNKET
KLFTAKLNVNENVECGNNTCTNNEVHNLTECKNASVSISHNSCTAPDKT
LILDVPPGVEKFQLHDCTQVEKADTTICLKWKNIETFTCDTQNITYRFQ
CGNMIFDNKEIKLENLEPEHEYKCDSEILYNNHKFTNASKIIKTDFGSP
GEPQIIFCRSEAAHQGVITWNPPQRSFHNFTLCYIKETEKDCLNLDKNL
IKYDLQNLKPYTKYVLSLHAYIIAKVQRNGSAAMCHFTTKSAPPSQVWN
MTVSMTSDNSMHVKCRPPRDRNGPHERYHLEVEAGNTLVRNESHKNCDF
RVKDLQYSTDYTFKAYFHNGDYPGEPFILHHSTSYNSKALIAFLAFLII
VTSIALLVVLYKIYDLHKKRSCNLDEQQELVERDDEKQLMNVEPIHADI
LLETYKRKIADEGRLFLAEFQSIPRVFSKFPIKEARKPFNQNKNRYVDI
LPYDYNRVELSEINGDAGSNYINASYIDGFKEPRKYIAAQGPRDETVDD
FWRMIWEQKATVIVMVTRCEEGNRNKCAEYWPSMEEGTRAFGDVVVKIN
QHKRCPDYIIQKLNIVNKKEKATGREVTHIQFTSWPDHGVPEDPHLLLK
LRRRVNAFSNFFSGPIVVHCSAGVGRTGTYIGIDAMLEGLEAENKVDVY
GYVVKLRRQRCLMVQVEAQYILIHQALVEYNQFGETEVNLSELHPYLHN
MKKRDPPSEPSPLEAEFQRLPSYRSWRTQHIGNQEENKSKNRNSNVIPY
DYNRVPLKHELEMSKESEHDSDESSDDDSDSEEPSKYINASFIMSYWKP
EVMIAAQGPLKETIGDFWQMIFQRKVKVIVMLTELKHGDQEICAQYWGE
GKQTYGDIEVDLKDTDKSSTYTLRVFELRHSKRKDSRTVYQYQYTNWSV
EQLPAEPKELISMIQVVKQKLPQKNSSEGNKHHKSTPLLIHCRDGSQQT
GIFCALLNLLESAETEEVVDIFQVVKALRKARPGMVSTFEQYQFLYDVI
ASTYPAQNGQVKKNNHQEDKIEFDNEVDKVKQDANCVNPLGAPEKLPEA
KEQAEGSEPTSGTEGPEHSVNGPASPALNQGS
(wherein SEQ ID NO: 23 is underlined)

In one embodiment, a sequence comprising SEQ ID NO: 23 is the sequence of hCD45RC set forth in SEQ ID NO: 104, corresponding to UniProt Accession P08575-10 (version 10, modified Mar. 28, 2018—Checksum: F92C874C9A114890).

SEQ ID NO: 104
MTMYLWLKLLAFGFAFLDTEVFVTGQSPTPSPTDVPGERSTASTFPTDP
VSPLTTTLSLAHHSSAALPARTSNTTITANTSDAYLNASETTTLSPSGS
AVISTTTIATTPSKPTCDEKYANITVDYLYNKETKLFTAKLNVNENVEC
GNNTCTNNEVHNLTECKNASVSISHNSCTAPDKTLILDVPPGVEKFQLH
DCTQVEKADTTICLKWKNIETFTCDTQNITYRFQCGNMIFDNKEIKLEN
LEPEHEYKCDSEILYNNHKFTNASKIIKTDFGSPGEPQIIFCRSEAAHQ
GVITWNPPQRSFHNFTLCYIKETEKDCLNLDKNLIKYDLQNLKPYTKYV
LSLHAYIIAKVQRNGSAAMCHFTTKSAPPSQVWNMTVSMTSDNSMHVKC
RPPRDRNGPHERYHLEVEAGNTLVRNESHKNCDFRVKDLQYSTDYTFKA
YFHNGDYPGEPFILHHSTSYNSKALIAFLAFLIIVTSIALLVVLYKIYD
LHKKRSCNLDEQQELVERDDEKQLMNVEPIHADILLETYKRKIADEGRL
FLAEFQSIPRVFSKFPIKEARKPFNQNKNRYVDILPYDYNRVELSEING
DAGSNYINASYIDGFKEPRKYIAAQGPRDETVDDFWRMIWEQKATVIVM
VTRCEEGNRNKCAEYWPSMEEGTRAFGDVVVKINQHKRCPDYIIQKLNI
VNKKEKATGREVTHIQFTSWPDHGVPEDPHLLLKLRRRVNAFSNFFSGP
IVVHCSAGVGRTGTYIGIDAMLEGLEAENKVDVYGYVVKLRRQRCLMVQ
VEAQYILIHQALVEYNQFGETEVNLSELHPYLHNMKKRDPPSEPSPLEA
EFQRLPSYRSWRTQHIGNQEENKSKNRNSNVIPYDYNRVPLKHELEMSK
ESEHDSDESSDDDSDSEEPSKYINASFIMSYWKPEVMIAAQGPLKETIG
DFWQMIFQRKVKVIVMLTELKHGDQEICAQYWGEGKQTYGDIEVDLKDT
DKSSTYTLRVFELRHSKRKDSRTVYQYQYTNWSVEQLPAEPKELISMIQ
VVKQKLPQKNSSEGNKHHKSTPLLIHCRDGSQQTGIFCALLNLLESAET
EEVVDIFQVVKALRKARPGMVSTFEQYQFLYDVIASTYPAQNGQVKKNN
HQEDKIEFDNEVDKVKQDANCVNPLGAPEKLPEAKEQAEGSEPTSGTEG
PEHSVNGPASPALNQGS
(wherein SEQ ID NO: 23 is underlined)

In one embodiment, the antibody or binding fragment thereof does not bind to the A determinant encoded by exon 4 of hCD45. In one embodiment, the antibody or binding fragment thereof does not bind to at least one epitope on the A determinant encoded by exon 4 of hCD45.

In one embodiment, the amino acid sequence of the A determinant encoded by exon 4 of hCD45 comprises or consists of SEQ ID NO: 105.

SEQ ID NO: 105
GLTTAKMPSVPLSSDPLPTHTTAFSPASTFERENDFSETTTSLSPDNTS
TQVSPDSLDNASAFNTT

In one embodiment, the antibody or binding fragment thereof does not bind to the B determinant encoded by exon 5 of hCD45. In one embodiment, the antibody or binding fragment thereof does not bind to at least one epitope on the B determinant encoded by exon 5 of hCD45.

In one embodiment, the amino acid sequence of the B determinant encoded by exon 5 of hCD45 comprises or consists of SEQ ID NO: 106.

SEQ ID NO: 106
GVSSVQTPHLPTHADSQTPSAGTDTQTFSGSAANAKLNPTPGSNAIS

In one embodiment, the antibody or binding fragment thereof does not bind to hCD45RA. In one embodiment, the antibody or binding fragment thereof does not bind to at least one epitope of hCD45RA.

In one embodiment, the amino acid sequence of hCD45RA comprises or consists of SEQ ID NO: 107, corresponding to UniProt Accession P08575-8 (version 8, modified Mar. 28, 2018—Checksum: F42C1FEC9EDE4BC0).

SEQ ID NO: 107
MTMYLWLKLLAFGFAFLDTEVFVTGQSPTPSPTGLTTAKMPSVPLSSD
PLPTHTTAFSPASTFERENDFSETTTSLSPDNTSTQVSPDSLDNASAF
NTTDAYLNASETTTLSPSGSAVISTTTIATTPSKPTCDEKYANITVDY
LYNKETKLFTAKLNVNENVECGNNTCTNNEVHNLTECKNASVSISHNS
CTAPDKTLILDVPPGVEKFQLHDCTQVEKADTTICLKWKNIETFTCDT
QNITYRFQCGNMIFDNKEIKLENLEPEHEYKCDSEILYNNHKFTNASK
IIKTDFGSPGEPQIIFCRSEAAHQGVITWNPPQRSFHNFTLCYIKETE
KDCLNLDKNLIKYDLQNLKPYTKYVLSLHAYIIAKVQRNGSAAMCHFT
TKSAPPSQVWNMTVSMTSDNSMHVKCRPPRDRNGPHERYHLEVEAGNT
LVRNESHKNCDFRVKDLQYSTDYTFKAYFHNGDYPGEPFILHHSTSYN
SKALIAFLAFLIIVTSIALLVVLYKIYDLHKKRSCNLDEQQELVERDD
EKQLMNVEPIHADILLETYKRKIADEGRLFLAEFQSIPRVFSKFPIKE
ARKPFNQNKNRYVDILPYDYNRVELSEINGDAGSNYINASYIDGFKEP
RKYIAAQGPRDETVDDFWRMIWEQKATVIVMVTRCEEGNRNKCAEYWP
SMEEGTRAFGDVVVKINQHKRCPDYIIQKLNIVNKKEKATGREVTHIQ
FTSWPDHGVPEDPHLLLKLRRRVNAFSNFFSGPIVVHCSAGVGRTGTY
IGIDAMLEGLEAENKVDVYGYVVKLRRQRCLMVQVEAQYILIHQALVE
YNQFGETEVNLSELHPYLHNMKKRDPPSEPSPLEAEFQRLPSYRSWRT
QHIGNQEENKSKNRNSNVIPYDYNRVPLKHELEMSKESEHDSDESSDD
DSDSEEPSKYINASFIMSYWKPEVMIAAQGPLKETIGDFWQMIFQRKV
KVIVMLTELKHGDQEICAQYWGEGKQTYGDIEVDLKDTDKSSTYTLRV
FELRHSKRKDSRTVYQYQYTNWSVEQLPAEPKELISMIQVVKQKLPQK
NSSEGNKHHKSTPLLIHCRDGSQQTGIFCALLNLLESAETEEVVDIFQ
VVKALRKARPGMVSTFEQYQFLYDVIASTYPAQNGQVKKNNHQEDKIE
FDNEVDKVKQDANCVNPLGAPEKLPEAKEQAEGSEPTSGTEGPEHSVN
GPASPALNQGS

In one embodiment, the antibody or binding fragment thereof does not bind to hCD45RB.

In one embodiment, the antibody or binding fragment thereof does not bind to at least one epitope of hCD45RB.

In one embodiment, the amino acid sequence of hCD45RB comprises or consists of SEQ ID NO: 108, corresponding to UniProt Accession P08575-9 (version 9, modified Mar. 28, 2018—Checksum: 745870037910C575).

SEQ ID NO: 108
MTMYLWLKLLAFGFAFLDTEVFVTGQSPTPSPTGV
SSVQTPHLPTHADSQTPSAGTDTQTFSGSAANAKL
NPTPGSNAISDAYLNASETTTLSPSGSAVISTTTI
ATTPSKPTCDEKYANITVDYLYNKETKLFTAKLNV
NENVECGNNTCTNNEVHNLTECKNASVSISHNSCT
APDKTLILDVPPGVEKFQLHDCTQVEKADTTICLK
WKNIETFTCDTQNITYRFQCGNMIFDNKEIKLENL
EPEHEYKCDSEILYNNHKFTNASKIIKTDFGSPGE
PQIIFCRSEAAHQGVITWNPPQRSFHNFTLCYIKE
TEKDCLNLDKNLIKYDLQNLKPYTKYVLSLHAYII
AKVQRNGSAAMCHFTTKSAPPSQVWNMTVSMTSDN
SMHVKCRPPRDRNGPHERYHLEVEAGNTLVRNESH
KNCDFRVKDLQYSTDYTFKAYFHNGDYPGEPFILH
HSTSYNSKALIAFLAFLIIVTSIALLVVLYKIYDL
HKKRSCNLDEQQELVERDDEKQLMNVEPIHADILL
ETYKRKIADEGRLFLAEFQSIPRVFSKFPIKEARK
PFNQNKNRYVDILPYDYNRVELSEINGDAGSNYIN
ASYIDGFKEPRKYIAAQGPRDETVDDFWRMIWEQK
ATVIVMVTRCEEGNRNKCAEYWPSMEEGTRAFGDV
VVKINQHKRCPDYIIQKLNIVNKKEKATGREVTHI
QFTSWPDHGVPEDPHLLLKLRRRVNAFSNFFSGPI
VVHCSAGVGRTGTYIGIDAMLEGLEAENKVDVYGY
VVKLRRQRCLMVQVEAQYILIHQALVEYNQFGETE
VNLSELHPYLHNMKKRDPPSEPSPLEAEFQRLPSY
RSWRTQHIGNQEENKSKNRNSNVIPYDYNRVPLKH
ELEMSKESEHDSDESSDDDSDSEEPSKYINASFIM
SYWKPEVMIAAQGPLKETIGDFWQMIFQRKVKVIV
MLTELKHGDQEICAQYWGEGKQTYGDIEVDLKDTD
KSSTYTLRVFELRHSKRKDSRTVYQYQYTNWSVEQ
LPAEPKELISMIQVVKQKLPQKNSSEGNKHHKSTP
LLIHCRDGSQQTGIFCALLNLLESAETEEVVDIFQ
VVKALRKARPGMVSTFEQYQFLYDVIASTYPAQNG
QVKKNNHQEDKIEFDNEVDKVKQDANCVNPLGAPE
KLPEAKEQAEGSEPTSGTEGPEHSVNGPASPALNQ
GS

In one embodiment, the antibody or binding fragment thereof does not bind to hCD45RAB. In one embodiment, the antibody or binding fragment thereof does not bind to at least one epitope of hCD45RAB.

In one embodiment, the amino acid sequence of hCD45RAB comprises or consists of SEQ ID NO: 109, corresponding to UniProt Accession P08575-5 (version 5, modified Mar. 28, 2018—Checksum: EA40BE995CD98F7C).

SEQ ID NO: 109
MTMYLWLKLLAFGFAFLDTEVFVTGQSPTPSPTGL
TTAKMPSVPLSSDPLPTHTTAFSPASTFERENDFS
ETTTSLSPDNTSTQVSPDSLDNASAFNTTGVSSVQ
TPHLPTHADSQTPSAGTDTQTFSGSAANAKLNPTP
GSNAISDAYLNASETTTLSPSGSAVISTTTIATTP
SKPTCDEKYANITVDYLYNKETKLFTAKLNVNENV
ECGNNTCTNNEVHNLTECKNASVSISHNSCTAPDK
TLILDVPPGVEKFQLHDCTQVEKADTTICLKWKNI
ETFTCDTQNITYRFQCGNMIFDNKEIKLENLEPEH
EYKCDSEILYNNHKFTNASKIIKTDFGSPGEPQII
FCRSEAAHQGVITWNPPQRSFHNFTLCYIKETEKD
CLNLDKNLIKYDLQNLKPYTKYVLSLHAYIIAKVQ
RNGSAAMCHFTTKSAPPSQVWNMTVSMTSDNSMHV
KCRPPRDRNGPHERYHLEVEAGNTLVRNESHKNCD
FRVKDLQYSTDYTFKAYFHNGDYPGEPFILHHSTS
YNSKALIAFLAFLIIVTSIALLVVLYKIYDLHKKR
SCNLDEQQELVERDDEKQLMNVEPIHADILLETYK
RKIADEGRLFLAEFQSIPRVFSKFPIKEARKPFNQ
NKNRYVDILPYDYNRVELSEINGDAGSNYINASYI
DGFKEPRKYIAAQGPRDETVDDFWRMIWEQKATVI
VMVTRCEEGNRNKCAEYWPSMEEGTRAFGDVVVKI
NQHKRCPDYIIQKLNIVNKKEKATGREVTHIQFTS
WPDHGVPEDPHLLLKLRRRVNAFSNFFSGPIVVHC
SAGVGRTGTYIGIDAMLEGLEAENKVDVYGYVVKL
RRQRCLMVQVEAQYILIHQALVEYNQFGETEVNLS
ELHPYLHNMKKRDPPSEPSPLEAEFQRLPSYRSWR
TQHIGNQEENKSKNRNSNVIPYDYNRVPLKHELEM
SKESEHDSDESSDDDSDSEEPSKYINASFIMSYWK
PEVMIAAQGPLKETIGDFWQMIFQRKVKVIVMLTE
LKHGDQEICAQYWGEGKQTYGDIEVDLKDTDKSST
YTLRVFELRHSKRKDSRTVYQYQYTNWSVEQLPAE
PKELISMIQVVKQKLPQKNSSEGNKHHKSTPLLIH
CRDGSQQTGIFCALLNLLESAETEEVVDIFQVVKA
LRKARPGMVSTFEQYQFLYDVIASTYPAQNGQVKK
NNHQEDKIEFDNEVDKVKQDANCVNPLGAPEKLPE
AKEQAEGSEPTSGTEGPEHSVNGPASPALNQGS

In one embodiment, the antibody or binding fragment thereof does not bind to hCD45R0.

In one embodiment, the antibody or binding fragment thereof does not bind to at least one epitope of hCD45R0.

In one embodiment, the amino acid sequence of hCD45R0 comprises or consists of SEQ ID NO: 110, corresponding to UniProt Accession P08575-4 (version 4, modified Mar. 28, 2018—Checksum: D3CB364EF4243384).

SEQ ID NO: 110
MTMYLWLKLLAFGFAFLDTEVFVTGQSPTPSPTDA
YLNASETTTLSPSGSAVISTTTIATTPSKPTCDEK
YANITVDYLYNKETKLFTAKLNVNENVECGNNTCT
NNEVHNLTECKNASVSISHNSCTAPDKTLILDVPP
GVEKFQLHDCTQVEKADTTICLKWKNIETFTCDTQ
NITYRFQCGNMIFDNKEIKLENLEPEHEYKCDSEI
LYNNHKFTNASKIIKTDFGSPGEPQIIFCRSEAAH
QGVITWNPPQRSFHNFTLCYIKETEKDCLNLDKNL
IKYDLQNLKPYTKYVLSLHAYIIAKVQRNGSAAMC
HFTTKSAPPSQVWNMTVSMTSDNSMHVKCRPPRDR
NGPHERYHLEVEAGNTLVRNESHKNCDFRVKDLQY
STDYTFKAYFHNGDYPGEPFILHHSTSYNSKALIA
FLAFLIIVTSIALLVVLYKIYDLHKKRSCNLDEQQ
ELVERDDEKQLMNVEPIHADILLETYKRKIADEGR
LFLAEFQSIPRVFSKFPIKEARKPFNQNKNRYVDI
LPYDYNRVELSEINGDAGSNYINASYIDGFKEPRK
YIAAQGPRDETVDDFWRMIWEQKATVIVMVTRCEE
GNRNKCAEYWPSMEEGTRAFGDVVVKINQHKRCPD
YIIQKLNIVNKKEKATGREVTHIQFTSWPDHGVPE
DPHLLLKLRRRVNAFSNFFSGPIVVHCSAGVGRTG
TYIGIDAMLEGLEAENKVDVYGYVVKLRRQRCLMV
QVEAQYILIHQALVEYNQFGETEVNLSELHPYLHN
MKKRDPPSEPSPLEAEFQRLPSYRSWRTQHIGNQE
ENKSKNRNSNVIPYDYNRVPLKHELEMSKESEHDS
DESSDDDSDSEEPSKYINASFIMSYWKPEVMIAAQ
GPLKETIGDFWQMIFQRKVKVIVMLTELKHGDQEI
CAQYWGEGKQTYGDIEVDLKDTDKSSTYTLRVFEL
RHSKRKDSRTVYQYQYTNWSVEQLPAEPKELISMI
QVVKQKLPQKNSSEGNKHHKSTPLLIHCRDGSQQT
GIFCALLNLLESAETEEVVDIFQVVKALRKARPGM
VSTFEQYQFLYDVIASTYPAQNGQVKKNNHQEDKI
EFDNEVDKVKQDANCVNPLGAPEKLPEAKEQAEGS
EPTSGTEGPEHSVNGPASPALNQGS

In one embodiment, the at least one epitope is a conformational epitope. In another embodiment, the at least one epitope is a sequential epitope.

In one embodiment, the antibody or binding fragment thereof binds to hCD45RC with an equilibrium dissociation constant (K_{d}) of about 5×10⁻⁷ M or less, preferably of about 2.5×10⁻⁷ M or less, about 1×10⁻⁷ M or less, about 7.5×10⁻⁸ M or less, about 5×10⁻⁸ M or less, about 1×10⁻⁸ M or less.

In one embodiment, the antibody or binding fragment thereof binds to hCD45RC with an association rate (Kₒₙ) of about 1×10⁴ M⁻¹ sec⁻¹ or more, preferably of about 5×10⁴ M⁻¹ sec⁻¹ or more, about 1×10⁵ M⁻¹ sec⁻¹ or more, about 2.5×10⁵ M⁻¹ sec⁻¹ or more, about 5×10⁵ M⁻¹ sec⁻¹ or more.

In one embodiment, the antibody or binding fragment thereof binds to hCD45RC with a dissociation rate (K_{off}) of about 5×10⁻² sec⁻¹ or less, preferably of about 4×10⁻² sec⁻¹ or less, about 3×10⁻² sec⁻¹ or less, about 2×10⁻² sec⁻¹ or less, about 1.5×10⁻² sec⁻¹ or less.

In one embodiment, the antibody or binding fragment thereof binds to hCD45RC with at least one of, preferably at least two of, more preferably the three of:
an equilibrium dissociation constant (K_{d}) of about 5×10⁻⁷ M or less, preferably of about 2.5×10⁻⁷ M or less, about 1×10⁻⁷ M or less, about 7.5×10⁻⁸ M or less, about 5×10⁻⁸ M or less, about 1×10⁻⁸ M or less;
an association rate (Kₒₙ) of about 1×10⁴ M⁻¹ sec⁻¹ or more, preferably of about 5×10⁴ M⁻¹ sec⁻¹ or more, about 1×10⁵ M⁻¹ sec⁻¹ or more, about 2.5×10⁵ M⁻¹ sec⁻¹ or more, about 5×10⁵ M⁻¹ sec⁻¹ or more; and
a dissociation rate (K_{off}) of about 5×10⁻² sec⁻¹ or less, preferably of about 4×10⁻² sec⁻¹ or less, about 3×10⁻² sec⁻¹ or less, about 2×10⁻² sec⁻¹ or less, about 1.5×10⁻² sec⁻¹ or less.

Methods for determining the affinity (including, for example, determining the K_{d}, k_{off} and kₒₙ) of an antibody or binding fragment thereof for its ligand are well-known in the art, and include, without limitation, surface plasmon resonance (SPR), fluorescence-activated cell sorting (FACS), enzyme-linked immunosorbent assay (ELISA), AlphaLISA and KinExA.

A preferred method is BIAcore®, which relies on SPR using immobilized CD45RC to determine the affinity of an antibody or binding fragment thereof. A way of implementing this method will be further illustrated in the Examples section.

In one embodiment, the antibody or binding fragment thereof is a polyclonal antibody or binding fragment thereof.

In a preferred embodiment, the antibody or binding fragment thereof is a monoclonal antibody or binding fragment thereof.

In one embodiment, the antibody or binding fragment thereof is a molecule selected from the group comprising or consisting of a whole antibody, a single-chain antibody, a dimeric single chain antibody, a single-domain antibody, a Fv, a Fab, a Fab′, a Fab′-SH, a F(ab)′2, a Fd, a defucosylated antibody, a bi-specific antibody, a diabody, a triabody and a tetrabody.

Antibody binding fragments can be obtained using standard methods. For instance, Fab or F(ab′)2 fragments may be produced by protease digestion of the isolated antibodies, according to conventional techniques. It will also be appreciated that antibodies or binding fragments thereof can be modified using known methods. For example, to slow clearance in vivo and obtain a more desirable pharmacokinetic profile, the antibody or binding fragment thereof may be modified with polyethylene glycol (PEG). Methods for coupling and site-specifically conjugating PEG to an antibody or binding fragment thereof are described in, e.g., Leong et al., 2001. Cytokine. 16(3):106-19; Delgado et al., 1996. Br J Cancer. 73(2):175-82.

In one embodiment, the antibody or binding fragment thereof is a molecule selected from the group comprising or consisting of a unibody, a domain antibody, and a nanobody.

In one embodiment, the antibody or binding fragment thereof is a mimetic selected from the group comprising or consisting of an affibody, an affilin, an affitin, an adnectin, an atrimer, an evasin, a DARPin, an anticalin, an avimer, a fynomer, a versabody and a duocalin.

In one embodiment, the antibody or binding fragment thereof also encompasses multispecific antibodies or binding fragments thereof, i.e., being immunospecific for more than one, such as at least two, different antigens, one of which being hCD45RC according to the present invention.

In one embodiment, the antibody or binding fragment thereof also encompasses polymers of antibodies or binding fragments thereof, i.e., more than one, such as at least two, antibodies or binding fragments thereof, whether identical or different, being covalently linked together, directly or indirectly.

In the following, and unless explicitly mentioned otherwise, CDR numbering and definitions are according to the Kabat/Chothia definition.

In one embodiment, the antibody or binding fragment thereof comprises a heavy chain variable region (abbreviated herein as HCVR or V_{H}) which comprises at least one, preferably at least two, more preferably the following three complementary-determining regions (CDRs):

V_{H}-CDR1:
(SEQ ID NO: 1)
NYYIG;
V_{H}-CDR2:
(SEQ ID NO: 2)
X-IF-X₂-GG-X₃-Y-X₄-N-X₅-X₆-X₇-X₈-X₉-X₁₀-G;
and
V_{H}-CDR3:
(SEQ ID NO: 3)
RNFDY,

with:

X₁ being selected from Asp (D), Ile (I) and Arg (R);

X₂ being selected from Pro (P) and Ser (S);

X₃ being selected from Asp (D), Ser (S) and Gly (G);

X₄ being selected from Ala (A) and Thr (T);

X₅ being selected from Ser (S) and Tyr (Y);

X₆ being selected from Asn (N), Ala (A) and Ser (S);

X₇ being selected from Glu (E), Asp (D), Pro (P) and Gln (Q);

X₈ being selected from Lys (K) and Ser (S);

X₉ being selected from Phe (F) and Val (V); and

X₁₀ being selected from Lys (K) and Gln (Q).

In one embodiment, the antibody or binding fragment thereof comprises a HCVR which comprises the following three CDRs:

V_{H}-CDR1:
(SEQ ID NO: 1)
NYYIG;
V_{H}-CDR2:
(SEQ ID NO: 2)
X₁-IF-X₂-GG-X₃-Y-X₄-N-X₅-X₆-X₇-X₈-X₉-X₁₀-G;
and
V_{H}-CDR3:
(SEQ ID NO: 3)
RNFDY,

with: DIFPGGDYANSNEKFKG

X₁ being selected from Asp (D), Ile (I) and Arg (R);

X₂ being selected from Pro (P) and Ser (S);

X₃ being selected from Asp (D), Ser (S) and Gly (G);

X₄ being selected from Ala (A) and Thr (T);

X₅ being selected from Ser (S) and Tyr (Y);

X₆ being selected from Asn (N), Ala (A) and Ser (S);

X₇ being selected from Glu (E), Asp (D), Pro (P) and Gln (Q);

X₈ being selected from Lys (K) and Ser (S);

X₉ being selected from Phe (F) and Val (V); and

X₁₀ being selected from Lys (K) and Gln (Q).

In one embodiment, the antibody or binding fragment thereof comprises a HCVR which comprises at least one, preferably at least two, more preferably the following three CDRs:

V_{H}-CDR1:
(SEQ ID NO: 1)
NYYIG;
V_{H}-CDR2:
(SEQ ID NO: 4)
DIFPGGDYANSNEKFKG;
and
V_{H}-CDR3:
(SEQ ID NO: 3)
RNFDY.

In one embodiment, the antibody or binding fragment thereof comprises a HCVR which comprises the following three CDRs:

V_{H}-CDR1:
(SEQ ID NO: 1)
NYYIG;
V_{H}-CDR2:
(SEQ ID NO: 4)
DIFPGGDYANSNEKFKG;
and
V_{H}-CDR3:
(SEQ ID NO: 3)
RNFDY.

In one embodiment, the antibody or binding fragment thereof comprises a HCVR which comprises at least one, preferably at least two, more preferably the following three CDRs:

V_{H}-CDR1:
(SEQ ID NO: 1)
NYYIG;
V_{H}-CDR2:
(SEQ ID NO: 5)
DIFPGGDYANSNEKVKG;
and
V_{H}-CDR3:
(SEQ ID NO: 3)
RNFDY.

In one embodiment, the antibody or binding fragment thereof comprises a HCVR which comprises the following three CDRs:

V_{H}-CDR1:
(SEQ ID NO: 1)
NYYIG;
V_{H}-CDR2:
(SEQ ID NO: 5)
DIFPGGDYANSNEKVKG;
and
V_{H}-CDR3:
(SEQ ID NO: 3)
RNFDY.

In one embodiment, the antibody or binding fragment thereof comprises a HCVR which comprises at least one, preferably at least two, more preferably the following three CDRs:

V_{H}-CDR1:
(SEQ ID NO: 1)
NYYIG;
V_{H}-CDR2:
(SEQ ID NO: 6)
DIFPGGGYTNYAEKFQG;
and
V_{H}-CDR3:
(SEQ ID NO: 3)
RNFDY.

In one embodiment, the antibody or binding fragment thereof comprises a HCVR which comprises the following three CDRs:

V_{H}-CDR1:
(SEQ ID NO: 1)
NYYIG;
V_{H}-CDR2:
(SEQ ID NO: 6)
DIFPGGGYTNYAEKFQG;
and
V_{H}-CDR3:
(SEQ ID NO: 3)
RNFDY

In one embodiment, the antibody or binding fragment thereof comprises a HCVR which comprises at least one, preferably at least two, more preferably the following three CDRs:

V_{H}-CDR1:
(SEQ ID NO: 1)
NYYIG;
V_{H}-CDR2:
(SEQ ID NO: 7)
DIFPGGSYTNYSESFQG;
and
V_{H}-CDR3:
(SEQ ID NO: 3)
RNFDY.

In one embodiment, the antibody or binding fragment thereof comprises a HCVR which comprises the following three CDRs:

V_{H}-CDR1:
(SEQ ID NO: 1)
NYYIG;
V_{H}-CDR2:
(SEQ ID NO: 7)
DIFPGGSYTNYSESFQG;
and
V_{H}-CDR3:
(SEQ ID NO: 3)
RNFDY.

In one embodiment, the antibody or binding fragment thereof comprises a HCVR which comprises at least one, preferably at least two, more preferably the following three CDRs:

V_{H}-CDR1:
(SEQ ID NO: 1)
NYYIG;
V_{H}-CDR2:
(SEQ ID NO: 8)
DIFPGGSYTNYADSVKG;
and
V_{H}-CDR3:
(SEQ ID NO: 3)
RNFDY.

In one embodiment, the antibody or binding fragment thereof comprises a HCVR which comprises the following three CDRs:

V_{H}-CDR1:
(SEQ ID NO: 1)
NYYIG;
V_{H}-CDR2:
(SEQ ID NO: 8)
DIFPGGSYTNYADSVKG;
and
V_{H}-CDR3:
(SEQ ID NO: 3)
RNFDY.

In one embodiment, the antibody or binding fragment thereof comprises a HCVR which comprises at least one, preferably at least two, more preferably the following three CDRs:

V_{H}-CDR1:
(SEQ ID NO: 1)
NYYIG;
V_{H}-CDR2:
(SEQ ID NO: 9)
RIFPGGGYTNYAQKFQG;
and
V_{H}-CDR3:
(SEQ ID NO: 3)
RNFDY.

In one embodiment, the antibody or binding fragment thereof comprises a HCVR which comprises the following three CDRs:

V_{H}-CDR1:
(SEQ ID NO: 1)
NYYIG;
V_{H}-CDR2:
(SEQ ID NO: 9)
RIFPGGGYTNYAQKFQG;
and
V_{H}-CDR3:
(SEQ ID NO: 3)
RNFDY.

In one embodiment, the antibody or binding fragment thereof comprises a HCVR which comprises at least one, preferably at least two, more preferably the following three CDRs:

V_{H}-CDR1:
(SEQ ID NO: 1)
NYYIG;
V_{H}-CDR2:
(SEQ ID NO: 10)
IIFPGGSYTNYSPSFQG;
and
V_{H}-CDR3:
(SEQ ID NO: 3)
RNFDY.

In one embodiment, the antibody or binding fragment thereof comprises a HCVR which comprises the following three CDRs:

V_{H}-CDR1:
(SEQ ID NO: 1)
NYYIG;
V_{H}-CDR2:
(SEQ ID NO: 10)
IIFPGGSYTNYSPSFQG;
and
V_{H}-CDR3:
(SEQ ID NO: 3)
RNFDY.

In one embodiment, the antibody or binding fragment thereof comprises a HCVR which comprises at least one, preferably at least two, more preferably the following three CDRs:

V_{H}-CDR1:
(SEQ ID NO: 1)
NYYIG;
V_{H}-CDR2:
(SEQ ID NO: 11)
DIFSGGSYTNYADSVKG;
and
V_{H}-CDR3:
(SEQ ID NO: 3)
RNFDY.

In one embodiment, the antibody or binding fragment thereof comprises a HCVR which comprises the following three CDRs:

V_{H}-CDR1:
(SEQ ID NO: 1)
NYYIG;
V_{H}-CDR2:
(SEQ ID NO: 11)
DIFSGGSYTNYADSVKG;
and
V_{H}-CDR3:
(SEQ ID NO: 3)
RNFDY.

In one embodiment, the antibody or binding fragment thereof comprises a HCVR which comprises at least one, preferably at least two, more preferably the following three CDRs: V_{H}-CDR1: NYYIG (SEQ ID NO: 1);

V_{H}-CDR1:
(SEQ ID NO: 1)
NYYIG;
V_{H}-CDR2:
(SEQ ID NO: 100)
DIFPGGDYTNYAEKFQG;
and
V_{H}-CDR3:
(SEQ ID NO: 3)
RNFDY.

In one embodiment, the antibody or binding fragment thereof comprises a HCVR which comprises the following three CDRs:

V_{H}-CDR1:
(SEQ ID NO: 1)
NYYIG;
V_{H}-CDR2:
(SEQ ID NO: 100)
DIFPGGDYTNYAEKFQG;
and
V_{H}-CDR3:
(SEQ ID NO: 3)
RNFDY.

In one embodiment, the antibody or binding fragment thereof comprises a HCVR which comprises at least one, preferably at least two, more preferably the following three CDRs:

V_{H}-CDR1:
(SEQ ID NO: 1)
NYYIG;
V_{H}-CDR2:
(SEQ ID NO: 116)
DIFPGGGYANYAEKFQG;
and
V_{H}-CDR3:
(SEQ ID NO: 3)
RNFDY.

In one embodiment, the antibody or binding fragment thereof comprises a HCVR which comprises the following three CDRs:

V_{H}-CDR1:
(SEQ ID NO: 1)
NYYIG;
V_{H}-CDR2:
(SEQ ID NO: 116)
DIFPGGGYANYAEKFQG;
and
V_{H}-CDR3:
(SEQ ID NO: 3)
RNFDY.

In one embodiment, the antibody or binding fragment thereof comprises a HCVR which comprises at least one, preferably at least two, more preferably the following three CDRs:

V_{H}-CDR1:
(SEQ ID NO: 1)
NYYIG;
V_{H}-CDR2:
(SEQ ID NO: 117)
DIFPGGGYTNYAEKFKG;
and
V_{H}-CDR3:
(SEQ ID NO: 3)
RNFDY.

In one embodiment, the antibody or binding fragment thereof comprises a HCVR which comprises the following three CDRs:

V_{H}-CDR1:
(SEQ ID NO: 1)
NYYIG;
V_{H}-CDR2:
(SEQ ID NO: 117)
DIFPGGGYTNYAEKFKG;
and
V_{H}-CDR3:
(SEQ ID NO: 3)
RNFDY.

In one embodiment, the antibody or binding fragment thereof comprises a HCVR which comprises at least one, preferably at least two, more preferably the following three CDRs:

V_{H}-CDR1:
(SEQ ID NO: 1)
NYYIG;
V_{H}-CDR2:
(SEQ ID NO: 118)
DIFPGGGYTNYNEKFQG;
and
V_{H}-CDR3:
(SEQ ID NO: 3)
RNFDY

In one embodiment, the antibody or binding fragment thereof comprises a HCVR which comprises the following three CDRs:

V_{H}-CDR1:
(SEQ ID NO: 1)
NYYIG;
V_{H}-CDR2:
(SEQ ID NO: 118)
DIFPGGGYTNYNEKFQG;
and
V_{H}-CDR3:
(SEQ ID NO: 3)
RNFDY.

In one embodiment, the antibody or binding fragment thereof comprises a HCVR which comprises at least one, preferably at least two, more preferably the following three CDRs:

V_{H}-CDR1:
(SEQ ID NO: 1)
NYYIG;
V_{H}-CDR2:
(SEQ ID NO: 119)
DIFPGGGYTNSAEKFQG;
and
V_{H}-CDR3:
(SEQ ID NO: 3)
RNFDY.

In one embodiment, the antibody or binding fragment thereof comprises a HCVR which comprises the following three CDRs:

V_{H}-CDR1:
(SEQ ID NO: 1)
NYYIG;
V_{H}-CDR2:
(SEQ ID NO: 119)
DIFPGGGYTNSAEKFQG;
and
V_{H}-CDR3:
(SEQ ID NO: 3)
RNFDY.

In one embodiment, the antibody or binding fragment thereof comprises a light chain variable region (abbreviated herein as LCVR or V_{L}) which comprises at least one, preferably at least two, more preferably the following three complementary-determining regions (CDRs):

V_{L}-CDR1:
(SEQ ID NO: 12)
X₁₁-ASSSVS-X₁₂-YMH;
V_{L}-CDR2:
(SEQ ID NO: 13)
X₁₃-TSN-X₁₄-X₁₅-X₁₆;
and
V_{L}-CDR3:
(SEQ ID NO: 14)
X₁₇-QRSSYPLTF,

with:

X₁₁ being selected from Ser (S) and Arg (R);

X₁₂ being absent or being selected from Asn (N), Ser (S) and Gly (G);

X₁₃ being selected from Asn (N) and Ala (A); or X₁₃ being any amino acid but Ala (A) or Asn (N);

X₁₄ being selected from Leu (L), Ser (S) and Arg (R);

X₁₅ being selected from Pro (P), Ala (A) and Gln (Q);

X₁₆ being selected from Ser (S) and Thr (T); and

X₁₇ being selected from Gln (Q) and His (H).

In one embodiment, the antibody or binding fragment thereof comprises a LCVR which comprises the following three CDRs:

V_{L}-CDR1:
(SEQ ID NO: 12)
X₁₁-ASSSVS-X₁₂-YMH;
V_{L}-CDR2:
(SEQ ID NO: 13)
X₁₃-TSN-X₁₄-X₁₅-X₁₆;
and
V_{L}-CDR3:
(SEQ ID NO: 14)
X₁₇-QRSSYPLTF,

with:

X₁₁ being selected from Ser (S) and Arg (R);

X₁₂ being absent or being selected from Asn (N), Ser (S) and Gly (G);

X₁₃ being selected from Asn (N) and Ala (A); or X₁₃ being any amino acid but Ala (A) or Asn (N);

X₁₄ being selected from Leu (L), Ser (S) and Arg (R);

X₁₅ being selected from Pro (P), Ala (A) and Gln (Q);

X₁₆ being selected from Ser (S) and Thr (T); and

X₁₇ being selected from Gln (Q) and His (H).

In one embodiment, the antibody or binding fragment thereof comprises a LCVR which comprises at least one, preferably at least two, more preferably the following three complementary-determining regions (CDRs):

V_{L}-CDR1:
(SEQ ID NO: 12)
X₁₁-ASSSVS-X₁₂-YMH;
V_{L}-CDR2:
(SEQ ID NO: 13)
X₁₃-TSN-X₁₄-X₁₅-X₁₆;
and
V_{L}-CDR3:
(SEQ ID NO: 14)
X₁₇-QRSSYPLTF,

with:

X₁₁ being Ser (S);

X₁₂ being absent or being selected from Asn (N), Ser (S) and Gly (G);

X₁₃ being Asn (N); or X₁₃ being any amino acid but Ala (A) or Asn (N);

X₁₄ being Leu (L);

X₁₅ being Pro (P);

X₁₆ being Ser (S); and

X₁₇ being Gln (Q).

In one embodiment, the antibody or binding fragment thereof comprises a LCVR which comprises the following three CDRs:

V_{L}-CDR1:
(SEQ ID NO: 12)
X₁₁-ASSSVS-X₁₂-YMH;
V_{L}-CDR2:
(SEQ ID NO: 13)
X₁₃-TSN-X₁₄-X₁₅-X₁₆;
and
V_{L}-CDR3:
(SEQ ID NO: 14)
X₁₇-QRSSYPLTF,

with:

X₁₁ being Ser (S);

X₁₂ being absent or being selected from Asn (N), Ser (S) and Gly (G);

X₁₃ being Asn (N); or X₁₃ being any amino acid but Ala (A) or Asn (N);

X₁₄ being Leu (L);

X₁₅ being Pro (P);

X₁₆ being Ser (S); and

X₁₇ being Gln (Q).

In one embodiment, the antibody or binding fragment thereof comprises a LCVR which comprises at least one, preferably at least two, more preferably the following three CDRs:

V_{L}-CDR1:
(SEQ ID NO: 15)
SASSSVS-X₁₂-YMH;
V_{L}-CDR2:
(SEQ ID NO: 16)
NTSNLPS;
and
V_{L}-CDR3:
(SEQ ID NO: 17)
QQRSSYPLTF,

with:

X₁₂ being absent or being selected from Asn (N), Ser (S) and Gly (G).

In one embodiment, the antibody or binding fragment thereof comprises a LCVR which comprises the following three CDRs:

V_{L}-CDR1:
(SEQ ID NO: 15)
SASSSVS-X₁₂-YMH;
V_{L}-CDR2:
(SEQ ID NO: 16)
NTSNLPS;
and
V_{L}-CDR3:
(SEQ ID NO: 17)
QQRSSYPLTF,

with:

X₁₂ being absent or being selected from Asn (N), Ser (S) and Gly (G).

In one embodiment, the antibody or binding fragment thereof comprises a LCVR which comprises at least one, preferably at least two, more preferably the following three CDRs:

V_{L}-CDR1:
(SEQ ID NO: 15)
SASSSVSYMH;
V_{L}-CDR2:
(SEQ ID NO: 16)
NTSNLPS;
and
V_{L}-CDR3:
(SEQ ID NO: 17)
QQRSSYPLTF.

In one embodiment, the antibody or binding fragment thereof comprises a LCVR which comprises the following three CDRs:

V_{L}-CDR1:
(SEQ ID NO: 15)
SASSSVSYMH;
V_{L}-CDR2:
(SEQ ID NO: 16)
NTSNLPS;
and
V_{L}-CDR3:
(SEQ ID NO: 17)
QQRSSYPLTF.

In one embodiment, the antibody or binding fragment thereof comprises a LCVR which comprises at least one, preferably at least two, more preferably the following three CDRs:

V_{L}-CDR1:
(SEQ ID NO: 15)
SASSSVS-X₁₂-YMH;
V_{L}-CDR2:
(SEQ ID NO: 16)
NTSNLPS;
and
V_{L}-CDR3:
(SEQ ID NO: 17)
QQRSSYPLTF,

with:

X₁₂ being selected from Asn (N), Ser (S) and Gly (G).

In one embodiment, the antibody or binding fragment thereof comprises a LCVR which comprises the following three CDRs:

V_{L}-CDR1:
(SEQ ID NO: 15)
SASSSVS-X₁₂-YMH;
V_{L}-CDR2:
(SEQ ID NO: 16)
NTSNLPS;
and
V_{L}-CDR3:
(SEQ ID NO: 17)
QQRSSYPLTF,

with:

X₁₂ being selected from Asn (N), Ser (S) and Gly (G).

In one embodiment, the antibody or binding fragment thereof comprises a LCVR which comprises at least one, preferably at least two, more preferably the following three CDRs:

V_{L}-CDR1:
(SEQ ID NO: 18)
RASSSVS-X₁₂-YMH;
V_{L}-CDR2:
(SEQ ID NO: 16)
NTSNLPS;
and
V_{L}-CDR3:
(SEQ ID NO: 17)
QQRSSYPLTF,

with:

X₁₂ being absent or being selected from Asn (N), Ser (S) and Gly (G).

In one embodiment, the antibody or binding fragment thereof comprises a LCVR which comprises the following three CDRs:

V_{L}-CDR1:
(SEQ ID NO: 18)
RASSSVS-X₁₂-YMH;
V_{L}-CDR2:
(SEQ ID NO: 16)
NTSNLPS;
and
V_{L}-CDR3:
(SEQ ID NO: 17)
QQRSSYPLTF,

with:

X₁₂ being absent or being selected from Asn (N), Ser (S) and Gly (G).

In one embodiment, the antibody or binding fragment thereof comprises a LCVR which comprises at least one, preferably at least two, more preferably the following three CDRs:

V_{L}-CDR1:
(SEQ ID NO: 18)
RASSSVSYMH;
V_{L}-CDR2:
(SEQ ID NO: 16)
NTSNLPS;
and
V_{L}-CDR3:
(SEQ ID NO: 17)
QQRSSYPLTF.

In one embodiment, the antibody or binding fragment thereof comprises a LCVR which comprises the following three CDRs:

V_{L}-CDR1:
(SEQ ID NO: 18)
RASSSVSYMH;
V_{L}-CDR2:
(SEQ ID NO: 16)
NTSNLPS;
and
V_{L}-CDR3:
(SEQ ID NO: 17)
QQRSSYPLTF.

In one embodiment, the antibody or binding fragment thereof comprises a LCVR which comprises at least one, preferably at least two, more preferably the following three CDRs:

V_{L}-CDR1:
(SEQ ID NO: 18)
RASSSVS-X₁₂-YMH;
V_{L}-CDR2:
(SEQ ID NO: 16)
NTSNLPS;
and
V_{L}-CDR3:
(SEQ ID NO: 17)
QQRSSYPLTF,

with:

X₁₂ being selected from Asn (N), Ser (S) and Gly (G).

In one embodiment, the antibody or binding fragment thereof comprises a LCVR which comprises the following three CDRs:

V_{L}-CDR1:
(SEQ ID NO: 18)
RASSSVS-X₁₂-YMH;
V_{L}-CDR2:
(SEQ ID NO: 16)
NTSNLPS;
and
V_{L}-CDR3:
(SEQ ID NO: 17)
QQRSSYPLTF,

with:

X₁₂ being selected from Asn (N), Ser (S) and Gly (G).

In one embodiment, the antibody or binding fragment thereof comprises a LCVR which comprises at least one, preferably at least two, more preferably the following three CDRs:

V_{L}-CDR1:
(SEQ ID NO: 18)
RASSSVS-X₁₂-YMH;
V_{L}-CDR2:
(SEQ ID NO: 19)
NTSNSPS;
and
V_{L}-CDR3:
(SEQ ID NO: 17)
QQRSSYPLTF,

with:

X₁₂ being absent or being selected from Asn (N), Ser (S) and Gly (G).

In one embodiment, the antibody or binding fragment thereof comprises a LCVR which comprises the following three CDRs:

V_{L}-CDR1:
(SEQ ID NO: 18)
RASSSVS-X₁₂-YMH;
V_{L}-CDR2:
(SEQ ID NO: 19)
NTSNSPS;
and
V_{L}-CDR3:
(SEQ ID NO: 17)
QQRSSYPLTF,

with:

X₁₂ being absent or being selected from Asn (N), Ser (S) and Gly (G).

In one embodiment, the antibody or binding fragment thereof comprises a LCVR which comprises at least one, preferably at least two, more preferably the following three CDRs:

V_{L}-CDR1:
(SEQ ID NO: 18)
RASSSVS-X₁₂-YMH;
V_{L}-CDR2:
(SEQ ID NO: 19)
NTSNSPS;
and
V_{L}-CDR3:
(SEQ ID NO: 17)
QQRSSYPLTF,

In one embodiment, the antibody or binding fragment thereof comprises a LCVR which comprises the following three CDRs:

V_{L}-CDR1:
(SEQ ID NO: 18)
RASSSVS-X₁₂-YMH;
V_{L}-CDR2:
(SEQ ID NO: 19)
NTSNSPS;
and
V_{L}-CDR3:
(SEQ ID NO: 17)
QQRSSYPLTF,

In one embodiment, the antibody or binding fragment thereof comprises a LCVR which comprises at least one, preferably at least two, more preferably the following three CDRs:

V_{L}-CDR1:
(SEQ ID NO: 18)
RASSSVS-X₁₂-YMH;
V_{L}-CDR2:
(SEQ ID NO: 19)
NTSNSPS;
and
V_{L}-CDR3:
(SEQ ID NO: 17)
QQRSSYPLTF,

with:

X₁₂ being selected from Asn (N), Ser (S) and Gly (G).

In one embodiment, the antibody or binding fragment thereof comprises a LCVR which comprises the following three CDRs:

V_{L}-CDR1:
(SEQ ID NO: 18)
RASSSVS-X₁₂-YMH;
V_{L}-CDR2:
(SEQ ID NO: 19)
NTSNSPS;
and
V_{L}-CDR3:
(SEQ ID NO: 17)
QQRSSYPLTF,

with:

X₁₂ being selected from Asn (N), Ser (S) and Gly (G).

In one embodiment, the antibody or binding fragment thereof comprises a LCVR which comprises at least one, preferably at least two, more preferably the following three CDRs:

V_{L}-CDR1:
(SEQ ID NO: 18)
RASSSVS-X₁₂-YMH;
V_{L}-CDR2:
(SEQ ID NO: 20)
ATSNLQS;
and
V_{L}-CDR3:
(SEQ ID NO: 17)
QQRSSYPLTF,

with:

X₁₂ being absent or being selected from Asn (N), Ser (S) and Gly (G).

In one embodiment, the antibody or binding fragment thereof comprises a LCVR which comprises the following three CDRs:

V_{L}-CDR1:
(SEQ ID NO: 18)
RASSSVS-X₁₂-YMH;
V_{L}-CDR2:
(SEQ ID NO: 20)
ATSNLQS;
and
V_{L}-CDR3:
(SEQ ID NO: 17)
QQRSSYPLTF,

with:

X₁₂ being absent or being selected from Asn (N), Ser (S) and Gly (G).

In one embodiment, the antibody or binding fragment thereof comprises a LCVR which comprises at least one, preferably at least two, more preferably the following three CDRs:

V_{L}-CDR1:
(SEQ ID NO: 18)
RASSSVS-X₁₂-YMH;
V_{L}-CDR2:
(SEQ ID NO: 20)
ATSNLQS;
and
V_{L}-CDR3:
(SEQ ID NO: 17)
QQRSSYPLTF.

In one embodiment, the antibody or binding fragment thereof comprises a LCVR which comprises the following three CDRs:

V_{L}-CDR1:
(SEQ ID NO: 18)
RASSSVS-X₁₂-YMH;
V_{L}-CDR2:
(SEQ ID NO: 20)
ATSNLQS;
and
V_{L}-CDR3:
(SEQ ID NO: 17)
QQRSSYPLTF.

In one embodiment, the antibody or binding fragment thereof comprises a LCVR which comprises at least one, preferably at least two, more preferably the following three CDRs:

V_{L}-CDR1:
(SEQ ID NO: 18)
RASSSVS-X₁₂-YMH;
V_{L}-CDR2:
(SEQ ID NO: 20)
ATSNLQS;
and
V_{L}-CDR3:
(SEQ ID NO: 17)
QQRSSYPLTF,

with:

X₁₂ being selected from Asn (N), Ser (S) and Gly (G).

In one embodiment, the antibody or binding fragment thereof comprises a LCVR which comprises the following three CDRs:

V_{L}-CDR1:
(SEQ ID NO: 18)
RASSSVS-X₁₂-YMH;
V_{L}-CDR2:
(SEQ ID NO: 20)
ATSNLQS;
and
V_{L}-CDR3:
(SEQ ID NO: 17)
QQRSSYPLTF,

with:

X₁₂ being selected from Asn (N), Ser (S) and Gly (G).

In one embodiment, the antibody or binding fragment thereof comprises a LCVR which comprises at least one, preferably at least two, more preferably the following three CDRs:

V_{L}-CDR1:
(SEQ ID NO: 18)
RASSSVS-X₁₂-YMH;
V_{L}-CDR2:
(SEQ ID NO: 19)
NTSNSPS;
and
V_{L}-CDR3:
(SEQ ID NO: 21)
HQRSSYPLTF,

with:

X₁₂ being absent or being selected from Asn (N), Ser (S) and Gly (G).

In one embodiment, the antibody or binding fragment thereof comprises a LCVR which comprises the following three CDRs:

V_{L}-CDR1:
(SEQ ID NO: 18)
RASSSVS-X₁₂-YMH;
V_{L}-CDR2:
(SEQ ID NO: 19)
NTSNSPS;
and
V_{L}-CDR3:
(SEQ ID NO: 21)
HQRSSYPLTF,

with:

X₁₂ being absent or being selected from Asn (N), Ser (S) and Gly (G).

In one embodiment, the antibody or binding fragment thereof comprises a LCVR which comprises at least one, preferably at least two, more preferably the following three CDRs:

V_{L}-CDR1:
(SEQ ID NO: 18)
RASSSVSYMH;
V_{L}-CDR2:
(SEQ ID NO: 19)
NTSNSPS;
and
V_{L}-CDR3:
(SEQ ID NO: 21)
HQRSSYPLTF.

In one embodiment, the antibody or binding fragment thereof comprises a LCVR which comprises the following three CDRs:

V_{L}-CDR1:
(SEQ ID NO: 18)
RASSSVSYMH;
V_{L}-CDR2:
(SEQ ID NO: 19)
NTSNSPS;
and
V_{L}-CDR3:
(SEQ ID NO: 21)
HQRSSYPLTF.

In one embodiment, the antibody or binding fragment thereof comprises a LCVR which comprises at least one, preferably at least two, more preferably the following three CDRs:

V_{L}-CDR1:
(SEQ ID NO: 18)
RASSSVS-X₁₂₋YMH;
V_{L}-CDR2:
(SEQ ID NO: 19)
NTSNSPS;
and
V_{L}-CDR3:
(SEQ ID NO: 21)
HQRSSYPLTF,

with:

X₁₂ being selected from Asn (N), Ser (S) and Gly (G).

In one embodiment, the antibody or binding fragment thereof comprises a LCVR which comprises the following three CDRs:

V_{L}-CDR1:
(SEQ ID NO: 18)
RASSSVS-X₁₂-YMH;
V_{L}-CDR2:
(SEQ ID NO: 19)
NTSNSPS;
and
V_{L}-CDR3:
(SEQ ID NO: 21)
HQRSSYPLTF,

with:

X₁₂ being selected from Asn (N), Ser (S) and Gly (G).

In one embodiment, the antibody or binding fragment thereof comprises a LCVR which comprises at least one, preferably at least two, more preferably the following three CDRs:

V_{L}-CDR1:
(SEQ ID NO: 18)
RASSSVS-X₁₂-YMH;
V_{L}-CDR2:
(SEQ ID NO: 22)
NTSNRAT;
and
V_{L}-CDR3:
(SEQ ID NO: 17)
QQRSSYPLTF,

with:

X₁₂ being absent or being selected from Asn (N), Ser (S) and Gly (G).

In one embodiment, the antibody or binding fragment thereof comprises a LCVR which comprises the following three CDRs:

V_{L}-CDR1:
(SEQ ID NO: 18)
RASSSVS-X₁₂-YMH;
V_{L}-CDR2:
(SEQ ID NO: 22)
NTSNRAT;
and
V_{L}-CDR3:
(SEQ ID NO: 17)
QQRSSYPLTF,

with:

X₁₂ being absent or being selected from Asn (N), Ser (S) and Gly (G).

In one embodiment, the antibody or binding fragment thereof comprises a LCVR which comprises at least one, preferably at least two, more preferably the following three CDRs:

V_{L}-CDR1:
(SEQ ID NO: 18)
RASSSVSYMH;
V_{L}-CDR2:
(SEQ ID NO: 22)
NTSNRAT;
and
V_{L}-CDR3:
(SEQ ID NO: 17)
QQRSSYPLTF.

In one embodiment, the antibody or binding fragment thereof comprises a LCVR which comprises the following three CDRs:

V_{L}-CDR1:
(SEQ ID NO: 18)
RASSSVSYMH;
V_{L}-CDR2:
(SEQ ID NO: 22)
NTSNRAT;
and
V_{L}-CDR3:
(SEQ ID NO: 17)
QQRSSYPLTF.

In one embodiment, the antibody or binding fragment thereof comprises a LCVR which comprises at least one, preferably at least two, more preferably the following three CDRs:

V_{L}-CDR1:
(SEQ ID NO: 18)
RASSSVS-X₁₂-YMH;
V_{L}-CDR2:
(SEQ ID NO: 22)
NTSNRAT;
and
V_{L}-CDR3:
(SEQ ID NO: 17)
QQRSSYPLTF,

with:

X₁₂ being selected from Asn (N), Ser (S) and Gly (G).

In one embodiment, the antibody or binding fragment thereof comprises a LCVR which comprises the following three CDRs:

V_{L}-CDR1:
(SEQ ID NO: 18)
RASSSVS-X₁₂-YMH;
V_{L}-CDR2:
(SEQ ID NO: 22)
NTSNRAT;
and
V_{L}-CDR3:
(SEQ ID NO: 17)
QQRSSYPLTF,

with:

X₁₂ being selected from Asn (N), Ser (S) and Gly (G).

In one embodiment, the antibody or binding fragment thereof comprises a LCVR which comprises at least one, preferably at least two, more preferably the following three CDRs:

V_{L}-CDR1:
(SEQ ID NO: 18)
RASSSVS-X₁₂-YMH;
V_{L}-CDR2:
(SEQ ID NO: 111)
ATSNLPS;
and
V_{L}-CDR3:
(SEQ ID NO: 17)
QQRSSYPLTF,

with:

X₁₂ being absent or being selected from Asn (N), Ser (S) and Gly (G).

In one embodiment, the antibody or binding fragment thereof comprises a LCVR which comprises the following three CDRs:

V_{L}-CDR1:
(SEQ ID NO: 18)
RASSSVS-X₁₂-YMH;
V_{L}-CDR2:
(SEQ ID NO: 111)
ATSNLPS;
and
V_{L}-CDR3:
(SEQ ID NO: 17)
QQRSSYPLTF,

with:

X₁₂ being absent or being selected from Asn (N), Ser (S) and Gly (G).

In one embodiment, the antibody or binding fragment thereof comprises a LCVR which comprises at least one, preferably at least two, more preferably the following three CDRs:

V_{L}-CDR1:
(SEQ ID NO: 18)
RASSSVSYMH;
V_{L}-CDR2:
(SEQ ID NO: 111)
ATSNLPS;
and
V_{L}-CDR3:
(SEQ ID NO: 17)
QQRSSYPLTF.

In one embodiment, the antibody or binding fragment thereof comprises a LCVR which comprises the following three CDRs:

V_{L}-CDR1:
(SEQ ID NO: 18)
RASSSVSYMH;
V_{L}-CDR2:
(SEQ ID NO: 111)
ATSNLPS;
and
V_{L}-CDR3:
(SEQ ID NO: 17)
QQRSSYPLTF.

In one embodiment, the antibody or binding fragment thereof comprises a LCVR which comprises at least one, preferably at least two, more preferably the following three CDRs:

V_{L}-CDR1:
(SEQ ID NO: 18)
RASSSVS-X₁₂-YMH;
V_{L}-CDR2:
(SEQ ID NO: 111)
ATSNLPS;
and
V_{L}-CDR3:
(SEQ ID NO: 17)
QQRSSYPLTF,

with:

X₁₂ being selected from Asn (N), Ser (S) and Gly (G).

In one embodiment, the antibody or binding fragment thereof comprises a LCVR which comprises the following three CDRs:

V_{L}-CDR1:
(SEQ ID NO: 18)
RASSSVS-X₁₂-YMH;
V_{L}-CDR2:
(SEQ ID NO: 111)
ATSNLPS;
and
V_{L}-CDR3:
(SEQ ID NO: 17)
QQRSSYPLTF,

with:

X₁₂ being selected from Asn (N), Ser (S) and Gly (G).

In one embodiment, the antibody or binding fragment thereof comprises a LCVR which comprises at least one, preferably at least two, more preferably the following three CDRs:

V_{L}-CDR1:
(SEQ ID NO: 15)
SASSSVS-X₁₂-YMH;
V_{L}-CDR2:
(SEQ ID NO: 120)
NTANLPS;
and
V_{L}-CDR3:
(SEQ ID NO: 17)
QQRSSYPLTF,

with:

X₁₂ being absent or being selected from Asn (N), Ser (S) and Gly (G).

In one embodiment, the antibody or binding fragment thereof comprises a LCVR which comprises the following three CDRs:

V_{L}-CDR1:
(SEQ ID NO: 15)
SASSSVS-X₁₂-YMH;
V_{L}-CDR2:
(SEQ ID NO: 120)
NTANLPS;
and
V_{L}-CDR3:
(SEQ ID NO: 17)
QQRSSYPLTF,

with:

X₁₂ being absent or being selected from Asn (N), Ser (S) and Gly (G).

In one embodiment, the antibody or binding fragment thereof comprises a LCVR which comprises at least one, preferably at least two, more preferably the following three CDRs:

V_{L}-CDR1:
(SEQ ID NO: 15)
SASSSVS-X₁₂-YMH;
V_{L}-CDR2:
(SEQ ID NO: 120)
NTANLPS;
and
V_{L}-CDR3:
(SEQ ID NO: 17)
QQRSSYPLTF,

In one embodiment, the antibody or binding fragment thereof comprises a LCVR which comprises the following three CDRs:

V_{L}-CDR1:
(SEQ ID NO: 15)
SASSSVS-X₁₂-YMH;
V_{L}-CDR2:
(SEQ ID NO: 120)
NTANLPS;
and
V_{L}-CDR3:
(SEQ ID NO: 17)
QQRSSYPLTF,

In one embodiment, the antibody or binding fragment thereof comprises a LCVR which comprises at least one, preferably at least two, more preferably the following three CDRs:

V_{L}-CDR1:
(SEQ ID NO: 15)
SASSSVS-X₁₂-YMH;
V_{L}-CDR2:
(SEQ ID NO: 120)
NTANLPS;
and
V_{L}-CDR3:
(SEQ ID NO: 17)
QQRSSYPLTF,

with:

X₁₂ being selected from Asn (N), Ser (S) and Gly (G).

In one embodiment, the antibody or binding fragment thereof comprises a LCVR which comprises the following three CDRs:

V_{L}-CDR1:
(SEQ ID NO: 15)
SASSSVS-Xi2-YMH;
V_{L}-CDR2:
(SEQ ID NO: 120)
NTANLPS;
and
V_{L}-CDR3:
(SEQ ID NO: 17)
QQRSSYPLTF,

with:

X₁₂ being selected from Asn (N), Ser (S) and Gly (G).

In one embodiment, the antibody or binding fragment thereof comprises a LCVR which comprises at least one, preferably at least two, more preferably the following three CDRs:

V_{L}-CDR1:
(SEQ ID NO: 18)
RASSSVS-X₁₂-YMH;
V_{L}-CDR2:
(SEQ ID NO: 127)
X₁₃-TSNLPS;
and
V_{L}-CDR3:
(SEQ ID NO: 17)
QQRSSYPLTF,

with:

X₁₂ being absent or being selected from Asn (N), Ser (S) and Gly (G), and

X₁₃ being any amino acid but Ala (A) or Asn (N).

In one embodiment, the antibody or binding fragment thereof comprises a LCVR which comprises the following three CDRs:

V_{L}-CDR1:
(SEQ ID NO: 18)
RASSSVS-X₁₂-YMH;
V_{L}-CDR2:
(SEQ ID NO: 127)
X₁₃-TSNLPS;
and
V_{L}-CDR3:
(SEQ ID NO: 17)
QQRSSYPLTF,

with:

X₁₂ being absent or being selected from Asn (N), Ser (S) and Gly (G), and

X₁₃ being any amino acid but Ala (A) or Asn (N).

In one embodiment, the antibody or binding fragment thereof comprises a LCVR which comprises at least one, preferably at least two, more preferably the following three CDRs:

V_{L}-CDR1:
(SEQ ID NO: 18)
RASSSVSYMH;
V_{L}-CDR2:
(SEQ ID NO: 127)
X₁₃-TSNLPS;
and
V_{L}-CDR3:
(SEQ ID NO: 17)
QQRSSYPLTF.

with:

X₁₃ being any amino acid but Ala (A) or Asn (N).

In one embodiment, the antibody or binding fragment thereof comprises a LCVR which comprises the following three CDRs:

V_{L}-CDR1:
(SEQ ID NO: 18)
RASSSVSYMH;
V_{L}-CDR2:
(SEQ ID NO: 127)
X₁₃-TSNLPS;
and
V_{L}-CDR3:
(SEQ ID NO: 17)
QQRSSYPLTF.

with:

X₁₃ being any amino acid but Ala (A) or Asn (N).

In one embodiment, the antibody or binding fragment thereof comprises a LCVR which comprises at least one, preferably at least two, more preferably the following three CDRs:

V_{L}-CDR1:
(SEQ ID NO: 18)
RASSSVS-X₁₂-YMH;
V_{L}-CDR2:
(SEQ ID NO: 127)
X₁₃-TSNLPS;
and
V_{L}-CDR3:
(SEQ ID NO: 17)
QQRSSYPLTF,

with:

X₁₂ being absent or being selected from Asn (N), Ser (S) and Gly (G), and

X₁₃ being any amino acid but Ala (A) or Asn (N).

In one embodiment, the antibody or binding fragment thereof comprises a LCVR which comprises the following three CDRs:

V_{L}-CDR1:
(SEQ ID NO: 18)
RASSSVS-X₁₂-YMH;
V_{L}-CDR2:
(SEQ ID NO: 127)
X₁₃-TSNLPS;
and
V_{L}-CDR3:
(SEQ ID NO: 17)
QQRSSYPLTF,

with:

X₁₂ being absent or being selected from Asn (N), Ser (S) and Gly (G), and

X₁₃ being any amino acid but Ala (A) or Asn (N).

In one embodiment, the antibody or binding fragment thereof comprises:
a HCVR which comprises at least one, preferably at least two, more preferably the following three CDRs:

VH-CDR1:
(SEQ ID NO: 1)
NYYIG;
VH-CDR2:
(SEQ ID NO: 2)
X₁-IF-X₂-GG-X₃-Y-X₄-N-X₅-X₆-X₇-X₈-X₉-X₁₀-G;
and
VH-CDR3:
(SEQ ID NO: 3)
RNFDY;

and
a LCVR which comprises at least one, preferably at least two, more preferably the following three CDRs:

V_{L}-CDR1:
(SEQ ID NO: 12)
X₁₁-ASSSVS-X₁₂-YMH;
V_{L}-CDR2:
(SEQ ID NO: 13)
X₁₃-TSN-X₁₄-X₁₅-X₁₆;
and
V_{L}-CDR3:
(SEQ ID NO: 14)
X₁₇-QRSSYPLTF,

with:

X₁ being selected from Asp (D), Ile (I) and Arg (R);

X₂ being selected from Pro (P) and Ser (S);

X₃ being selected from Asp (D), Ser (S) and Gly (G);

X₄ being selected from Ala (A) and Thr (T);

X₅ being selected from Ser (S) and Tyr (Y);

X₆ being selected from Asn (N), Ala (A) and Ser (S);

X₇ being selected from Glu (E), Asp (D), Pro (P) and Gln (Q);

X₈ being selected from Lys (K) and Ser (S);

X₉ being selected from Phe (F) and Val (V);

X₁₀ being selected from Lys (K) and Gln (Q);

X₁₁ being selected from Ser (S) and Arg (R);

X₁₂ being absent or being selected from Asn (N), Ser (S) and Gly (G);

X₁₃ being selected from Asn (N) and Ala (A); or X₁₃ being any amino acid but Ala (A) or Asn (N);

X₁₄ being selected from Leu (L), Ser (S) and Arg (R);

X₁₅ being selected from Pro (P), Ala (A) and Gln (Q);

X₁₆ being selected from Ser (S) and Thr (T); and

X₁₇ being selected from Gln (Q) and His (H).

In one embodiment, the antibody or binding fragment thereof comprises:
a HCVR which comprises the following three CDRs:

V_{H}-CDR1:
(SEQ ID NO: 1)
NYYIG;
V_{H}-CDR2:
(SEQ ID NO: 2)
X₁-IF-X₂-GG-X₃-Y-X₄-N-X₅-X₆-X₇-X₈-X₉-X₁₀-G;
and
V_{H}-CDR3:
(SEQ ID NO: 3)
RNFDY;

and
a LCVR which comprises the following three CDRs:

V_{L}-CDR1:
(SEQ ID NO: 12)
X₁₁-ASSSVS-X₁₂-YMH;
V_{L}-CDR2:
(SEQ ID NO: 13)
X₁₃-TSN-X₁₄-X₁₅-X₁₆;
and
V_{L}-CDR3:
(SEQ ID NO: 14)
X₁₇-QRSSYPLTF,

with:

X₁ being selected from Asp (D), Ile (I) and Arg (R);

X₂ being selected from Pro (P) and Ser (S);

X₃ being selected from Asp (D), Ser (S) and Gly (G);

X₄ being selected from Ala (A) and Thr (T);

X₅ being selected from Ser (S) and Tyr (Y);

X₆ being selected from Asn (N), Ala (A) and Ser (S);

X₇ being selected from Glu (E), Asp (D), Pro (P) and Gln (Q);

X₈ being selected from Lys (K) and Ser (S);

X₉ being selected from Phe (F) and Val (V);

X₁₀ being selected from Lys (K) and Gln (Q);

X₁₁ being selected from Ser (S) and Arg (R);

X₁₂ being absent or being selected from Asn (N), Ser (S) and Gly (G);

X₁₃ being selected from Asn (N) and Ala (A); or X₁₃ being any amino acid but Ala (A) or Asn (N);

X₁₄ being selected from Leu (L), Ser (S) and Arg (R);

X₁₅ being selected from Pro (P), Ala (A) and Gln (Q);

X₁₆ being selected from Ser (S) and Thr (T); and

X₁₇ being selected from Gln (Q) and His (H).

In one embodiment, the antibody or binding fragment thereof comprises a combination of (i) at least one, preferably at least two, more preferably three HCVR's CDRs and (ii) at least one, preferably at least two, more preferably three LCVR's CDRs, said combination being as defined in Table 2.

In one embodiment, the antibody or binding fragment thereof comprises a combination of (i) three HCVR's CDRs and (ii) three LCVR's CDRs, said combination being as defined in Table 2.

TABLE 2. Preferred combinations of HCVR's and LCVR's CDRs. The CDRs are defined by their SEQ ID NOs (with, wherever applicable, X₁₂ being absent or being

TABLE 2
Preferred combinations of HCVR's and LCVR's CDRs. The CDRs are
defined by their SEQ ID NOs (with, wherever applicable, X₁₂ being
absent or being selected from Asn (N), Ser (S) and Gly (G); and X₁₃
being any amino acid but Ala (A) or Asn (N)).

| CDRs' | | | | | | |
|---|---|---|---|---|---|---|
| combination | V_{H}- | V_{H}- | V_{H}- | V_{L}- | V_{L}- | V_{L}- |
| # | CDR1 | CDR2 | CDR3 | CDR1 | CDR2 | CDR3 |
| #1 | 1 | 4 | 3 | 15 | 16 | 17 |
| #2 | 1 | 4 | 3 | 18 | 16 | 17 |
| #3 | 1 | 4 | 3 | 18 | 19 | 17 |
| #4 | 1 | 4 | 3 | 18 | 20 | 17 |
| #5 | 1 | 4 | 3 | 18 | 19 | 21 |
| #6 | 1 | 4 | 3 | 18 | 22 | 17 |
| #7 | 1 | 5 | 3 | 15 | 16 | 17 |
| #8 | 1 | 5 | 3 | 18 | 16 | 17 |
| #9 | 1 | 5 | 3 | 18 | 19 | 17 |
| #10 | 1 | 5 | 3 | 18 | 20 | 17 |
| #11 | 1 | 5 | 3 | 18 | 19 | 21 |
| #12 | 1 | 5 | 3 | 18 | 22 | 17 |
| #13 | 1 | 6 | 3 | 15 | 16 | 17 |
| #14 | 1 | 6 | 3 | 18 | 16 | 17 |
| #15 | 1 | 6 | 3 | 18 | 19 | 17 |
| #16 | 1 | 6 | 3 | 18 | 20 | 17 |
| #17 | 1 | 6 | 3 | 18 | 19 | 21 |
| #18 | 1 | 6 | 3 | 18 | 22 | 17 |
| #19 | 1 | 7 | 3 | 15 | 16 | 17 |
| #20 | 1 | 7 | 3 | 18 | 16 | 17 |
| #21 | 1 | 7 | 3 | 18 | 19 | 17 |
| #22 | 1 | 7 | 3 | 18 | 20 | 17 |
| #23 | 1 | 7 | 3 | 18 | 19 | 21 |
| #24 | 1 | 7 | 3 | 18 | 22 | 17 |
| #25 | 1 | 8 | 3 | 15 | 16 | 17 |
| #26 | 1 | 8 | 3 | 18 | 16 | 17 |
| #27 | 1 | 8 | 3 | 18 | 19 | 17 |
| #28 | 1 | 8 | 3 | 18 | 20 | 17 |
| #29 | 1 | 8 | 3 | 18 | 19 | 21 |
| #30 | 1 | 8 | 3 | 18 | 22 | 17 |
| #31 | 1 | 9 | 3 | 15 | 16 | 17 |
| #32 | 1 | 9 | 3 | 18 | 16 | 17 |
| #33 | 1 | 9 | 3 | 18 | 19 | 17 |
| #34 | 1 | 9 | 3 | 18 | 20 | 17 |
| #35 | 1 | 9 | 3 | 18 | 19 | 21 |
| #36 | 1 | 9 | 3 | 18 | 22 | 17 |
| #37 | 1 | 10 | 3 | 15 | 16 | 17 |
| #38 | 1 | 10 | 3 | 18 | 16 | 17 |
| #39 | 1 | 10 | 3 | 18 | 19 | 17 |
| #40 | 1 | 10 | 3 | 18 | 20 | 17 |
| #41 | 1 | 10 | 3 | 18 | 19 | 21 |
| #42 | 1 | 10 | 3 | 18 | 22 | 17 |
| #43 | 1 | 11 | 3 | 15 | 16 | 17 |
| #44 | 1 | 11 | 3 | 18 | 16 | 17 |
| #45 | 1 | 11 | 3 | 18 | 19 | 17 |
| #46 | 1 | 11 | 3 | 18 | 20 | 17 |
| #47 | 1 | 11 | 3 | 18 | 19 | 21 |
| #48 | 1 | 11 | 3 | 18 | 22 | 17 |
| #49 | 1 | 100 | 3 | 15 | 16 | 17 |
| #50 | 1 | 100 | 3 | 18 | 16 | 17 |
| #51 | 1 | 100 | 3 | 18 | 19 | 17 |
| #52 | 1 | 100 | 3 | 18 | 20 | 17 |
| #53 | 1 | 100 | 3 | 18 | 19 | 21 |
| #54 | 1 | 100 | 3 | 18 | 22 | 17 |
| #55 | 1 | 4 | 3 | 18 | 111 | 17 |
| #56 | 1 | 5 | 3 | 18 | 111 | 17 |
| #57 | 1 | 6 | 3 | 18 | 111 | 17 |
| #58 | 1 | 7 | 3 | 18 | 111 | 17 |
| #59 | 1 | 8 | 3 | 18 | 111 | 17 |
| #60 | 1 | 9 | 3 | 18 | 111 | 17 |
| #61 | 1 | 10 | 3 | 18 | 111 | 17 |
| #62 | 1 | 11 | 3 | 18 | 111 | 17 |
| #63 | 1 | 100 | 3 | 18 | 111 | 17 |
| #64 | 1 | 116 | 3 | 15 | 16 | 17 |
| #65 | 1 | 116 | 3 | 18 | 16 | 17 |
| #66 | 1 | 116 | 3 | 18 | 19 | 17 |
| #67 | 1 | 116 | 3 | 18 | 20 | 17 |
| #68 | 1 | 116 | 3 | 18 | 19 | 21 |
| #69 | 1 | 116 | 3 | 18 | 22 | 17 |
| #70 | 1 | 116 | 3 | 18 | 111 | 17 |
| #71 | 1 | 117 | 3 | 15 | 16 | 17 |
| #72 | 1 | 117 | 3 | 18 | 16 | 17 |
| #73 | 1 | 117 | 3 | 18 | 19 | 17 |
| #74 | 1 | 117 | 3 | 18 | 20 | 17 |
| #75 | 1 | 117 | 3 | 18 | 19 | 21 |
| #76 | 1 | 117 | 3 | 18 | 22 | 17 |
| #77 | 1 | 117 | 3 | 18 | 111 | 17 |
| #78 | 1 | 118 | 3 | 15 | 16 | 17 |
| #79 | 1 | 118 | 3 | 18 | 16 | 17 |
| #80 | 1 | 118 | 3 | 18 | 19 | 17 |
| #81 | 1 | 118 | 3 | 18 | 20 | 17 |
| #82 | 1 | 118 | 3 | 18 | 19 | 21 |
| #83 | 1 | 118 | 3 | 18 | 22 | 17 |
| #84 | 1 | 118 | 3 | 18 | 111 | 17 |
| #85 | 1 | 119 | 3 | 15 | 16 | 17 |
| #86 | 1 | 119 | 3 | 18 | 16 | 17 |
| #87 | 1 | 119 | 3 | 18 | 19 | 17 |
| #88 | 1 | 119 | 3 | 18 | 20 | 17 |
| #89 | 1 | 119 | 3 | 18 | 19 | 21 |
| #90 | 1 | 119 | 3 | 18 | 22 | 17 |
| #91 | 1 | 119 | 3 | 18 | 111 | 17 |
| #92 | 1 | 4 | 3 | 15 | 120 | 17 |
| #93 | 1 | 5 | 3 | 15 | 120 | 17 |
| #94 | 1 | 6 | 3 | 15 | 120 | 17 |
| #95 | 1 | 7 | 3 | 15 | 120 | 17 |
| #96 | 1 | 8 | 3 | 15 | 120 | 17 |
| #97 | 1 | 9 | 3 | 15 | 120 | 17 |
| #98 | 1 | 10 | 3 | 15 | 120 | 17 |
| #99 | 1 | 11 | 3 | 15 | 120 | 17 |
| #100 | 1 | 100 | 3 | 15 | 120 | 17 |
| #101 | 1 | 116 | 3 | 15 | 120 | 17 |
| #102 | 1 | 117 | 3 | 15 | 120 | 17 |
| #103 | 1 | 118 | 3 | 15 | 120 | 17 |
| #104 | 1 | 119 | 3 | 15 | 120 | 17 |
| #105 | 1 | 4 | 3 | 15 | 127 | 17 |
| #106 | 1 | 5 | 3 | 15 | 127 | 17 |
| #107 | 1 | 6 | 3 | 15 | 127 | 17 |
| #108 | 1 | 7 | 3 | 15 | 127 | 17 |
| #109 | 1 | 8 | 3 | 15 | 127 | 17 |
| #110 | 1 | 9 | 3 | 15 | 127 | 17 |
| #111 | 1 | 10 | 3 | 15 | 127 | 17 |
| #112 | 1 | 11 | 3 | 15 | 127 | 17 |
| #113 | 1 | 100 | 3 | 15 | 127 | 17 |
| #114 | 1 | 116 | 3 | 15 | 127 | 17 |
| #115 | 1 | 117 | 3 | 15 | 127 | 17 |
| #116 | 1 | 118 | 3 | 15 | 127 | 17 |
| #117 | 1 | 119 | 3 | 15 | 127 | 17 |

In one embodiment, any of V_{H}-CDR1, V_{H}-CDR2, V_{H}-CDR3, V_{L}-CDR1, V_{L}-CDR2 and/or V_{L}-CDR3 as defined hereinabove can be characterized as having 1, 2, 3, 4, 5 or more amino acids being substituted by a different amino acid.

In one embodiment, any of V_{H}-CDR1, V_{H}-CDR2, V_{H}-CDR3, V_{L}-CDR1, V_{L}-CDR2 and/or V_{L}-CDR3 as defined hereinabove can be characterized as having an amino acid sequence that shares at least 70%, 75%, 80%, 85%, 90%, 95%, 96%, 97%, 98%, 99% or more of identity with the particular CDR or sets of CDRs as defined hereinabove.

In one embodiment, the antibody or binding fragment thereof comprises a combination of (i) at least one, preferably at least two, more preferably three HCVR's CDRs and (ii) at least one, preferably at least two, more preferably three LCVR's CDRs, said combination being selected from combinations #1, #2, #7, #14, #20, #26, #49, #50, #63, #65, #72, #79, #86 and #92 as defined in Table 2.

In one embodiment, the antibody or binding fragment thereof comprises a combination of (i) three HCVR's CDRs and (ii) three LCVR's CDRs, said combination being selected from combinations #1, #2, #7, #14, #20, #26, #49, #50, #63, #65, #72, #79, #86 and #92 as defined in Table 2.

In one embodiment, the antibody or binding fragment thereof comprises:
a HCVR which comprises the following three CDRs:
   V_{H}-CDR1 of sequence SEQ ID NO: 1;
   V_{H}-CDR2 selected from the group comprising or consisting of sequences SEQ ID NOs: 4, 5, 6, 7, 8, 100, 116, 117, 118 and 119; and
   V_{H}-CDR3 of sequence SEQ ID NO: 3; and
a LCVR which comprises the following three CDRs:
   V_{L}-CDR1 selected from the group comprising or consisting of sequences SEQ ID NOs: 15 and 18, wherein X₁₂ is absent or is selected from Asn (N), Ser (S) and Gly (G), preferably X₁₂ is absent;
   V_{L}-CDR2 selected from the group comprising or consisting of sequences SEQ ID NOs: 16, 111 and 120; and
   V_{L}-CDR3 of sequence SEQ ID NO: 17.

In one embodiment, the antibody or binding fragment thereof comprises:
a HCVR which comprises the following three CDRs:
   V_{H}-CDR1 of sequence SEQ ID NO: 1;
   V_{H}-CDR2 selected from the group comprising or consisting of sequences SEQ ID NOs: 4 and 5; and
   V_{H}-CDR3 of sequence SEQ ID NO: 3; and
a LCVR which comprises the following three CDRs:
   V_{L}-CDR1 of sequence SEQ ID NO: 15, wherein X₁₂ is absent or is selected from Asn (N), Ser (S) and Gly (G), preferably X₁₂ is absent;
   V_{L}-CDR2 of sequence SEQ ID NO: 16; and
   V_{L}-CDR3 of sequence SEQ ID NO: 17.

In one embodiment, the antibody or binding fragment thereof comprises:
a HCVR which comprises the following three CDRs:
   V_{H}-CDR1 of sequence SEQ ID NO: 1;
   V_{H}-CDR2 selected from the group comprising or consisting of sequences SEQ ID NOs: 4, 5, 6 and 100; and
   V_{H}-CDR3 of sequence SEQ ID NO: 3; and
a LCVR which comprises the following three CDRs:
   V_{L}-CDR1 selected from the group comprising or consisting of sequences SEQ ID NOs: 15 and 18, wherein X₁₂ is absent or is selected from Asn (N), Ser (S) and Gly (G), preferably X₁₂ is absent;
   V_{L}-CDR2 of sequence SEQ ID NO: 16; and
   V_{L}-CDR3 of sequence SEQ ID NO: 17.

In one embodiment, the antibody or binding fragment thereof comprises:
a HCVR which comprises the following three CDRs:
   V_{H}-CDR1 of sequence SEQ ID NO: 1;
   V_{H}-CDR2 selected from the group comprising or consisting of sequences SEQ ID NOs: 4, 6 and 100; and
   V_{H}-CDR3 of sequence SEQ ID NO: 3; and
a LCVR which comprises the following three CDRs:
   V_{L}-CDR1 selected from the group comprising or consisting of sequences SEQ ID NOs: 15 and 18, wherein X₁₂ is absent or is selected from Asn (N), Ser (S) and Gly (G), preferably X₁₂ is absent;
   V_{L}-CDR2 of sequence SEQ ID NO: 16; and
   V_{L}-CDR3 of sequence SEQ ID NO: 17.

In one embodiment, the antibody or binding fragment thereof comprises a combination of (i) at least one, preferably at least two, more preferably three HCVR's CDRs and (ii) at least one, preferably at least two, more preferably three LCVR's CDRs, said combination being combination #1 as defined in Table 2.

In one embodiment, the antibody or binding fragment thereof comprises a combination of (i) three HCVR's CDRs and (ii) three LCVR's CDRs, said combination being combination #1 as defined in Table 2.

In one embodiment, the antibody or binding fragment thereof comprises a combination of (i) three HCVR's CDRs set forth as SEQ ID NOs: 1, 4 and 3; and (ii) three LCVR's CDRs set forth as SEQ ID NOs: 15, 16 and 17; wherein X₁₂ in SEQ ID NOs: 15 is absent or is selected from Asn (N), Ser (S) and Gly (G), preferably X₁₂ is absent.

In one embodiment, the antibody or binding fragment thereof comprises a combination of (i) at least one, preferably at least two, more preferably three HCVR's CDRs and (ii) at least one, preferably at least two, more preferably three LCVR's CDRs, said combination being combination #2 as defined in Table 2.

In one embodiment, the antibody or binding fragment thereof comprises a combination of (i) three HCVR's CDRs and (ii) three LCVR's CDRs, said combination being combination #2 as defined in Table 2.

In one embodiment, the antibody or binding fragment thereof comprises a combination of (i) three HCVR's CDRs set forth as SEQ ID NOs: 1, 4 and 3; and (ii) three LCVR's CDRs set forth as SEQ ID NOs: 18, 16 and 17; wherein X₁₂ in SEQ ID NOs: 18 is absent or is selected from Asn (N), Ser (S) and Gly (G), preferably X₁₂ is absent.

In one embodiment, the antibody or binding fragment thereof comprises a combination of (i) at least one, preferably at least two, more preferably three HCVR's CDRs and (ii) at least one, preferably at least two, more preferably three LCVR's CDRs, said combination being combination #7 as defined in Table 2.

In one embodiment, the antibody or binding fragment thereof comprises a combination of (i) three HCVR's CDRs and (ii) three LCVR's CDRs, said combination being combination #7 as defined in Table 2.

In one embodiment, the antibody or binding fragment thereof comprises a combination of (i) three HCVR's CDRs set forth as SEQ ID NOs: 1, 5 and 3; and (ii) three LCVR's CDRs set forth as SEQ ID NOs: 15, 16 and 17; wherein X₁₂ in SEQ ID NOs: 15 is absent or is selected from Asn (N), Ser (S) and Gly (G), preferably X₁₂ is absent.

In one embodiment, the antibody or binding fragment thereof comprises a combination of (i) at least one, preferably at least two, more preferably three HCVR's CDRs and (ii) at least one, preferably at least two, more preferably three LCVR's CDRs, said combination being combination #14 as defined in Table 2.

In one embodiment, the antibody or binding fragment thereof comprises a combination of (i) three HCVR's CDRs and (ii) three LCVR's CDRs, said combination being combination #14 as defined in Table 2.

In one embodiment, the antibody or binding fragment thereof comprises a combination of (i) three HCVR's CDRs set forth as SEQ ID NOs: 1, 6 and 3; and (ii) three LCVR's CDRs set forth as SEQ ID NOs: 18, 16 and 17; wherein X₁₂ in SEQ ID NOs: 18 is absent or is selected from Asn (N), Ser (S) and Gly (G), preferably X₁₂ is absent.

In one embodiment, the antibody or binding fragment thereof comprises a combination of (i) at least one, preferably at least two, more preferably three HCVR's CDRs and (ii) at least one, preferably at least two, more preferably three LCVR's CDRs, said combination being combination #20 as defined in Table 2.

In one embodiment, the antibody or binding fragment thereof comprises a combination of (i) three HCVR's CDRs and (ii) three LCVR's CDRs, said combination being combination #20 as defined in Table 2.

In one embodiment, the antibody or binding fragment thereof comprises a combination of (i) three HCVR's CDRs set forth as SEQ ID NOs: 1, 7 and 3; and (ii) three LCVR's CDRs set forth as SEQ ID NOs: 18, 16 and 17; wherein X₁₂ in SEQ ID NOs: 18 is absent or is selected from Asn (N), Ser (S) and Gly (G), preferably X₁₂ is absent.

In one embodiment, the antibody or binding fragment thereof comprises a combination of (i) at least one, preferably at least two, more preferably three HCVR's CDRs and (ii) at least one, preferably at least two, more preferably three LCVR's CDRs, said combination being combination #26 as defined in Table 2.

In one embodiment, the antibody or binding fragment thereof comprises a combination of (i) three HCVR's CDRs and (ii) three LCVR's CDRs, said combination being combination #26 as defined in Table 2.

In one embodiment, the antibody or binding fragment thereof comprises a combination of (i) three HCVR's CDRs set forth as SEQ ID NOs: 1, 8 and 3; and (ii) three LCVR's CDRs set forth as SEQ ID NOs: 18, 16 and 17; wherein X₁₂ in SEQ ID NOs: 18 is absent or is selected from Asn (N), Ser (S) and Gly (G), preferably X₁₂ is absent.

In one embodiment, the antibody or binding fragment thereof comprises a combination of (i) at least one, preferably at least two, more preferably three HCVR's CDRs and (ii) at least one, preferably at least two, more preferably three LCVR's CDRs, said combination being combination #49 as defined in Table 2.

In one embodiment, the antibody or binding fragment thereof comprises a combination of (i) three HCVR's CDRs and (ii) three LCVR's CDRs, said combination being combination #49 as defined in Table 2.

In one embodiment, the antibody or binding fragment thereof comprises a combination of (i) three HCVR's CDRs set forth as SEQ ID NOs: 1, 100 and 3; and (ii) three LCVR's CDRs set forth as SEQ ID NOs: 15, 16 and 17; wherein X₁₂ in SEQ ID NOs: 15 is absent or is selected from Asn (N), Ser (S) and Gly (G), preferably X₁₂ is absent.

In one embodiment, the antibody or binding fragment thereof comprises a combination of (i) at least one, preferably at least two, more preferably three HCVR's CDRs and (ii) at least one, preferably at least two, more preferably three LCVR's CDRs, said combination being combination #50 as defined in Table 2.

In one embodiment, the antibody or binding fragment thereof comprises a combination of (i) three HCVR's CDRs and (ii) three LCVR's CDRs, said combination being combination #50 as defined in Table 2.

In one embodiment, the antibody or binding fragment thereof comprises a combination of (i) three HCVR's CDRs set forth as SEQ ID NOs: 1, 100 and 3; and (ii) three LCVR's CDRs set forth as SEQ ID NOs: 18, 16 and 17; wherein X₁₂ in SEQ ID NOs: 18 is absent or is selected from Asn (N), Ser (S) and Gly (G), preferably X₁₂ is absent.

In one embodiment, the antibody or binding fragment thereof comprises a combination of (i) at least one, preferably at least two, more preferably three HCVR's CDRs and (ii) at least one, preferably at least two, more preferably three LCVR's CDRs, said combination being combination #63 as defined in Table 2.

In one embodiment, the antibody or binding fragment thereof comprises a combination of (i) three HCVR's CDRs and (ii) three LCVR's CDRs, said combination being combination #63 as defined in Table 2.

In one embodiment, the antibody or binding fragment thereof comprises a combination of (i) three HCVR's CDRs set forth as SEQ ID NOs: 1, 100 and 3; and (ii) three LCVR's CDRs set forth as SEQ ID NOs: 18, 111 and 17; wherein X₁₂ in SEQ ID NOs: 18 is absent or is selected from Asn (N), Ser (S) and Gly (G), preferably X₁₂ is absent.

In one embodiment, the antibody or binding fragment thereof comprises a combination of (i) at least one, preferably at least two, more preferably three HCVR's CDRs and (ii) at least one, preferably at least two, more preferably three LCVR's CDRs, said combination being combination #65 as defined in Table 2.

In one embodiment, the antibody or binding fragment thereof comprises a combination of (i) three HCVR's CDRs and (ii) three LCVR's CDRs, said combination being combination #65 as defined in Table 2.

In one embodiment, the antibody or binding fragment thereof comprises a combination of (i) three HCVR's CDRs set forth as SEQ ID NOs: 1, 116 and 3; and (ii) three LCVR's CDRs set forth as SEQ ID NOs: 18, 16 and 17; wherein X₁₂ in SEQ ID NOs: 18 is absent or is selected from Asn (N), Ser (S) and Gly (G), preferably X₁₂ is absent.

In one embodiment, the antibody or binding fragment thereof comprises a combination of (i) at least one, preferably at least two, more preferably three HCVR's CDRs and (ii) at least one, preferably at least two, more preferably three LCVR's CDRs, said combination being combination #72 as defined in Table 2.

In one embodiment, the antibody or binding fragment thereof comprises a combination of (i) three HCVR's CDRs and (ii) three LCVR's CDRs, said combination being combination #72 as defined in Table 2.

In one embodiment, the antibody or binding fragment thereof comprises a combination of (i) three HCVR's CDRs set forth as SEQ ID NOs: 1, 117 and 3; and (ii) three LCVR's CDRs set forth as SEQ ID NOs: 18, 16 and 17; wherein X₁₂ in SEQ ID NOs: 18 is absent or is selected from Asn (N), Ser (S) and Gly (G), preferably X₁₂ is absent.

In one embodiment, the antibody or binding fragment thereof comprises a combination of (i) at least one, preferably at least two, more preferably three HCVR's CDRs and (ii) at least one, preferably at least two, more preferably three LCVR's CDRs, said combination being combination #79 as defined in Table 2.

In one embodiment, the antibody or binding fragment thereof comprises a combination of (i) three HCVR's CDRs and (ii) three LCVR's CDRs, said combination being combination #79 as defined in Table 2.

In one embodiment, the antibody or binding fragment thereof comprises a combination of (i) three HCVR's CDRs set forth as SEQ ID NOs: 1, 118 and 3; and (ii) three LCVR's CDRs set forth as SEQ ID NOs: 18, 16 and 17; wherein X₁₂ in SEQ ID NOs: 18 is absent or is selected from Asn (N), Ser (S) and Gly (G), preferably X₁₂ is absent.

In one embodiment, the antibody or binding fragment thereof comprises a combination of (i) at least one, preferably at least two, more preferably three HCVR's CDRs and (ii) at least one, preferably at least two, more preferably three LCVR's CDRs, said combination being combination #86 as defined in Table 2.

In one embodiment, the antibody or binding fragment thereof comprises a combination of (i) three HCVR's CDRs and (ii) three LCVR's CDRs, said combination being combination #86 as defined in Table 2.

In one embodiment, the antibody or binding fragment thereof comprises a combination of (i) three HCVR's CDRs set forth as SEQ ID NOs: 1, 119 and 3; and (ii) three LCVR's CDRs set forth as SEQ ID NOs: 18, 16 and 17; wherein X₁₂ in SEQ ID NOs: 18 is absent or is selected from Asn (N), Ser (S) and Gly (G), preferably X₁₂ is absent.

In one embodiment, the antibody or binding fragment thereof comprises a combination of (i) at least one, preferably at least two, more preferably three HCVR's CDRs and (ii) at least one, preferably at least two, more preferably three LCVR's CDRs, said combination being combination #92 as defined in Table 2.

In one embodiment, the antibody or binding fragment thereof comprises a combination of (i) three HCVR's CDRs and (ii) three LCVR's CDRs, said combination being combination #92 as defined in Table 2.

In one embodiment, the antibody or binding fragment thereof comprises a combination of (i) three HCVR's CDRs set forth as SEQ ID NOs: 1, 4 and 3; and (ii) three LCVR's CDRs set forth as SEQ ID NOs: 15, 120 and 17; wherein X₁₂ in SEQ ID NOs: 15 is absent or is selected from Asn (N), Ser (S) and Gly (G), preferably X₁₂ is absent.

In one embodiment, the antibody or binding fragment thereof comprises a HCVR which comprises at least one, preferably at least two, more preferably at least three, even more preferably the four following framework regions (FRs):

V_{H}-FR1:
(SEQ ID NO: 25)
QVQLQQSGAELVRPGTSVKMSCKAAGYTFT;
V_{H}-FR2:
(SEQ ID NO: 26)
WVKQRPGHGLEWIG;
V_{H}-FR3:
(SEQ ID NO: 27)
KATLTADTSSSTAYMQLSSLTSEDSAIYYCVR;
V_{H}-FR4:
(SEQ ID NO: 28)
WGQGTTLTVSS.

In one embodiment, the antibody or binding fragment thereof comprises a HCVR which comprises the four following FRs:

V_{H}-FR1:
(SEQ ID NO: 25)
QVQLQQSGAELVRPGTSVKMSCKAAGYTFT;
V_{H}-FR2:
(SEQ ID NO: 26)
WVKQRPGHGLEWIG;
V_{H}-FR3:
(SEQ ID NO: 27)
KATLTADTSSSTAYMQLSSLTSEDSAIYYCVR;
V_{H}-FR4:
(SEQ ID NO: 28)
WGQGTTLTVSS.

In one embodiment, the antibody or binding fragment thereof comprises a HCVR which comprises at least one, preferably at least two, more preferably at least three, even more preferably the four following FRs:

V_{H}-FR1:
(SEQ ID NO: 29)
QVQLVQSGAEVKKPGASVKVSCKASGYTFT;
V_{H}-FR2:
(SEQ ID NO: 30)
WVRQAPGQGLEWIG;
V_{H}-FR3:
(SEQ ID NO: 31)
RVTLTADTSISTAYMELSRLRSDDTVVYYCVR;
V_{H}-FR4:
(SEQ ID NO: 32)
WGQGTLVTVSS.

In one embodiment, the antibody or binding fragment thereof comprises a HCVR which comprises the four following FRs:

V_{H}-FR1:
(SEQ ID NO: 29)
QVQLVQSGAEVKKPGASVKVSCKASGYTFT;
V_{H}-FR2:
(SEQ ID NO: 30)
WVRQAPGQGLEWIG;
V_{H}-FR3:
(SEQ ID NO: 31)
RVTLTADTSISTAYMELSRLRSDDTVVYYCVR;
V_{H}-FR4:
(SEQ ID NO: 32)
WGQGTLVTVSS.

In one embodiment, the antibody or binding fragment thereof comprises a HCVR which comprises at least one, preferably at least two, more preferably at least three, even more preferably the four following FRs:

V_{H}-FR1:
(SEQ ID NO: 33)
EVQLVQSGAEVKKPGESLKISCKASGYTFT;
V_{H}-FR2:
(SEQ ID NO: 34)
WVRQMPGKGLEWIG;
V_{H}-FR3:
(SEQ ID NO: 35)
QVTLSADKSISTAYLQLSSLKASDTAMYYCVR;
V_{H}-FR4:
(SEQ ID NO: 32)
WGQGTLVTVSS.

In one embodiment, the antibody or binding fragment thereof comprises a HCVR which comprises the four following FRs:

V_{H}-FR1:
(SEQ ID NO: 33)
EVQLVQSGAEVKKPGESLKISCKASGYTFT;
V_{H}-FR2:
(SEQ ID NO: 34)
WVRQMPGKGLEWIG;
V_{H}-FR3:
(SEQ ID NO: 35)
QVTLSADKSISTAYLQLSSLKASDTAMYYCVR;
V_{H}-FR4:
(SEQ ID NO: 32)
WGQGTLVTVSS.

In one embodiment, the antibody or binding fragment thereof comprises a HCVR which comprises at least one, preferably at least two, more preferably at least three, even more preferably the four following FRs:

V_{H}-FR1:
(SEQ ID NO: 36)
QVQLVESGGGLVKPGGSLRLSCAASGYTFT;
V_{H}-FR2:
(SEQ ID NO: 37)
WIRQAPGKGLEWIG;
V_{H}-FR3:
(SEQ ID NO: 38)
RFTLSADTAKNSAYLQMNSLRAEDTAVYYCVR;
V_{H}-FR4:
(SEQ ID NO: 32)
WGQGTLVTVSS.

In one embodiment, the antibody or binding fragment thereof comprises a HCVR which comprises the four following FRs:

V_{H}-FR1:
(SEQ ID NO: 36)
QVQLVESGGGLVKPGGSLRLSCAASGYTFT;
V_{H}-FR2:
(SEQ ID NO: 37)
WIRQAPGKGLEWIG;
V_{H}-FR3:
(SEQ ID NO: 38)
RFTLSADTAKNSAYLQMNSLRAEDTAVYYCVR;
V_{H}-FR4:
(SEQ ID NO: 32)
WGQGTLVTVSS.

In one embodiment, the antibody or binding fragment thereof comprises a HCVR which comprises at least one, preferably at least two, more preferably at least three, even more preferably the four following FRs:

V_{H}-FR1:
(SEQ ID NO: 39)
EVQLVQSGAEVKKPGESLKISCKGSGYTFT;
V_{H}-FR2:
(SEQ ID NO: 34)
WVRQMPGKGLEWIG;
V_{H}-FR3:
(SEQ ID NO: 35)
QVTLSADKSISTAYLQLSSLKASDTAMYYCVR;
V_{H}-FR4:
(SEQ ID NO: 32)
WGQGTLVTVSS.

In one embodiment, the antibody or binding fragment thereof comprises a HCVR which comprises the four following FRs:

V_{H}-FR1:
(SEQ ID NO: 39)
EVQLVQSGAEVKKPGESLKISCKGSGYTFT;
V_{H}-FR2:
(SEQ ID NO: 34)
WVRQMPGKGLEWIG;
V_{H}-FR3:
(SEQ ID NO: 35)
QVTLSADKSISTAYLQLSSLKASDTAMYYCVR;
V_{H}-FR4:
(SEQ ID NO: 32)
WGQGTLVTVSS.

In one embodiment, the antibody or binding fragment thereof comprises a HCVR which comprises at least one, preferably at least two, more preferably at least three, even more preferably the four following FRs:

V_{H}-FR1:
(SEQ ID NO: 40)
QVQLVESGGGLVKPGGSLRLSCAASGFTFS;
V_{H}-FR2:
(SEQ ID NO: 37)
WIRQAPGKGLEWIG;
V_{H}-FR3:
(SEQ ID NO: 41)
RFTLSADTAKNSLYLQMNSLRAEDTAVYYCVR;
V_{H}-FR4:
(SEQ ID NO: 32)
WGQGTLVTVSS.

In one embodiment, the antibody or binding fragment thereof comprises a HCVR which comprises the four following FRs:

V_{H}-FR1:
(SEQ ID NO: 40)
QVQLVESGGGLVKPGGSLRLSCAASGFTFS;
V_{H}-FR2:
(SEQ ID NO: 37)
WIRQAPGKGLEWIG;
V_{H}-FR3:
(SEQ ID NO: 41)
RFTLSADTAKNSLYLQMNSLRAEDTAVYYCVR;
V_{H}-FR4:
(SEQ ID NO: 32)
WGQGTLVTVSS.

In one embodiment, the antibody or binding fragment thereof comprises a HCVR which comprises at least one, preferably at least two, more preferably at least three, even more preferably the four following FRs:

V_{H}-FR1:
(SEQ ID NO: 42)
EVQLVQSGAEVKKPGESLKISCKGSGYSFT;
V_{H}-FR2:
(SEQ ID NO: 34)
WVRQMPGKGLEWIG;
V_{H}-FR3:
(SEQ ID NO: 35)
QVTLSADKSISTAYLQLSSLKASDTAMYYCVR;
V_{H}-FR4:
(SEQ ID NO: 32)
WGQGTLVTVSS.

In one embodiment, the antibody or binding fragment thereof comprises a HCVR which comprises the four following FRs:

V_{H}-FR1:
(SEQ ID NO: 42)
EVQLVQSGAEVKKPGESLKISCKGSGYSFT;
V_{H}-FR2:
(SEQ ID NO: 34)
WVRQMPGKGLEWIG;
V_{H}-FR3:
(SEQ ID NO: 35)
QVTLSADKSISTAYLQLSSLKASDTAMYYCVR;
V_{H}-FR4:
(SEQ ID NO: 32)
WGQGTLVTVSS.

In one embodiment, the antibody or binding fragment thereof comprises a HCVR which comprises at least one, preferably at least two, more preferably at least three, even more preferably the four following FRs:

V_{H}-FR1:
(SEQ ID NO: 40)
QVQLVESGGGLVKPGGSLRLSCAASGFTFS;
V_{H}-FR2:
(SEQ ID NO: 43)
WIRQAPGKGLEWVG;
V_{H}-FR3:
(SEQ ID NO: 41)
RFTLSADTAKNSLYLQMNSLRAEDTAVYYCVR;
V_{H}-FR4:
(SEQ ID NO: 32)
WGQGTLVTVSS.

In one embodiment, the antibody or binding fragment thereof comprises a HCVR which comprises the four following FRs:

V_{H}-FR1:
(SEQ ID NO: 40)
QVQLVESGGGLVKPGGSLRLSCAASGFTFS;
V_{H}-FR2:
(SEQ ID NO: 43)
WIRQAPGKGLEWVG;
V_{H}-FR3:
(SEQ ID NO: 41)
RFTLSADTAKNSLYLQMNSLRAEDTAVYYCVR;
V_{H}-FR4:
(SEQ ID NO: 32)
WGQGTLVTVSS.

In one embodiment, the antibody or binding fragment thereof comprises a LCVR which comprises at least one, preferably at least two, more preferably at least three, even more preferably the four following FRs:

V_{L}-FR1:
(SEQ ID NO: 44)
QIVLTQSPTIMSASPGEKVTITC;
V_{L}-FR2:
(SEQ ID NO: 45)
WFQQKTGTSPRLWIY;
V_{L}-FR3:
(SEQ ID NO: 46)
GVPARFSGSGSGTS-X₁₈-SLTISRMEAEDAATYYC;
V_{L}-FR4:
(SEQ ID NO: 47)
GAGTKLELK,

with:

X₁ₛ being selected from Tyr (Y) and Phe (F), preferably X₁ₛ being Tyr (Y).

In one embodiment, the antibody or binding fragment thereof comprises a LCVR which comprises the four following FRs:

V_{L}-FR1:
(SEQ ID NO: 44)
QIVLTQSPTIMSASPGEKVTITC;
V_{L}-FR2:
(SEQ ID NO: 45)
WFQQKTGTSPRLWIY;
V_{L}-FR3:
(SEQ ID NO: 46)
GVPARFSGSGSGTS-X₁₈-SLTISRMEAEDAATYYC;
V_{L}-FR4:
(SEQ ID NO: 47)
GAGTKLELK

with:

X₁ₛ being selected from Tyr (Y) and Phe (F), preferably X₁ₛ being Tyr (Y).

In one embodiment, the antibody or binding fragment thereof comprises a LCVR which comprises at least one, preferably at least two, more preferably at least three, even more preferably the four following FRs:

V_{L}-FR1:
(SEQ ID NO: 48)
DIQLTQSPSFLSASVGDRVTITC;
V_{L}-FR2:
(SEQ ID NO: 49)
WFQQKPGKAPKLWIY;
V_{L}-FR3:
(SEQ ID NO: 50)
GVPSRFSGSGSGTE-X₁₈-TLTISSLQPEDFATYYC;
V_{L}-FR4:
(SEQ ID NO: 51)
GGGTKVEIK,

with:

X₁₈ being selected from Tyr (Y) and Phe (F), preferably X₁₈ being Tyr (Y).

In one embodiment, the antibody or binding fragment thereof comprises a LCVR which comprises the four following FRs:

V_{L}-FR1:
(SEQ ID NO: 48)
DIQLTQSPSFLSASVGDRVTITC;
V_{L}-FR2:
(SEQ ID NO: 49)
WFQQKPGKAPKLWIY;
V_{L}-FR3:
(SEQ ID NO: 50)
GVPSRFSGSGSGTE-X₁₈-TLTISSLQPEDFATYYC;
V_{L}-FR4:
(SEQ ID NO: 51)
GGGTKVEIK,

with:

X₁₈ being selected from Tyr (Y) and Phe (F), preferably X₁ₛ being Tyr (Y).

In one embodiment, the antibody or binding fragment thereof comprises a LCVR which comprises at least one, preferably at least two, more preferably at least three, even more preferably the four following FRs:

V_{L}-FR1:
(SEQ ID NO: 52)
EIVLTQSPDFQSVTPKEKVTITC;
V_{L}-FR2:
(SEQ ID NO: 53)
WFQQKPDQSPKLWIY;
V_{L}-FR3:
(SEQ ID NO: 54)
GVPSRFSGSGSGTD-X₁₈-TLTINSLEAEDAATYYC;
V_{L}-FR4:
(SEQ ID NO: 51)
GGGTKVEIK,

with:

X₁₈ being selected from Tyr (Y) and Phe (F), preferably X₁₈ being Tyr (Y).

In one embodiment, the antibody or binding fragment thereof comprises a LCVR which comprises the four following FRs:

V_{L}-FR1:
(SEQ ID NO: 52)
EIVLTQSPDFQSVTPKEKVTITC;
V_{L}-FR2:
(SEQ ID NO: 53)
WFQQKPDQSPKLWIY;
V_{L}-FR3:
(SEQ ID NO: 54)
GVPSRFSGSGSGTD-X₁₈-TLTINSLEAEDAATYYC;
V_{L}-FR4:
(SEQ ID NO: 51)
GGGTKVEIK,

with:

X₁₈ being selected from Tyr (Y) and Phe (F), preferably X₁₈ being Tyr (Y).

In one embodiment, the antibody or binding fragment thereof comprises a LCVR which comprises at least one, preferably at least two, more preferably at least three, even more preferably the four following FRs:

V_{L}-FR1:
(SEQ ID NO: 55)
EIVLTQSPATLSLSPGERATLSC;
V_{L}-FR2:
(SEQ ID NO: 56)
WFQQKPGQAPRLWIY;
V_{L}-FR3:
(SEQ ID NO: 57)
GIPARFSGSGSGTD-X₁₈-TLTISSLEPEDFAVYYC;
V_{L}-FR4:
(SEQ ID NO: 51)
GGGTKVEIK,

with:

X₁ₛ being selected from Tyr (Y) and Phe (F), preferably X₁ₛ being Tyr (Y).

In one embodiment, the antibody or binding fragment thereof comprises a LCVR which comprises the four following FRs:

with:

V_{L}-FR1:
(SEQ ID NO: 55)
EIVLTQSPATLSLSPGERATLSC;
V_{L}-FR2:
(SEQ ID NO: 56)
WFQQKPGQAPRLWIY;
V_{L}-FR3:
(SEQ ID NO: 57)
GIPARFSGSGSGTD-X₁₈-TLTISSLEPEDFAVYYC;
V_{L}-FR4:
(SEQ ID NO: 51)
GGGTKVEIK,

X₁ₛ being selected from Tyr (Y) and Phe (F), preferably X₁ₛ being Tyr (Y).

In one embodiment, the antibody or binding fragment thereof comprises a LCVR which comprises at least one, preferably at least two, more preferably at least three, even more preferably the four following FRs:

V_{L}-FR1:
(SEQ ID NO: 48)
DIQLTQSPSFLSASVGDRVTITC;
V_{L}-FR2:
(SEQ ID NO: 58)
WYQQKPGKAPKLWIY;
V_{L}-FR3:
(SEQ ID NO: 50)
GVPSRFSGSGSGTE-X₁₈-TLTISSLQPEDFATYYC;
V_{L}-FR4:
(SEQ ID NO: 51)
GGGTKVEIK,

with:

X₁ₛ being selected from Tyr (Y) and Phe (F), preferably X₁ₛ being Tyr (Y).

In one embodiment, the antibody or binding fragment thereof comprises a LCVR which comprises the four following FRs:

V_{L}-FR1:
(SEQ ID NO: 48)
DIQLTQSPSFLSASVGDRVTITC;
V_{L}-FR2:
(SEQ ID NO: 58)
WYQQKPGKAPKLWIY;
V_{L}-FR3:
(SEQ ID NO: 50)
GVPSRFSGSGSGTE-X₁₈-TLTISSLQPEDFATYYC;
V_{L}-FR4:
(SEQ ID NO: 51)
GGGTKVEIK,

with:

X₁₈ being selected from Tyr (Y) and Phe (F), preferably X₁₈ being Tyr (Y).

In one embodiment, the antibody or binding fragment thereof comprises a LCVR which comprises at least one, preferably at least two, more preferably at least three, even more preferably the four following FRs:

V_{L}-FR1:
(SEQ ID NO: 48)
DIQLTQSPSFLSASVGDRVTITC;
V_{L}-FR2:
(SEQ ID NO: 58)
WYQQKPGKAPKLWIY;
V_{L}-FR3:
(SEQ ID NO: 50)
GVPSRFSGSGSGTE-X₁₈-TLTISSLQPEDFATYYC;
V_{L}-FR4:
(SEQ ID NO: 51)
GGGTKVEIK,

with:

X₁₈ being selected from Tyr (Y) and Phe (F), preferably X₁₈ being Phe (F).

In one embodiment, the antibody or binding fragment thereof comprises a LCVR which comprises the four following FRs:

V_{L}-FR1:
(SEQ ID NO: 48)
DIQLTQSPSFLSASVGDRVTITC;
V_{L}-FR2:
(SEQ ID NO: 58)
WYQQKPGKAPKLWIY;
V_{L}-FR3:
(SEQ ID NO: 50)
GVPSRFSGSGSGTE-X₁₈-TLTISSLQPEDFATYYC;
V_{L}-FR4:
(SEQ ID NO: 51)
GGGTKVEIK,

with:

X₁ₛ being selected from Tyr (Y) and Phe (F), preferably X₁ₛ being Phe (F).

In one embodiment, the antibody or binding fragment thereof comprises a LCVR which comprises at least one, preferably at least two, more preferably at least three, even more preferably the four following FRs:

V_{L}-FR1:
(SEQ ID NO: 52)
EIVLTQSPDFQSVTPKEKVTITC;
V_{L}-FR2:
(SEQ ID NO: 59)
WYQQKPDQSPKLWIY;
V_{L}-FR3:
(SEQ ID NO: 54)
GVPSRFSGSGSGTD-X₁₈-TLTINSLEAEDAATYYC;
V_{L}-FR4:
(SEQ ID NO: 51)
GGGTKVEIK,

with:

X₁₈ being selected from Tyr (Y) and Phe (F), preferably X₁₈ being Phe (F).

In one embodiment, the antibody or binding fragment thereof comprises a LCVR which comprises the four following FRs:

V_{L}-FR1:
(SEQ ID NO: 52)
EIVLTQSPDFQSVTPKEKVTITC;
V_{L}-FR2:
(SEQ ID NO: 59)
WYQQKPDQSPKLWIY;
V_{L}-FR3:
(SEQ ID NO: 54)
GVPSRFSGSGSGTD-X₁₈-TLTINSLEAEDAATYYC;
V_{L}-FR4:
(SEQ ID NO: 51)
GGGTKVEIK,

with:

X₁ₛ being selected from Tyr (Y) and Phe (F), preferably X₁ₛ being Phe (F).

In one embodiment, the antibody or binding fragment thereof comprises a LCVR which comprises at least one, preferably at least two, more preferably at least three, even more preferably the four following FRs:

V_{L}-FR1:
(SEQ ID NO: 55)
EIVLTQSPATLSLSPGERATLSC;
V_{L}-FR2:
(SEQ ID NO: 60)
WYQQKPGQAPRLWIY;
V_{L}-FR3:
(SEQ ID NO: 57)
GIPARFSGSGSGTD-X₁₈-TLTISSLEPEDFAVYYC;
V_{L}-FR4:
(SEQ ID NO: 51)
GGGTKVEIK,

with:

X₁ₛ being selected from Tyr (Y) and Phe (F), preferably X₁ₛ being Phe (F).

In one embodiment, the antibody or binding fragment thereof comprises a LCVR which comprises the four following FRs:

V_{L}-FR1:
(SEQ ID NO: 55)
EIVLTQSPATLSLSPGERATLSC;
V_{L}-FR2:
(SEQ ID NO: 60)
WYQQKPGQAPRLWIY;
V_{L}-FR3:
(SEQ ID NO: 57)
GIPARFSGSGSGTD-X₁₈-TLTISSLEPEDFAVYYC;
V_{L}-FR4:
(SEQ ID NO: 51)
GGGTKVEIK,

with:

X₁ₛ being selected from Tyr (Y) and Phe (F), preferably X₁₈ being Phe (F).

In one embodiment, the antibody or binding fragment thereof comprises a LCVR which comprises at least one, preferably at least two, more preferably at least three, even more preferably the four following FRs:

V_{L}-FR1:
(SEQ ID NO: 48)
DIQLTQSPSFLSASVGDRVTITC;
V_{L}-FR2:
(SEQ ID NO: 49)
WFQQKPGKAPKLWIY;
V_{L}-FR3:
(SEQ ID NO: 50)
GVPSRFSGSGSGTE-X₁₈-TLTISSLQPEDFATYYC;
V_{L}-FR4:
(SEQ ID NO: 51)
GGGTKVEIK,

with:

X₁ₛ being selected from Tyr (Y) and Phe (F), preferably X₁ₛ being Phe (F).

In one embodiment, the antibody or binding fragment thereof comprises a LCVR which comprises the four following FRs:

V_{L}-FR1:
(SEQ ID NO: 48)
DIQLTQSPSFLSASVGDRVTITC;
V_{L}-FR2:
(SEQ ID NO: 49)
WFQQKPGKAPKLWIY;
V_{L}-FR3:
(SEQ ID NO: 50)
GVPSRFSGSGSGTE-X₁₈-TLTISSLQPEDFATYYC;
V_{L}-FR4:
(SEQ ID NO: 51)
GGGTKVEIK,

with:

X₁ₛ being selected from Tyr (Y) and Phe (F), preferably X₁ₛ being Phe (F).

In one embodiment, the antibody or binding fragment thereof comprises a combination of (i) at least one, preferably at least two, more preferably at least three, even more preferably four HCVR's FRs and (ii) at least one, preferably at least two, more preferably at least three, even more preferably four LCVR's FRs, said combination being as defined in Table 3.

In one embodiment, the antibody or binding fragment thereof comprises a combination of (i) four HCVR's FRs and (ii) four LCVR's FRs, said combination being as defined in Table 3.

TABLE 3
Preferred combinations of HCVR's and LCVR's FRs. The FRs
are defined by their SEQ ID NOs (with, wherever applicable,
X₁₈ being selected from Tyr (Y) and Phe (F)).

| FRs' | | | | | | | | |
|---|---|---|---|---|---|---|---|---|
| combination | V_{H}- | V_{H}- | V_{H}- | V_{H}- | V_{L}- | V_{L}- | V_{L}- | V_{L}- |
| # | FR1 | FR2 | FR3 | FR4 | FR1 | FR2 | FR3 | FR4 |
| #1 | 25 | 26 | 27 | 28 | 44 | 45 | 46 | 47 |
| #2 | 25 | 26 | 27 | 28 | 48 | 49 | 50 | 51 |
| #3 | 25 | 26 | 27 | 28 | 52 | 53 | 54 | 51 |
| #4 | 25 | 26 | 27 | 28 | 55 | 56 | 57 | 51 |
| #5 | 25 | 26 | 27 | 28 | 48 | 58 | 50 | 51 |
| #6 | 25 | 26 | 27 | 28 | 52 | 59 | 54 | 51 |
| #7 | 25 | 26 | 27 | 28 | 55 | 60 | 57 | 51 |
| #8 | 29 | 30 | 31 | 32 | 44 | 45 | 46 | 47 |
| #9 | 29 | 30 | 31 | 32 | 48 | 49 | 50 | 51 |
| #10 | 29 | 30 | 31 | 32 | 52 | 53 | 54 | 51 |
| #11 | 29 | 30 | 31 | 32 | 55 | 56 | 57 | 51 |
| #12 | 29 | 30 | 31 | 32 | 48 | 58 | 50 | 51 |
| #13 | 29 | 30 | 31 | 32 | 52 | 59 | 54 | 51 |
| #14 | 29 | 30 | 31 | 32 | 55 | 60 | 57 | 51 |
| #15 | 33 | 34 | 35 | 32 | 44 | 45 | 46 | 47 |
| #16 | 33 | 34 | 35 | 32 | 48 | 49 | 50 | 51 |
| #17 | 33 | 34 | 35 | 32 | 52 | 53 | 54 | 51 |
| #18 | 33 | 34 | 35 | 32 | 55 | 56 | 57 | 51 |
| #19 | 33 | 34 | 35 | 32 | 48 | 58 | 50 | 51 |
| #20 | 33 | 34 | 35 | 32 | 52 | 59 | 54 | 51 |
| #21 | 33 | 34 | 35 | 32 | 55 | 60 | 57 | 51 |
| #22 | 36 | 37 | 38 | 32 | 44 | 45 | 46 | 47 |
| #23 | 36 | 37 | 38 | 32 | 48 | 49 | 50 | 51 |
| #24 | 36 | 37 | 38 | 32 | 52 | 53 | 54 | 51 |
| #25 | 36 | 37 | 38 | 32 | 55 | 56 | 57 | 51 |
| #26 | 36 | 37 | 38 | 32 | 48 | 58 | 50 | 51 |
| #27 | 36 | 37 | 38 | 32 | 52 | 59 | 54 | 51 |
| #28 | 36 | 37 | 38 | 32 | 55 | 60 | 57 | 51 |
| #29 | 39 | 34 | 35 | 32 | 44 | 45 | 46 | 47 |
| #30 | 39 | 34 | 35 | 32 | 48 | 49 | 50 | 51 |
| #31 | 39 | 34 | 35 | 32 | 52 | 53 | 54 | 51 |
| #32 | 39 | 34 | 35 | 32 | 55 | 56 | 57 | 51 |
| #33 | 39 | 34 | 35 | 32 | 48 | 58 | 50 | 51 |
| #34 | 39 | 34 | 35 | 32 | 52 | 59 | 54 | 51 |
| #35 | 39 | 34 | 35 | 32 | 55 | 60 | 57 | 51 |
| #36 | 40 | 37 | 41 | 32 | 44 | 45 | 46 | 47 |
| #37 | 40 | 37 | 41 | 32 | 48 | 49 | 50 | 51 |
| #38 | 40 | 37 | 41 | 32 | 52 | 53 | 54 | 51 |
| #39 | 40 | 37 | 41 | 32 | 55 | 56 | 57 | 51 |
| #40 | 40 | 37 | 41 | 32 | 48 | 58 | 50 | 51 |
| #41 | 40 | 37 | 41 | 32 | 52 | 59 | 54 | 51 |
| #42 | 40 | 37 | 41 | 32 | 55 | 60 | 57 | 51 |
| #43 | 42 | 34 | 35 | 32 | 44 | 45 | 46 | 47 |
| #44 | 42 | 34 | 35 | 32 | 48 | 49 | 50 | 51 |
| #45 | 42 | 34 | 35 | 32 | 52 | 53 | 54 | 51 |
| #46 | 42 | 34 | 35 | 32 | 55 | 56 | 57 | 51 |
| #47 | 42 | 34 | 35 | 32 | 48 | 58 | 50 | 51 |
| #48 | 42 | 34 | 35 | 32 | 52 | 59 | 54 | 51 |
| #49 | 42 | 34 | 35 | 32 | 55 | 60 | 57 | 51 |
| #50 | 40 | 43 | 41 | 32 | 44 | 45 | 46 | 47 |
| #51 | 40 | 43 | 41 | 32 | 48 | 49 | 50 | 51 |
| #52 | 40 | 43 | 41 | 32 | 52 | 53 | 54 | 51 |
| #53 | 40 | 43 | 41 | 32 | 55 | 56 | 57 | 51 |
| #54 | 40 | 43 | 41 | 32 | 48 | 58 | 50 | 51 |
| #55 | 40 | 43 | 41 | 32 | 52 | 59 | 54 | 51 |
| #56 | 40 | 43 | 41 | 32 | 55 | 60 | 57 | 51 |

In one embodiment, any of V_{H}-FR1, V_{H}-FR2, V_{H}-FR3, V_{H}-FR4, V_{L}-FR1, V_{L}-FR2, V_{L}-FR3 and/or V_{L}-FR4 as defined hereinabove can be characterized as having 1, 2, 3, 4, 5, 6, 7, 8, 9, 10 or more amino acids being substituted by a different amino acid.

In one embodiment, any of V_{H}-FR1, V_{H}-FR2, V_{H}-FR3, V_{H}-FR4, V_{L}-FR1, V_{L}-FR2, V_{L}-FR3 and/or V_{L}-FR4 as defined hereinabove can be characterized as having an amino acid sequence that shares at least 70%, 75%, 80%, 85%, 90%, 95%, 96%, 97%, 98%, 99% or more of identity with the particular FR or sets of FRs as defined hereinabove.

In one embodiment, the antibody or binding fragment thereof comprises a HCVR comprising or consisting of:
a V_{H}-FR1 as described hereinabove,
a V_{H}-CDR1 as described hereinabove,
a V_{H}-FR2 as described hereinabove,
a V_{H}-CDR2 as described hereinabove,
a V_{H}-FR3 as described hereinabove,
a V_{H}-CDR3 as described hereinabove, and
a V_{H}-FR4 as described hereinabove.

In one embodiment, the antibody or binding fragment thereof comprises a HCVR comprising or consisting of:
a V_{H}-FR1 selected from SEQ ID NOs 25, 29, 33, 36, 39, 40 and 42;
a V_{H}-CDR1 selected from SEQ ID NO 1;
a V_{H}-FR2 selected from SEQ ID NOs 26, 30, 34, 37 and 43;
a V_{H}-CDR2 selected from SEQ ID NO 2;
a V_{H}-FR3 selected from SEQ ID NOs 27, 31, 35, 38 and 41;
a V_{H}-CDR3 selected from SEQ ID NO 3; and
a V_{H}-FR4 selected from SEQ ID NOs 28 and 32.

In one embodiment, the antibody or binding fragment thereof comprises a HCVR comprising or consisting of:
a V_{H}-FR1 selected from SEQ ID NOs 25, 29, 33, 36, 39, 40 and 42;
a V_{H}-CDR1 selected from SEQ ID NO 1;
a V_{H}-FR2 selected from SEQ ID NOs 26, 30, 34, 37 and 43;
a V_{H}-CDR2 selected from SEQ ID NOs 4, 5, 6, 7, 8, 9, 10, 11, 100, 116, 117, 118 and 119;
a V_{H}-FR3 selected from SEQ ID NOs 27, 31, 35, 38 and 41;
a V_{H}-CDR3 selected from SEQ ID NO 3; and
a V_{H}-FR4 selected from SEQ ID NOs 28 and 32.

In a preferred embodiment, the antibody or binding fragment thereof comprises a HCVR comprising or consisting of a combination of a V_{H}-FR1, a V_{H}-CDR1, a V_{H}-FR2, a V_{H}-CDR2, a V_{H}-FR3, a V_{H}-CDR3 and a V_{H}-FR4, said combination being as defined in Table 4.

TABLE 4
Preferred HCVR. The CDRs and FRs are defined by their SEQ ID NOs.
The penultimate column refers to the SEQ ID NOs of the whole HCVR.

| HCVR | | V_{H}- | | V_{H}- | V_{H}- | V_{H}- | | HCVR | |
|---|---|---|---|---|---|---|---|---|---|
| # | V_{H}-FR1 | CDR1 | V_{H}-FR2 | CDR2 | FR3 | CDR3 | V_{H}-FR4 | sequence | Name |
| #1 | 25 | 1 | 26 | 4 | 27 | 3 | 28 | 61 | Mouse |
| | | | | | | | | | VH |
| #2 | 29 | 1 | 30 | 4 | 31 | 3 | 32 | 62 | 12VHA |
| #3 | 33 | 1 | 34 | 4 | 35 | 3 | 32 | 63 | 551VHA |
| #4 | 36 | 1 | 37 | 5 | 38 | 3 | 32 | 64 | 311VHA |
| #5 | 29 | 1 | 30 | 6 | 31 | 3 | 32 | 65 | 12VHB |
| #6 | 39 | 1 | 34 | 7 | 35 | 3 | 32 | 66 | 551VHB |
| #7 | 40 | 1 | 37 | 8 | 41 | 3 | 32 | 67 | 311VHB |
| #8 | 29 | 1 | 30 | 9 | 31 | 3 | 32 | 68 | 12VHC |
| #9 | 42 | 1 | 34 | 10 | 35 | 3 | 32 | 69 | 551VHC |
| #10 | 40 | 1 | 43 | 11 | 41 | 3 | 32 | 70 | 331VHC |
| #11 | 29 | 1 | 30 | 100 | 31 | 3 | 32 | 101 | 12VHD |
| #12 | 29 | 1 | 30 | 116 | 31 | 3 | 32 | 121 | 12VHE |
| #13 | 29 | 1 | 30 | 117 | 31 | 3 | 32 | 122 | 12VHF |
| #14 | 29 | 1 | 30 | 118 | 31 | 3 | 32 | 123 | 12VHG |
| #15 | 29 | 1 | 30 | 119 | 31 | 3 | 32 | 124 | 12VHH |

In a preferred embodiment, the antibody or binding fragment thereof comprises a HCVR comprising or consisting of the sequence SEQ ID NO: 61; or a HCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 61.

SEQ ID NO: 61
QVQLQQSGAELVRPGTSVKMSCKAAGYTFTNYYIGWVKQRPGHGLEWIGD
IFPGGDYANSNEKFKGKATLTADTSSSTAYMQLSSLTSEDSAIYYCVRRN
FDYWGQGTTLTVSS

In a preferred embodiment, the antibody or binding fragment thereof comprises a HCVR comprising or consisting of the sequence SEQ ID NO: 62; or a HCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 62.

SEQ ID NO: 62
QVQLVQSGAEVKKPGASVKVSCKASGYTFTNYYIGWVRQAPGQGLEWIGD
IFPGGDYANSNEKFKGRVTLTADTSISTAYMELSRLRSDDTVVYYCVRRN
FDYWGQGTLVTVSS

In a preferred embodiment, the antibody or binding fragment thereof comprises a HCVR comprising or consisting of the sequence SEQ ID NO: 63; or a HCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 63.

SEQ ID NO: 63
EVQLVQSGAEVKKPGESLKISCKASGYTFTNYYIGWVRQMPGKGLEWIGD
IFPGGDYANSNEKFKGQVTLSADKSISTAYLQLSSLKASDTAMYYCVRRN
FDYWGQGTLVTVSS

In a preferred embodiment, the antibody or binding fragment thereof comprises a HCVR comprising or consisting of the sequence SEQ ID NO: 64; or a HCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 64.

SEQ ID NO: 64
QVQLVESGGGLVKPGGSLRLSCAASGYTFTNYYIGWIRQAPGKGLEWIGD
IFPGGDYANSNEKVKGRFTLSADTAKNSAYLQMNSLRAEDTAVYYCVRRN
FDYWGQGTLVTVSS

In a preferred embodiment, the antibody or binding fragment thereof comprises a HCVR comprising or consisting of the sequence SEQ ID NO: 65; or a HCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 65.

SEQ ID NO: 65
QVQLVQSGAEVKKPGASVKVSCKASGYTFTNYYIGWVRQAPGQGLEWIGD
IFPGGGYTNYAEKFQGRVTLTADTSISTAYMELSRLRSDDTVVYYCVRRN
FDYWGQGTLVTVSS

In a preferred embodiment, the antibody or binding fragment thereof comprises a HCVR comprising or consisting of the sequence SEQ ID NO: 66; or a HCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 66.

SEQ ID NO: 66
EVQLVQSGAEVKKPGESLKISCKGSGYTFTNYYIGWVRQMPGKGLEWIGD
IFPGGSYTNYSESFQGQVTLSADKSISTAYLQLSSLKASDTAMYYCVRRN
FDYWGQGTLVTVSS

In a preferred embodiment, the antibody or binding fragment thereof comprises a HCVR comprising or consisting of the sequence SEQ ID NO: 67; or a HCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 67.

SEQ ID NO: 67
QVQLVESGGGLVKPGGSLRLSCAASGFTFSNYYIGWIRQAPGKGLEWIGD
IFPGGSYTNYADSVKGRFTLSADTAKNSLYLQMNSLRAEDTAVYYCVRRN
FDYWGQGTLVTVSS

In a preferred embodiment, the antibody or binding fragment thereof comprises a HCVR comprising or consisting of the sequence SEQ ID NO: 68; or a HCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 68.

SEQ ID NO: 68
QVQLVQSGAEVKKPGASVKVSCKASGYTFTNYYIGWVRQAPGQGLEWIGR
IFPGGGYTNYAQKFQGRVTLTADTSISTAYMELSRLRSDDTVVYYCVRRN
FDYWGQGTLVTVSS

In a preferred embodiment, the antibody or binding fragment thereof comprises a HCVR comprising or consisting of the sequence SEQ ID NO: 69; or a HCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 69.

SEQ ID NO: 69
EVQLVQSGAEVKKPGESLKISCKGSGYSFTNYYIGWVRQMPGKGLEWIGI
IFPGGSYTNYSPSFQGQVTLSADKSISTAYLQLSSLKASDTAMYYCVRRN
FDYWGQGTLVTVSS

In a preferred embodiment, the antibody or binding fragment thereof comprises a HCVR comprising or consisting of the sequence SEQ ID NO: 70; or a HCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 70.

SEQ ID NO: 70
QVQLVESGGGLVKPGGSLRLSCAASGFTFSNYYIGWIRQAPGKGLEWVGD
IFSGGSYTNYADSVKGRFTLSADTAKNSLYLQMNSLRAEDTAVYYCVRRN
FDYWGQGTLVTVSS

In a preferred embodiment, the antibody or binding fragment thereof comprises a HCVR comprising or consisting of the sequence SEQ ID NO: 101; or a HCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 101.

SEQ ID NO: 101
QVQLVQSGAEVKKPGASVKVSCKASGYTFTNYYIGWVRQAPGQGLEWIGD
IFPGGDYTNYAEKFQGRVTLTADTSISTAYMELSRLRSDDTVVYYCVRRN
FDYWGQGTLVTVSS

In a preferred embodiment, the antibody or binding fragment thereof comprises a HCVR comprising or consisting of the sequence SEQ ID NO: 121; or a HCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 121.

SEQ ID NO: 121
QVQLVQSGAEVKKPGASVKVSCKASGYTFTNYYIGWVRQAPGQGLEWIGD
IFPGGGYANYAEKFQGRVTLTADTSISTAYMELSRLRSDDTVVYYCVRRN
FDYWGQGTLVTVSS

In a preferred embodiment, the antibody or binding fragment thereof comprises a HCVR comprising or consisting of the sequence SEQ ID NO: 122; or a HCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 122.

SEQ ID NO: 122
QVQLVQSGAEVKKPGASVKVSCKASGYTFTNYYIGWVRQAPGQGLEWIGD
IFPGGGYTNYAEKFKGRVTLTADTSISTAYMELSRLRSDDTVVYYCVRRN
FDYWGQGTLVTVSS

In a preferred embodiment, the antibody or binding fragment thereof comprises a HCVR comprising or consisting of the sequence SEQ ID NO: 123; or a HCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 123.

SEQ ID NO: 123
QVQLVQSGAEVKKPGASVKVSCKASGYTFTNYYIGWVRQAPGQGLEWIGD
IFPGGGYTNYNEKFQGRVTLTADTSISTAYMELSRLRSDDTVVYYCVRRN
FDYWGQGTLVTVSS

In a preferred embodiment, the antibody or binding fragment thereof comprises a HCVR comprising or consisting of the sequence SEQ ID NO: 124; or a HCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 124.

SEQ ID NO: 124
QVQLVQSGAEVKKPGASVKVSCKASGYTFTNYYIGWVRQAPGQGLEWIGD
IFPGGGYTNSAEKFQGRVTLTADTSISTAYMELSRLRSDDTVVYYCVRRN
FDYWGQGTLVTVSS

In one embodiment, the antibody or binding fragment thereof comprises a LCVR comprising or consisting of:
a V_{L}-FR1 as described hereinabove,
a V_{L}-CDR1 as described hereinabove,
a V_{L}-FR2 as described hereinabove,
a V_{L}-CDR2 as described hereinabove,
a V_{L}-FR3 as described hereinabove,
a V_{L}-CDR3 as described hereinabove, and
a V_{L}-FR4 as described hereinabove.

In one embodiment, the antibody or binding fragment thereof comprises a HCVR comprising or consisting of:
a V_{L}-FR1 selected from SEQ ID NOs 44, 48, 52 and 55;
a V_{L}-CDR1 selected from SEQ ID NO 12;
a V_{L}-FR2 selected from SEQ ID NOs 45, 49, 53, 56, 58, 59 and 60;
a V_{L}-CDR2 selected from SEQ ID NO 13;
a V_{L}-FR3 selected from SEQ ID NOs 46, 50, 54 and 57;
a V_{L}-CDR3 selected from SEQ ID NO 14; and
a V_{L}-FR4 selected from SEQ ID NOs 47 and 51.

In one embodiment, the antibody or binding fragment thereof comprises a HCVR comprising or consisting of:
a V_{L}-FR1 selected from SEQ ID NOs 44, 48, 52 and 55;
a V_{L}-CDR1 selected from SEQ ID NOs 15 and 18;
a V_{L}-FR2 selected from SEQ ID NOs 45, 49, 53, 56, 58, 59 and 60;
a V_{L}-CDR2 selected from SEQ ID NOs 16, 19, 20, 22, 111 and 120;
a V_{L}-FR3 selected from SEQ ID NOs 46, 50, 54 and 57;
a V_{L}-CDR3 selected from SEQ ID NOs 17 and 21; and
a V_{L}-FR4 selected from SEQ ID NOs 47 and 51.

In a preferred embodiment, the antibody or binding fragment thereof comprises a LCVR comprising or consisting of a combination of a V_{L}-FR1, a V_{L}-CDR1, a V_{L}-FR2, a V_{L}-CDR2, a V_{L}-FR3, a V_{L}-CDR3 and a V_{L}-FR4, said combination being as defined in Table 5.

TABLE 5
Preferred LCVR. The CDRs and FRs are defined by their SEQ ID NOs.
The penultimate column refers to the SEQ ID NOs of the whole LCVR (with
a first sequence number with X₁₂ being absent or being selected from Asn (N),
Ser (S) and Gly (G); and X₁₈ being selected from Tyr (Y) and Phe (F); and a
second sequence number where preferred X₁₂ and X₁₈ are defined).

| LCVR | V_{L}- | V_{L}- | V_{L}- | V_{L}- | V_{L}- | V_{L}- | V_{L}- | LCVR | |
|---|---|---|---|---|---|---|---|---|---|
| # | FR1 | CDR1 | FR2 | CDR2 | FR3 | CDR3 | FR4 | sequences | Name |
| #1 | 44 | 15 | 45 | 16 | 46 | 17 | 47 | 71/81 | Mouse |
| | | | | | | | | | VL |
| #2 | 48 | 15 | 49 | 16 | 50 | 17 | 51 | 72/82 | 19VLA |
| #3 | 52 | 15 | 53 | 16 | 54 | 17 | 51 | 73/83 | 621VLA |
| #4 | 55 | 15 | 56 | 16 | 57 | 17 | 51 | 74/84 | 311VLA |
| #5 | 48 | 18 | 58 | 16 | 50 | 17 | 51 | 75/85 | 19VLB |
| #6 | 52 | 18 | 59 | 19 | 54 | 17 | 51 | 76/86 | 621VLB |
| #7 | 55 | 18 | 56 | 16 | 57 | 17 | 51 | 77/87 | 311VLB |
| #8 | 48 | 18 | 58 | 20 | 50 | 17 | 51 | 78/88 | 19VLC |
| #9 | 52 | 18 | 59 | 19 | 54 | 21 | 51 | 79/89 | 621VLC |
| #10 | 55 | 18 | 60 | 22 | 57 | 17 | 51 | 80/90 | 311VLC |
| #11 | 48 | 18 | 49 | 16 | 50 | 17 | 51 | 102/103 | 19VLD |
| #12 | 48 | 18 | 58 | ill | 50 | 17 | 51 | 112/113 | 19VLB |
| | | | | | | | | | “N50A” |
| #13 | 44 | 15 | 45 | 120 | 46 | 17 | 47 | 125/126 | Mouse |
| | | | | | | | | | “S52A” |
| #14 | 44 | 15 | 45 | 127 | 46 | 17 | 47 | 128/129 | 19VLB |
| | | | | | | | | | “N50X” |

In a one embodiment, the antibody or binding fragment thereof comprises a LCVR comprising or consisting of a combination of a V_{L}-FR1, a V_{L}-CDR1, a V_{L}-FR2, a V_{L}-CDR2, a V_{L}-FR3, a V_{L}-CDR3 and a V_{L}-FR4 as defined hereinabove, wherein X₁₈ is Phe (F) if X₁₂ is not absent (i.e., if X₁₂ is any of Asn (N), Ser (S) or Gly (G)). In a one embodiment, the antibody or binding fragment thereof comprises a LCVR comprising or consisting of a combination of a V_{L}-FR1, a V_{L}-CDR1, a V_{L}-FR2, a V_{L}-CDR2, a V_{L}-FR3, a V_{L}-CDR3 and a V_{L}-FR4 as defined hereinabove, wherein X₁₈ is selected from Tyr (Y) and Phe (F) if X₁₂ is absent.

In a preferred embodiment, the antibody or binding fragment thereof comprises a LCVR comprising or consisting of the sequence SEQ ID NO: 71, with X₁₂ being selected from Asn (N), Ser (S) and Gly (G); or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 71.

SEQ ID NO: 71
QIVLTQSPTIMSASPGEKVTITCSASSSVS-X₁₂-YMHWFQQKTGTSPRL
WIYNTSNLPSGVPARFSGSGSGTSFSLTISRMEAEDAATYYCQQRSSYPL
TFGAGTKLELK

In a preferred embodiment, the antibody or binding fragment thereof comprises a LCVR comprising or consisting of the sequence SEQ ID NO: 72, with X₁₂ being selected from Asn (N), Ser (S) and Gly (G); or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 72.

SEQ ID NO: 72
DIQLTQSPSFLSASVGDRVTITCSASSSVS-X₁₂-YMHWFQQKPGKAPKL
WIYNTSNLPSGVPSRFSGSGSGTEFTLTISSLQPEDFATYYCQQRSSYPL
TFGGGTKVEIK

In a preferred embodiment, the antibody or binding fragment thereof comprises a LCVR comprising or consisting of the sequence SEQ ID NO: 73, with X₁₂ being selected from Asn (N), Ser (S) and Gly (G); or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 73.

SEQ ID NO: 73
EIVLTQSPDFQSVTPKEKVTITCSASSSVS-X₁₂-YMHWFQQKPDQSPKL
WIYNTSNLPSGVPSRFSGSGSGTDFTLTINSLEAEDAATYYCQQRSSYPL
TFGGGTKVEIK

In a preferred embodiment, the antibody or binding fragment thereof comprises a LCVR comprising or consisting of the sequence SEQ ID NO: 74, with X₁₂ being selected from Asn (N), Ser (S) and Gly (G); or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 74.

SEQ ID NO: 74
EIVLTQSPATLSLSPGERATLSCSASSSVS-X₁₂-YMHWFQQKPGQAPRL
WIYNTSNLPSGIPARFSGSGSGTDFTLTISSLEPEDFAVYYCQQRSSYPL
TFGGGTKVEIK

In a preferred embodiment, the antibody or binding fragment thereof comprises a LCVR comprising or consisting of the sequence SEQ ID NO: 75, with X₁₂ being selected from Asn (N), Ser (S) and Gly (G); or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 75.

SEQ ID NO: 75
DIQLTQSPSFLSASVGDRVTITCRASSSVS-X₁₂-YMHWYQQKPGKAPKL
WIYNTSNLPSGVPSRFSGSGSGTEFTLTISSLQPEDFATYYCQQRSSYPL
TFGGGTKVEIK

In a preferred embodiment, the antibody or binding fragment thereof comprises a LCVR comprising or consisting of the sequence SEQ ID NO: 76, with X₁₂ being selected from Asn (N), Ser (S) and Gly (G); or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 76.

SEQ ID NO: 76
EIVLTQSPDFQSVTPKEKVTITCRASSSVS-X₁₂-YMHWYQQKPDQSPKL
WIYNTSNSPSGVPSRFSGSGSGTDFTLTINSLEAEDAATYYCQQRSSYPL
TFGGGTKVEIK

In a preferred embodiment, the antibody or binding fragment thereof comprises a LCVR comprising or consisting of the sequence SEQ ID NO: 77, with X₁₂ being selected from Asn (N), Ser (S) and Gly (G); or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 77.

SEQ ID NO: 77
EIVLTQSPATLSLSPGERATLSCRASSSVS-X₁₂-YMHWFQQKPGQAPRL
WIYNTSNLPSGIPARFSGSGSGTDFTLTISSLEPEDFAVYYCQQRSSYPL
TFGGGTKVEIK

In a preferred embodiment, the antibody or binding fragment thereof comprises a LCVR comprising or consisting of the sequence SEQ ID NO: 78, with X₁₂ being selected from Asn (N), Ser (S) and Gly (G); or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 78.

SEQ ID NO: 78
DIQLTQSPSFLSASVGDRVTITCRASSSVS-X₁₂-YMHWYQQKPGKAPKL
WIYATSNLQSGVPSRFSGSGSGTEFTLTISSLQPEDFATYYCQQRSSYPL
TFGGGTKVEIK

In a preferred embodiment, the antibody or binding fragment thereof comprises a LCVR comprising or consisting of the sequence SEQ ID NO: 79, with X₁₂ being selected from Asn (N), Ser (S) and Gly (G); or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 79.

SEQ ID NO: 79
EIVLTQSPDFQSVTPKEKVTITCRASSSVS-X₁₂-
YMHWYQQKPDQSPKLWIYNTSNSPSGVPSRFSGSG
SGTDFTLTINSLEAEDAATYYCHQRSSYPLTFGGG
TKVEIK

In a preferred embodiment, the antibody or binding fragment thereof comprises a LCVR comprising or consisting of the sequence SEQ ID NO: 80, with X₁₂ being selected from Asn (N), Ser (S) and Gly (G); or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 80.

SEQ ID NO: 80
EIVLTQSPATLSLSPGERATLSCRASSSVS-X₁₂-
YMHWYQQKPGQAPRLWIYNTSNRATGIPARFSGSG
SGTDFTLTISSLEPEDFAVYYCQQRSSYPLTFGGG
TKVEIK

In a preferred embodiment, the antibody or binding fragment thereof comprises a LCVR comprising or consisting of the sequence SEQ ID NO: 102, with X₁₂ being selected from Asn (N), Ser (S) and Gly (G); or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 102.

SEQ ID NO: 102
DIQLTQSPSFLSASVGDRVTITCRASSSVS-X₁₂-
YMHWFQQKPGKAPKLWIYNTSNLPSGVPSRFSGSG
SGTEFTLTISSLQPEDFATYYCQQRSSYPLTFGGG
TKVEIK

In a preferred embodiment, the antibody or binding fragment thereof comprises a LCVR comprising or consisting of the sequence SEQ ID NO: 112, with X₁₂ being selected from Asn (N), Ser (S) and Gly (G); or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 112.

SEQ ID NO: 112
DIQLTQSPSFLSASVGDRVTITCRASSSVS-X₁₂-
YMHWYQQKPGKAPKLWIYATSNLPSGVPSRFSGSG
SGTEFTLTISSLQPEDFATYYCQQRSSYPLTFGGG
TKVEIK

In a preferred embodiment, the antibody or binding fragment thereof comprises a LCVR comprising or consisting of the sequence SEQ ID NO: 125, with X₁₂ being selected from Asn (N), Ser (S) and Gly (G); or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 125.

SEQ ID NO: 125
QIVLTQSPTIMSASPGEKVTITCSASSSVS-X₁₂-
YMHWFQQKTGTSPRLWIYNTANLPSGVPARFSGSG
SGTSFSLTISRMEAEDAATYYCQQRSSYPLTFGAG
TKLELK

In a preferred embodiment, the antibody or binding fragment thereof comprises a LCVR comprising or consisting of the sequence SEQ ID NO: 128, with X₁₂ being selected from Asn (N), Ser (S) and Gly (G); and X₁₃ being any amino acid but Ala (A) or Asn (N); or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 128.

SEQ ID NO: 128
DIQLTQSPSFLSASVGDRVTITCRASSSVS-X₁₂-
YMHWYQQKPGKAPKLWIY-X₁₃-TSNLPSGVPSRF
SGSGSGTEFTLTISSLQPEDFATYYCQQRSSYPLT
FGGGTKVEIK

In one embodiment, the antibody or binding fragment thereof comprises a LCVR comprising or consisting of sequences SEQ ID NO: 71-80, 102, 112, 125 or 128, wherein

X₁₂ is absent.

In a preferred embodiment, the antibody or binding fragment thereof comprises a LCVR comprising or consisting of the sequence SEQ ID NO: 81; or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 81.

SEQ ID NO: 81
QIVLTQSPTIMSASPGEKVTITCSASSSVSYMHWF
QQKTGTSPRLWIYNTSNLPSGVPARFSGSGSGTSY
SLTISRMEAEDAATYYCQQRSSYPLTFGAGTKLEL
K

In a preferred embodiment, the antibody or binding fragment thereof comprises a LCVR comprising or consisting of the sequence SEQ ID NO: 82; or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 82.

SEQ ID NO: 82
DIQLTQSPSFLSASVGDRVTITCSASSSVSYMHWF
QQKPGKAPKLWIYNTSNLPSGVPSRFSGSGSGTEY
TLTISSLQPEDFATYYCQQRSSYPLTFGGGTKVEI
K

In a preferred embodiment, the antibody or binding fragment thereof comprises a LCVR comprising or consisting of the sequence SEQ ID NO: 83; or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 83.

SEQ ID NO: 83
EIVLTQSPDFQSVTPKEKVTITCSASSSVSYMHWF
QQKPDQSPKLWIYNTSNLPSGVPSRFSGSGSGTDY
TLTINSLEAEDAATYYCQQRSSYPLTFGGGTKVEI
K

In a preferred embodiment, the antibody or binding fragment thereof comprises a LCVR comprising or consisting of the sequence SEQ ID NO: 84; or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 84.

SEQ ID NO: 84
EIVLTQSPATLSLSPGERATLSCSASSSVSYMHWF
QQKPGQAPRLWIYNTSNLPSGIPARFSGSGSGTDY
TLTISSLEPEDFAVYYCQQRSSYPLTFGGGTKVEI
K

In a preferred embodiment, the antibody or binding fragment thereof comprises a LCVR comprising or consisting of the sequence SEQ ID NO: 85; or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 85.

SEQ ID NO: 85
DIQLTQSPSFLSASVGDRVTITCRASSSVSYMHWY
QQKPGKAPKLWIYNTSNLPSGVPSRFSGSGSGTEY
TLTISSLQPEDFATYYCQQRSSYPLTFGGGTKVEI
K

In a preferred embodiment, the antibody or binding fragment thereof comprises a LCVR comprising or consisting of the sequence SEQ ID NO: 86; or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 86.

SEQ ID NO: 86
EIVLTQSPDFQSVTPKEKVTITCRASSSVSYMHWY
QQKPDQSPKLWIYNTSNSPSGVPSRFSGSGSGTDF
TLTINSLEAEDAATYYCQQRSSYPLTFGGGTKVEI
K

In a preferred embodiment, the antibody or binding fragment thereof comprises a LCVR comprising or consisting of the sequence SEQ ID NO: 87; or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 87.

SEQ ID NO: 87
EIVLTQSPATLSLSPGERATLSCRASSSVSYMHWF
QQKPGQAPRLWIYNTSNLPSGIPARFSGSGSGTDY
TLTISSLEPEDFAVYYCQQRSSYPLTFGGGTKVEI
K

In a preferred embodiment, the antibody or binding fragment thereof comprises a LCVR comprising or consisting of the sequence SEQ ID NO: 88; or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 88.

SEQ ID NO: 88
DIQLTQSPSFLSASVGDRVTITCRASSSVSYMHWY
QQKPGKAPKLWIYATSNLQSGVPSRFSGSGSGTEF
TLTISSLQPEDFATYYCQQRSSYPLTFGGGTKVEI
K

In a preferred embodiment, the antibody or binding fragment thereof comprises a LCVR comprising or consisting of the sequence SEQ ID NO: 89; or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 89.

SEQ ID NO: 89
EIVLTQSPDFQSVTPKEKVTITCRASSSVSYMHWY
QQKPDQSPKLWIYNTSNSPSGVPSRFSGSGSGTDF
TLTINSLEAEDAATYYCHQRSSYPLTFGGGTKVEI
K

In a preferred embodiment, the antibody or binding fragment thereof comprises a LCVR comprising or consisting of the sequence SEQ ID NO: 90; or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 90.

SEQ ID NO: 90
EIVLTQSPATLSLSPGERATLSCRASSSVSYMHWY
QQKPGQAPRLWIYNTSNRATGIPARFSGSGSGTDF
TLTISSLEPEDFAVYYCQQRSSYPLTFGGGTKVEI
K

In a preferred embodiment, the antibody or binding fragment thereof comprises a LCVR comprising or consisting of the sequence SEQ ID NO: 103; or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 103.

SEQ ID NO: 103
DIQLTQSPSFLSASVGDRVTITCRASSSVSSYMHW
FQQKPGKAPKLWIYNTSNLPSGVPSRFSGSGSGTE
FTLTISSLQPEDFATYYCQQRSSYPLTFGGGTKVE
IK

In a preferred embodiment, the antibody or binding fragment thereof comprises a LCVR comprising or consisting of the sequence SEQ ID NO: 113; or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 113.

SEQ ID NO: 113
DIQLTQSPSFLSASVGDRVTITCRASSSVSYMHWY
QQKPGKAPKLWIYATSNLPSGVPSRFSGSGSGTEY
TLTISSLQPEDFATYYCQQRSSYPLTFGGGTKVEI
K

In a preferred embodiment, the antibody or binding fragment thereof comprises a LCVR comprising or consisting of the sequence SEQ ID NO: 126; or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 126.

SEQ ID NO: 126
QIVLTQSPTIMSASPGEKVTITCSASSSVSYMHWF
QQKTGTSPRLWIYNTANLPSGVPARFSGSGSGTSY
SLTISRMEAEDAATYYCQQRSSYPLTFGAGTKLEL
K

In a preferred embodiment, the antibody or binding fragment thereof comprises a LCVR comprising or consisting of the sequence SEQ ID NO: 129, with X₁₃ being any amino acid but Ala (A) or Asn (N); or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NO: 129.

SEQ ID NO: 129
DIQLTQSPSFLSASVGDRVTITCRASSSVSYMHWY
QQKPGKAPKLWIY-X13-TSNLPSGVPSRFSGSGS
GTEYTLTISSLQPEDFATYYCQQRSSYPLTFGGGT
KVEIK

In one embodiment, the antibody or binding fragment thereof comprises:
a HCVR as defined hereinabove; and
a LCVR as defined hereinabove.

In one embodiment, the antibody or binding fragment thereof comprises:
a HCVR selected from SEQ ID NOs: 61, 62, 63, 64, 65, 66, 67, 68, 69, 70, 101, 121, 122, 123 and 124; or a HCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NOs: 61, 62, 63, 64, 65, 66, 67, 68, 69, 70, 101, 121, 122, 123 or 124; and
a LCVR selected from SEQ ID NOs: 71, 72, 73, 74, 75, 76, 77, 78, 79, 80, 102, 112, 125 and 128, with X₁₂ being selected from Asn (N), Ser (S) and Gly (G), and X₁₃ being any amino acid but Ala (A) or Asn (N); or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NOs: 71, 72, 73, 74, 75, 76, 77, 78, 79, 80, 102, 112, 125 or 128.

In one embodiment, the antibody or binding fragment thereof comprises:
a HCVR selected from SEQ ID NOs: 61, 62, 63, 64, 65, 66, 67, 68, 69, 70, 101, 121, 122, 123 and 124; or a HCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NOs: 61, 62, 63, 64, 65, 66, 67, 68, 69, 70, 101, 121, 122, 123 or 124; and
a LCVR selected from SEQ ID NOs: 81, 82, 83, 84, 85, 86, 87, 88, 89, 90, 103, 113, 126 and 129, with X₁₃ being any amino acid but Ala (A) or Asn (N); or a LCVR comprising or consisting of a sequence of the non-CDR regions sharing at least 70%, 75%, 80%, 85%, 90%, 95%, 96%, 97%, 98%, 99% or more of identity with the sequence of the non-CDR regions of SEQ ID NOs: 81, 82, 83, 84, 85, 86, 87, 88, 89, 90, 103, 113, 126 or 129.

In one embodiment, the antibody or binding fragment thereof comprises a HCVR of SEQ ID NO: 61 and a LCVR of SEQ ID NO: 71, with X₁₂ being selected from Asn (N), Ser (S) and Gly (G).

In one embodiment, the antibody or binding fragment thereof comprises a HCVR of SEQ ID NO: 61 and a LCVR of SEQ ID NO: 72, with X₁₂ being selected from Asn (N), Ser (S) and Gly (G).

In one embodiment, the antibody or binding fragment thereof comprises a HCVR of SEQ ID NO: 61 and a LCVR of SEQ ID NO: 73, with X₁₂ being selected from Asn (N), Ser (S) and Gly (G).

In one embodiment, the antibody or binding fragment thereof comprises a HCVR of SEQ ID NO: 61 and a LCVR of SEQ ID NO: 74, with X₁₂ being selected from Asn (N), In one embodiment, the antibody or binding fragment thereof comprises a HCVR of SEQ ID NO: 61 and a LCVR of SEQ ID NO: 75, with X₁₂ being selected from Asn (N), Ser (S) and Gly (G).

In one embodiment, the antibody or binding fragment thereof comprises a HCVR of SEQ ID NO: 61 and a LCVR of SEQ ID NO: 76, with X₁₂ being selected from Asn (N), Ser (S) and Gly (G).

In one embodiment, the antibody or binding fragment thereof comprises a HCVR of SEQ ID NO: 61 and a LCVR of SEQ ID NO: 77, with X₁₂ being selected from Asn (N), Ser (S) and Gly (G).

In one embodiment, the antibody or binding fragment thereof comprises a HCVR of SEQ ID NO: 61 and a LCVR of SEQ ID NO: 78, with X₁₂ being selected from Asn (N), Ser (S) and Gly (G).

In one embodiment, the antibody or binding fragment thereof comprises a HCVR of SEQ ID NO: 61 and a LCVR of SEQ ID NO: 79, with X₁₂ being selected from Asn (N), Ser (S) and Gly (G).

In one embodiment, the antibody or binding fragment thereof comprises a HCVR of SEQ ID NO: 61 and a LCVR of SEQ ID NO: 80, with X₁₂ being selected from Asn (N), Ser (S) and Gly (G).

In one embodiment, the antibody or binding fragment thereof comprises a HCVR of SEQ ID NO: 61 and a LCVR of SEQ ID NO: 102, with X₁₂ being selected from Asn (N), Ser (S) and Gly (G).

In one embodiment, the antibody or binding fragment thereof comprises a HCVR of SEQ ID NO: 61 and a LCVR of SEQ ID NO: 112, with X₁₂ being selected from Asn (N), Ser (S) and Gly (G).

In one embodiment, the antibody or binding fragment thereof comprises a HCVR of SEQ ID NO: 61 and a LCVR of SEQ ID NO: 125, with X₁₂ being selected from Asn (N), In one embodiment, the antibody or binding fragment thereof comprises a HCVR of SEQ ID NO: 62 and a LCVR of SEQ ID NO: 71, with X₁₂ being selected from Asn (N), Ser (S) and Gly (G).

In one embodiment, the antibody or binding fragment thereof comprises a HCVR of SEQ ID NO: 62 and a LCVR of SEQ ID NO: 72, with X₁₂ being selected from Asn (N), Ser (S) and Gly (G).

In one embodiment, the antibody or binding fragment thereof comprises a HCVR of SEQ ID NO: 62 and a LCVR of SEQ ID NO: 73, with X₁₂ being selected from Asn (N), Ser (S) and Gly (G).

In one embodiment, the antibody or binding fragment thereof comprises a HCVR of SEQ ID NO: 62 and a LCVR of SEQ ID NO: 74, with X₁₂ being selected from Asn (N), Ser (S) and Gly (G).

In one embodiment, the antibody or binding fragment thereof comprises a HCVR of SEQ ID NO: 62 and a LCVR of SEQ ID NO: 75, with X₁₂ being selected from Asn (N), Ser (S) and Gly (G).

In one embodiment, the antibody or binding fragment thereof comprises a HCVR of SEQ ID NO: 62 and a LCVR of SEQ ID NO: 76, with X₁₂ being selected from Asn (N), Ser (S) and Gly (G).

In one embodiment, the antibody or binding fragment thereof comprises a HCVR of SEQ ID NO: 62 and a LCVR of SEQ ID NO: 77, with X₁₂ being selected from Asn (N), Ser (S) and Gly (G).

In one embodiment, the antibody or binding fragment thereof comprises a HCVR of SEQ ID NO: 62 and a LCVR of SEQ ID NO: 78, with X₁₂ being selected from Asn (N), Ser (S) and Gly (G).

In one embodiment, the antibody or binding fragment thereof comprises a HCVR of SEQ ID NO: 62 and a LCVR of SEQ ID NO: 79, with X₁₂ being selected from Asn (N), In one embodiment, the antibody or binding fragment thereof comprises a HCVR of SEQ ID NO: 62 and a LCVR of SEQ ID NO: 80, with X₁₂ being selected from Asn (N), Ser (S) and Gly (G).

In one embodiment, the antibody or binding fragment thereof comprises a HCVR of SEQ ID NO: 62 and a LCVR of SEQ ID NO: 102, with X₁₂ being selected from Asn (N), Ser (S) and Gly (G).

In one embodiment, the antibody or binding fragment thereof comprises a HCVR of SEQ ID NO: 62 and a LCVR of SEQ ID NO: 112, with X₁₂ being selected from Asn (N), Ser (S) and Gly (G).

In one embodiment, the antibody or binding fragment thereof comprises a HCVR of SEQ ID NO: 62 and a LCVR of SEQ ID NO: 125, with X₁₂ being selected from Asn (N), Ser (S) and Gly (G).

In one embodiment, the antibody or binding fragment thereof comprises a HCVR of SEQ ID NO: 63 and a LCVR of SEQ ID NO: 71, with X₁₂ being selected from Asn (N), Ser (S) and Gly (G).

In one embodiment, the antibody or binding fragment thereof comprises a HCVR of SEQ ID NO: 63 and a LCVR of SEQ ID NO: 72, with X₁₂ being selected from Asn (N), Ser (S) and Gly (G).

In one embodiment, the antibody or binding fragment thereof comprises a HCVR of SEQ ID NO: 63 and a LCVR of SEQ ID NO: 73, with X₁₂ being selected from Asn (N), Ser (S) and Gly (G).

In one embodiment, the antibody or binding fragment thereof comprises a HCVR of SEQ ID NO: 63 and a LCVR of SEQ ID NO: 74, with X₁₂ being selected from Asn (N), Ser (S) and Gly (G).

In one embodiment, the antibody or binding fragment thereof comprises a HCVR of SEQ ID NO: 63 and a LCVR of SEQ ID NO: 75, with X₁₂ being selected from Asn (N), In one embodiment, the antibody or binding fragment thereof comprises a HCVR of SEQ ID NO: 63 and a LCVR of SEQ ID NO: 76, with X₁₂ being selected from Asn (N), Ser (S) and Gly (G).

In one embodiment, the antibody or binding fragment thereof comprises a HCVR of SEQ ID NO: 63 and a LCVR of SEQ ID NO: 77, with X₁₂ being selected from Asn (N), Ser (S) and Gly (G).

In one embodiment, the antibody or binding fragment thereof comprises a HCVR of SEQ ID NO: 63 and a LCVR of SEQ ID NO: 78, with X₁₂ being selected from Asn (N), Ser (S) and Gly (G).

In one embodiment, the antibody or binding fragment thereof comprises a HCVR of SEQ ID NO: 63 and a LCVR of SEQ ID NO: 79, with X₁₂ being selected from Asn (N), Ser (S) and Gly (G).

In one embodiment, the antibody or binding fragment thereof comprises a HCVR of SEQ ID NO: 63 and a LCVR of SEQ ID NO: 80, with X₁₂ being selected from Asn (N), Ser (S) and Gly (G).

In one embodiment, the antibody or binding fragment thereof comprises a HCVR of SEQ ID NO: 63 and a LCVR of SEQ ID NO: 102, with X₁₂ being selected from Asn (N), Ser (S) and Gly (G).

In one embodiment, the antibody or binding fragment thereof comprises a HCVR of SEQ ID NO: 63 and a LCVR of SEQ ID NO: 112, with X₁₂ being selected from Asn (N), Ser (S) and Gly (G).

In one embodiment, the antibody or binding fragment thereof comprises a HCVR of SEQ ID NO: 63 and a LCVR of SEQ ID NO: 125, with X₁₂ being selected from Asn (N), Ser (S) and Gly (G).

In one embodiment, the antibody or binding fragment thereof comprises a HCVR of SEQ ID NO: 64 and a LCVR of SEQ ID NO: 71, with X₁₂ being selected from Asn (N), In one embodiment, the antibody or binding fragment thereof comprises a HCVR of SEQ ID NO: 64 and a LCVR of SEQ ID NO: 72, with X₁₂ being selected from Asn (N), Ser (S) and Gly (G).

In one embodiment, the antibody or binding fragment thereof comprises a HCVR of SEQ ID NO: 64 and a LCVR of SEQ ID NO: 73, with X₁₂ being selected from Asn (N), Ser (S) and Gly (G).

In one embodiment, the antibody or binding fragment thereof comprises a HCVR of SEQ ID NO: 64 and a LCVR of SEQ ID NO: 74, with X₁₂ being selected from Asn (N), Ser (S) and Gly (G).

In one embodiment, the antibody or binding fragment thereof comprises a HCVR of SEQ ID NO: 64 and a LCVR of SEQ ID NO: 75, with X₁₂ being selected from Asn (N), Ser (S) and Gly (G).

In one embodiment, the antibody or binding fragment thereof comprises a HCVR of SEQ ID NO: 64 and a LCVR of SEQ ID NO: 76, with X₁₂ being selected from Asn (N), Ser (S) and Gly (G).

In one embodiment, the antibody or binding fragment thereof comprises a HCVR of SEQ ID NO: 64 and a LCVR of SEQ ID NO: 77, with X₁₂ being selected from Asn (N), Ser (S) and Gly (G).

In one embodiment, the antibody or binding fragment thereof comprises a HCVR of SEQ ID NO: 64 and a LCVR of SEQ ID NO: 78, with X₁₂ being selected from Asn (N), Ser (S) and Gly (G).

In one embodiment, the antibody or binding fragment thereof comprises a HCVR of SEQ ID NO: 64 and a LCVR of SEQ ID NO: 79, with X₁₂ being selected from Asn (N), Ser (S) and Gly (G).

In one embodiment, the antibody or binding fragment thereof comprises a HCVR of SEQ ID NO: 64 and a LCVR of SEQ ID NO: 80, with X₁₂ being selected from Asn (N), In one embodiment, the antibody or binding fragment thereof comprises a HCVR of SEQ ID NO: 64 and a LCVR of SEQ ID NO: 102, with X₁₂ being selected from Asn (N), Ser (S) and Gly (G).

In one embodiment, the antibody or binding fragment thereof comprises a HCVR of SEQ ID NO: 64 and a LCVR of SEQ ID NO: 112, with X₁₂ being selected from Asn (N), Ser (S) and Gly (G).

In one embodiment, the antibody or binding fragment thereof comprises a HCVR of SEQ ID NO: 64 and a LCVR of SEQ ID NO: 125, with X₁₂ being selected from Asn (N), Ser (S) and Gly (G).

In one embodiment, the antibody or binding fragment thereof comprises a HCVR of SEQ ID NO: 65 and a LCVR of SEQ ID NO: 71, with X₁₂ being selected from Asn (N), Ser (S) and Gly (G).

In one embodiment, the antibody or binding fragment thereof comprises a HCVR of SEQ ID NO: 65 and a LCVR of SEQ ID NO: 72, with X₁₂ being selected from Asn (N), Ser (S) and Gly (G).

In one embodiment, the antibody or binding fragment thereof comprises a HCVR of SEQ ID NO: 65 and a LCVR of SEQ ID NO: 73, with X₁₂ being selected from Asn (N), Ser (S) and Gly (G).

In one embodiment, the antibody or binding fragment thereof comprises a HCVR of SEQ ID NO: 65 and a LCVR of SEQ ID NO: 74, with X₁₂ being selected from Asn (N), Ser (S) and Gly (G).

In one embodiment, the antibody or binding fragment thereof comprises a HCVR of SEQ ID NO: 65 and a LCVR of SEQ ID NO: 75, with X₁₂ being selected from Asn (N), Ser (S) and Gly (G).

In one embodiment, the antibody or binding fragment thereof comprises a HCVR of SEQ ID NO: 65 and a LCVR of SEQ ID NO: 76, with X₁₂ being selected from Asn (N), In one embodiment, the antibody or binding fragment thereof comprises a HCVR of SEQ ID NO: 65 and a LCVR of SEQ ID NO: 77, with X₁₂ being selected from Asn (N), Ser (S) and Gly (G).

In one embodiment, the antibody or binding fragment thereof comprises a HCVR of SEQ ID NO: 65 and a LCVR of SEQ ID NO: 78, with X₁₂ being selected from Asn (N), Ser (S) and Gly (G).

In one embodiment, the antibody or binding fragment thereof comprises a HCVR of SEQ ID NO: 65 and a LCVR of SEQ ID NO: 79, with X₁₂ being selected from Asn (N), Ser (S) and Gly (G).

In one embodiment, the antibody or binding fragment thereof comprises a HCVR of SEQ ID NO: 65 and a LCVR of SEQ ID NO: 80, with X₁₂ being selected from Asn (N), Ser (S) and Gly (G).

In one embodiment, the antibody or binding fragment thereof comprises a HCVR of SEQ ID NO: 65 and a LCVR of SEQ ID NO: 102, with X₁₂ being selected from Asn (N), Ser (S) and Gly (G).

In one embodiment, the antibody or binding fragment thereof comprises a HCVR of SEQ ID NO: 65 and a LCVR of SEQ ID NO: 112, with X₁₂ being selected from Asn (N), Ser (S) and Gly (G).

In one embodiment, the antibody or binding fragment thereof comprises a HCVR of SEQ ID NO: 65 and a LCVR of SEQ ID NO: 125, with X₁₂ being selected from Asn (N), Ser (S) and Gly (G).

In one embodiment, the antibody or binding fragment thereof comprises a HCVR of SEQ ID NO: 66 and a LCVR of SEQ ID NO: 71, with X₁₂ being selected from Asn (N), Ser (S) and Gly (G).

In one embodiment, the antibody or binding fragment thereof comprises a HCVR of SEQ ID NO: 66 and a LCVR of SEQ ID NO: 72, with X₁₂ being selected from Asn (N), In one embodiment, the antibody or binding fragment thereof comprises a HCVR of SEQ ID NO: 66 and a LCVR of SEQ ID NO: 73, with X₁₂ being selected from Asn (N), Ser (S) and Gly (G).

In one embodiment, the antibody or binding fragment thereof comprises a HCVR of SEQ ID NO: 66 and a LCVR of SEQ ID NO: 74, with X₁₂ being selected from Asn (N), Ser (S) and Gly (G).

In one embodiment, the antibody or binding fragment thereof comprises a HCVR of SEQ ID NO: 66 and a LCVR of SEQ ID NO: 75, with X₁₂ being selected from Asn (N), Ser (S) and Gly (G).

In one embodiment, the antibody or binding fragment thereof comprises a HCVR of SEQ ID NO: 66 and a LCVR of SEQ ID NO: 76, with X₁₂ being selected from Asn (N), Ser (S) and Gly (G).

In one embodiment, the antibody or binding fragment thereof comprises a HCVR of SEQ ID NO: 66 and a LCVR of SEQ ID NO: 77, with X₁₂ being selected from Asn (N), Ser (S) and Gly (G).

In one embodiment, the antibody or binding fragment thereof comprises a HCVR of SEQ ID NO: 66 and a LCVR of SEQ ID NO: 78, with X₁₂ being selected from Asn (N), Ser (S) and Gly (G).

In one embodiment, the antibody or binding fragment thereof comprises a HCVR of SEQ ID NO: 66 and a LCVR of SEQ ID NO: 79, with X₁₂ being selected from Asn (N), Ser (S) and Gly (G).

In one embodiment, the antibody or binding fragment thereof comprises a HCVR of SEQ ID NO: 66 and a LCVR of SEQ ID NO: 80, with X₁₂ being selected from Asn (N), Ser (S) and Gly (G).

In one embodiment, the antibody or binding fragment thereof comprises a HCVR of SEQ ID NO: 66 and a LCVR of SEQ ID NO: 102, with X₁₂ being selected from Asn (N), In one embodiment, the antibody or binding fragment thereof comprises a HCVR of SEQ ID NO: 66 and a LCVR of SEQ ID NO: 112, with X₁₂ being selected from Asn (N), Ser (S) and Gly (G).

In one embodiment, the antibody or binding fragment thereof comprises a HCVR of SEQ ID NO: 66 and a LCVR of SEQ ID NO: 125, with X₁₂ being selected from Asn (N), Ser (S) and Gly (G).

In one embodiment, the antibody or binding fragment thereof comprises a HCVR of SEQ ID NO: 67 and a LCVR of SEQ ID NO: 71, with X₁₂ being selected from Asn (N), Ser (S) and Gly (G).

In one embodiment, the antibody or binding fragment thereof comprises a HCVR of SEQ ID NO: 67 and a LCVR of SEQ ID NO: 72, with X₁₂ being selected from Asn (N), Ser (S) and Gly (G).

In one embodiment, the antibody or binding fragment thereof comprises a HCVR of SEQ ID NO: 67 and a LCVR of SEQ ID NO: 73, with X₁₂ being selected from Asn (N), Ser (S) and Gly (G).

In one embodiment, the antibody or binding fragment thereof comprises a HCVR of SEQ ID NO: 67 and a LCVR of SEQ ID NO: 74, with X₁₂ being selected from Asn (N), Ser (S) and Gly (G).

In one embodiment, the antibody or binding fragment thereof comprises a HCVR of SEQ ID NO: 67 and a LCVR of SEQ ID NO: 75, with X₁₂ being selected from Asn (N), Ser (S) and Gly (G).

In one embodiment, the antibody or binding fragment thereof comprises a HCVR of SEQ ID NO: 67 and a LCVR of SEQ ID NO: 76, with X₁₂ being selected from Asn (N), Ser (S) and Gly (G).

In one embodiment, the antibody or binding fragment thereof comprises a HCVR of SEQ ID NO: 67 and a LCVR of SEQ ID NO: 77, with X₁₂ being selected from Asn (N), In one embodiment, the antibody or binding fragment thereof comprises a HCVR of SEQ ID NO: 67 and a LCVR of SEQ ID NO: 78, with X₁₂ being selected from Asn (N), Ser (S) and Gly (G).

In one embodiment, the antibody or binding fragment thereof comprises a HCVR of SEQ ID NO: 67 and a LCVR of SEQ ID NO: 79, with X₁₂ being selected from Asn (N), Ser (S) and Gly (G).

In one embodiment, the antibody or binding fragment thereof comprises a HCVR of SEQ ID NO: 67 and a LCVR of SEQ ID NO: 80, with X₁₂ being selected from Asn (N), Ser (S) and Gly (G).

In one embodiment, the antibody or binding fragment thereof comprises a HCVR of SEQ ID NO: 67 and a LCVR of SEQ ID NO: 102, with X₁₂ being selected from Asn (N), Ser (S) and Gly (G).

In one embodiment, the antibody or binding fragment thereof comprises a HCVR of SEQ ID NO: 67 and a LCVR of SEQ ID NO: 112, with X₁₂ being selected from Asn (N), Ser (S) and Gly (G).

In one embodiment, the antibody or binding fragment thereof comprises a HCVR of SEQ ID NO: 67 and a LCVR of SEQ ID NO: 125, with X₁₂ being selected from Asn (N), Ser (S) and Gly (G).

In one embodiment, the antibody or binding fragment thereof comprises a HCVR of SEQ ID NO: 68 and a LCVR of SEQ ID NO: 71, with X₁₂ being selected from Asn (N), Ser (S) and Gly (G).

In one embodiment, the antibody or binding fragment thereof comprises a HCVR of SEQ ID NO: 68 and a LCVR of SEQ ID NO: 72, with X₁₂ being selected from Asn (N), Ser (S) and Gly (G).

In one embodiment, the antibody or binding fragment thereof comprises a HCVR of SEQ ID NO: 68 and a LCVR of SEQ ID NO: 73, with X₁₂ being selected from Asn (N), In one embodiment, the antibody or binding fragment thereof comprises a HCVR of SEQ ID NO: 68 and a LCVR of SEQ ID NO: 74, with X₁₂ being selected from Asn (N), Ser (S) and Gly (G).

In one embodiment, the antibody or binding fragment thereof comprises a HCVR of SEQ ID NO: 68 and a LCVR of SEQ ID NO: 75, with X₁₂ being selected from Asn (N), Ser (S) and Gly (G).

In one embodiment, the antibody or binding fragment thereof comprises a HCVR of SEQ ID NO: 68 and a LCVR of SEQ ID NO: 76, with X₁₂ being selected from Asn (N), Ser (S) and Gly (G).

In one embodiment, the antibody or binding fragment thereof comprises a HCVR of SEQ ID NO: 68 and a LCVR of SEQ ID NO: 77, with X₁₂ being selected from Asn (N), Ser (S) and Gly (G).

In one embodiment, the antibody or binding fragment thereof comprises a HCVR of SEQ ID NO: 68 and a LCVR of SEQ ID NO: 78, with X₁₂ being selected from Asn (N), Ser (S) and Gly (G).

In one embodiment, the antibody or binding fragment thereof comprises a HCVR of SEQ ID NO: 68 and a LCVR of SEQ ID NO: 79, with X₁₂ being selected from Asn (N), Ser (S) and Gly (G).

In one embodiment, the antibody or binding fragment thereof comprises a HCVR of SEQ ID NO: 68 and a LCVR of SEQ ID NO: 80, with X₁₂ being selected from Asn (N), Ser (S) and Gly (G).

In one embodiment, the antibody or binding fragment thereof comprises a HCVR of SEQ ID NO: 68 and a LCVR of SEQ ID NO: 102, with X₁₂ being selected from Asn (N), Ser (S) and Gly (G).

In one embodiment, the antibody or binding fragment thereof comprises a HCVR of SEQ ID NO: 68 and a LCVR of SEQ ID NO: 112, with X₁₂ being selected from Asn (N), In one embodiment, the antibody or binding fragment thereof comprises a HCVR of SEQ ID NO: 68 and a LCVR of SEQ ID NO: 125, with X₁₂ being selected from Asn (N), Ser (S) and Gly (G).

In one embodiment, the antibody or binding fragment thereof comprises a HCVR of SEQ ID NO: 69 and a LCVR of SEQ ID NO: 71, with X₁₂ being selected from Asn (N), Ser (S) and Gly (G).

In one embodiment, the antibody or binding fragment thereof comprises a HCVR of SEQ ID NO: 69 and a LCVR of SEQ ID NO: 72, with X₁₂ being selected from Asn (N), Ser (S) and Gly (G).

In one embodiment, the antibody or binding fragment thereof comprises a HCVR of SEQ ID NO: 69 and a LCVR of SEQ ID NO: 73, with X₁₂ being selected from Asn (N), Ser (S) and Gly (G).

In one embodiment, the antibody or binding fragment thereof comprises a HCVR of SEQ ID NO: 69 and a LCVR of SEQ ID NO: 74, with X₁₂ being selected from Asn (N), Ser (S) and Gly (G).

In one embodiment, the antibody or binding fragment thereof comprises a HCVR of SEQ ID NO: 69 and a LCVR of SEQ ID NO: 75, with X₁₂ being selected from Asn (N), Ser (S) and Gly (G).

In one embodiment, the antibody or binding fragment thereof comprises a HCVR of SEQ ID NO: 69 and a LCVR of SEQ ID NO: 76, with X₁₂ being selected from Asn (N), Ser (S) and Gly (G).

In one embodiment, the antibody or binding fragment thereof comprises a HCVR of SEQ ID NO: 69 and a LCVR of SEQ ID NO: 77, with X₁₂ being selected from Asn (N), Ser (S) and Gly (G).

In one embodiment, the antibody or binding fragment thereof comprises a HCVR of SEQ ID NO: 69 and a LCVR of SEQ ID NO: 78, with X₁₂ being selected from Asn (N), In one embodiment, the antibody or binding fragment thereof comprises a HCVR of SEQ ID NO: 69 and a LCVR of SEQ ID NO: 79, with X₁₂ being selected from Asn (N), Ser (S) and Gly (G).

In one embodiment, the antibody or binding fragment thereof comprises a HCVR of SEQ ID NO: 69 and a LCVR of SEQ ID NO: 80, with X₁₂ being selected from Asn (N), Ser (S) and Gly (G).

In one embodiment, the antibody or binding fragment thereof comprises a HCVR of SEQ ID NO: 69 and a LCVR of SEQ ID NO: 102, with X₁₂ being selected from Asn (N), Ser (S) and Gly (G).

In one embodiment, the antibody or binding fragment thereof comprises a HCVR of SEQ ID NO: 69 and a LCVR of SEQ ID NO: 112, with X₁₂ being selected from Asn (N), Ser (S) and Gly (G).

In one embodiment, the antibody or binding fragment thereof comprises a HCVR of SEQ ID NO: 69 and a LCVR of SEQ ID NO: 125, with X₁₂ being selected from Asn (N), Ser (S) and Gly (G).

In one embodiment, the antibody or binding fragment thereof comprises a HCVR of SEQ ID NO: 70 and a LCVR of SEQ ID NO: 71, with X₁₂ being selected from Asn (N), Ser (S) and Gly (G).

In one embodiment, the antibody or binding fragment thereof comprises a HCVR of SEQ ID NO: 70 and a LCVR of SEQ ID NO: 72, with X₁₂ being selected from Asn (N), Ser (S) and Gly (G).

In one embodiment, the antibody or binding fragment thereof comprises a HCVR of SEQ ID NO: 70 and a LCVR of SEQ ID NO: 73, with X₁₂ being selected from Asn (N), Ser (S) and Gly (G).

In one embodiment, the antibody or binding fragment thereof comprises a HCVR of SEQ ID NO: 70 and a LCVR of SEQ ID NO: 74, with X₁₂ being selected from Asn (N), In one embodiment, the antibody or binding fragment thereof comprises a HCVR of SEQ ID NO: 70 and a LCVR of SEQ ID NO: 75, with X₁₂ being selected from Asn (N), Ser (S) and Gly (G).

In one embodiment, the antibody or binding fragment thereof comprises a HCVR of SEQ ID NO: 70 and a LCVR of SEQ ID NO: 76, with X₁₂ being selected from Asn (N), Ser (S) and Gly (G).

In one embodiment, the antibody or binding fragment thereof comprises a HCVR of SEQ ID NO: 70 and a LCVR of SEQ ID NO: 77, with X₁₂ being selected from Asn (N), Ser (S) and Gly (G).

In one embodiment, the antibody or binding fragment thereof comprises a HCVR of SEQ ID NO: 70 and a LCVR of SEQ ID NO: 78, with X₁₂ being selected from Asn (N), Ser (S) and Gly (G).

In one embodiment, the antibody or binding fragment thereof comprises a HCVR of SEQ ID NO: 70 and a LCVR of SEQ ID NO: 79, with X₁₂ being selected from Asn (N), Ser (S) and Gly (G).

In one embodiment, the antibody or binding fragment thereof comprises a HCVR of SEQ ID NO: 70 and a LCVR of SEQ ID NO: 80, with X₁₂ being selected from Asn (N), Ser (S) and Gly (G).

In one embodiment, the antibody or binding fragment thereof comprises a HCVR of SEQ ID NO: 70 and a LCVR of SEQ ID NO: 102, with X₁₂ being selected from Asn (N), Ser (S) and Gly (G).

In one embodiment, the antibody or binding fragment thereof comprises a HCVR of SEQ ID NO: 70 and a LCVR of SEQ ID NO: 112, with X₁₂ being selected from Asn (N), Ser (S) and Gly (G).

In one embodiment, the antibody or binding fragment thereof comprises a HCVR of SEQ ID NO: 70 and a LCVR of SEQ ID NO: 125, with X₁₂ being selected from Asn (N), In one embodiment, the antibody or binding fragment thereof comprises a HCVR of SEQ ID NO: 101 and a LCVR of SEQ ID NO: 71, with X₁₂ being selected from Asn (N), Ser (S) and Gly (G).

In one embodiment, the antibody or binding fragment thereof comprises a HCVR of SEQ ID NO: 101 and a LCVR of SEQ ID NO: 72, with X₁₂ being selected from Asn (N), Ser (S) and Gly (G).

In one embodiment, the antibody or binding fragment thereof comprises a HCVR of SEQ ID NO: 101 and a LCVR of SEQ ID NO: 73, with X₁₂ being selected from Asn (N), Ser (S) and Gly (G).

In one embodiment, the antibody or binding fragment thereof comprises a HCVR of SEQ ID NO: 101 and a LCVR of SEQ ID NO: 74, with X₁₂ being selected from Asn (N), Ser (S) and Gly (G).

In one embodiment, the antibody or binding fragment thereof comprises a HCVR of SEQ ID NO: 101 and a LCVR of SEQ ID NO: 75, with X₁₂ being selected from Asn (N), Ser (S) and Gly (G).

In one embodiment, the antibody or binding fragment thereof comprises a HCVR of SEQ ID NO: 101 and a LCVR of SEQ ID NO: 76, with X₁₂ being selected from Asn (N), Ser (S) and Gly (G).

In one embodiment, the antibody or binding fragment thereof comprises a HCVR of SEQ ID NO: 101 and a LCVR of SEQ ID NO: 77, with X₁₂ being selected from Asn (N), Ser (S) and Gly (G).

In one embodiment, the antibody or binding fragment thereof comprises a HCVR of SEQ ID NO: 101 and a LCVR of SEQ ID NO: 78, with X₁₂ being selected from Asn (N), Ser (S) and Gly (G).

In one embodiment, the antibody or binding fragment thereof comprises a HCVR of SEQ ID NO: 101 and a LCVR of SEQ ID NO: 79, with X₁₂ being selected from Asn (N), In one embodiment, the antibody or binding fragment thereof comprises a HCVR of SEQ ID NO: 101 and a LCVR of SEQ ID NO: 80, with X₁₂ being selected from Asn (N), Ser (S) and Gly (G).

In one embodiment, the antibody or binding fragment thereof comprises a HCVR of SEQ ID NO: 101 and a LCVR of SEQ ID NO: 102, with X₁₂ being selected from Asn (N), Ser (S) and Gly (G).

In one embodiment, the antibody or binding fragment thereof comprises a HCVR of SEQ ID NO: 101 and a LCVR of SEQ ID NO: 112, with X₁₂ being selected from Asn (N), Ser (S) and Gly (G).

In one embodiment, the antibody or binding fragment thereof comprises a HCVR of SEQ ID NO: 101 and a LCVR of SEQ ID NO: 125, with X₁₂ being selected from Asn (N), Ser (S) and Gly (G).

In one embodiment, the antibody or binding fragment thereof comprises a HCVR of SEQ ID NO: 121 and a LCVR of SEQ ID NO: 71, with X₁₂ being selected from Asn (N), Ser (S) and Gly (G).

In one embodiment, the antibody or binding fragment thereof comprises a HCVR of SEQ ID NO: 121 and a LCVR of SEQ ID NO: 72, with X₁₂ being selected from Asn (N), Ser (S) and Gly (G).

In one embodiment, the antibody or binding fragment thereof comprises a HCVR of SEQ ID NO: 121 and a LCVR of SEQ ID NO: 73, with X₁₂ being selected from Asn (N), Ser (S) and Gly (G).

In one embodiment, the antibody or binding fragment thereof comprises a HCVR of SEQ ID NO: 121 and a LCVR of SEQ ID NO: 74, with X₁₂ being selected from Asn (N), Ser (S) and Gly (G).

In one embodiment, the antibody or binding fragment thereof comprises a HCVR of SEQ ID NO: 121 and a LCVR of SEQ ID NO: 75, with X₁₂ being selected from Asn (N), In one embodiment, the antibody or binding fragment thereof comprises a HCVR of SEQ ID NO: 121 and a LCVR of SEQ ID NO: 76, with X₁₂ being selected from Asn (N), Ser (S) and Gly (G).

In one embodiment, the antibody or binding fragment thereof comprises a HCVR of SEQ ID NO: 121 and a LCVR of SEQ ID NO: 77, with X₁₂ being selected from Asn (N), Ser (S) and Gly (G).

In one embodiment, the antibody or binding fragment thereof comprises a HCVR of SEQ ID NO: 121 and a LCVR of SEQ ID NO: 78, with X₁₂ being selected from Asn (N), Ser (S) and Gly (G).

In one embodiment, the antibody or binding fragment thereof comprises a HCVR of SEQ ID NO: 121 and a LCVR of SEQ ID NO: 79, with X₁₂ being selected from Asn (N), Ser (S) and Gly (G).

In one embodiment, the antibody or binding fragment thereof comprises a HCVR of SEQ ID NO: 121 and a LCVR of SEQ ID NO: 80, with X₁₂ being selected from Asn (N), Ser (S) and Gly (G).

In one embodiment, the antibody or binding fragment thereof comprises a HCVR of SEQ ID NO: 121 and a LCVR of SEQ ID NO: 102, with X₁₂ being selected from Asn (N), Ser (S) and Gly (G).

In one embodiment, the antibody or binding fragment thereof comprises a HCVR of SEQ ID NO: 121 and a LCVR of SEQ ID NO: 112, with X₁₂ being selected from Asn (N), Ser (S) and Gly (G).

In one embodiment, the antibody or binding fragment thereof comprises a HCVR of SEQ ID NO: 121 and a LCVR of SEQ ID NO: 125, with X₁₂ being selected from Asn (N), Ser (S) and Gly (G).

In one embodiment, the antibody or binding fragment thereof comprises a HCVR of SEQ ID NO: 122 and a LCVR of SEQ ID NO: 71, with X₁₂ being selected from Asn (N), In one embodiment, the antibody or binding fragment thereof comprises a HCVR of SEQ ID NO: 122 and a LCVR of SEQ ID NO: 72, with X₁₂ being selected from Asn (N), Ser (S) and Gly (G).

In one embodiment, the antibody or binding fragment thereof comprises a HCVR of SEQ ID NO: 122 and a LCVR of SEQ ID NO: 73, with X₁₂ being selected from Asn (N), Ser (S) and Gly (G).

In one embodiment, the antibody or binding fragment thereof comprises a HCVR of SEQ ID NO: 122 and a LCVR of SEQ ID NO: 74, with X₁₂ being selected from Asn (N), Ser (S) and Gly (G).

In one embodiment, the antibody or binding fragment thereof comprises a HCVR of SEQ ID NO: 122 and a LCVR of SEQ ID NO: 75, with X₁₂ being selected from Asn (N), Ser (S) and Gly (G).

In one embodiment, the antibody or binding fragment thereof comprises a HCVR of SEQ ID NO: 122 and a LCVR of SEQ ID NO: 76, with X₁₂ being selected from Asn (N), Ser (S) and Gly (G).

In one embodiment, the antibody or binding fragment thereof comprises a HCVR of SEQ ID NO: 122 and a LCVR of SEQ ID NO: 77, with X₁₂ being selected from Asn (N), Ser (S) and Gly (G).

In one embodiment, the antibody or binding fragment thereof comprises a HCVR of SEQ ID NO: 122 and a LCVR of SEQ ID NO: 78, with X₁₂ being selected from Asn (N), Ser (S) and Gly (G).

In one embodiment, the antibody or binding fragment thereof comprises a HCVR of SEQ ID NO: 122 and a LCVR of SEQ ID NO: 79, with X₁₂ being selected from Asn (N), Ser (S) and Gly (G).

In one embodiment, the antibody or binding fragment thereof comprises a HCVR of SEQ ID NO: 122 and a LCVR of SEQ ID NO: 80, with X₁₂ being selected from Asn (N), In one embodiment, the antibody or binding fragment thereof comprises a HCVR of SEQ ID NO: 122 and a LCVR of SEQ ID NO: 102, with X₁₂ being selected from Asn (N), Ser (S) and Gly (G).

In one embodiment, the antibody or binding fragment thereof comprises a HCVR of SEQ ID NO: 122 and a LCVR of SEQ ID NO: 112, with X₁₂ being selected from Asn (N), Ser (S) and Gly (G).

In one embodiment, the antibody or binding fragment thereof comprises a HCVR of SEQ ID NO: 122 and a LCVR of SEQ ID NO: 125, with X₁₂ being selected from Asn (N), Ser (S) and Gly (G).

In one embodiment, the antibody or binding fragment thereof comprises a HCVR of SEQ ID NO: 123 and a LCVR of SEQ ID NO: 71, with X₁₂ being selected from Asn (N), Ser (S) and Gly (G).

In one embodiment, the antibody or binding fragment thereof comprises a HCVR of SEQ ID NO: 123 and a LCVR of SEQ ID NO: 72, with X₁₂ being selected from Asn (N), Ser (S) and Gly (G).

In one embodiment, the antibody or binding fragment thereof comprises a HCVR of SEQ ID NO: 123 and a LCVR of SEQ ID NO: 73, with X₁₂ being selected from Asn (N), Ser (S) and Gly (G).

In one embodiment, the antibody or binding fragment thereof comprises a HCVR of SEQ ID NO: 123 and a LCVR of SEQ ID NO: 74, with X₁₂ being selected from Asn (N), Ser (S) and Gly (G).

In one embodiment, the antibody or binding fragment thereof comprises a HCVR of SEQ ID NO: 123 and a LCVR of SEQ ID NO: 75, with X₁₂ being selected from Asn (N), Ser (S) and Gly (G).

In one embodiment, the antibody or binding fragment thereof comprises a HCVR of SEQ ID NO: 123 and a LCVR of SEQ ID NO: 76, with X₁₂ being selected from Asn (N), In one embodiment, the antibody or binding fragment thereof comprises a HCVR of SEQ ID NO: 123 and a LCVR of SEQ ID NO: 77, with X₁₂ being selected from Asn (N), Ser (S) and Gly (G).

In one embodiment, the antibody or binding fragment thereof comprises a HCVR of SEQ ID NO: 123 and a LCVR of SEQ ID NO: 78, with X₁₂ being selected from Asn (N), Ser (S) and Gly (G).

In one embodiment, the antibody or binding fragment thereof comprises a HCVR of SEQ ID NO: 123 and a LCVR of SEQ ID NO: 79, with X₁₂ being selected from Asn (N), Ser (S) and Gly (G).

In one embodiment, the antibody or binding fragment thereof comprises a HCVR of SEQ ID NO: 123 and a LCVR of SEQ ID NO: 80, with X₁₂ being selected from Asn (N), Ser (S) and Gly (G).

In one embodiment, the antibody or binding fragment thereof comprises a HCVR of SEQ ID NO: 123 and a LCVR of SEQ ID NO: 102, with X₁₂ being selected from Asn (N), Ser (S) and Gly (G).

In one embodiment, the antibody or binding fragment thereof comprises a HCVR of SEQ ID NO: 123 and a LCVR of SEQ ID NO: 112, with X₁₂ being selected from Asn (N), Ser (S) and Gly (G).

In one embodiment, the antibody or binding fragment thereof comprises a HCVR of SEQ ID NO: 123 and a LCVR of SEQ ID NO: 125, with X₁₂ being selected from Asn (N), Ser (S) and Gly (G).

In one embodiment, the antibody or binding fragment thereof comprises a HCVR of SEQ ID NO: 124 and a LCVR of SEQ ID NO: 71, with X₁₂ being selected from Asn (N), Ser (S) and Gly (G).

In one embodiment, the antibody or binding fragment thereof comprises a HCVR of SEQ ID NO: 124 and a LCVR of SEQ ID NO: 72, with X₁₂ being selected from Asn (N), In one embodiment, the antibody or binding fragment thereof comprises a HCVR of SEQ ID NO: 124 and a LCVR of SEQ ID NO: 73, with X₁₂ being selected from Asn (N), Ser (S) and Gly (G).

In one embodiment, the antibody or binding fragment thereof comprises a HCVR of SEQ ID NO: 124 and a LCVR of SEQ ID NO: 74, with X₁₂ being selected from Asn (N), Ser (S) and Gly (G).

In one embodiment, the antibody or binding fragment thereof comprises a HCVR of SEQ ID NO: 124 and a LCVR of SEQ ID NO: 75, with X₁₂ being selected from Asn (N), Ser (S) and Gly (G).

In one embodiment, the antibody or binding fragment thereof comprises a HCVR of SEQ ID NO: 124 and a LCVR of SEQ ID NO: 76, with X₁₂ being selected from Asn (N), Ser (S) and Gly (G).

In one embodiment, the antibody or binding fragment thereof comprises a HCVR of SEQ ID NO: 124 and a LCVR of SEQ ID NO: 77, with X₁₂ being selected from Asn (N), Ser (S) and Gly (G).

In one embodiment, the antibody or binding fragment thereof comprises a HCVR of SEQ ID NO: 124 and a LCVR of SEQ ID NO: 78, with X₁₂ being selected from Asn (N), Ser (S) and Gly (G).

In one embodiment, the antibody or binding fragment thereof comprises a HCVR of SEQ ID NO: 124 and a LCVR of SEQ ID NO: 79, with X₁₂ being selected from Asn (N), Ser (S) and Gly (G).

In one embodiment, the antibody or binding fragment thereof comprises a HCVR of SEQ ID NO: 124 and a LCVR of SEQ ID NO: 80, with X₁₂ being selected from Asn (N), Ser (S) and Gly (G).

In one embodiment, the antibody or binding fragment thereof comprises a HCVR of SEQ ID NO: 124 and a LCVR of SEQ ID NO: 102, with X₁₂ being selected from Asn (N), Ser (S) and Gly (G).

In one embodiment, the antibody or binding fragment thereof comprises a HCVR of SEQ ID NO: 124 and a LCVR of SEQ ID NO: 112, with X₁₂ being selected from Asn (N), Ser (S) and Gly (G).

In one embodiment, the antibody or binding fragment thereof comprises a HCVR of SEQ ID NO: 124 and a LCVR of SEQ ID NO: 125, with X₁₂ being selected from Asn (N), Ser (S) and Gly (G).

In one embodiment, the antibody or binding fragment thereof comprises a HCVR of SEQ ID NO: 61 and a LCVR of SEQ ID NO: 81.

In one embodiment, the antibody or binding fragment thereof comprises a HCVR of SEQ ID NO: 61 and a LCVR of SEQ ID NO: 82.

In one embodiment, the antibody or binding fragment thereof comprises a HCVR of SEQ ID NO: 61 and a LCVR of SEQ ID NO: 83.

In one embodiment, the antibody or binding fragment thereof comprises a HCVR of SEQ ID NO: 61 and a LCVR of SEQ ID NO: 84.

In one embodiment, the antibody or binding fragment thereof comprises a HCVR of SEQ ID NO: 61 and a LCVR of SEQ ID NO: 85.

In one embodiment, the antibody or binding fragment thereof comprises a HCVR of SEQ ID NO: 61 and a LCVR of SEQ ID NO: 86.

In one embodiment, the antibody or binding fragment thereof comprises a HCVR of SEQ ID NO: 61 and a LCVR of SEQ ID NO: 87.

In one embodiment, the antibody or binding fragment thereof comprises a HCVR of SEQ ID NO: 61 and a LCVR of SEQ ID NO: 88.

In one embodiment, the antibody or binding fragment thereof comprises a HCVR of SEQ ID NO: 61 and a LCVR of SEQ ID NO: 89.

In one embodiment, the antibody or binding fragment thereof comprises a HCVR of SEQ ID NO: 61 and a LCVR of SEQ ID NO: 90.

In one embodiment, the antibody or binding fragment thereof comprises a HCVR of SEQ ID NO: 61 and a LCVR of SEQ ID NO: 103.

In one embodiment, the antibody or binding fragment thereof comprises a HCVR of SEQ ID NO: 61 and a LCVR of SEQ ID NO: 113.

In one embodiment, the antibody or binding fragment thereof comprises a HCVR of SEQ ID NO: 61 and a LCVR of SEQ ID NO: 126.

In one embodiment, the antibody or binding fragment thereof comprises a HCVR of SEQ ID NO: 62 and a LCVR of SEQ ID NO: 81.

In one embodiment, the antibody or binding fragment thereof comprises a HCVR of SEQ ID NO: 62 and a LCVR of SEQ ID NO: 82.

In one embodiment, the antibody or binding fragment thereof comprises a HCVR of SEQ ID NO: 62 and a LCVR of SEQ ID NO: 83.

In one embodiment, the antibody or binding fragment thereof comprises a HCVR of SEQ ID NO: 62 and a LCVR of SEQ ID NO: 84.

In one embodiment, the antibody or binding fragment thereof comprises a HCVR of SEQ ID NO: 62 and a LCVR of SEQ ID NO: 85.

In one embodiment, the antibody or binding fragment thereof comprises a HCVR of SEQ ID NO: 62 and a LCVR of SEQ ID NO: 86.

In one embodiment, the antibody or binding fragment thereof comprises a HCVR of SEQ ID NO: 62 and a LCVR of SEQ ID NO: 87.

In one embodiment, the antibody or binding fragment thereof comprises a HCVR of SEQ ID NO: 62 and a LCVR of SEQ ID NO: 88.

In one embodiment, the antibody or binding fragment thereof comprises a HCVR of SEQ ID NO: 62 and a LCVR of SEQ ID NO: 89.

In one embodiment, the antibody or binding fragment thereof comprises a HCVR of SEQ ID NO: 62 and a LCVR of SEQ ID NO: 90.

In one embodiment, the antibody or binding fragment thereof comprises a HCVR of SEQ ID NO: 62 and a LCVR of SEQ ID NO: 103.

In one embodiment, the antibody or binding fragment thereof comprises a HCVR of SEQ ID NO: 62 and a LCVR of SEQ ID NO: 113.

In one embodiment, the antibody or binding fragment thereof comprises a HCVR of SEQ ID NO: 62 and a LCVR of SEQ ID NO: 126.

In one embodiment, the antibody or binding fragment thereof comprises a HCVR of SEQ ID NO: 63 and a LCVR of SEQ ID NO: 81.

In one embodiment, the antibody or binding fragment thereof comprises a HCVR of SEQ ID NO: 63 and a LCVR of SEQ ID NO: 82.

In one embodiment, the antibody or binding fragment thereof comprises a HCVR of SEQ ID NO: 63 and a LCVR of SEQ ID NO: 83.

In one embodiment, the antibody or binding fragment thereof comprises a HCVR of SEQ ID NO: 63 and a LCVR of SEQ ID NO: 84.

In one embodiment, the antibody or binding fragment thereof comprises a HCVR of SEQ ID NO: 63 and a LCVR of SEQ ID NO: 85.

In one embodiment, the antibody or binding fragment thereof comprises a HCVR of SEQ ID NO: 63 and a LCVR of SEQ ID NO: 86.

In one embodiment, the antibody or binding fragment thereof comprises a HCVR of SEQ ID NO: 63 and a LCVR of SEQ ID NO: 87.

In one embodiment, the antibody or binding fragment thereof comprises a HCVR of SEQ ID NO: 63 and a LCVR of SEQ ID NO: 88.

In one embodiment, the antibody or binding fragment thereof comprises a HCVR of SEQ ID NO: 63 and a LCVR of SEQ ID NO: 89.

In one embodiment, the antibody or binding fragment thereof comprises a HCVR of SEQ ID NO: 63 and a LCVR of SEQ ID NO: 90.

In one embodiment, the antibody or binding fragment thereof comprises a HCVR of SEQ ID NO: 63 and a LCVR of SEQ ID NO: 103.

In one embodiment, the antibody or binding fragment thereof comprises a HCVR of SEQ ID NO: 63 and a LCVR of SEQ ID NO: 113.

In one embodiment, the antibody or binding fragment thereof comprises a HCVR of SEQ ID NO: 63 and a LCVR of SEQ ID NO: 126.

In one embodiment, the antibody or binding fragment thereof comprises a HCVR of SEQ ID NO: 64 and a LCVR of SEQ ID NO: 81.

In one embodiment, the antibody or binding fragment thereof comprises a HCVR of SEQ ID NO: 64 and a LCVR of SEQ ID NO: 82.

In one embodiment, the antibody or binding fragment thereof comprises a HCVR of SEQ ID NO: 64 and a LCVR of SEQ ID NO: 83.

In one embodiment, the antibody or binding fragment thereof comprises a HCVR of SEQ ID NO: 64 and a LCVR of SEQ ID NO: 84.

In one embodiment, the antibody or binding fragment thereof comprises a HCVR of SEQ ID NO: 64 and a LCVR of SEQ ID NO: 85.

In one embodiment, the antibody or binding fragment thereof comprises a HCVR of SEQ ID NO: 64 and a LCVR of SEQ ID NO: 86.

In one embodiment, the antibody or binding fragment thereof comprises a HCVR of SEQ ID NO: 64 and a LCVR of SEQ ID NO: 87.

In one embodiment, the antibody or binding fragment thereof comprises a HCVR of SEQ ID NO: 64 and a LCVR of SEQ ID NO: 88.

In one embodiment, the antibody or binding fragment thereof comprises a HCVR of SEQ ID NO: 64 and a LCVR of SEQ ID NO: 89.

In one embodiment, the antibody or binding fragment thereof comprises a HCVR of SEQ ID NO: 64 and a LCVR of SEQ ID NO: 90.

In one embodiment, the antibody or binding fragment thereof comprises a HCVR of SEQ ID NO: 64 and a LCVR of SEQ ID NO: 103.

In one embodiment, the antibody or binding fragment thereof comprises a HCVR of SEQ ID NO: 64 and a LCVR of SEQ ID NO: 113.

In one embodiment, the antibody or binding fragment thereof comprises a HCVR of SEQ ID NO: 64 and a LCVR of SEQ ID NO: 126.

In one embodiment, the antibody or binding fragment thereof comprises a HCVR of SEQ ID NO: 65 and a LCVR of SEQ ID NO: 81.

In one embodiment, the antibody or binding fragment thereof comprises a HCVR of SEQ ID NO: 65 and a LCVR of SEQ ID NO: 82.

In one embodiment, the antibody or binding fragment thereof comprises a HCVR of SEQ ID NO: 65 and a LCVR of SEQ ID NO: 83.

In one embodiment, the antibody or binding fragment thereof comprises a HCVR of SEQ ID NO: 65 and a LCVR of SEQ ID NO: 84.

In one embodiment, the antibody or binding fragment thereof comprises a HCVR of SEQ ID NO: 65 and a LCVR of SEQ ID NO: 85.

In one embodiment, the antibody or binding fragment thereof comprises a HCVR of SEQ ID NO: 65 and a LCVR of SEQ ID NO: 86.

In one embodiment, the antibody or binding fragment thereof comprises a HCVR of SEQ ID NO: 65 and a LCVR of SEQ ID NO: 87.

In one embodiment, the antibody or binding fragment thereof comprises a HCVR of SEQ ID NO: 65 and a LCVR of SEQ ID NO: 88.

In one embodiment, the antibody or binding fragment thereof comprises a HCVR of SEQ ID NO: 65 and a LCVR of SEQ ID NO: 89.

In one embodiment, the antibody or binding fragment thereof comprises a HCVR of SEQ ID NO: 65 and a LCVR of SEQ ID NO: 90.

In one embodiment, the antibody or binding fragment thereof comprises a HCVR of SEQ ID NO: 65 and a LCVR of SEQ ID NO: 103.

In one embodiment, the antibody or binding fragment thereof comprises a HCVR of SEQ ID NO: 65 and a LCVR of SEQ ID NO: 113.

In one embodiment, the antibody or binding fragment thereof comprises a HCVR of SEQ ID NO: 65 and a LCVR of SEQ ID NO: 126.

In one embodiment, the antibody or binding fragment thereof comprises a HCVR of SEQ ID NO: 66 and a LCVR of SEQ ID NO: 81.

In one embodiment, the antibody or binding fragment thereof comprises a HCVR of SEQ ID NO: 66 and a LCVR of SEQ ID NO: 82.

In one embodiment, the antibody or binding fragment thereof comprises a HCVR of SEQ ID NO: 66 and a LCVR of SEQ ID NO: 83.

In one embodiment, the antibody or binding fragment thereof comprises a HCVR of SEQ ID NO: 66 and a LCVR of SEQ ID NO: 84.

In one embodiment, the antibody or binding fragment thereof comprises a HCVR of SEQ ID NO: 66 and a LCVR of SEQ ID NO: 85.

In one embodiment, the antibody or binding fragment thereof comprises a HCVR of SEQ ID NO: 66 and a LCVR of SEQ ID NO: 86.

In one embodiment, the antibody or binding fragment thereof comprises a HCVR of SEQ ID NO: 66 and a LCVR of SEQ ID NO: 87.

In one embodiment, the antibody or binding fragment thereof comprises a HCVR of SEQ ID NO: 66 and a LCVR of SEQ ID NO: 88.

In one embodiment, the antibody or binding fragment thereof comprises a HCVR of SEQ ID NO: 66 and a LCVR of SEQ ID NO: 89.

In one embodiment, the antibody or binding fragment thereof comprises a HCVR of SEQ ID NO: 66 and a LCVR of SEQ ID NO: 90.

In one embodiment, the antibody or binding fragment thereof comprises a HCVR of SEQ ID NO: 66 and a LCVR of SEQ ID NO: 103.

In one embodiment, the antibody or binding fragment thereof comprises a HCVR of SEQ ID NO: 66 and a LCVR of SEQ ID NO: 113.

In one embodiment, the antibody or binding fragment thereof comprises a HCVR of SEQ ID NO: 66 and a LCVR of SEQ ID NO: 126.

In one embodiment, the antibody or binding fragment thereof comprises a HCVR of SEQ ID NO: 67 and a LCVR of SEQ ID NO: 81.

In one embodiment, the antibody or binding fragment thereof comprises a HCVR of SEQ ID NO: 67 and a LCVR of SEQ ID NO: 82.

In one embodiment, the antibody or binding fragment thereof comprises a HCVR of SEQ ID NO: 67 and a LCVR of SEQ ID NO: 83.

In one embodiment, the antibody or binding fragment thereof comprises a HCVR of SEQ ID NO: 67 and a LCVR of SEQ ID NO: 84.

In one embodiment, the antibody or binding fragment thereof comprises a HCVR of SEQ ID NO: 67 and a LCVR of SEQ ID NO: 85.

In one embodiment, the antibody or binding fragment thereof comprises a HCVR of SEQ ID NO: 67 and a LCVR of SEQ ID NO: 86.

In one embodiment, the antibody or binding fragment thereof comprises a HCVR of SEQ ID NO: 67 and a LCVR of SEQ ID NO: 87.

In one embodiment, the antibody or binding fragment thereof comprises a HCVR of SEQ ID NO: 67 and a LCVR of SEQ ID NO: 88.

In one embodiment, the antibody or binding fragment thereof comprises a HCVR of SEQ ID NO: 67 and a LCVR of SEQ ID NO: 89.

In one embodiment, the antibody or binding fragment thereof comprises a HCVR of SEQ ID NO: 67 and a LCVR of SEQ ID NO: 90.

In one embodiment, the antibody or binding fragment thereof comprises a HCVR of SEQ ID NO: 67 and a LCVR of SEQ ID NO: 103.

In one embodiment, the antibody or binding fragment thereof comprises a HCVR of SEQ ID NO: 67 and a LCVR of SEQ ID NO: 113.

In one embodiment, the antibody or binding fragment thereof comprises a HCVR of SEQ ID NO: 67 and a LCVR of SEQ ID NO: 126.

In one embodiment, the antibody or binding fragment thereof comprises a HCVR of SEQ ID NO: 68 and a LCVR of SEQ ID NO: 81.

In one embodiment, the antibody or binding fragment thereof comprises a HCVR of SEQ ID NO: 68 and a LCVR of SEQ ID NO: 82.

In one embodiment, the antibody or binding fragment thereof comprises a HCVR of SEQ ID NO: 68 and a LCVR of SEQ ID NO: 83.

In one embodiment, the antibody or binding fragment thereof comprises a HCVR of SEQ ID NO: 68 and a LCVR of SEQ ID NO: 84.

In one embodiment, the antibody or binding fragment thereof comprises a HCVR of SEQ ID NO: 68 and a LCVR of SEQ ID NO: 85.

In one embodiment, the antibody or binding fragment thereof comprises a HCVR of SEQ ID NO: 68 and a LCVR of SEQ ID NO: 86.

In one embodiment, the antibody or binding fragment thereof comprises a HCVR of SEQ ID NO: 68 and a LCVR of SEQ ID NO: 87.

In one embodiment, the antibody or binding fragment thereof comprises a HCVR of SEQ ID NO: 68 and a LCVR of SEQ ID NO: 88.

In one embodiment, the antibody or binding fragment thereof comprises a HCVR of SEQ ID NO: 68 and a LCVR of SEQ ID NO: 89.

In one embodiment, the antibody or binding fragment thereof comprises a HCVR of SEQ ID NO: 68 and a LCVR of SEQ ID NO: 90.

In one embodiment, the antibody or binding fragment thereof comprises a HCVR of SEQ ID NO: 68 and a LCVR of SEQ ID NO: 103.

In one embodiment, the antibody or binding fragment thereof comprises a HCVR of SEQ ID NO: 68 and a LCVR of SEQ ID NO: 113.

In one embodiment, the antibody or binding fragment thereof comprises a HCVR of SEQ ID NO: 68 and a LCVR of SEQ ID NO: 126.

In one embodiment, the antibody or binding fragment thereof comprises a HCVR of SEQ ID NO: 69 and a LCVR of SEQ ID NO: 81.

In one embodiment, the antibody or binding fragment thereof comprises a HCVR of SEQ ID NO: 69 and a LCVR of SEQ ID NO: 82.

In one embodiment, the antibody or binding fragment thereof comprises a HCVR of SEQ ID NO: 69 and a LCVR of SEQ ID NO: 83.

In one embodiment, the antibody or binding fragment thereof comprises a HCVR of SEQ ID NO: 69 and a LCVR of SEQ ID NO: 84.

In one embodiment, the antibody or binding fragment thereof comprises a HCVR of SEQ ID NO: 69 and a LCVR of SEQ ID NO: 85.

In one embodiment, the antibody or binding fragment thereof comprises a HCVR of SEQ ID NO: 69 and a LCVR of SEQ ID NO: 86.

In one embodiment, the antibody or binding fragment thereof comprises a HCVR of SEQ ID NO: 69 and a LCVR of SEQ ID NO: 87.

In one embodiment, the antibody or binding fragment thereof comprises a HCVR of SEQ ID NO: 69 and a LCVR of SEQ ID NO: 88.

In one embodiment, the antibody or binding fragment thereof comprises a HCVR of SEQ ID NO: 69 and a LCVR of SEQ ID NO: 89.

In one embodiment, the antibody or binding fragment thereof comprises a HCVR of SEQ ID NO: 69 and a LCVR of SEQ ID NO: 90.

In one embodiment, the antibody or binding fragment thereof comprises a HCVR of SEQ ID NO: 69 and a LCVR of SEQ ID NO: 103.

In one embodiment, the antibody or binding fragment thereof comprises a HCVR of SEQ ID NO: 69 and a LCVR of SEQ ID NO: 113.

In one embodiment, the antibody or binding fragment thereof comprises a HCVR of SEQ ID NO: 69 and a LCVR of SEQ ID NO: 126.

In one embodiment, the antibody or binding fragment thereof comprises a HCVR of SEQ ID NO: 70 and a LCVR of SEQ ID NO: 81.

In one embodiment, the antibody or binding fragment thereof comprises a HCVR of SEQ ID NO: 70 and a LCVR of SEQ ID NO: 82.

In one embodiment, the antibody or binding fragment thereof comprises a HCVR of SEQ ID NO: 70 and a LCVR of SEQ ID NO: 83.

In one embodiment, the antibody or binding fragment thereof comprises a HCVR of SEQ ID NO: 70 and a LCVR of SEQ ID NO: 84.

In one embodiment, the antibody or binding fragment thereof comprises a HCVR of SEQ ID NO: 70 and a LCVR of SEQ ID NO: 85.

In one embodiment, the antibody or binding fragment thereof comprises a HCVR of SEQ ID NO: 70 and a LCVR of SEQ ID NO: 86.

In one embodiment, the antibody or binding fragment thereof comprises a HCVR of SEQ ID NO: 70 and a LCVR of SEQ ID NO: 87.

In one embodiment, the antibody or binding fragment thereof comprises a HCVR of SEQ ID NO: 70 and a LCVR of SEQ ID NO: 88.

In one embodiment, the antibody or binding fragment thereof comprises a HCVR of SEQ ID NO: 70 and a LCVR of SEQ ID NO: 89.

In one embodiment, the antibody or binding fragment thereof comprises a HCVR of SEQ ID NO: 70 and a LCVR of SEQ ID NO: 90.

In one embodiment, the antibody or binding fragment thereof comprises a HCVR of SEQ ID NO: 70 and a LCVR of SEQ ID NO: 103.

In one embodiment, the antibody or binding fragment thereof comprises a HCVR of SEQ ID NO: 70 and a LCVR of SEQ ID NO: 113.

In one embodiment, the antibody or binding fragment thereof comprises a HCVR of SEQ ID NO: 70 and a LCVR of SEQ ID NO: 126.

In one embodiment, the antibody or binding fragment thereof comprises a HCVR of SEQ ID NO: 101 and a LCVR of SEQ ID NO: 81.

In one embodiment, the antibody or binding fragment thereof comprises a HCVR of SEQ ID NO: 101 and a LCVR of SEQ ID NO: 82.

In one embodiment, the antibody or binding fragment thereof comprises a HCVR of SEQ ID NO: 101 and a LCVR of SEQ ID NO: 83.

In one embodiment, the antibody or binding fragment thereof comprises a HCVR of SEQ ID NO: 101 and a LCVR of SEQ ID NO: 84.

In one embodiment, the antibody or binding fragment thereof comprises a HCVR of SEQ ID NO: 101 and a LCVR of SEQ ID NO: 85.

In one embodiment, the antibody or binding fragment thereof comprises a HCVR of SEQ ID NO: 101 and a LCVR of SEQ ID NO: 86.

In one embodiment, the antibody or binding fragment thereof comprises a HCVR of SEQ ID NO: 101 and a LCVR of SEQ ID NO: 87.

In one embodiment, the antibody or binding fragment thereof comprises a HCVR of SEQ ID NO: 101 and a LCVR of SEQ ID NO: 88.

In one embodiment, the antibody or binding fragment thereof comprises a HCVR of SEQ ID NO: 101 and a LCVR of SEQ ID NO: 89.

In one embodiment, the antibody or binding fragment thereof comprises a HCVR of SEQ ID NO: 101 and a LCVR of SEQ ID NO: 90.

In one embodiment, the antibody or binding fragment thereof comprises a HCVR of SEQ ID NO: 101 and a LCVR of SEQ ID NO: 103.

In one embodiment, the antibody or binding fragment thereof comprises a HCVR of SEQ ID NO: 101 and a LCVR of SEQ ID NO: 113.

In one embodiment, the antibody or binding fragment thereof comprises a HCVR of SEQ ID NO: 101 and a LCVR of SEQ ID NO: 126.

In one embodiment, the antibody or binding fragment thereof comprises a HCVR of SEQ ID NO: 121 and a LCVR of SEQ ID NO: 81.

In one embodiment, the antibody or binding fragment thereof comprises a HCVR of SEQ ID NO: 121 and a LCVR of SEQ ID NO: 82.

In one embodiment, the antibody or binding fragment thereof comprises a HCVR of SEQ ID NO: 121 and a LCVR of SEQ ID NO: 83.

In one embodiment, the antibody or binding fragment thereof comprises a HCVR of SEQ ID NO: 121 and a LCVR of SEQ ID NO: 84.

In one embodiment, the antibody or binding fragment thereof comprises a HCVR of SEQ ID NO: 121 and a LCVR of SEQ ID NO: 85.

In one embodiment, the antibody or binding fragment thereof comprises a HCVR of SEQ ID NO: 121 and a LCVR of SEQ ID NO: 86.

In one embodiment, the antibody or binding fragment thereof comprises a HCVR of SEQ ID NO: 121 and a LCVR of SEQ ID NO: 87.

In one embodiment, the antibody or binding fragment thereof comprises a HCVR of SEQ ID NO: 121 and a LCVR of SEQ ID NO: 88.

In one embodiment, the antibody or binding fragment thereof comprises a HCVR of SEQ ID NO: 121 and a LCVR of SEQ ID NO: 89.

In one embodiment, the antibody or binding fragment thereof comprises a HCVR of SEQ ID NO: 121 and a LCVR of SEQ ID NO: 90.

In one embodiment, the antibody or binding fragment thereof comprises a HCVR of SEQ ID NO: 121 and a LCVR of SEQ ID NO: 103.

In one embodiment, the antibody or binding fragment thereof comprises a HCVR of SEQ ID NO: 121 and a LCVR of SEQ ID NO: 113.

In one embodiment, the antibody or binding fragment thereof comprises a HCVR of SEQ ID NO: 121 and a LCVR of SEQ ID NO: 126.

In one embodiment, the antibody or binding fragment thereof comprises a HCVR of SEQ ID NO: 122 and a LCVR of SEQ ID NO: 81.

In one embodiment, the antibody or binding fragment thereof comprises a HCVR of SEQ ID NO: 122 and a LCVR of SEQ ID NO: 82.

In one embodiment, the antibody or binding fragment thereof comprises a HCVR of SEQ ID NO: 122 and a LCVR of SEQ ID NO: 83.

In one embodiment, the antibody or binding fragment thereof comprises a HCVR of SEQ ID NO: 122 and a LCVR of SEQ ID NO: 84.

In one embodiment, the antibody or binding fragment thereof comprises a HCVR of SEQ ID NO: 122 and a LCVR of SEQ ID NO: 85.

In one embodiment, the antibody or binding fragment thereof comprises a HCVR of SEQ ID NO: 122 and a LCVR of SEQ ID NO: 86.

In one embodiment, the antibody or binding fragment thereof comprises a HCVR of SEQ ID NO: 122 and a LCVR of SEQ ID NO: 87.

In one embodiment, the antibody or binding fragment thereof comprises a HCVR of SEQ ID NO: 122 and a LCVR of SEQ ID NO: 88.

In one embodiment, the antibody or binding fragment thereof comprises a HCVR of SEQ ID NO: 122 and a LCVR of SEQ ID NO: 89.

In one embodiment, the antibody or binding fragment thereof comprises a HCVR of SEQ ID NO: 122 and a LCVR of SEQ ID NO: 90.

In one embodiment, the antibody or binding fragment thereof comprises a HCVR of SEQ ID NO: 122 and a LCVR of SEQ ID NO: 103.

In one embodiment, the antibody or binding fragment thereof comprises a HCVR of SEQ ID NO: 122 and a LCVR of SEQ ID NO: 113.

In one embodiment, the antibody or binding fragment thereof comprises a HCVR of SEQ ID NO: 122 and a LCVR of SEQ ID NO: 126.

In one embodiment, the antibody or binding fragment thereof comprises a HCVR of SEQ ID NO: 123 and a LCVR of SEQ ID NO: 81.

In one embodiment, the antibody or binding fragment thereof comprises a HCVR of SEQ ID NO: 123 and a LCVR of SEQ ID NO: 82.

In one embodiment, the antibody or binding fragment thereof comprises a HCVR of SEQ ID NO: 123 and a LCVR of SEQ ID NO: 83.

In one embodiment, the antibody or binding fragment thereof comprises a HCVR of SEQ ID NO: 123 and a LCVR of SEQ ID NO: 84.

In one embodiment, the antibody or binding fragment thereof comprises a HCVR of SEQ ID NO: 123 and a LCVR of SEQ ID NO: 85.

In one embodiment, the antibody or binding fragment thereof comprises a HCVR of SEQ ID NO: 123 and a LCVR of SEQ ID NO: 86.

In one embodiment, the antibody or binding fragment thereof comprises a HCVR of SEQ ID NO: 123 and a LCVR of SEQ ID NO: 87.

In one embodiment, the antibody or binding fragment thereof comprises a HCVR of SEQ ID NO: 123 and a LCVR of SEQ ID NO: 88.

In one embodiment, the antibody or binding fragment thereof comprises a HCVR of SEQ ID NO: 123 and a LCVR of SEQ ID NO: 89.

In one embodiment, the antibody or binding fragment thereof comprises a HCVR of SEQ ID NO: 123 and a LCVR of SEQ ID NO: 90.

In one embodiment, the antibody or binding fragment thereof comprises a HCVR of SEQ ID NO: 123 and a LCVR of SEQ ID NO: 103.

In one embodiment, the antibody or binding fragment thereof comprises a HCVR of SEQ ID NO: 123 and a LCVR of SEQ ID NO: 113.

In one embodiment, the antibody or binding fragment thereof comprises a HCVR of SEQ ID NO: 123 and a LCVR of SEQ ID NO: 126.

In one embodiment, the antibody or binding fragment thereof comprises a HCVR of SEQ ID NO: 124 and a LCVR of SEQ ID NO: 81.

In one embodiment, the antibody or binding fragment thereof comprises a HCVR of SEQ ID NO: 124 and a LCVR of SEQ ID NO: 82.

In one embodiment, the antibody or binding fragment thereof comprises a HCVR of SEQ ID NO: 124 and a LCVR of SEQ ID NO: 83.

In one embodiment, the antibody or binding fragment thereof comprises a HCVR of SEQ ID NO: 124 and a LCVR of SEQ ID NO: 84.

In one embodiment, the antibody or binding fragment thereof comprises a HCVR of SEQ ID NO: 124 and a LCVR of SEQ ID NO: 85.

In one embodiment, the antibody or binding fragment thereof comprises a HCVR of SEQ ID NO: 124 and a LCVR of SEQ ID NO: 86.

In one embodiment, the antibody or binding fragment thereof comprises a HCVR of SEQ ID NO: 124 and a LCVR of SEQ ID NO: 87.

In one embodiment, the antibody or binding fragment thereof comprises a HCVR of SEQ ID NO: 124 and a LCVR of SEQ ID NO: 88.

In one embodiment, the antibody or binding fragment thereof comprises a HCVR of SEQ ID NO: 124 and a LCVR of SEQ ID NO: 89.

In one embodiment, the antibody or binding fragment thereof comprises a HCVR of SEQ ID NO: 124 and a LCVR of SEQ ID NO: 90.

In one embodiment, the antibody or binding fragment thereof comprises a HCVR of SEQ ID NO: 124 and a LCVR of SEQ ID NO: 103.

In one embodiment, the antibody or binding fragment thereof comprises a HCVR of SEQ ID NO: 124 and a LCVR of SEQ ID NO: 113.

In one embodiment, the antibody or binding fragment thereof comprises a HCVR of SEQ ID NO: 124 and a LCVR of SEQ ID NO: 126.

In one embodiment, any of the HCVR and/or LCVR as defined hereinabove can be characterized as having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more amino acids being substituted by a different amino acid.

In one embodiment, the sequence of the non-CDR regions of any of the HCVR and/or LCVR as defined hereinabove can be characterized as having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or more amino acids being substituted by a different amino acid.

In one embodiment, any of the HCVR and/or LCVR as defined hereinabove can be characterized as having an amino acid sequence that shares at least 70%, 75%, 80%, 85%, 90%, 95%, 96%, 97%, 98%, 99% or more of identity with the particular HCVR and/or LCVR as defined hereinabove.

In one embodiment, the sequence of the non-CDR regions of any of the HCVR and/or LCVR as defined hereinabove can be characterized as having an amino acid sequence that shares at least 70%, 75%, 80%, 85%, 90%, 95%, 96%, 97%, 98%, 99% or more of identity with the sequence of the non-CDR regions of the particular HCVR and/or LCVR as defined hereinabove.

The present specification also describes an isolated nucleic acid encoding the antibody or binding fragment thereof binding to hCD45RC disclosed hereabove in the “antibody or antigen-binding fragment” section.

In one embodiment, the isolated nucleic acid is purified.

In one embodiment, the isolated nucleic acid is purified to:
(1) greater than 80%, 85%, 90%, 91%, 92%, 93%, 94%, 95% or more by weight of nucleic acid as determined by absorbance methods or fluorescence methods (such as, e.g., by measuring the ratio of absorbance at 260 and 280 nm (A_{260/280})), and most preferably more than 96%, 97%, 98% or 99% by weight; or
(2) homogeneity as shown by agarose gel electrophoresis and using an intercalating agent such as ethidium bromide, SYBR Green, GelGreen or the like.

In one embodiment, the nucleic acid encoding the antigen-binding fragment comprises or consists of a sequence encoding the HCVR of the antibody or binding fragment thereof disclosed in the “antibody or antigen-binding fragment” section.

In one embodiment, the nucleic acid encoding the antigen-binding fragment comprises or consists of a sequence SEQ ID NO: 95 or any sequence sharing at least 70%, 75%, 80%, 85%, 90%, 95%, 96%, 97%, 98%, 99% or more identity with SEQ ID NO: 95, encoding the HCVR of the murine or chimeric antibody or binding fragment thereof with SEQ ID NO: 61.

SEQ ID NO: 95
CAGGTCCAGCTGCAACAGTCTGGCGCTGAGCTGGT
TAGGCCTGGGACTTCAGTGAAGATGTCCTGCAAGG
CCGCTGGATACACCTTCACTAACTACTACATAGGT
TGGGTAAAGCAGAGGCCTGGACATGGCCTTGAGTG
GATCGGAGATATTTTCCCTGGAGGTGACTATGCCA
ACAGCAATGAGAAGTTCAAGGGCAAAGCCACACTG
ACTGCAGACACATCCTCCAGCACAGCCTACATGCA
GCTCAGCAGCCTGACATCTGAGGACTCTGCCATCT
ATTACTGTGTGAGAAGGAACTTTGACTACTGGGGC
CAAGGCACCACTCTCACAGTGTCCTCA

In one embodiment, the nucleic acid encoding the antigen-binding fragment comprises or consists of a sequence encoding the LCVR of the antibody or binding fragment thereof disclosed in the “antibody or antigen-binding fragment” section.

In one embodiment, the nucleic acid encoding the antigen-binding fragment comprises or consists of a sequence SEQ ID NO: 96 or any sequence sharing at least 70%, 75%, 80%, 85%, 90%, 95%, 96%, 97%, 98%, 99% or more identity with SEQ ID NO: 96, encoding the LCVR of the murine or chimeric antibody or binding fragment thereof with SEQ ID NO: 81.

SEQ ID NO: 96
CAAATTGTTCTCACCCAGTCTCCAACAATCATGTC
TGCATCTCCAGGGGAGAAGGTGACCATAACCTGCA
GTGCCAGCTCAAGTGTAAGTTACATGCACTGGTTC
CAGCAGAAGACAGGCACTTCTCCCAGACTCTGGAT
TTATAACACATCCAACCTGCCTTCTGGAGTCCCCG
CTCGCTTCAGTGGCAGTGGATCTGGGACCTCTTAC
TCTCTCACAATCAGCCGAATGGAGGCTGAAGATGC
TGCCACTTATTACTGCCAGCAAAGGAGTAGTTACC
CACTCACGTTCGGTGCTGGGACCAAGCTGGAGCTG
AAA

In one embodiment, the nucleic acid encoding the antigen-binding fragment comprises or consists of:
a sequence encoding the HCVR of the antibody or binding fragment thereof disclosed in the “antibody or antigen-binding fragment” section, and
a sequence encoding the LCVR of the antibody or binding fragment thereof disclosed in the “antibody or antigen-binding fragment” section.

In one embodiment, the nucleic acid encoding the antigen-binding fragment comprises or consists of:
a sequence SEQ ID NO: 95 or any sequence sharing at least 70%, 75%, 80%, 85%, 90%, 95%, 96%, 97%, 98%, 99% or more identity with SEQ ID NO: 95 and
a sequence SEQ ID NO: 96 or any sequence sharing at least 70%, 75%, 80%, 85%, 90%, 95%, 96%, 97%, 98%, 99% or more identity with SEQ ID NO: 96.

It will be readily understood that the one skilled in the art can design nucleic acid sequences encoding all other HCVRs and LCVRs herein disclosed, in particular for the humanized antibodies or binding fragment thereof disclosed in the “antibody or antigen-binding fragment” section.

It is further understood that the one skilled in the art is familiar with molecular biology methods aiming at modifying a nucleic acid sequence in order to improve, e.g., recombinant production rates, such as by codon optimization. Ultimately, the present application encompasses any nucleic acid encoding any HCVRs and/or LCVRs as herein disclosed.

The present invention provides a chimeric receptor specific for human CD45RC, wherein said CAR comprises:
(a) at least one extracellular binding domain, wherein said binding domain binds to said human CD45RC
(b) optionally at least one extracellular hinge domain,
(c) at least one transmembrane domain, and
(d) at least one intracellular signaling domain, wherein the intracellular domain comprises at least one T cell primary signaling domain and optionally at least one T cell costimulatory signaling domain.

As used herein, the term “chimeric receptor” (CR) or “chimeric antigen receptor” (CAR) refers to one polypeptide or to a set of polypeptides, typically two in the simplest embodiments, which when in an immune cell, provides the cell with specificity for a target ligand and with intracellular signal generation. In some embodiments, the set of polypeptides are contiguous with each other. In some embodiments, the chimeric receptor is a chimeric fusion protein comprising the set of polypeptides. In some embodiments, the set of polypeptides include a dimerization switch that, upon the presence of a dimerization molecule, can couple the polypeptides to one another, e.g., can couple a ligand binding domain to an intracellular signaling domain. In one embodiment, the chimeric receptor comprises an optional leader sequence at the amino-terminus (N-ter) of the chimeric receptor fusion protein. In one embodiment, the chimeric receptor further comprises a leader sequence at the N-terminus of the extracellular ligand binding domain, wherein the leader sequence is optionally cleaved from the ligand binding domain during cellular processing and localization of the chimeric receptor to the cellular membrane.

In some embodiments, the chimeric receptor comprises one or more polypeptides.

In some embodiments, the extracellular binding domain is an antigen-binding domain, and the chimeric receptor thus may also be referred to as a chimeric antigen receptor (or CAR).

The chimeric receptor or chimeric antigen receptor of the invention comprises at least one extracellular binding domain, wherein said binding domain binds to human CD45RC.

In some embodiments, the extracellular domain of the chimeric receptor of the invention comprises at least one ligand-binding domain or antigen-binding domain. In some embodiments, the antigen-binding domain is an antibody or an antigen-binding fragment.

In some embodiments, the chimeric receptor of the invention comprises an antibody, or an antigen-binding fragment thereof, directed to human CD45RC, such as e.g. any antibody, or an antigen binding fragment thereof, described here above in the “Antibody or an antigen binding fragment” section.

In some embodiments, the extracellular binding domain of the CAR of the invention comprises an antibody directed to human CD45RC described here above in the “Antibody or an antigen binding fragment” section.

In some embodiments, the extracellular binding domain of the CAR of the invention comprises an antigen-binding fragment directed to human CD45RC described here above in the “Antibody or an antigen binding fragment” section.

In one embodiment, the extracellular binding domain comprises at least one antigen-binding fragment comprising:
(a) a HCVR which comprises the following three CDRs:
   (i) V_{H}-CDR1 of sequence SEQ ID NO: 1;
   (ii) V_{H}-CDR2 with a sequence selected from the group comprising sequences SEQ ID NOs: 4, 5, 6, 8, 100, 116, 117, 118 and 119; and
   (iii) V_{H}-CDR3 of sequence SEQ ID NO: 3; and
(b) a LCVR which comprises the following three CDRs:
   (i) V_{L}-CDR1 with a sequence selected from the group comprising sequences SEQ ID NO: 15 (SASSSVS-X₁₂-YMH) and 18 (RASSSVS-X₁₂-YMH), wherein X₁₂ is absent or is selected from Asn (N), Ser (S) and Gly (G);
   (ii) V_{L}-CDR2 of sequence SEQ ID NO: 16; and
   (iii) V_{L}-CDR3 of sequence SEQ ID NO: 17.

In one embodiment, the extracellular binding domain comprises at least one antigen-binding fragment comprising:
(a) a HCVR which comprises the following three CDRs:
   (i) V_{H}-CDR1 of sequence SEQ ID NO: 1;
   (ii) V_{H}-CDR2 with a sequence selected from the group comprising sequences SEQ ID NOs: 4 and 5; and
   (iii) V_{H}-CDR3 of sequence SEQ ID NO: 3; and
(b) a LCVR which comprises the following three CDRs:
   (i) V_{L}-CDR1 of sequence SEQ ID NO: 15, wherein X₁₂ is absent;
   (ii) V_{L}-CDR2 of sequence SEQ ID NO: 16; and
   (iii) V_{L}-CDR3 of sequence SEQ ID NO: 17.

In one embodiment, the extracellular binding domain comprises at least one antigen-binding fragment comprising:
(a) a HCVR which comprises the following three CDRs:
   (i) V_{H}-CDR1 of sequence SEQ ID NO: 1;
   (ii) V_{H}-CDR2 of sequence 4; and
   (iii) V_{H}-CDR3 of sequence SEQ ID NO: 3; and
(b) a LCVR which comprises the following three CDRs:
   (i) V_{L}-CDR1 of sequence SEQ ID NO: 15, wherein X₁₂ is absent;
   (ii) V_{L}-CDR2 of sequence SEQ ID NO: 16; and
   (iii) V_{L}-CDR3 of sequence SEQ ID NO: 17.

In one embodiment, the extracellular binding domain comprises at least one antigen-binding fragment comprising:
(a) a HCVR which comprises the following three CDRs:
   (i) V_{H}-CDR1 of sequence SEQ ID NO: 1;
   (ii) V_{H}-CDR2 with a sequence selected from the group comprising sequences SEQ ID NOs: 4, 6, and 100; and
   (iii) V_{H}-CDR3 of sequence SEQ ID NO: 3; and
(b) a LCVR which comprises the following three CDRs:
   (i) V_{L}-CDR1 with a sequence selected from the group comprising sequences SEQ ID NOs: 15 and 18, wherein X₁₂ is absent;
   (ii) V_{L}-CDR2 of sequence SEQ ID NO: 16; and
   (iii) V_{L}-CDR3 of sequence SEQ ID NO: 17.

In one embodiment, the extracellular binding domain comprises at least one antigen-binding fragment comprising:
1) a HCVR of sequence SEQ ID NO: 61 and a LCVR of sequence SEQ ID NO: 81;
2) a HCVR of sequence SEQ ID NO: 62 and a LCVR of sequence SEQ ID NO: 82;
3) a HCVR of sequence SEQ ID NO: 62 and a LCVR of sequence SEQ ID NO: 83;
4) a HCVR of sequence SEQ ID NO: 62 and a LCVR of sequence SEQ ID NO: 84;
5) a HCVR of sequence SEQ ID NO: 63 and a LCVR of sequence SEQ ID NO: 82;
6) a HCVR of sequence SEQ ID NO: 63 and a LCVR of sequence SEQ ID NO: 83;
7) a HCVR of sequence SEQ ID NO: 63 and a LCVR of sequence SEQ ID NO: 84;
8) a HCVR of sequence SEQ ID NO: 64 and a LCVR of sequence SEQ ID NO: 82;
9) a HCVR of sequence SEQ ID NO: 64 and a LCVR of sequence SEQ ID NO: 83;
10) a HCVR of sequence SEQ ID NO: 64 and a LCVR of sequence SEQ ID NO: 84;
11) a HCVR of sequence SEQ ID NO: 101 and a LCVR of sequence SEQ ID NO: 85;
12) a HCVR of sequence SEQ ID NO: 101 and a LCVR of sequence SEQ ID NO: 103;
13) a HCVR of sequence SEQ ID NO: 65 and a LCVR of sequence SEQ ID NO: 85;
14) a HCVR of sequence SEQ ID NO: 65 and a LCVR of sequence SEQ ID NO: 103;
15) a HCVR of sequence SEQ ID NO: 62 and a LCVR of sequence SEQ ID NO: 85;
16) a HCVR of sequence SEQ ID NO: 101 and a LCVR of sequence SEQ ID NO: 82;
17) a HCVR of sequence SEQ ID NO: 121 and a LCVR of sequence SEQ ID NO: 85;
18) a HCVR of sequence SEQ ID NO: 122 and a LCVR of sequence SEQ ID NO: 85;
19) a HCVR of sequence SEQ ID NO: 123 and a LCVR of sequence SEQ ID NO: 85;
20) a HCVR of sequence SEQ ID NO: 124 and a LCVR of sequence SEQ ID NO: 85;
21) a HCVR of sequence SEQ ID NO: 63 and a LCVR of sequence SEQ ID NO: 85;
22) a HCVR of sequence SEQ ID NO: 67 and a LCVR of sequence SEQ ID NO: 85;
23) a HCVR of sequence SEQ ID NO: 67 and a LCVR of sequence SEQ ID NO: 103; or
24) a HCVR and a LCVR comprising a sequence of the non-CDR regions sharing at least 70% of identity with the sequence of the non-CDR regions of the HCVR and LCVR according to 1) to 23).

In one embodiment, the extracellular binding domain comprises at least one antigen-binding fragment comprising:
(a) a HCVR which comprises the following three CDRs:
   (i) V_{H}-CDR1 of sequence SEQ ID NO: 1;
   (ii) V_{H}-CDR2 with a sequence selected from the group comprising sequences SEQ ID NOs: 4, 5, 6, 8, 100, 116, 117, 118 and 119; and
   (iii) V_{H}-CDR3 of sequence SEQ ID NO: 3; and
(b) a LCVR which comprises the following three CDRs:
   (i) V_{L}-CDR1 with a sequence selected from the group comprising sequences SEQ ID NOs: 15 and 18, wherein X₁₂ in SEQ ID NOs: 15 and 18 is selected from Asn (N), Ser (S) and Gly (G);
   (ii) V_{L}-CDR2 of sequence SEQ ID NO: 16; and
   (iii) V_{L}-CDR3 of sequence SEQ ID NO: 17;
preferably wherein the amino acid residue at Kabat position L71 of the LCVR is Phe (F).

The antibody or antigen-binding fragment thereof comprised in the CAR of the invention may exist in a variety of forms where the ligand binding domain is expressed as part of a contiguous polypeptide chain including, for example, a single domain antibody fragment (sdAb), a single chain antibody (scFv), a humanized antibody or a bispecific antibody (Harlow et al., 1999, In: Using Antibodies: A Laboratory Manual, Cold Spring Harbor Laboratory Press, N.Y.; Harlow et al., 1989, In: Antibodies: A Laboratory Manual, Cold Spring Harbor, N.Y.; Houston et al., Proc. Natl. Acad. Sci. USA 85:5879-5883 (1988); Bird et al., Science 242:423-426 (1988)). In some aspects, the antigen-binding domain of a chimeric receptor of the invention comprises an antibody fragment or an antigen-binding fragment. In some aspects, the chimeric receptor comprises an antigen-binding fragment that comprises a scFv.

In some embodiments, said antibody or antigen-binding fragment is an antibody molecule selected from the group consisting of a humanized antibody, a single chain antibody, a dimeric single chain antibody, a Fv, a scFv, a Fab, a F(ab)′₂, a defucosylated antibody, a bi-specific antibody, a diabody, a triabody, and a tetrabody.

“Single chain antibody” refers to any antibody or fragment thereof that is a protein having a primary structure comprising or consisting of one uninterrupted sequence of contiguous amino acid residues, including without limitation (1) single-chain Fv molecules (scFv); (2) single chain proteins containing only one light chain variable domain, or a fragment thereof that contains the three CDRs of the light chain variable domain, without an associated heavy chain moiety; and (3) single chain proteins containing only one heavy chain variable region, or a fragment thereof containing the three CDRs of the heavy chain variable region, without an associated light chain moiety.

“Single-chain Fv”, also abbreviated as “sFv” or “scFv”, refers to antibody fragments that comprise the V_{H} and V_{L} antibody domains connected into a single amino acid chain. Preferably, the scFv amino acid sequence further comprises a peptide linker between the V_{H} and V_{L} domains that enables the scFv to form the desired structure for antigen binding (Pluckthun, 1994. Antibodies from Escherichia coli. In Rosenberg & Moore (Eds.), The pharmacology of monoclonal antibodies. Handbook of Experimental Pharmacology, 113:269-315. Springer: Berlin, Heidelberg).

“Fv” refers to the minimum antibody fragment that contains a complete antigen-recognition and -binding site. This fragment consists of a dimer of one HCVR and one LCVR in tight, non-covalent association. From the folding of these two domains emanate six hypervariable loops (three loops each from the heavy and light chain) that contribute to antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Diabodies” refers to small antibody fragments prepared by constructing scFv fragments with short linkers (about 5-10 residues) between the HCVR and LCVR such that inter-chain but not intra-chain pairing of the variable domains is achieved, resulting in a bivalent fragment, i.e., fragment having two antigen-binding sites. Bispecific diabodies are heterodimers of two “crossover” scFv fragments in which the HCVR and LCVR of the two antibodies are present on different polypeptide chains. Diabodies are described more fully in Patent EP0404097, Patent application WO1993011161; and Holliger et al., 1993. Proc Natl Acad Sci USA. 90(14):6444-8.

In some embodiments, said antibody is an antibody fragment selected from the group consisting of a unibody, a single domain antibody, and a nanobody.

“Unibodies” are well-known in the art and refer to antibody fragments lacking the hinge region of IgG4 antibodies. The deletion of the hinge region results in a molecule that is essentially half the size of traditional IgG4 antibodies and has a univalent binding region rather than the bivalent biding region of IgG4 antibodies.

“Domain antibodies” are well-known in the art and refer to the smallest functional binding units of antibodies, corresponding to the variable regions of either the heavy or light chains of antibodies.

“Single-domain antibodies” are well-known in the art and refer to antibody-derived proteins that contain the unique structural and functional properties of naturally-occurring heavy chain antibodies (Muyldermans, 2013. Annu Rev Biochem. 82:775-97).

These heavy chain antibodies may contain a single variable domain (V_{H}H)—one such example is nanobodies®—, or a single variable domain (V_{H}H) and two constant domains (C_{H}2 and C_{H}3)—such as camelid antibodies-, or a single variable domain (V_{H}H) and five constant domains (C_{H}1, C_{H}2, C_{H}3, C_{H}4 and C_{H}5)—such as shark antibodies.

In some embodiments, said antibody is an antibody mimetic selected from the group consisting of an affibody, an affilin, an affitin, an adnectin, an atrimer, an evasin, a DARPin, an anticalin, an avimer, a fynomer, a versabody and a duocalin.

“Affibodies” are well-known in the art and refer to affinity proteins based on a 58 amino acid residue protein domain, derived from one of the IgG binding domain of staphylococcal protein A (Frejd & Kim, 2017. Exp Mol Med. 49(3):e306; Patent U.S. Pat. No. 5,831,012).

“DARPins” (Designed Ankyrin Repeat Proteins) are well-known in the art and refer to an antibody mimetic DRP (designed repeat protein) technology developed to exploit the binding abilities of non-antibody proteins (Binz et al., 2003. J Mol Biol. 332(2):489-503; Pluchthun, 2015. Annu Rev Pharmacol Toxicol. 55:489-511).

“Anticalins” are well-known in the art and refer to another antibody mimetic technology, wherein the binding specificity is derived from lipocalins (Skerra, 2008. FEBS J. 275(11):2677-83). Anticalins may also be formatted as dual targeting protein, called “duocalins” (Schlehuber & Skerra, 2001. Biol Chem. 382(9):1335-42).

“Avimers” are well-known in the art and refer to another antibody mimetic technology (Silverman et al., 2005. Nat Biotechnol. 23(12):1556-61).

“Versabodies” are well-known in the art and refer to another antibody mimetic technology (Patent Application US20070191272). They are small proteins of 3-5 kDa with >15% cysteines, which form a high disulfide density scaffold, replacing the hydrophobic core the typical proteins have. The replacement of a large number of hydrophobic amino acids, comprising the hydrophobic core, with a small number of disulfides results in a protein that is smaller, more hydrophilic (less aggregation and non-specific binding), more resistant to proteases and heat, and has a lower density of T cell epitopes, because the residues that contribute most to MHC presentation are hydrophobic. All four of these properties are well-known to affect immunogenicity, and together they are expected to cause a large decrease in immunogenicity.

In one embodiment, the antibody or binding fragment thereof also encompasses multispecific antibodies or binding fragments thereof, i.e., being immunospecific for more than one, such as at least two, different antigens, one of which being hCD45RC according to the present invention.

In one embodiment, the antibody or binding fragment thereof also encompasses polymers of antibodies or binding fragments thereof, i.e., more than one, such as at least two, antibodies or binding fragments thereof, whether identical or different, being covalently linked together, directly or indirectly.

Fragments and derivatives of antibodies (which are encompassed by the term “antibody” as used in this application, unless otherwise stated or clearly contradicted by context), can be produced by techniques that are known in the art. “Fragments” comprise a portion of the intact antibody, generally the antigen binding site or variable region. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv molecules, (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific antibodies formed from antibody fragments. Fragments of the present antibodies can be obtained using standard methods. The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., JMB 273:927-948 (1997) (“Chothia” numbering scheme), or a combination thereof.

In some embodiments, the antigen-binding domain of a CAR of the invention comprises or consists of an antibody fragment, such as, for example, a scFv. In particular embodiments, the antigen-binding domain is a scFv.

In some embodiments, a CAR of the invention comprises an extracellular binding domain against a first antigen, hCD45RC, and at least one other extracellular binding domain against another antigen. Such a CAR is capable of binding to at least 2 different antigens.

In one embodiment, said at least one other extracellular binding domain is an antibody directed to a specific antigen or an antigen binding fragment thereof.

In one embodiment, said at least one other extracellular binding domain comprises or consists of an antibody fragment, such as, for example, an scFv.

In one embodiment, the scFv comprises a linker that links the V_{H} and the V_{L} chains.

In one embodiment, the linker is a short oligo- or polypeptide, preferably having a length ranging from 2 to 10 amino acids.

For example, a glycine-serine doublet provides a particularly suitable linker (GS linker). Examples of Gly/Ser linkers include, but are not limited to, GS linkers, G₂S linkers, G₃S linkers, G₄S linkers.

A non-limiting example of G₂S linker is GGS.

G₃S linkers comprise the amino acid sequence (Gly-Gly-Gly-Ser)ₙ also referred to as (GGGS)ₙ or (SEQ ID NO: 130)ₙ, where n is a positive integer equal to or greater than 1 (such as, example, n=1, n=2, n=3. n=4, n=5, n=6, n=7, n=8, n=9 or n=10). Examples of G₃S linkers include, but are not limited to, GGGSGGGSGGGSGGGS (SEQ ID NO: 131).

Examples of G₄S linkers include, but are not limited to, (Gly₄ Ser) corresponding to GGGGS (SEQ ID NO: 132); (Gly₄ Ser)₂ corresponding to GGGGSGGGGS (SEQ ID NO: 133); (Gly₄Ser)₃ corresponding to GGGGSGGGGSGGGGS (SEQ ID NO: 134); and (Gly₄ Ser)₄ corresponding to GGGGSGGGGSGGGGSGGGGS (SEQ ID NO: 135).

In one embodiment, the linker is a (G₄S)₃ linker (SEQ ID NO: 134), that may be encoded by a sequence SEQ ID NO: 136.

SEQ ID NO: 136
GGAGGTGGAGGCTCTGGCGGTGGAGGAAGTGGTGG
GGGAGGCTCT

In one embodiment, the linker is a (G₄S)₃ linker (SEQ ID NO: 134), that may be encoded by a sequence SEQ ID NO: 137.

SEQ ID NO: 137
GGTGGCGGTGGCTCGGGCGGTGGTGGGTCGGGTGG
CGGCGGATCT

In one embodiment, the scFv comprises or consists in the nucleic acid sequence SEQ ID NO: 138 encoding the amino acid sequence SEQ ID NO: 173.

SEQ ID NO: 138
AAGGTCCAGCTGCAACAGTCTGGCGCTGAGCTGGT
TAGGCCTGGGACTTCAGTGAAGATGTCCTGCAAGG
CCGCTGGATACACCTTCACTAACTACTACATAGGT
TGGGTAAAGCAGAGGCCTGGACATGGCCTTGAGTG
GATCGGAGATATTTTCCCTGGAGGTGACTATGCCA
ACAGCAATGAGAAGTTCAAGGGCAAAGCCACACTG
ACTGCAGACACATCCTCCAGCACAGCCTACATGCA
GCTCAGCAGCCTGACATCTGAGGACTCTGCCATCT
ATTACTGTGTGAGAAGGAACTTTGACTACTGGGGC
CAAGGCACCACTCTCACAGTGTCCTCAGGTGGCGG
TGGCTCGGGCGGTGGTGGGTCGGGTGGCGGCGGAT
CTCAAATTGTTCTCACCCAGTCTCCAACAATCATG
TCTGCATCTCCAGGGGAGAAGGTGACCATAACCTG
CAGTGCCAGCTCAAGTGTAAGTTACATGCACTGGT
TCCAGCAGAAGACAGGCACTTCTCCCAGACTCTGG
ATTTATAACACATCCAACCTGCCTTCTGGAGTCCC
CGCTCGCTTCAGTGGCAGTGGATCTGGGACCTCTT
ACTCTCTCACAATCAGCCGAATGGAGGCTGAAGAT
GCTGCCACTTATTACTGCCAGCAAAGGAGTAGTTA
CCCACTCACGTTCGGTGCTGGGACCAAGCTGGAGC
TGAAA
SEQ ID NO: 173
KVQLQQSGAELVRPGTSVKMSCKAAGYTFTNYYIG
WVKQRPGHGLEWIGDIFPGGDYANSNEKFKGKATL
TADTSSSTAYMQLSSLTSEDSAIYYCVRRNFDYWG
QGTTLTVSSGGGGSGGGGSGGGGSQIVLTQSPTIM
SASPGEKVTITCSASSSVSYMHWFQQKTGTSPRLW
IYNTSNLPSGVPARFSGSGSGTSYSLTISRMEAED
AATYYCQQRSSYPLTFGAGTKLELK

In some embodiments, the antibody comprised in a CAR of the invention is a multispecific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope.

In some embodiments, the multispecific antibody molecule is a bispecific antibody molecule. A bispecific antibody has specificity for two antigens, and is characterized by a first immunoglobulin variable domain sequence that has binding specificity for a first epitope and a second immunoglobulin variable domain sequence that has binding specificity for a second epitope.

In some embodiments, the extracellular domain comprises an antigen-binding domain, (e.g. an antigen-binding fragment) as described herein in the “Antibody or antigen-binding fragment” section.

In some embodiments, the extracellular binding domain is connected to a transmembrane domain by a hinge domain.

In one embodiment, the hinge domain is a short oligo- or polypeptide linker, preferably having a length ranging from 2 to 10 amino acids, as described hereinabove.

In some embodiments, the hinge domain is a Gly/Ser linker as described hereabove.

Another example of hinge domain that may be used in the present invention is described in WO2012/138475, incorporated herein by reference.

In one embodiment, the hinge domain comprises an amino acid sequence selected from the group comprising the amino acid sequence AGSSSSGGSTTGGSTT (SEQ ID NO: 139), the amino acid sequence GTTAASGSSGGSSSGA (SEQ ID NO: 140), the amino acid sequence SSATATAGTGSSTGST (SEQ ID NO: 141), and the amino acid sequence TSGSTGTAASSTSTST (SEQ ID NO: 142).

In one embodiment, the hinge domain is encoded by a nucleotide sequence of GGTGGCGGAGGTTCTGGAGGTGGAGGTTCC (SEQ ID NO: 143).

In another embodiment, the hinge domain is a KIR₂DS₂ hinge corresponding to KIRRDSS (SEQ ID NO: 144).

In one embodiment, the hinge domain comprises or consists in the amino acid sequence of a CD8 hinge (SEQ ID NO: 145) or an amino acid sequence with at least 70%, 75%, 80%, 85%, 90%, 91%, 92%, 93%, 94%, 95%, 96%, 97%, 98% or 99% identity to SEQ ID NO: 145. In one embodiment, the hinge domain is a CD8 hinge encoded by the nucleic acid sequence (SEQ ID NO: 146) or a nucleic acid sequence with at least 70%, 75%, 80%, 85%, 90%, 91%, 92%, 93%, 94%, 95%, 96%, 97%, 98% or 99% identity to SEQ ID NO: 146.

SEQ ID NO: 145
TTTPAPRPPTPAPTIASQPLSLRPEACRPAAGGAV
HTRGLDFACD
SEQ ID NO: 146
ACCACGACGCCAGCGCCGCGACCACCAACACCGGC
GCCCACCATCGCGTCGCAGCCCCTGTCCCTGCGCC
CAGAGGCGTGCCGGCCAGCGGCGGGGGGCGCAGTG
CACACGAGGGGGCTGGACTTCGCCTGTGAT

In another embodiment, the hinge domain comprises or consists in the amino acid sequence of a IgG4 hinge (SEQ ID NO: 147), or an amino acid sequence with at least 70%, 75%, 80%, 85%, 90%, 91%, 92%, 93%, 94%, 95%, 96%, 97%, 98% or 99% identity to SEQ ID NO: 147.

SEQ ID NO: 147
ESKYGPPCPPCPAPEFLGGPSVFLFPPKPKDTLMI
SRTPEVTCVVVDVSQEDPEVQFNWYVDGVEVHNAK
TKPREEQFNSTYRVVSVLTVLHQDWLNGKEYKCKV
SNKGLPSSIEKTISKAKGQPREPQVYTLPPSQEEM
TKNQVSLTCLVKGFYPSDIAVEWESNGQPENNYKT
TPPVLDSDGSFFLYSRLTVDKSRWQEGNVFSCSVM
HEALHNHYTQKSLSLSLGKM

In one embodiment, the hinge domain is an IgG4 hinge encoded by the nucleic acid sequence (SEQ ID NO: 148) or a nucleic acid sequence with at least 70%, 75%, 80%, 85%, 90%, 91%, 92%, 93%, 94%, 95%, 96%, 97%, 98% or 99% identity to SEQ ID NO: 148.

SEQ ID NO: 148
GAGAGCAAGTACGGCCCTCCCTGCCCCCCTTGCCC
TGCCCCCGAGTTCCTGGGCGGACCCAGCGTGTTCC
TGTTCCCCCCCAAGCCCAAGGACACCCTGATGATC
AGCCGGACCCCCGAGGTGACCTGTGTGGTGGTGGA
CGTGTCCCAGGAGGACCCCGAGGTCCAGTTCAACT
GGTACGTGGACGGCGTGGAGGTGCACAACGCCAAG
ACCAAGCCCCGGGAGGAGCAGTTCAATAGCACCTA
CCGGGTGGTGTCCGTGCTGACCGTGCTGCACCAGG
ACTGGCTGAACGGCAAGGAATACAAGTGTAAGGTG
TCCAACAAGGGCCTGCCCAGCAGCATCGAGAAAAC
CATCAGCAAGGCCAAGGGCCAGCCTCGGGAGCCCC
AGGTGTACACCCTGCCCCCTAGCCAAGAGGAGATG
ACCAAGAACCAGGTGTCCCTGACCTGCCTGGTGAA
GGGCTTCTACCCCAGCGACATCGCCGTGGAGTGGG
AGAGCAACGGCCAGCCCGAGAACAACTACAAGACC
ACCCCCCCTGTGCTGGACAGCGACGGCAGCTTCTT
CCTGTACAGCCGGCTGACCGTGGACAAGAGCCGGT
GGCAGGAGGGCAACGTCTTTAGCTGCTCCGTGATG
CACGAGGCCCTGCACAACCACTACACCCAGAAGAG
CCTGAGCCTGTCCCTGGGCAAGATG

In another embodiment, the hinge domain comprises or consists in the amino acid sequence of a IgD hinge (SEQ ID NO: 149) or an amino acid sequence with at least 70%, 75%, 80%, 85%, 90%, 91%, 92%, 93%, 94%, 95%, 96%, 97%, 98% or 99% identity to SEQ ID NO: 149.

SEQ ID NO: 149
RWPESPKAQASSVPTAQPQAEGSLAKATTAPATTR
NTGRGGEEKKKEKEKEEQEERETKTPECPSHTQPL
GVYLLTPAVQDLWLRDKATFTCFVVGSDLKDAHLT
WEVAGKVPTGGVEEGLLERHSNGSQSQHSRLTLPR
SLWNAGTSVTCTLNHPSLPPQRLMALREPAAQAPV
KLSLNLLASSDPPEAASWLLCEVSGFSPPNILLMW
LEDQREVNTSGFAPARPPPQPGSTTFWAWSVLRVP
APPSPQPATYTCVVSHEDSRTLLNASRSLEVSYVT
DH

In one embodiment, the hinge domain is an IgD hinge encoded by the nucleic acid sequence SEQ ID NO: 150 or a nucleic acid sequence with at least 70%, 75%, 80%, 85%, 90%, 91%, 92%, 93%, 94%, 95%, 96%, 97%, 98% or 99% identity to SEQ ID NO: 150.

SEQ ID NO: 150
AGGTGGCCCGAAAGTCCCAAGGCCCAGGCATCTAG
TGTTCCTACTGCACAGCCCCAGGCAGAAGGCAGCC
TAGCCAAAGCTACTACTGCACCTGCCACTACGCGC
AATACTGGCCGTGGCGGGGAGGAGAAGAAAAAGGA
GAAAGAGAAAGAAGAACAGGAAGAGAGGGAGACCA
AGACCCCTGAATGTCCATCCCATACCCAGCCGCTG
GGCGTCTATCTCTTGACTCCCGCAGTACAGGACTT
GTGGCTTAGAGATAAGGCCACCTTTACATGTTTCG
TCGTGGGCTCTGACCTGAAGGATGCCCATTTGACT
TGGGAGGTTGCCGGAAAGGTACCCACAGGGGGGGT
TGAGGAAGGGTTGCTGGAGCGCCATTCCAATGGCT
CTCAGAGCCAGCACTCAAGACTCACCCTTCCGAGA
TCCCTGTGGAACGCCGGGACCTCTGTCACATGTAC
TCTAAATCATCCTAGCCTGCCCCCACAGCGTCTGA
TGGCCCTTAGAGAGCCAGCCGCCCAGGCACCAGTT
AAGCTTAGCCTGAATCTGCTCGCCAGTAGTGATCC
CCCAGAGGCCGCCAGCTGGCTCTTATGCGAAGTGT
CCGGCTTTAGCCCGCCCAACATCTTGCTCATGTGG
CTGGAGGACCAGCGAGAAGTGAACACCAGCGGCTT
CGCTCCAGCCCGGCCCCCACCCCAGCCGGGTTCTA
CCACATTCTGGGCCTGGAGTGTCTTAAGGGTCCCA
GCACCACCTAGCCCCCAGCCAGCCACATACACCTG
TGTTGTGTCCCATGAAGATAGCAGGACCCTGCTAA
ATGCTTCTAGGAGTCTGGAGGTTTCCTACGTGACT
GACCATT

In another embodiment, the hinge region comprises or consists in the amino acid sequence of a CD28 hinge (SEQ ID NO: 151) or an amino acid sequence with at least 70%, 75%, 80%, 85%, 90%, 91%, 92%, 93%, 94%, 95%, 96%, 97%, 98% or 99% identity to SEQ ID NO: 151.

SEQ ID NO: 151
IEVMYPPPYLDNEKSNGTIIHVKGKHLCPSPLFPGPSKP

In one embodiment, the hinge domain is a CD28 hinge encoded by the nucleic acid SEQ ID NO: 152 or a nucleic acid sequence with at least 70%, 75%, 80%, 85%, 90%, 91%, 92%, 93%, 94%, 95%, 96%, 97%, 98% or 99% identity to SEQ ID NO: 152.

SEQ ID NO: 152
ATTGAAGTTATGTATCCTCCTCCTTACCTAGACAA
TGAGAAGAGCAATGGAACCATTATCCATGTGAAAG
GGAAACACCTTTGTCCAAGTCCCCTATTTCCCGGA
CCTTCTAAGCCC

Examples of transmembrane domains that may be used in a chimeric receptor of the invention include, but are not limited to, transmembrane domains of an alpha, beta or zeta chain of a T-cell receptor, or of CD28, CD3 gamma, CD3 delta, CD3 epsilon, CD3 zeta, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, PD1, ITGAX, CDl1c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C.

In some embodiments, the transmembrane domain may comprise the entire transmembrane domain of the molecule from which it is derived, or it may comprise a functional fragment or variant thereof.

In one embodiment, the transmembrane domain comprises or consists in the amino acid sequence of a CD8 transmembrane domain (SEQ ID NO: 153), or an amino acid sequence with at least 70%, 75%, 80%, 85%, 90%, 91%, 92%, 93%, 94%, 95%, 96%, 97%, 98% or 99% identity to SEQ ID NO: 153. In another embodiment, the transmembrane domain comprises or consists in an amino acid sequence having at least one, two or three modifications but not more than 20, 10 or 5 modifications of an amino acid sequence of SEQ ID NO: 153, or an amino acid sequence with at least 70%, 75%, 80%, 85%, 90%, 91%, 92%, 93%, 94%, 95%, 96%, 97%, 98% or 99% identity to SEQ ID NO: 153.

SEQ ID NO: 153
IYIWAPLAGTCGVLLLSLVITLYC

In another embodiment, the transmembrane domain is encoded by the nucleotide sequence of a CD8 transmembrane domain (SEQ ID NO: 154), or a nucleotide sequence with at least 70%, 75%, 80%, 85%, 90%, 91%, 92%, 93%, 94%, 95%, 96%, 97%, 98% or 99% identity to SEQ ID NO: 154.

SEQ ID NO: 154
ATCTACATCTGGGCGCCCTTGGCCGGGACTTGTGG
GGTCCTTCTCCTGTCACTGGTTATCACCCTTTACT
GC

In another embodiment, the transmembrane domain comprises or consists in the amino acid sequence of a CD28 transmembrane domain (SEQ ID NO: 155) or an amino acid sequence with at least 70%, 75%, 80%, 85%, 90%, 91%, 92%, 93%, 94%, 95%, 96%, 97%, 98% or 99% identity to SEQ ID NO: 155.

SEQ ID NO: 155
FWVLVVVGGVLACYSLLVTVAFIIFWV

In one embodiment, the transmembrane domain is a CD28 transmembrane domain encoded by the nucleic acid sequence SEQ ID NO: 156 or a nucleic acid sequence with at least 70%, 75%, 80%, 85%, 90%, 91%, 92%, 93%, 94%, 95%, 96%, 97%, 98% or 99% identity to SEQ ID NO: 156.

SEQ ID NO: 156
TTTTGGGTGCTGGTGGTGGTTGGTGGAGTCCTGGC
TTGCTATAGCTTGCTAGTAACAGTGGCCTTTATTA
TTTTCTGGGTG

In some embodiments, the transmembrane domain may be recombinant. In certain embodiments, the recombinant transmembrane domain comprises predominantly hydrophobic amino acids such as valine or leucine.

As used herein, the term “intracellular signaling domain” refers to an intracellular portion of a molecule. The intracellular signaling domain generates a signal that promotes an immune effector function of the chimeric receptor containing cell. Examples of immune effector function in a chimeric receptor-T cell may include cytolytic activity, suppressive activity, regulatory activity and helper activity, including the secretion of cytokines.

In some embodiments, the intracellular domain of a CAR of the invention comprises at least one T cell primary signaling domain (or a sequence derived therefrom) and optionally one or more intracellular domain(s) of a T cell costimulatory molecule (or sequence(s) derived therefrom).

As used herein, the term “costimulatory molecule” or “Costimulatory intracellular signaling domain” refers to a cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that contribute to an efficient immune response. A costimulatory signaling domain can be the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors.

In some embodiments, the intracellular domain may comprise the entire intracellular portion, or the entire native intracellular signaling domain, of the molecule from which it is derived, or a functional fragment or variant thereof.

In some embodiments, the intracellular signaling domain consists of at least one primary signaling domain (e.g., a T cell primary signaling domain) or a fragment or variant thereof.

In some embodiments, the intracellular signaling domain consists of at least one costimulatory signaling domain (e.g., a T cell costimulatory molecule intracellular domain) or a fragment or variant thereof.

In some embodiments, the intracellular signaling domain comprises one or more intracellular domain(s) of a T cell costimulatory molecule or a fragment or variant thereof.

In some embodiments, the intracellular signaling domain consists of one or more intracellular domain(s) of a T cell costimulatory molecule or a fragment or variant thereof.

In one embodiment, the intracellular signaling domain of the CAR of the invention comprises at least one costimulatory domain or fragments or variants thereof and at least one primary signaling domain or fragments or variants thereof.

In one embodiment, the intracellular signaling domain of the CAR of the invention consists of one costimulatory domain or fragments or variants thereof and one primary signaling domain or fragments or variants thereof.

In one embodiment, the intracellular signaling domain of the CAR of the invention comprises at least one costimulatory domain or fragments or variants thereof and one primary signaling domain or fragments or variants thereof, and further comprises a expression system allowing expression of a recombinant protein. In one embodiment, the recombinant protein is a pro-inflammatory cytokine, such as e.g. IL-12. In one embodiment, said pro-inflammatory cytokine is released by the CAR-engineered cell.

As used herein, an “expression system” refers to a linear or a circular DNA molecule composed of a fragment encoding the nucleic acid sequence encoding the recombinant peptide, polypeptide or protein of interest operably linked to an additional fragment for the transcription of the system.

In some embodiments, the expression system comprises a nucleic acid sequence encoding a pro-inflammatory cytokine. In one embodiment, the expression system comprises a nucleic acid sequence encoding IL-12.

The additional fragment may include a promoter and a stop codon sequence. The expression system may further contain one or more origins of replication, one or more selection markers and a sequence encoding a ribosome binding site.

“Operably linked” means that fragments are arranged to be functioning as they are supposed to be, for example once transcription starts at the promoter, it goes through coded fragment to stop codon.

“Promoter” in the meaning of the present invention is an expression control element that permits binding of RNA polymerase and the initiation of transcription.

In one embodiment of the invention, the nucleic acid sequence is under the control of a “strong” promoter. A strong promoter is characterized by a high binding affinity of the promoter sequence to an RNA polymerase, usually the naturally occurring corresponding RNA polymerase, on the one hand and by a high rate of formation of mRNA by that RNA polymerase on the other hand.

In another embodiment, the nucleic acid sequence is under the control of an “inducible promoter”. An “inducible promoter” is a promoter that may be regulated by external factors, e.g. the presence of an inductor (also termed “inducer”) molecule or the absence of a repressor molecule, or physical factors like increased or decreased temperature, osmolarity, or pH value.

In one embodiment of the invention, the promoter may also be constitutive, i.e. a promoter which controls expression without the need for induction on the one hand, or the possibility of repression on the other hand. Hence, there is continuous and steady expression at a certain level.

Advantageously, the expression of the recombinant peptide, polypeptide or protein of interest is induced in particular conditions, such as, for example, under selection.

In one embodiment, the expression system is a constitutive expression system. In another embodiment, the expression system is an inducible expression system.

In one embodiment, the expression system is an expression cassette.

In some embodiments, the intracellular signaling domain of a CAR of the invention comprises at least one or two costimulatory domains or a fragment or variant thereof and at least one primary signaling domain or a fragment or variant thereof. In certain embodiments, one or more of the costimulatory domains are intracellular domains of a T cell costimulatory molecule. In certain embodiments, the at least one primary signaling domain is a T cell primary signaling domain.

In some embodiments, the intracellular signaling domain of a CAR of the invention comprises at least one or two costimulatory domains or a fragment or variant thereof and at least one primary signaling domain or a fragment or variant thereof and at least one expression system allowing expression of a recombinant pro-inflammatory cytokine.

Thus, in one embodiment, the CAR-engineered cell expresses a recombinant pro-inflammatory cytokine, such as IL-12.

In some embodiments of the invention, the primary signaling domain comprises a signaling domain of a protein selected from the group consisting of CD3 zeta, CD3 gamma, CD3 delta, CD3 epsilon, common FcR gamma (FCER1G), FcR beta (Fc Epsilon Rib), CD79a, CD79b, Fcgamma RIIa, DAP10, and DAP12, and sequences derived therefrom.

In some embodiments, the primary signaling domain is a T cell primary signaling domain that comprises or consists of at least one functional signaling domain of CD3 zeta or a fragment or variant thereof.

In some embodiments, the T cell primary signaling domain comprises or consists of the CD3 zeta amino acid sequence of SEQ ID NO: 157 or an amino acid sequence with at least about 70%, 75%, 80%, 85%, 90%, 91%, 92%, 93%, 94%, 95%, 96%, 97%, 98% or 99% identity to SEQ ID NO: 157.

SEQ ID NO: 157
RVKFSRSADAPAYKQGQNQLYNELNLGRREEYDVLDK
RRGRDPEMGGKPRRKNPQEGLYNELQKDKMAEAYSEI
GMKGERRRGKGHDGLYQGLSTATKDTYDALHMQALPPR

In some embodiments, the CD3 zeta primary signaling domain comprises or consists of an amino acid sequence having at least one, two or three modifications but not more than 20, 10 or 5 modifications, compared to an amino acid sequence of SEQ ID NO: 157, or an amino acid sequence with at least about 70%, 75%, 80%, 85%, 90%, 91%, 92%, 93%, 94%, 95%, 96%, 97%, 98% or 99% identity to SEQ ID NO: 157.

Thus, in some embodiments, the nucleic acid sequence encoding the T cell primary signaling domain comprises or consists of the CD3 zeta domain nucleic acid sequence of SEQ ID NO: 162 or a nucleotide sequence with at least about 70%, 75%, 80%, 85%, 90%, 91%, 92%, 93%, 94%, 95%, 96%, 97%, 98% or 99% identity to SEQ ID NO: 162.

In some embodiments, the CD3 zeta primary signaling domain comprises at least 2, 10, 20, 30, 40, 50, 60, 70, 80, 90, 100, 110 or 112 amino acids from the sequence of SEQ ID NO: 157 or from a sequence having at least about 70% identity with SEQ ID NO: 157, e.g., at least 2, 10, 20, 30, 40, 50, 60, 70, 80, 90, 100, 110 or 112 contiguous amino acids from SEQ ID NO: 157.

In some embodiments, the CD3 zeta primary signaling domain is encoded by a nucleotide sequence of at least 6, 30, 60, 90, 120, 150, 180, 210, 240, 270, 300, 330 or 336 nucleotides from the sequence of SEQ ID NO: 162, or from a sequence having at least about 70%, 75%, 80%, 85%, 90%, 91%, 92%, 93%, 94%, 95%, 96%, 97%, 98% or 99% identity with SEQ ID NO: 162, e.g., at least 6, 30, 60, 90, 120, 150, 180, 210, 240, 270, 300, 330 or 336 contiguous nucleotides from SEQ ID NO: 162.

SEQ ID NO: 162
AGAGTGAAGTTCAGCAGGAGCGCAGACGCCCCCGC
GTACCAGCAGGGCCAGAACCAGCTCTATAACGAGC
TCAATCTAGGACGAAGAGAGGAGTACGATGTTTTG
GACAAGAGACGTGGCCGGGACCCTGAGATGGGGGG
AAAGCCGAGAAGGAAGAACCCTCAGGAAGGCCTGT
ACAATGAACTGCAGAAAGATAAGATGGCGGAGGCC
TACAGTGAGATTGGGATGAAAGGCGAGCGCCGGAG
GGGCAAGGGGCACGATGGCCTTTACCAGGGTCTCA
GTACAGCCACCAAGGACACCTACGACGCCCTTCAC
ATGCAGGCCCTGCCCCCTCGC

In some embodiments, T cell primary signaling domains that act in a stimulatory manner may comprise signaling motifs known as immunoreceptor tyrosine-based activation motifs (ITAMS). Examples of ITAM-containing T cell primary intracellular signaling domains that are of particular use in the invention include, but are not limited to, those of (or that are derived from) CD3 zeta, common FcR gamma (FCER1G), Fc gamma RIIa, FcR beta (Fc Epsilon Rib), CD3 gamma, CD3 delta, CD3 epsilon, CD5, CD22, CD66b, CD79a, CD79b, DAP10, and DAP12.

In some embodiments, the T cell primary signaling domain comprises a modified ITAM domain, e.g., a mutated ITAM domain that has altered (e.g., increased or decreased) activity as compared to the native ITAM domain. In some embodiments, a primary signaling domain comprises a modified ITAM-containing primary intracellular signaling domain, e.g., an optimized and/or truncated ITAM-containing primary intracellular signaling domain. In certain embodiments, a primary signaling domain may comprise one, two, three, four or more ITAM motifs.

In some embodiments, the intracellular signaling domain of a CAR of the invention comprises a T cell primary signaling domain (such as, for example, a CD3 zeta signaling domain or a fragment or variant thereof), combined with one or more costimulatory signaling domains, wherein said costimulatory signaling domains are entire costimulatory intracellular signaling domains or a fragment or variant thereof.

In some embodiments, the costimulatory signaling domain is the intracellular or cytoplasmic domain of a T cell costimulatory molecule.

In some embodiments, the costimulatory signaling domain is the signaling domain of a T cell costimulatory molecule.

Examples of costimulatory signaling domains include, but are not limited to, the intracellular or cytoplasmic signaling domains of proteins selected from the group consisting of 4-1BB (CD137), ICOS (CD278), CD27, CD28, CTLA-4 (CD152), PD-1, an MHC class I molecule, BTLA, a Toll ligand receptor, OX40, CD30, CD40, lymphocyte function-associated antigen-1 (LFA-1), CD2, CD7, LIGHT, NKG2C, B7-H3, a ligand that specifically binds with CD83, CDS, ICAM-1, GITR, ARHR, BAFFR, HVEM (LIGHTR), SLAMF7, NKp80 (KLRF1), NKp44, NKp30, NKp46, CD160 (BY55), CD19, CD19a, CD4, CD8alpha, CD8beta, IL2ra, IL6Ra, IL2R beta, IL2R gamma, IL7R alpha, IL-13RA1/RA2, IL-33R(IL1RL1), IL-IORA/RB, IL-4R, IL-5R (CSF2RB), IL-21R, ITGA4, VLA1, CD49a, ITGA4, IA4, CD49D, ITGA6, VLA-6, CD49f, ITGAD, CD11d, ITGAE, CD103, ITGAL, CDIIa/CD18, ITGAM, CD11b, ITGAX, CD11c, ITGB1, CD29, ITGB2, CD18, ITGB7, NKG2D, NKG2C, CD95, TGFbRI/2/3, TRANCE/RANKL, DNAM1 (CD226), SLAMF4 (CD244, 2B4), CD84, CD96 (Tactile), CEACAM1, CRTAM, Ly9 (CD229), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, common gamma chain, a ligand that specifically binds with CD83, NKp44, NKp30, NKp46, NKG2D, and any combination thereof.

In some embodiments, the chimeric receptor comprises at least one intracellular or cytoplasmic signaling domain of a T cell costimulatory molecule selected from the group consisting of CD28, 4-1BB, OX40, ICOS, CD27 and DAP10.

In some embodiments, the chimeric receptor comprises at least one costimulatory signaling domain, wherein said costimulatory signaling domain is an entire costimulatory signaling domain or a fragment or variant thereof.

In some embodiments, the T cell costimulatory signaling domain comprises or consists of the amino acid sequence of a 4-1BB costimulatory intracellular signaling domain (e.g., comprising or consisting of the amino acid sequence of SEQ ID NO: 163) or an amino acid sequence with at least about 70%, 75%, 80%, 85%, 90%, 91%, 92%, 93%, 94%, 95%, 96%, 97%, 98% or 99% identity to SEQ ID NO: 163. In some embodiments, the T cell costimulatory signaling domain comprises or consists of an amino acid sequence having at least one, two or three modifications but not more than 20, 10 or modifications compared to the amino acid sequence of SEQ ID NO: 163.

SEQ ID NO: 163
KRGRKKLLYIFKQPFMRPVQTTQEEDGCSCRFPEEEEGGCEL

In some embodiments, the T cell costimulatory signaling domain is encoded by a 4-1BB costimulatory intracellular signaling domain nucleotide sequence (e.g., comprising or consisting of the sequence of SEQ ID NO: 164), or a nucleotide sequence with at least about 70%, 75%, 80%, 85%, 90%, 91%, 92%, 93%, 94%, 95%, 96%, 97%, 98% or 99% identity to SEQ ID NO: 164.

SEQ ID NO: 164
AAACGGGGCAGAAAGAAACTCCTGTATATATTCAA
ACAACCATTTATGAGACCAGTACAAACTACTCAAG
AGGAAGATGGCTGTAGCTGCCGATTTCCAGAAGAA
GAAGAAGGAGGATGTGAACTG

In some embodiments, the 4-1BB costimulatory intracellular signaling domain comprises at least 2, 3, 6, 9, 12, 15, 18, 21, 24, 27, 30, 33, 36, 39, or 42 amino acids from the sequence of SEQ ID NO: 163 or from a sequence having at least about 70%, 75%, 80%, 85%, 90%, 91%, 92%, 93%, 94%, 95%, 96%, 97%, 98% or 99% identity with SEQ ID NO: 163, e.g., at least 2, 3, 6, 9, 12, 15, 18, 21, 24, 27, 30, 33, 36, 39, or 42 contiguous amino acids from SEQ ID NO: 163.

In some embodiments, the 4-1BB costimulatory intracellular signaling domain is encoded by a nucleotide sequence of at least 6, 18, 27, 36, 45, 54, 63, 72, 81, 96, 99, 108, 117 or 126 nucleotides from the sequence of SEQ ID NO: 164 or from a sequence having at least about 70%, 75%, 80%, 85%, 90%, 91%, 92%, 93%, 94%, 95%, 96%, 97%, 98% or 99% identity with SEQ ID NO: 164, e.g., at least 6, 18, 27, 36, 45, 54, 63, 72, 81, 96, 99, 108, 117 or 126 contiguous nucleotides from SEQ ID NO: 164.

In some embodiments, the T cell costimulatory signaling domain comprises or consists of the amino acid sequence of a CD28 costimulatory intracellular signaling domain (e.g., comprising or consisting of the amino acid sequence of SEQ ID NO: 167) or an amino acid sequence with at least about 70%, 75%, 80%, 85%, 90%, 91%, 92%, 93%, 94%, 95%, 96%, 97%, 98% or 99% identity to SEQ ID NO: 167. In some embodiments, the T cell costimulatory signaling domain comprises or consists of an amino acid sequence having at least one, two or three modifications but not more than 20, 10 or modifications compared to the amino acid sequence of SEQ ID NO: 167.

SEQ ID NO: 167
RSKRSRLLHSDYMNMTPRRPGPTRKHYQPYAPPRDFAAYRS

In some embodiments, the T cell costimulatory signaling domain is encoded by a CD28 costimulatory intracellular signaling domain nucleotide sequence (e.g., comprising or consisting of the sequence of SEQ ID NO: 168), or a nucleotide sequence with at least about 70%, 75%, 80%, 85%, 90%, 91%, 92%, 93%, 94%, 95%, 96%, 97%, 98% or 99% identity to SEQ ID NO: 168.

SEQ ID NO: 168
AGGAGTAAGAGGAGCAGGCTCCTGCACAGTGACTA
CATGAACATGACTCCCCGCCGCCCCGGGCCCACCC
GCAAGCATTACCAGCCCTATGCCCCACCACGCGAC
TTCGCAGCCTATCGCTCC

In some embodiments, the CD28 costimulatory intracellular signaling domain comprises at least 2, 3, 6, 9, 12, 15, 18, 21, 24, 27, 30, 33, 36, 39 or 41 amino acids from the sequence of SEQ ID NO: 167 or from a sequence having at least about 70%, 75%, 80%, 85%, 90%, 91%, 92%, 93%, 94%, 95%, 96%, 97%, 98% or 99% identity with SEQ ID NO: 167, e.g., at least 2, 3, 6, 9, 12, 15, 18, 21, 24, 27, 30, 33, 36, 39 or 41 contiguous amino acids from SEQ ID NO: 167.

In some embodiments, the CD28 costimulatory intracellular signaling domain is encoded by a nucleotide sequence of at least 6, 18, 27, 36, 45, 54, 63, 72, 81, 96, 99, 108, 117 or 123 nucleotides from the sequence of SEQ ID NO: 168 or from a sequence having at least about 70%, 75%, 80%, 85%, 90%, 91%, 92%, 93%, 94%, 95%, 96%, 97%, 98% or 99% identity with SEQ ID NO: 168, e.g., at least 6, 18, 27, 36, 45, 54, 63, 72, 81, 96, 99, 108, 117 or 123 contiguous nucleotides from SEQ ID NO: 168.

In some embodiments of the invention, the chimeric receptor comprises at least one intracellular domain of a T cell costimulatory molecule. In certain embodiments, the at least one intracellular domain may be selected from an intracellular domain of 4-1BB and an intracellular domain of CD28. In particular embodiments, the said costimulatory intracellular signaling domains are entire costimulatory intracellular signaling domain or a fragment or variant thereof.

In some embodiments of the invention, the chimeric receptor comprises a combination of at least two intracellular domains of a T cell costimulatory molecule. In particular embodiments, the said costimulatory intracellular signaling domains are entire costimulatory intracellular signaling domains or a fragment or variant thereof.

In some embodiments, the chimeric receptor comprises the amino acid sequence of a 4-1BB costimulatory intracellular signaling domain (e.g., comprising or consisting of the amino acid sequence of SEQ ID NO: 163) or a fragment or variant thereof and the amino acid sequence of a CD28 costimulatory intracellular signaling domain (e.g., comprising or consisting of the amino acid sequence of SEQ ID NO: 167) or a fragment or variant thereof.

In some embodiments of the invention, the chimeric receptor may comprise at least three costimulatory intracellular signaling domains, wherein said domains are entire costimulatory intracellular signaling domains or a fragment or variant thereof.

In some embodiments, the intracellular signaling domain of a CAR of the invention comprises:
a 4-1BB costimulatory intracellular signaling domain with the amino acid sequence of SEQ ID NO: 163, or a fragment or variant thereof, and/or a CD28 costimulatory intracellular signaling domain with the amino acid sequence of SEQ ID NO: 167; and/or
a CD3 zeta primary intracellular signaling domain with the amino acid sequence of SEQ ID NO: 157 or a fragment or variant thereof;

wherein the sequences comprised in the intracellular domain are expressed in the same frame and as a single polypeptide chain.

Thus, in some embodiments, the nucleic acid sequence encoding the intracellular signaling domain of a CAR of the invention comprises:
a 4-1BB costimulatory intracellular signaling domain nucleic acid sequence of SEQ ID NO: 164 or a fragment or variant thereof, and/or a CD28 costimulatory intracellular signaling domain nucleic acid sequence of SEQ ID NO: 168 or a fragment or variant thereof; and/or
a CD3 zeta primary intracellular signaling domain of SEQ ID NO: 162 or a fragment or variant thereof;
wherein the sequences comprised in the intracellular domain are expressed in the same frame and as a single polypeptide chain.

In some embodiments, the intracellular signaling domain of a CAR of the invention comprises at least two different domains (e.g., a primary signaling domain or a fragment or variant thereof and at least one intracellular domain of a T cell costimulatory molecule or a fragment or variant thereof) that may be linked to each other in a random or in a specified order.

Optionally, a short oligo- or polypeptide linker, for example, between 2 and 10 amino acids (e.g., 2, 3, 4, 5, 6, 7, 8, 9, or 10 amino acids) in length may form the linkage between distinct signaling domains. In some embodiments, a glycine-serine doublet (GS) is used as a suitable linker. In some embodiments, a single amino acid, e.g., an alanine (A), a glycine (G), is used as a suitable linker. Other examples of linker are described herein.

In some embodiments, the intracellular signaling domain of a CAR of the invention comprises two or more (e.g., 2, 3, 4, 5, or more) costimulatory intracellular signaling domains. In some embodiments, any or all of the two or more (e.g., 2, 3, 4, 5, or more) costimulatory signaling domains are separated by a linker molecule, e.g., a linker molecule as described herein.

In some embodiments, the intracellular signaling domain of a chimeric receptor of the invention comprises the primary intracellular signaling domain of CD3 zeta (e.g., SEQ ID NO: 157 and the costimulatory intracellular signaling domain of 4-1BB (e.g., SEQ ID NO: 163).

In some embodiments, the intracellular signaling domain of a chimeric receptor of the invention comprises the primary intracellular signaling domain of CD3 zeta (e.g., SEQ ID NO: 157 and the costimulatory intracellular signaling domain of CD28 (e.g., SEQ ID NO: 167).

In some embodiments, a CAR of the invention comprises any combination of an extracellular binding domain as described herein, a transmembrane domain as described herein, an intracellular signaling domain as described herein, and optionally a spacer or hinge domain as described herein.

In some embodiments, a CAR of the invention further comprises a tag, such as, for example, a tag for quality control, enrichment, tracking in vivo and the like. Said tag may be localized N-terminally, C-terminally and/or internally. Examples of tags that may be used in a CAR of the invention are well known by the skilled artisan.

According to a first embodiment, the CAR of the invention comprises at least one extracellular CD45RC binding domain, optionally an extracellular hinge domain, at least one transmembrane domain, and at least one intracellular signaling domain.

In one embodiment, the CAR of the invention comprises an CD45RC binding domain; a transmembrane domain of CD8 (preferably SEQ ID NO: 153); and a CD3-zeta primary signaling domain (preferably SEQ ID NO: 157).

In one embodiment, the CAR of the invention comprises an CD45RC binding domain; a transmembrane domain of CD28 (preferably SEQ ID NO: 155); and a CD3-zeta primary signaling domain (preferably SEQ ID NO: 157).

In another embodiment, the CAR of the invention comprises an CD45RC binding domain; a hinge domain of CD8 (preferably SEQ ID NO: 145); a transmembrane domain of CD8 (preferably SEQ ID NO: 153); and a CD3-zeta primary signaling domain (preferably SEQ ID NO: 157).

In one embodiment, the CAR of the invention comprises an CD45RC binding domain; a hinge domain of CD8 (preferably SEQ ID NO: 145); a transmembrane domain of CD28 (preferably SEQ ID NO: 155); and a CD3-zeta primary signaling domain (preferably SEQ ID NO: 157).

In another embodiment, the CAR of the invention comprises an CD45RC binding domain; a hinge domain of IgG4 (preferably SEQ ID NO: 147); a transmembrane domain of CD8 (preferably SEQ ID NO: 153); and a CD3-zeta primary signaling domain (preferably SEQ ID NO: 157).

In one embodiment, the CAR of the invention comprises an CD45RC binding domain; a hinge domain of IgG4 (preferably SEQ ID NO: 147); a transmembrane domain of CD28 (preferably SEQ ID NO: 155); and a CD3-zeta primary signaling domain (preferably SEQ ID NO: 157).

In another embodiment, the CAR of the invention comprises an CD45RC binding domain; a hinge domain of IgD (preferably SEQ ID NO: 149); a transmembrane domain of CD8 (preferably SEQ ID NO: 153); and a CD3-zeta primary signaling domain (preferably SEQ ID NO: 157).

In one embodiment, the CAR of the invention comprises an CD45RC binding domain; a hinge domain of IgD (preferably SEQ ID NO: 149); a transmembrane domain of CD28 (preferably SEQ ID NO: 155); and a CD3-zeta primary signaling domain (preferably SEQ ID NO: 157).

In another embodiment, the CAR of the invention comprises an CD45RC binding domain; a hinge domain of CD28 (preferably SEQ ID NO: 151); a transmembrane domain of CD8 (preferably SEQ ID NO: 153); and a CD3-zeta primary signaling domain (preferably SEQ ID NO: 157).

In one embodiment, the CAR of the invention comprises an CD45RC binding domain; a hinge domain of CD28 (preferably SEQ ID NO: 151); a transmembrane domain of CD28 (preferably SEQ ID NO: 155); and a CD3-zeta primary signaling domain (preferably SEQ ID NO: 157).

According to a second embodiment, the CAR of the invention comprises an CD45RC binding domain, optionally an extracellular hinge domain, a transmembrane domain, a single intracellular domain of a T cell costimulatory molecule and a T cell primary signaling domain.

In one embodiment, the CAR of the invention comprises an CD45RC binding domain; a transmembrane domain of CD8 (preferably SEQ ID NO: 153); an intracellular domain of 4-1BB (preferably SEQ ID NO: 163); and a CD3-zeta primary signaling domain (preferably SEQ ID NO: 157).

In one embodiment, the CAR of the invention comprises an CD45RC binding domain; a transmembrane domain of CD8 (preferably SEQ ID NO: 153); an intracellular domain of CD28 (preferably SEQ ID NO: 167); and a CD3-zeta primary signaling domain (preferably SEQ ID NO: 157).

In one embodiment, the CAR of the invention comprises an CD45RC binding domain; a transmembrane domain of CD28 (preferably SEQ ID NO: 155); an intracellular domain of 4-1BB (preferably SEQ ID NO: 163); and a CD3-zeta primary signaling domain (preferably SEQ ID NO: 157).

In one embodiment, the CAR of the invention comprises an CD45RC binding domain; a transmembrane domain of CD28 (preferably SEQ ID NO: 155); an intracellular domain of CD28 (preferably SEQ ID NO: 167); and a CD3-zeta primary signaling domain (preferably SEQ ID NO: 157).

In another embodiment, the CAR of the invention comprises an CD45RC binding domain; a hinge domain of CD8 (preferably SEQ ID NO: 145); a transmembrane domain of CD8 (preferably SEQ ID NO: 153); an intracellular domain of 4-1BB (preferably SEQ ID NO: 163); and a CD3-zeta primary signaling domain (preferably SEQ ID NO: 157).

In one embodiment, the CAR of the invention comprises an CD45RC binding domain; a hinge domain of CD8 (preferably SEQ ID NO: 145); a transmembrane domain of CD8 (preferably SEQ ID NO: 153); an intracellular domain of CD28 (preferably SEQ ID NO: 155); and a CD3-zeta primary signaling domain (preferably SEQ ID NO: 157).

In one embodiment, the CAR of the invention comprises an CD45RC binding domain; a hinge domain of CD8 (preferably SEQ ID NO: 145); a transmembrane domain of CD28 (preferably SEQ ID NO: 155); an intracellular domain of 4-1BB (preferably SEQ ID NO: 163); and a CD3-zeta primary signaling domain (preferably SEQ ID NO: 157).

In one embodiment, the CAR of the invention comprises an CD45RC binding domain; a hinge domain of CD8 (preferably SEQ ID NO: 145); a transmembrane domain of CD28 (preferably SEQ ID NO: 155); an intracellular domain of CD28 (preferably SEQ ID NO: 167); and a CD3-zeta primary signaling domain (preferably SEQ ID NO: 157).

In another embodiment, the CAR of the invention comprises an CD45RC binding domain; a hinge domain of IgG4 (preferably SEQ ID NO: 147); a transmembrane domain of CD8 (preferably SEQ ID NO: 153); an intracellular domain of 4-1BB (preferably SEQ ID NO: 163); and a CD3-zeta primary signaling domain (preferably SEQ ID NO: 157).

In one embodiment, the CAR of the invention comprises an CD45RC binding domain; a hinge domain of IgG4 (preferably SEQ ID NO: 147); a transmembrane domain of CD8 (preferably SEQ ID NO: 153); an intracellular domain of CD28 (preferably SEQ ID NO: 167); and a CD3-zeta primary signaling domain (preferably SEQ ID NO: 157).

In one embodiment, the CAR of the invention comprises an CD45RC binding domain; a hinge domain of IgG4 (preferably SEQ ID NO: 147); a transmembrane domain of CD28 (preferably SEQ ID NO: 155); an intracellular domain of 4-1BB (preferably SEQ ID NO: 163); and a CD3-zeta primary signaling domain (preferably SEQ ID NO: 157).

In one embodiment, the CAR of the invention comprises an CD45RC binding domain; a hinge domain of IgG4 (preferably SEQ ID NO: 147); a transmembrane domain of CD28 (preferably SEQ ID NO: 155); an intracellular domain of CD28 (preferably SEQ ID NO: 167); and a CD3-zeta primary signaling domain (preferably SEQ ID NO: 157).

In another embodiment, the CAR of the invention comprises an CD45RC binding domain; a hinge domain of IgD (preferably SEQ ID NO: 149); a transmembrane domain of CD8 (preferably SEQ ID NO: 153); an intracellular domain of 4-1BB (preferably SEQ ID NO: 163); and a CD3-zeta primary signaling domain (preferably SEQ ID NO: 157).

In one embodiment, the CAR of the invention comprises an CD45RC binding domain; a hinge domain of IgD (preferably SEQ ID NO: 149); a transmembrane domain of CD8 (preferably SEQ ID NO: 153); an intracellular domain of CD28 (preferably SEQ ID NO: 167); and a CD3-zeta primary signaling domain (preferably SEQ ID NO: 157).

In one embodiment, the CAR of the invention comprises an CD45RC binding domain; a hinge domain of IgD (preferably SEQ ID NO: 149); a transmembrane domain of CD28 (preferably SEQ ID NO: 155); an intracellular domain of 4-1BB (preferably SEQ ID NO: 163); and a CD3-zeta primary signaling domain (preferably SEQ ID NO: 157).

In one embodiment, the CAR of the invention comprises an CD45RC binding domain; a hinge domain of IgD (preferably SEQ ID NO: 149); a transmembrane domain of CD28 (preferably SEQ ID NO: 155); an intracellular domain of CD28 (preferably SEQ ID NO: 167); and a CD3-zeta primary signaling domain (preferably SEQ ID NO: 157).

In another embodiment, the CAR of the invention comprises an CD45RC binding domain; a hinge domain of CD28 (preferably SEQ ID NO: 151); a transmembrane domain of CD8 (preferably SEQ ID NO: 153); an intracellular domain of 4-1BB (preferably SEQ ID NO: 163); and a CD3-zeta primary signaling domain (preferably SEQ ID NO: 157).

In one embodiment, the CAR of the invention comprises an CD45RC binding domain; a hinge domain of CD28 (preferably SEQ ID NO: 151); a transmembrane domain of CD8 (preferably SEQ ID NO: 153); an intracellular domain of CD28 (preferably SEQ ID NO: 167); and a CD3-zeta primary signaling domain (preferably SEQ ID NO: 157).

In one embodiment, the CAR of the invention comprises an CD45RC binding domain; a hinge domain of CD28 (preferably SEQ ID NO: 151); a transmembrane domain of CD28 (preferably SEQ ID NO: 155); an intracellular domain of 4-1BB (preferably SEQ ID NO: 163); and a CD3-zeta primary signaling domain (preferably SEQ ID NO: 157).

In one embodiment, the CAR of the invention comprises an CD45RC binding domain; a hinge domain of CD28 (preferably SEQ ID NO: 151); a transmembrane domain of CD28 (preferably SEQ ID NO: 155); an intracellular domain of CD28 (preferably SEQ ID NO: 167); and a CD3-zeta primary signaling domain (preferably SEQ ID NO: 157).

According to a third embodiment, the CAR of the invention comprises an CD45RC binding domain, optionally an extracellular hinge domain, a transmembrane domain, two intracellular domains of a T cell costimulatory molecule and a T cell primary signaling domain.

In one embodiment, the CAR of the invention comprises an CD45RC binding domain; a transmembrane domain of CD8 (preferably SEQ ID NO: 153); an intracellular domain of 4-1BB (preferably SEQ ID NO: 163); an intracellular domain of CD28 (preferably SEQ ID NO: 167); and a CD3-zeta primary signaling domain (preferably SEQ ID NO: 157).

In one embodiment, the CAR of the invention comprises an CD45RC binding domain; a transmembrane domain of CD28 (preferably SEQ ID NO: 155); an intracellular domain of 4-1BB (preferably SEQ ID NO: 163); an intracellular domain of CD28 (preferably SEQ ID NO: 167); and a CD3-zeta primary signaling domain (preferably SEQ ID NO: 157).

In one embodiment, the CAR of the invention comprises an CD45RC binding domain; a hinge domain of CD8 (preferably SEQ ID NO: 145); a transmembrane domain of CD8 (preferably SEQ ID NO: 153); an intracellular domain of 4-1BB (preferably SEQ ID NO: 163); an intracellular domain of CD28 (preferably SEQ ID NO: 167); and a CD3-zeta primary signaling domain (preferably SEQ ID NO: 157).

In one embodiment, the CAR of the invention comprises an CD45RC binding domain; a hinge domain of CD8 (preferably SEQ ID NO: 145); a transmembrane domain of CD28 (preferably SEQ ID NO: 155); an intracellular domain of 4-1BB (preferably SEQ ID NO: 163); an intracellular domain of CD28 (preferably SEQ ID NO: 167); and a CD3-zeta primary signaling domain (preferably SEQ ID NO: 157).

In one embodiment, the CAR of the invention comprises an CD45RC binding domain; a hinge domain of IgG4 (preferably SEQ ID NO: 147); a transmembrane domain of CD8 (preferably SEQ ID NO: 153); an intracellular domain of 4-1BB (preferably SEQ ID NO: 163); an intracellular domain of CD28 (preferably SEQ ID NO: 167); and a CD3-zeta primary signaling domain (preferably SEQ ID NO: 157).

In one embodiment, the CAR of the invention comprises an CD45RC binding domain; a hinge domain of IgG4 (preferably SEQ ID NO: 147); a transmembrane domain of CD28 (preferably SEQ ID NO: 155); an intracellular domain of 4-1BB (preferably SEQ ID NO: 163); an intracellular domain of CD28 (preferably SEQ ID NO: 167); and a CD3-zeta primary signaling domain (preferably SEQ ID NO: 157).

In one embodiment, the CAR of the invention comprises an CD45RC binding domain; a hinge domain of IgD (preferably SEQ ID NO: 149); a transmembrane domain of CD8 (preferably SEQ ID NO: 153); an intracellular domain of 4-1BB (preferably SEQ ID NO: 163); an intracellular domain of CD28 (preferably SEQ ID NO: 167); and a CD3-zeta primary signaling domain (preferably SEQ ID NO: 157).

In one embodiment, the CAR of the invention comprises an CD45RC binding domain; a hinge domain of IgD (preferably SEQ ID NO: 149); a transmembrane domain of CD28 (preferably SEQ ID NO: 155); an intracellular domain of 4-1BB (preferably SEQ ID NO: 163); an intracellular domain of CD28 (preferably SEQ ID NO: 167); and a CD3-zeta primary signaling domain (preferably SEQ ID NO: 157).

In one embodiment, the CAR of the invention comprises an CD45RC binding domain; a hinge domain of CD28 (preferably SEQ ID NO: 151); a transmembrane domain of CD8 (preferably SEQ ID NO: 153); an intracellular domain of 4-1BB (preferably SEQ ID NO: 163); an intracellular domain of CD28 (preferably SEQ ID NO: 167); and a CD3-zeta primary signaling domain (preferably SEQ ID NO: 157).

In one embodiment, the CAR of the invention comprises an CD45RC binding domain; a hinge domain of CD28 (preferably SEQ ID NO: 151); a transmembrane domain of CD28 (preferably SEQ ID NO: 155); an intracellular domain of 4-1BB (preferably SEQ ID NO: 163); an intracellular domain of CD28 (preferably SEQ ID NO: 167); and a CD3-zeta primary signaling domain (preferably SEQ ID NO: 157).

In one embodiment, the CAR of the invention comprises (i) an CD45RC binding domain, (ii) a hinge region of human CD8, (iii) a transmembrane domain of human CD8, (iv) an intracellular domain of human CD28 and (v) an intracellular domain of human CD3ζ chain.

In one embodiment, the part of the CAR comprising a hinge region of human CD8, a transmembrane domain of human CD8, an intracellular domain of human CD28 and an intracellular domain of human CD3ζ chain corresponds to the amino acid sequence of SEQ ID NO: 169 or an amino acid sequence with at least about 95, preferably about 96%, 97%, 98% or 99% identity to SEQ ID NO: 169.

In one embodiment, the CAR of the invention comprises an CD45RC binding domain, linked to an amino acid sequence of SEQ ID NO: 169 or a sequence or an amino acid sequence with at least about 95, preferably about 96%, 97%, 98% or 99% identity to SEQ ID NO: 169.

SEQ ID NO: 169
TTTPAPRPPTPAPTIASQPLSLRPEACRPAAGGAV
HTRGLDFACDIYIWAPLAGTCGVLLLSLVITLYCR
SKRSRLLHSDYMNMTPRRPGPTRKHYQPYAPPRDF
AAYRSRVKFSRSADAPAYQQGQNQLYNELNLGRRE
EYDVLDKRRGRDPEMGGKPRRKNPQEGLYNELQKD
KMAEAYSEIGMKGERRRGKGHDGLYQGLSTATKDT
YDALHMQALPPR

In one embodiment, the part of the CAR comprising a hinge region of human CD8, a transmembrane domain of human CD8, an intracellular domain of human CD28 and an intracellular domain of human CD3ζ chain corresponds to the nucleotide sequence of SEQ ID NO: 170 or a nucleotide sequence with at least about 95, preferably about 96%, 97%, 98% or 99% identity to SEQ ID NO: 170.

In another embodiment, the CAR of the invention comprises (i) an CD45RC binding domain, (ii) a hinge region of human CD8, (iii) a transmembrane domain of human CD8, (iv) an intracellular domain of human CD28 and (v) an intracellular domain of human CD3ζ

In one embodiment, the part of the CAR comprising a hinge region of human CD8, a transmembrane domain of human CD8, an intracellular domain of human CD28 and an intracellular domain of human CD3ζ comprises or consists in the amino acid sequence SEQ ID NO:169, or any amino acid sequence with at least about 95, preferably about 96%, 97%, 98% or 99% identity with SEQ ID NO: 169.

In one embodiment, the part of the CAR comprising a CD45RC binding domain, a hinge region of human CD8, a transmembrane domain of human CD8, an intracellular domain of human CD28 and an intracellular domain of human CD3ζ comprises or consists in the amino acid sequence SEQ ID NO: 171, or any amino acid sequence with at least about 95, preferably about 96%, 97%, 98% or 99% identity with SEQ ID NO: 171.

SEQ ID NO: 170
ACCACGACGCCAGCGCCGCGACCACCAACACCGGC
GCCCACCATCGCGTCGCAGCCCCTGTCCCTGCGCC
CAGAGGCGTGCCGGCCAGCGGCGGGGGGCGCAGTG
CACACGAGGGGGCTGGACTTCGCCTGTGATATCTA
CATCTGGGCGCCCTTGGCCGGGACTTGTGGGGTCC
TTCTCCTGTCACTGGTTATCACCCTTTACTGCAGG
AGTAAGAGGAGCAGGCTCCTGCACAGTGACTACAT
GAACATGACTCCCCGCCGCCCCGGGCCCACCCGCA
AGCATTACCAGCCCTATGCCCCACCACGCGACTTC
GCAGCCTATCGCTCCAGAGTGAAGTTCAGCAGGAG
CGCAGACGCCCCCGCGTACCAGCAGGGCCAGAACC
AGCTCTATAACGAGCTCAATCTAGGACGAAGAGAG
GAGTACGATGTTTTGGACAAGAGACGTGGCCGGGA
CCCTGAGATGGGGGGAAAGCCGAGAAGGAAGAACC
CTCAGGAAGGCCTGTACAATGAACTGCAGAAAGAT
AAGATGGCGGAGGCCTACAGTGAGATTGGGATGAA
AGGCGAGCGCCGGAGGGGCAAGGGGCACGATGGCC
TTTACCAGGGTCTCAGTACAGCCACCAAGGACACC
TACGACGCCCTTCACATGCAGGCCCTGCCCCCTCG
C
SEQ ID NO: 171
KVQLQQSGAELVRPGTSVKMSCKAAGYTFTNYYIG
WVKQRPGHGLEWIGDIFPGGDYANSNEKFKGKATL
TADTSSSTAYMQLSSLTSEDSAIYYCVRRNFDYWG
QGTTLTVSSGGGGSGGGGSGGGGSQIVLTQSPTIM
SASPGEKVTITCSASSSVSYMHWFQQKTGTSPRLW
IYNTSNLPSGVPARFSGSGSGTSYSLTISRMEAED
AATYYCQQRSSYPLTFGAGTKLELKTTTPAPRPPT
PAPTIASQPLSLRPEACRPAAGGAVHTRGLDFACD
IYIWAPLAGTCGVLLLSLVITLYCRSKRSRLLHSD
YMNMTPRRPGPTRKHYQPYAPPRDFAAYRSRVKFS
RSADAPAYQQGQNQLYNELNLGRREEYDVLDKRRG
RDPEMGGKPRRKNPQEGLYNELQKDKMAEAYSEIG
MKGERRRGKGHDGLYQGLSTATKDTYDALHMQALP
PR

The present invention also relates to a nucleic acid encoding the CAR of the invention.

Thus, the present invention further relates to a nucleic acid sequence encoding a CAR as described herein, wherein said nucleic acid sequence comprises:
at least one nucleic acid sequence of an extracellular binding domain, wherein said binding domain binds to said human CD45RC,
optionally at least one nucleic acid sequence of an extracellular hinge domain
at least one nucleic acid sequence of a transmembrane domain, and
at least one nucleic acid sequence of an intracellular domain, wherein the at least one nucleic acid sequence of the intracellular domain comprises at least one nucleic acid sequence of a primary intracellular signaling domain and optionally at least one nucleic acid sequence of a costimulatory intracellular signaling domain.

In some embodiments, the at least one nucleic acid sequence of the extracellular binding domain comprises, or consists of, a nucleic acid encoding an antibody or binding fragment thereof binding to hCD45RC disclosed hereabove in the “Antibody or antigen-binding fragment” section.

In some embodiments, the nucleic acid sequence encoding a CAR according to the invention has the sequence of SEQ ID NO: 172 or a sequence with at least 70%, 75%, 80%, 85%, 90%, 91%, 92%, 93%, 94%, 95%, 96%, 97%, 98% or 99% identity to SEQ ID NO: 172.

SEQ ID NO: 172
AAGGTCCAGCTGCAACAGTCTGGCGCTGAGCTGGT
TAGGCCTGGGACTTCAGTGAAGATGTCCTGCAAGG
CCGCTGGATACACCTTCACTAACTACTACATAGGT
TGGGTAAAGCAGAGGCCTGGACATGGCCTTGAGTG
GATCGGAGATATTTTCCCTGGAGGTGACTATGCCA
ACAGCAATGAGAAGTTCAAGGGCAAAGCCACACTG
ACTGCAGACACATCCTCCAGCACAGCCTACATGCA
GCTCAGCAGCCTGACATCTGAGGACTCTGCCATCT
ATTACTGTGTGAGAAGGAACTTTGACTACTGGGGC
CAAGGCACCACTCTCACAGTGTCCTCAGGTGGCGG
TGGCTCGGGCGGTGGTGGGTCGGGTGGCGGCGGAT
CTCAAATTGTTCTCACCCAGTCTCCAACAATCATG
TCTGCATCTCCAGGGGAGAAGGTGACCATAACCTG
CAGTGCCAGCTCAAGTGTAAGTTACATGCACTGGT
TCCAGCAGAAGACAGGCACTTCTCCCAGACTCTGG
ATTTATAACACATCCAACCTGCCTTCTGGAGTCCC
CGCTCGCTTCAGTGGCAGTGGATCTGGGACCTCTT
ACTCTCTCACAATCAGCCGAATGGAGGCTGAAGAT
GCTGCCACTTATTACTGCCAGCAAAGGAGTAGTTA
CCCACTCACGTTCGGTGCTGGGACCAAGCTGGAGC
TGAAAACCACGACGCCAGCGCCGCGACCACCAACA
CCGGCGCCCACCATCGCGTCGCAGCCCCTGTCCCT
GCGCCCAGAGGCGTGCCGGCCAGCGGCGGGGGGCG
CAGTGCACACGAGGGGGCTGGACTTCGCCTGTGAT
ATCTACATCTGGGCGCCCTTGGCCGGGACTTGTGG
GGTCCTTCTCCTGTCACTGGTTATCACCCTTTACT
GCAGGAGTAAGAGGAGCAGGCTCCTGCACAGTGAC
TACATGAACATGACTCCCCGCCGCCCCGGGCCCAC
CCGCAAGCATTACCAGCCCTATGCCCCACCACGCG
ACTTCGCAGCCTATCGCTCCAGAGTGAAGTTCAGC
AGGAGCGCAGACGCCCCCGCGTACCAGCAGGGCCA
GAACCAGCTCTATAACGAGCTCAATCTAGGACGAA
GAGAGGAGTACGATGTTTTGGACAAGAGACGTGGC
CGGGACCCTGAGATGGGGGGAAAGCCGAGAAGGAA
GAACCCTCAGGAAGGCCTGTACAATGAACTGCAGA
AAGATAAGATGGCGGAGGCCTACAGTGAGATTGGG
ATGAAAGGCGAGCGCCGGAGGGGCAAGGGGCACGA
TGGCCTTTACCAGGGTCTCAGTACAGCCACCAAGG
ACACCTACGACGCCCTTCACATGCAGGCCCTGCCC
CCTCGC

In one embodiment, the nucleic acid of the invention is an isolated nucleic acid.

In one embodiment, the isolated nucleic acid of the invention is purified.

In one embodiment, the isolated nucleic acid is purified to:
(1) greater than 80%, 85%, 90%, 91%, 92%, 93%, 94%, 95% or more by weight of nucleic acid as determined by absorbance methods or fluorescence methods (such as, e.g., by measuring the ratio of absorbance at 260 and 280 nm (A₂₆₀/2₈₀)), and most preferably more than 96%, 97%, 98% or 99% by weight; or
(2) homogeneity as shown by agarose gel electrophoresis and using an intercalating agent such as ethidium bromide, SYBR Green, GelGreen or the like.

It is further understood that the one skilled in the art is familiar with molecular biology methods aiming at modifying a nucleic acid sequence in order to improve, e.g., recombinant production rates, such as by codon optimization.

The invention also provides a vector comprising a CAR-encoding nucleic acid sequence as described herein.

Examples of vectors that may be used in the present invention include, but are not limited to, a DNA vector, a RNA vector, a plasmid, a phagemid, a phage derivative, a virus and a cosmid.

Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses that are useful as vectors include, but are not limited to, retroviruses, adenoviruses, adeno-associated viruses, herpes viruses, and lentiviruses.

As used herein, the term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

In general, a suitable vector contains an origin of replication functional in at least one organism, a promoter sequence, convenient restriction endonuclease sites, and one or more selectable markers, (see, e.g., PCT Patent Publications WO 01/96584 and WO01/29058 and U.S. Pat. No. 6,326,193, incorporated herein by reference).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. Further, a number of adenovirus vectors are known in the art. In some embodiments, lentivirus vectors are used.

Additional transcriptionally active elements, e.g., promoters and enhancers, regulate the frequency of transcriptional initiation. Typically core promoter, these are located in the region 30-110 bp upstream of the start site, although a number of promoters have recently been shown to contain functional elements downstream of the start site as well, and enhancer elements are generally located 500-2000 bp upstream of the start site. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor—1a (EF-1a). Another example of a suitable promoter is phosphoglycerate kinase (PGK) promoter. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, and a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence that it is operatively linked to when such expression is desired, or turning off the expression when expression is not desired. Examples of inducible promoters include, but are not limited to, a metallothionine promoter, a glucocorticoid promoter, a progesterone promoter, and a tetracycline promoter. In addition, bi-directional promoters allowing efficient and coordinated expression of two or more genes may also be of interest in the present invention. Examples of bi-directional promoters include, but are not limited to, the promoters described by Luigi Naldini in U.S. Patent Publication 2006/200869, incorporated herein by reference, disclosing a bi-directional promoter comprising i) a first minimal promoter sequence derived from cytomegalovirus (CMV) or mouse mammary tumor virus (MMTV) genomes and ii) a full efficient promoter sequence derived from an animal gene.

In order to assess the expression of a CAR polypeptide or portions thereof, the expression vector to be introduced into a T cell can also contain either a selectable marker gene such as CD34, CD271 or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in host cells. Useful selectable markers include, for example, antibiotic-resistance genes, such as neomycin and the like.

In some embodiments of the invention, suicide gene technology may be used. Different suicide gene technologies are described in the art depending on their mechanism of action (see, e.g., Jones et al., Frontiers in Pharmacology 5:254 (2014)). Examples of gene-directed enzyme prodrug therapy (GDEPT) converting a nontoxic drug to a toxic drug include herpes simplex virus thymidine kinase (HSV-TK) and cytosine deaminase (CD).

Other examples are chimeric proteins composed of a drug binding domain linked to apoptotic components such as for example the inducible Fas (iFas) or the inducible Caspase 9 (iCasp9) systems. Other examples include systems mediated by therapeutic antibodies such as inducing overexpression of c-myc at the surface of the engineered cell to induce its deletion by administration of an anti-c-myc antibody. The use of EGFR is described as a similar system compared to the c-myc system.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (see, e.g., Ui-Tei et al., FEBS Letters 479:79-82 (2000)). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., a mammalian, bacterial, yeast, or insect cell, by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). In some embodiments, of the invention, a polynucleotide is introduced into a host cell using calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances that may be naturally occurring or synthetic lipids. For example, lipids include the fatty droplets that naturally occur in the cytoplasm as well as the class of compounds that contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; and dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

In some embodiments, the immune cells of the invention are modified through the introduction of RNA. In some embodiments, an in vitro transcribed RNA CAR can be introduced to a cell as a form of transient transfection. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. In certain embodiments, the template for in vitro transcription is the CAR of the present invention.

In some embodiments, the DNA to be used for PCR contains an open reading frame. The DNA may be, e.g., from a naturally occurring DNA sequence from the genome of an organism. In some embodiments, the DNA is a full-length gene of interest or a portion of a gene. The gene can include some or all of the 5′ and/or 3′ untranslated regions (UTRs). The gene can include exons and introns. In some embodiments, the DNA to be used for PCR is a human gene. In some embodiments, the DNA to be used for PCR is a human gene including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

PCR may be used to generate a template for in vitro transcription of mRNA that is used for transfection. Methods for performing PCR are well known in the art. Primers for use in PCR are designed to have regions that are substantially complementary to regions of the DNA to be used as a template for the PCR. “Substantially complementary,” as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a gene that is normally transcribed in cells (the open reading frame), which may include 5′ and 3′ UTRs.

The primers can also be designed to amplify a portion of a gene that encodes a particular domain of interest. In some embodiments, the primers are designed to amplify the coding region of a human cDNA, including all or portions of the 5′ and 3′ UTRs. Primers useful for PCR are generated by synthetic methods that are well known in the art.

“Forward primers” are primers that contain a region of nucleotides that are substantially complementary to nucleotides on the DNA template that are upstream of the DNA sequence that is to be amplified. “Upstream” is used herein to refer to a location 5′ to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Any DNA polymerase useful for PCR can be used in the methods disclosed herein.

The reagents and polymerase are commercially available from a number of sources.

Chemical structures with the ability to promote stability and/or translation efficiency may also be used. In some embodiments, the RNA may have 5′ and 3′ UTRs. In some embodiments, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA. The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA.

For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In some embodiments, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but do not appear to be required for all RNAs to enable efficient translation. The requirement for Kozak sequences for many mRNAs is known in the art. In other embodiments, the 5′ UTR can be derived from an RNA virus whose RNA genome is stable in cells. In other embodiments, various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In some embodiments, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In some embodiments, the mRNA has both a cap on the 5′ end and a 3′ poly(A) tail that determine ribosome binding, initiation of translation and stability of the mRNA in the cell. On a circular DNA template, for instance, plasmid DNA, RNA polymerase produces a long concatemeric product that is not suitable for expression in eukaryotic cells. The transcription of plasmid DNA linearized at the end of the 3′ UTR results in normal sized mRNA that is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However, polyA/T sequences integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method that allows construction of DNA templates with polyA/T 3′ stretch without cloning is highly desirable.

The polyA/T segment of the transcriptional DNA template can be produced during PCR by using a reverse primer containing a polyT tail, such as 100T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In some embodiments, the poly(A) tail is between 100 and 5000 adenosines.

Poly(A) tails of RNAs can be further extended following in vitro transcription with the use of a poly(A) polymerase, such as E. coli polyA polymerase (E-PAP). In some embodiments, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps on RNAs may also provide stability to RNA molecules. In some embodiments, RNAs produced by the methods disclosed herein include a 5′ cap. The 5′ cap is provided using techniques known in the art and described herein (Cougot et al., Trends in Biochem. Sci. 29:436-444 (2001); Stepinski et al., RNA 7:1468-95 (2001); Elango, et al., Biochim. Biophys. Res. Commun. 330:958-966 (2005)).

The RNAs produced by the methods disclosed herein can also contain an internal ribosome entry site (IRES) sequence. The IRES sequence may be any viral, chromosomal or artificially designed sequence that initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants, can be included.

RNA can be introduced into target cells using any of a number of different methods, for instance, commercially available methods which include, but are not limited to, electroporation (e.g., Amaxa Nucleofector-II (Amaxa Biosystems, Cologne, Germany), ECM 830 (BTX) (Harvard Instruments, Boston, Mass.), Gene Pulser II (BioRad, Denver, Colo.), or Multiporator (Eppendort, Hamburg Germany)), cationic liposome mediated transfection using lipofection, polymer encapsulation, peptide mediated transfection, or biolistic particle delivery systems such as “gene guns” (see, for example, Nishikawa et al. Hum Gene Ther. 12(8):861-70 (2001)).

In some embodiments, CAR sequences described herein are delivered into immune cells of the invention by using a retroviral or lentiviral vector. CAR-expressing retroviral and lentiviral vectors can be delivered into different types of eukaryotic cells as well as into tissues and whole organisms using transduced cells as carriers or cell-free local or systemic delivery of encapsulated, bound or naked vectors. The method used can be for any purpose where stable expression is required or sufficient.

As used herein, the term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, the LENTIVECTOR@ gene delivery technology from Oxford BioMedica, the LENTIMAX™ vector system from Lentigen and the like. Nonclinical types of lentiviral vectors are also available and would be known to one skilled in the art.

In some embodiments, the CAR sequences are delivered into immune cells of the invention by using in vitro transcribed mRNA. In vitro transcribed mRNA CARs can be delivered into different types of eukaryotic cells as well as into tissues and whole organisms using transfected cells as carriers or cell-free local or systemic delivery of encapsulated, bound or naked mRNA. The method used can be for any purpose where transient expression is required or sufficient.

In some embodiments, the desired CAR can be expressed in the cells by way of transposons.

In some embodiments, an immune cell of the invention is, e.g., a T cell. Prior to expansion and genetic modification of T cells (such as Treg, Teff, memory T cells, NKT or MAIT cells) as described herein, the cells are obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art may be used. In certain embodiments of the present invention, T cells can be obtained from a unit of blood collected from a subject using any number of techniques known to the skilled artisan, such as Ficoll™ separation, centrifugation through a PERCOLL™ gradient following red blood cell lysis and monocyte depletion, counterflow centrifugal elutriation, leukapheresis, and subsequent cell surface marker-based magnetic or flow cytometric isolation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, cells from the circulating blood of an individual are obtained by leukapheresis.

In some embodiments, cells collected by leukapheresis may be washed to remove the plasma fraction and to place the cells in an appropriate buffer or media for subsequent processing steps. In some embodiments of the invention, the cells are washed with phosphate buffered saline (PBS). In some embodiments, the wash solution lacks calcium and may lack magnesium or may lack many if not all divalent cations. After washing, the cells may be resuspended in any of a variety of biocompatible buffers, such as, for example, Ca²⁺-free, Mg²⁺-free PBS, PlasmaLyte A, or other saline solutions with or without buffer. Alternatively, the undesirable components of the leukapheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells can be further isolated by positive or negative selection techniques. For example, in some embodiments, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In some embodiments, the time period is about 30 minutes. In a further embodiment, the time period ranges from 30 minutes to 36 hours or longer and all integer values therebetween. In a further embodiment, the time period is at least 1, 2, 3, 4, 5, or 6 hours. In some embodiments, the time period is 10 to 24 hours. In certain embodiments, the incubation time period is 24 hours. Longer incubation times may be used to isolate T cells in any situation where there are few T cells as compared to other cell types. Thus, by simply shortening or lengthening the time that T cells are allowed to bind to the anti-CD3/anti-CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention.

In some embodiments, it may be desirable to perform the selection procedure and use the “unselected” cells in the activation and expansion process. “Unselected” cells can also be subjected to further rounds of selection. Enrichment of a T cell population by negative selection can be accomplished with a combination of antibodies directed to surface markers unique to the negatively selected cells. One method is cell sorting and/or selection via negative magnetic immuno-adherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8. In certain embodiments, T regulatory cells are depleted by anti-CD25 conjugated beads or other similar method of selection.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in some embodiments, a concentration of 2 billion cells/mL is used. In some embodiments, a concentration of 1 billion cells/mL is used. In a further embodiment, greater than 100 million cells/mL is used. In a further embodiment, a concentration of cells of 10, 15, 20, 25, 30, 35, 40, 45, or 50 million cells/mL is used. In some embodiments, a concentration of cells of 75, 80, 85, 90, 95, or 100 million cells/mL is used. In further embodiments, concentrations of 125 or 150 million cells/mL can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and may be desirable to obtain.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In certain embodiments, cryopreserved cells are thawed and washed as described herein and allowed to rest for one hour at room temperature prior to activation.

Also contemplated in the context of the invention is the collection of blood samples or leukapheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In some embodiments, a blood sample or a leukapheresis product is taken from a generally healthy subject. In certain embodiments, a blood sample or a leukapheresis product is taken from a generally healthy subject who is at risk of developing a disease, but who has not yet developed a disease, and the cells of interest are isolated and frozen for later use. In certain embodiments, the T cells may be expanded, frozen, and used at a later time.

Whether prior to or after genetic modification of the T cells to express a desirable CAR, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041; and U.S. Patent Publication 2006/0121005, incorporated herein by reference.

Generally, the T cells (e.g., Treg, Teff, memory T cells, NKT or MAIT cells) of the invention are expanded by contact with a surface having attached thereto an agent that stimulates a CD3/TCR complex associated signal and a ligand that stimulates a co-stimulatory molecule on the surface of the cells. In particular, the T cells (e.g., Treg, Teff, memory T cells, NKT or MAIT cells) may be stimulated as described herein, such as by contact with an anti-CD3 antibody or antigen-binding fragment thereof or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of CD4⁺ T cells, an anti-CD3 antibody and an anti-CD28 antibody may be used. Examples of an anti-CD28 antibody include, without being limited to, 9.3, B-T3, XR-CD28 (Diaclone, Besancon, France). Other expansion methods commonly known in the art can be used (Berg et al., Transplant Proc. 30(8):3975-3977 (1998); Haanen et al., J. Exp. Med. 190(9):1319-1328 (1999); Garland et al., J. Immunol Meth. 227(1-2):53-63 (1999)).

In certain embodiments, the primary stimulatory signal and the co-stimulatory signal for the T cells (e.g., Tregs, Teff, memory T, NKT or MAIT cells) of the invention may be provided by different protocols. For example, the agents providing each signal may be in solution and/or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In some embodiments, the agent providing the co-stimulatory signal is bound to a cell surface and the agent providing the primary activation signal is in solution or coupled to a surface. In certain embodiments, both agents can be in solution. In some embodiments, the agents may be in soluble form, and then cross-linked to a surface, such as a cell expressing Fc receptors or an antibody or other binding agent that will bind to the agents. In this regard, see for example, U.S. Patent Publications 2004/0101519 and 2006/0034810, incorporated herein by reference, for artificial antigen presenting cells (aAPCs) that are contemplated for use in activating and expanding T cells in the present invention.

In some embodiments, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the co-stimulatory signal is an anti-CD28 antibody or antigen-binding fragment thereof; and both agents are co-immobilized to the same bead in equivalent molecular amounts. In some embodiments, a 1:1 ratio of each antibody bound to the beads for CD4⁺ T cell expansion and T cell growth is used. In certain aspects of the present invention, a ratio of anti CD3:anti-CD28 antibodies bound to the beads is used such that an increase in T cell expansion is observed as compared to the expansion observed using a ratio of 1:1. In some embodiments, an increase of from about 1 to about 3 fold is observed as compared to the expansion observed using a ratio of 1:1. In some embodiments, the ratio of anti-CD3:anti-CD28 antibody bound to the beads ranges from 100:1 to 1:100 and all integer values therebetween. In some embodiments, more anti-CD28 antibody is bound to the particles than anti-CD3 antibody, i.e., the ratio of CD3:CD28 is less than one. In certain embodiments of the invention, the ratio of anti-CD28 antibody to anti CD3 antibody bound to the beads is greater than 2:1. In one particular embodiment, a 1:100 CD3:CD28 ratio of antibody bound to beads is used. In some embodiments, a 1:75 CD3:CD28 ratio of antibody bound to beads is used. In a further embodiment, a 1:50 CD3:CD28 ratio of antibody bound to beads is used. In some embodiments, a 1:30 CD3:CD28 ratio of antibody bound to beads is used. In certain embodiments, a 1:10 CD3:CD28 ratio of antibody bound to beads is used. In some embodiments, a 1:3 CD3:CD28 ratio of antibody bound to the beads is used. In some embodiments, a 3:1 CD3:CD28 ratio of antibody bound to the beads is used.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads may only bind a few cells, while larger beads may bind many. In certain embodiments, T cells can be stimulated with a ratio of cells to particles ranging from 1:100 to 100:1 and any integer values in between. In particular embodiments, the ratio comprises 1:9 to 9:1 and any integer values in between. The ratio of anti-CD3- and anti-CD28-coupled particles to T cells that result in T cell stimulation can vary as noted above; however certain embodiments of values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one particular ratio being at least 1:1 particles per T cell. In some embodiments, a ratio of particles to cells of 1:1 or less is used. In certain embodiments, the particle: cell ratio is 1:5. In further embodiments, the ratio of particles to cells can be varied depending on the day of stimulation. For example, in some embodiments, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In one particular embodiment, the ratio of particles to cells is 1:1 on the first day of stimulation and adjusted to 1:5 on the third and fifth days of stimulation. In some embodiments, particles are added on a daily or every other day basis to a final ratio of 1:1 on the first day, and 1:5 on the third and fifth days of stimulation. In some embodiments, the ratio of particles to cells is 2:1 on the first day of stimulation and adjusted to 1:10 on the third and fifth days of stimulation. In some embodiments, particles are added on a daily or every other day basis to a final ratio of 1:1 on the first day, and 1:10 on the third and fifth days of stimulation. One of skill in the art will appreciate that a variety of other ratios may be suitable for use in the present invention. In particular, ratios will vary depending on particle size and on cell size and type.

In further embodiments of the present invention, the immune cells are combined with agent-coated beads, the beads and the cells are subsequently separated, and then the cells are cultured. In alternative embodiments, prior to culture, the agent-coated beads and cells are not separated but are cultured together. In some embodiments, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 antibodies are attached (3×28 beads) to contact the immune cells of the invention. In some embodiments, the cells (for example, 10⁴ to 10⁹ T cells) and beads (for example, DYNABEADS® M-450 CD3/CD28 T paramagnetic beads at a ratio of 1:1) are combined in a buffer such as PBS (e.g., without divalent cations such as calcium and magnesium). Again, those of ordinary skill in the art can readily appreciate that any cell concentration may be used depending on the circumstance. For example, the target cell may be very rare in the sample and comprise only 0.01% of the sample or the entire sample (i.e., 100%) may comprise the target cell of interest. Accordingly, any cell number is within the context of the present invention. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/mL is used. In another embodiment, greater than 100 million cells/mL is used. In a further embodiment, a concentration of cells of 10, 15, 20, 25, 30, 35, 40, 45, or 50 million cells/mL is used. In yet another embodiment, a concentration of cells from 75, 80, 85, 90, 95, or 100 million cells/mL is used. In further embodiments, concentrations of 125 or 150 million cells/mL can be used. Using high concentrations may result in increased cell yield, cell activation, and/or cell expansion. Further, use of high cell concentrations allows more efficient capture of cells that may weakly express target antigens of interest, such as CD28-negative T cells. Such populations of cells may have therapeutic value and may be desirable to obtain in certain embodiments.

In some embodiments of the present invention, the mixture may be cultured for several hours (e.g., about 3 hours) to about 14 days or any hourly integer value in between. In some embodiments, the mixture may be cultured for 21 days. In some embodiments of the invention, the beads and the T cells are cultured together for about eight days. In some embodiments, the beads and T cells are cultured together for 2-3 days. Several cycles of stimulation may also be desired such that culture time of the T cells may be 60 days or more. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, a-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

T cells that have been exposed to varied stimulation times may exhibit different characteristics. For example, typical blood or apheresed peripheral blood mononuclear cell products have a helper T cell population (Th, CD4⁺) that is greater than the cytotoxic or suppressor T cell population (Tc, CD8⁺). Ex vivo expansion of T cells by stimulating CD3 and CD28 receptors produces a population of T cells that prior to about days 8-9 consists predominately of Th cells, while after about days 8-9, the population of T cells comprises an increasingly greater population of Tc cells. Depending on the purpose of treatment, in some embodiments, infusing a subject with a T cell population comprising predominately of Th cells may be advantageous. In some embodiments, if an antigen-specific subset of Tc cells has been isolated, it may be beneficial to expand this subset to a greater degree.

Further, in addition to CD4 and CD8 markers, other phenotypic markers vary significantly, but in large part, reproducibly during the course of the cell expansion process. Such reproducibility enables the ability to tailor an activated T cell product for specific purposes.

In some embodiments of the invention, the T cells may be cultured in the presence of rapamycin in order to obtain regulatory T cells, as described for example in PCT Patent Publication WO 2007/110785 (incorporated herein by reference). Another method to generate regulatory T cells is described in U.S. Patent Publication 2016/024470 (incorporated herein by reference), wherein T cells are cultured with a T cell receptor (TCR)/CD3 activator such as for example TCR/CD3 antibodies, a TCR co-stimulator activator such as for example CD28, CD137 (4-1 BB), GITR, B7-½, CD5, ICOS, OX40, CD40 or CD137 antibodies, and rapamycin.

In some embodiments of the invention, T cells genetically modified by expression of a CAR as described herein may also have been genetically modified by expression of at least one intracellular factor such as ROR-C, FoxP3, Foxol, T-bet, or Gata 3, c-Maf, or AhR. In some embodiments, the genetically modified immune cell of the invention expresses FoxP3. In some embodiments, the genetically modified immune cell of the invention expresses Foxol.

In one embodiment, the genetically modified immune cell of the invention can be an allogeneic Treg, Teff, memory T cell, or NKT or MAIT cell. For example, the allogeneic T cell can be a T cell lacking expression of a functional human leukocyte antigen (HLA), e.g., HLA class I and/or HLA class II.

In one embodiment, the T cells as described herein can be engineered such that they do not express a functional HLA on its surface. For example, a T cell as described herein can be engineered such that cell surface expression HLA, e.g., HLA class 1 and/or HLA class II or non-classical HLA molecules is downregulated.

Modified immune cells that lack expression of a functional TCR and/or HLA can be obtained by any suitable means, including a knock out or knock down of one or more subunit of TCR and/or HLA. For example, the T cell can include a knock down of TCR and/or HLA using siRNA, shRNA, clustered regularly interspaced short palindromic repeats (CRISPR) transcription-activator like effector nuclease (TALEN), zinc finger endonuclease (ZFN), meganuclease (mn, also known as homing endonuclease), or megaTAL (combining a TAL effector with a mn cleavage domain).

In some embodiments, the nucleic acid encoding a CAR as described herein is inserted at a specific locus in the genome of an immune cell, such as, for example, at the locus of a gene to be deleted. In some embodiments, the nucleic acid encoding a CAR as described herein is inserted within a TCR and/or HLA locus, thereby resulting in the inhibition of TCR and/or HLA expression.

In some embodiments, TCR and/or HLA expression can be inhibited using siRNAs or shRNAs that targets a nucleic acid encoding a TCR and/or HLA in a T cell. Expression of siRNAs and shRNAs in T cells can be achieved using any conventional expression system, e.g., such as a lentiviral expression system. Exemplary siRNAs and shRNAs that downregulate expression of HLA class I and/or HLA class II genes are described, e.g., in U.S. Patent Publication 2007/0036773. Exemplary shRNAs that downregulate expression of components of the TCR are described, e.g., in U.S. Patent Publication 2012/0321667.

“CRISPR” or “CRISPR to TCR and/or HLA” or “CRISPR to inhibit TCR and/or HLA” as used herein refers to a set of clustered regularly interspaced short palindromic repeats, or a system comprising such a set of repeats. “Cas,” as used herein, refers to a CRISPR-associated protein. A “CRISPR/Cas” system refers to a system derived from CRISPR and Cas that can be used to silence or mutate a TCR and/or HLA gene.

Naturally-occurring CRISPR/Cas systems are found in approximately 40% of sequenced eubacteria genomes and 90% of sequenced archaea. See, e.g., Grissa et al. (BMC Bioinformatics 8:172 (2007)). This system is a type of prokaryotic immune system that confers resistance to foreign genetic elements such as plasmids and phages and provides a form of acquired immunity. See, e.g., Barrangou et al., Science 315:1709-1712 (2007); Marragini et al., Science 322:1843-1845 (2008). The CRISPR/Cas system has been modified for use in gene editing (silencing, enhancing or changing specific genes) in eukaryotes such as mice or primates. See, e.g., Wiedenheft et al., Nature 482: 331-8 (2012). This is accomplished by introducing into the eukaryotic cell a plasmid containing a specifically designed CRISPR and one or more appropriate Cas-encoding sequences. The CRISPR sequence, sometimes called a CRISPR locus, comprises alternating repeats and spacers. In naturally-occurring CRISPR, the spacers usually comprise sequences foreign to the bacterium such as a plasmid or phage sequence; in the TCR and/or HLA CRISPR/Cas system, the spacers are derived from the TCR and/or HLA gene sequence. RNA from the CRISPR locus is constitutively expressed and processed by Cas proteins into small RNAs. These comprise a spacer flanked by a repeat sequence. The RNAs guide other Cas proteins to silence exogenous genetic elements at the RNA or DNA level. See, e.g., Horvath et al., Science 327:167-170 (2010); Makarova et al., Biology Direct 1:7 (2006). The spacers thus serve as templates for RNA molecules, analogously to siRNAs. See, e.g., Pennisi, Science 341:833-836 (2013). The CRISPR/Cas system thus can be used to edit a TCR and/or HLA gene (adding or deleting a base pair), or introduce a premature stop that decreases expression of a TCR and/or HLA. The CRISPR/Cas system alternatively or additionally can be used like RNA interference, turning off the TCR and/or HLA gene in a reversible fashion. In a mammalian cell, for example, the RNA can guide the Cas protein to a TCR and/or HLA promoter, sterically blocking RNA polymerases.

Artificial CRISPR/Cas systems can be generated that inhibit TCR and/or HLA, using technology known in the art, e.g., that described in U.S. Patent Publication 2014/0068797, and Cong, Science 339:819-823 (2013). Other artificial CRISPR/Cas systems that are known in the art may also be generated to inhibit TCR and/or HLA, e.g., those described in Tsai, Nature Biotechnol. 32:6569-576 (2014) and U.S. Pat. Nos. 8,871,445; 8,865,406; 8,795,965; 8,771,945; and U.S. Pat. No. 8,697,359.

“TALEN” or “TALEN to TCR and/or HLA” or “TALEN to inhibit TCR and/or HLA” refers to a transcription activator-like effector nuclease, an artificial nuclease that can be used to edit the TCR and/or HLA gene. TALENs are produced artificially by fusing a TAL effector DNA binding domain to a DNA cleavage domain. Transcription activator-like effectors (TALEs) can be engineered to bind any desired DNA sequence, including a portion of the TCR and/or HLA gene. By combining an engineered TALE with a DNA cleavage domain, a restriction enzyme can be produced that is specific to any desired DNA sequence, including a TCR and/or HLA sequence. These can then be introduced into a cell, wherein they can be used for genome editing. See, e.g., Boch, Nature Biotech. 29:135-6 (2011); Boch et al., Science 326:1509-12 (2009); and Moscou et al., Science 326:3501 (2009).

TALEs are proteins secreted by Xanthomonas bacteria. The DNA binding domain contains a repeated 33-34 amino acid sequence that is highly conserved with the exception of the 12th and 13th amino acids. These two positions are highly variable, showing a strong correlation with specific nucleotide recognition. They can thus be engineered to bind to a desired DNA sequence. To produce a TALEN, a TALE protein is fused to a nuclease (N), which is a wild-type or mutated Fokl endonuclease. Several mutations to Fokl have been made for its use in TALENs; these, for example, improve cleavage specificity or activity. See, e.g., Cermak et al., Nucl. Acids Res. 39:e82 (2011); Miller et al., Nature Biotech. 29:143-8 (2011); Hockemeyer et al., Nature Biotech. 29:731-734 (2011); Wood et al., Science 333:307 (2011); Doyon et al., Nature Methods 8:74-79 (2010); Szczepek et al., Nature Biotech. 25:786-793 (2007); and Guo et al., J. Mol. Biol. 200:96 (2010). The Fokl domain functions as a dimer, requiring two constructs with unique DNA binding domains for sites in the target genome with proper orientation and spacing. Both the number of amino acid residues between the TALE DNA binding domain and the Fokl cleavage domain and the number of bases between the two individual TALEN binding sites appear to be important parameters for achieving high levels of activity (Miller et al., Nature Biotech. 29:143-8 (2011)). A TCR and/or HLA TALEN can be used inside a cell to produce a double-stranded break (DSB). A mutation can be introduced at the break site if the repair mechanisms improperly repair the break via non-homologous end joining. For example, improper repair may introduce a frame shift mutation. Alternatively, foreign DNA can be introduced into the cell along with the TALEN; depending on the sequences of the foreign DNA and chromosomal sequence, this process can be used to correct a defect in the TCR and/or HLA gene or introduce such a defect into a wt HLA gene, thus decreasing expression of TCR and/or HLA. TALENs specific to sequences in TCR and/or HLA can be constructed using any method known in the art, including various schemes using modular components (Zhang et al., Nature Biotech. 29:149-53 (2011); Geibler et al., PLoS ONE 6:e19509 (2011)).

“ZFN” or “Zinc Finger Nuclease” or “ZFN to TCR and/or HLA” or “ZFN to inhibit TCR and/or HLA” refers to a zinc finger nuclease, an artificial nuclease that can be used to edit the TCR and/or HLA gene. Like a TALEN, a ZFN comprises a Fokl nuclease domain (or derivative thereof) fused to a DNA-binding domain. In the case of a ZFN, the DNA-binding domain comprises one or more zinc fingers. See, e.g., Carroll et al., Genetics Society of America 188:773-782 (2011); and Kim et al., Proc. Natl. Acad. Sci. USA 93:1156-1160 (1996). A zinc finger is a small protein structural motif stabilized by one or more zinc ions. A zinc finger can comprise, for example, Cys₂His₂, and can recognize an approximately 3 bp sequence. Various zinc fingers of known specificity can be combined to produce multi-finger polypeptides that recognize about 6, 9, 12, 15 or 18 bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells.

Like a TALEN, a ZFN must dimerize to cleave DNA. Thus, a pair of ZFNs is required to target non-palindromic DNA sites. The two individual ZFNs must bind opposite strands of the DNA with their nucleases properly spaced apart (Bitinaite et al., Proc. Natl. Acad. Sci. USA 95:10570-5 (1998)). Also like a TALEN, a ZFN can create a double-stranded break in the DNA, which can create a frame-shift mutation if improperly repaired, leading to a decrease in the expression and amount of TCR and/or HLA in a cell. ZFNs can also be used with homologous recombination to mutate the TCR and/or HLA gene. ZFNs specific to sequences in TCR and/or HLA can be constructed using any method known in the art. See, e.g., Provasi, Nature Med. 18:807-815 (2011); Torikai, Blood 122:1341-1349 (2013); Cathomen et al., Mol. Ther. 16:1200-7 (2008); Quo et al., J. Mol. Biol. 400:96 (2010); and U.S. Patent Publications 2011/0158957 and 2012/0060230.

“Meganuclease” or “meganuclease to TCR and/or HLA” or “meganuclease to inhibit TCR and/or HLA” refers to a monomeric endonuclease with large (>14 base pairs) recognition sites, which can be used to edit the TCR and/or HLA gene. Meganucleases (mn) are monomeric proteins with innate nuclease activity that are derived from bacterial homing endonucleases and engineered for a unique target site. Homing endonucleases are DNA-cleaving enzymes that can generate double strand breaks at individual loci in their host genomes, and thereby drive site-specific gene conversion events (Stoddard, Structure 19(1):7-15 (2011)). Despite their small size, homing endonucleases recognize long DNA sequences (typically 20 to 30 base pairs). Homing endonucleases are extremely widespread and are found in microbes, as well as in phages and viruses. The LAGLIDADG and His-Cys box enzymes (which are the most sequence-specific of these enzymes) rely upon antiparallel β-sheets that dock into the major grooves of their DNA target sites (Pick et al., Nature 394(6688):96-101 (1998); Jurica et al., Mol. Cell. 2(4):469-76 (1998). There they establish a collection of sequence-specific and non-specific contacts that are distributed nonuniformly across multiple consecutive basepairs (Chevalier et al., J Mol Biol 329(2):253-269 (2003); Scalley-Kim et al., J Mol Biol. 372(5):1305-19 (2007).

The LAGLIDADG homing endonuclease (LHE) family is the primary source of the engineered enzymes used for gene targeting applications. The LHE family is primarily encoded within archaea and in the chloroplast and mitochondrial genomes of algae and fungi (Chevalier et al., in Homing Endonucleases and Inteins. Nucleic Acids and Molecular Biology, vol. 16 (2005); Dalgaard et al., Nucleic Acids Res. 25(22):4626-38 (1997); Sethuraman et al., Mol Biol Evol. 26(10):2299-315 (2009). Meganucleases that possess a single conserved LAGLIDADG motif (SEQ ID NO: 174) per protein chain form homodimeric proteins that cleave palindromic and nearly palindromic DNA target sequences, while those that contain two such motifs per protein chain form larger, pseudo-symmetric monomers that can target completely asymmetric DNA sequences.

Meganucleases can be engineered to target TCR and/or HLA and thus create a double-stranded break in the DNA, which can create a frame-shift mutation if improperly repaired, leading to a decrease in the expression and amount of TCR and/or HLA in a cell.

“MegaTAL” or “megaTAL to TCR and/or HLA” or “megaTAL to inhibit TCR and/or HLA” refers to an artificial nuclease, which can be used to edit the TCR and/or HLA gene. MegaTALs are hybrid monomeric nucleases obtained through the fusion of minimal TAL (transcription activator-like) effector domains to the N-terminus of meganucleases derived from the LAGLIDADG homing endonuclease family (Boissel et al., Nucleic Acids Res. 42(4):2591-601 (2014); Takeuchi et al, Methods Mol Biol. 1239:105-32 (2015)). MegaTALs thus consist of a site-specific meganuclease cleavage head with additional affinity and specificity provided by a TAL effector DNA binding domain.

MegaTALs can be engineered to target TCR and/or HLA and thus create a double-stranded break in the DNA, which can create a frame-shift mutation if improperly repaired, leading to a decrease in the expression and amount of TCR and/or HLA in a cell.

In some embodiments, transfection with a telomerase gene can lengthen the telomeres of a T cell and improve persistence of the T cell in the patient. See, e.g., June, Journal of Clinical Investigation 117: 1466-1476 (2007). Thus, in some embodiments, a genetically modified immune cell of the invention ectopically expresses a telomerase subunit, e.g., the catalytic subunit of telomerase, e.g., TERT, e.g., hTERT. In some aspects, this disclosure provides a method of producing a chimeric receptor-expressing cell of the invention, comprising contacting the cell with a nucleic acid encoding a telomerase subunit, e.g., the catalytic subunit of telomerase, e.g., TERT, e.g., hTERT. The cell may be contacted with the nucleic acid before, simultaneous with, or after being contacted with a construct encoding the chimeric receptor (e.g., a CAR as described herein).

The present invention further relates to a method for obtaining an immune cell of the invention, wherein said method comprises transducing at least one immune cell with a nucleic acid encoding a CAR as described herein, and optionally expanding the transduced cells. In some embodiments, the method is an ex vivo method.

In one embodiment, the method for obtaining immune cells of the invention comprises:
an isolation step of immune cells from a PBMC population (e.g., recovered by leukapheresis)
a genetic modification step wherein a nucleic acid sequence encoding a CAR as described hereinabove is introduced or transferred within the immune cells,
optionally an expansion step,
optionally a washing step and,
optionally a freezing step.

In one embodiment, the genetic modification step(s) correspond(s) to a gene disruption step, a gene correction step or a gene addition step, preferably a gene addition step. In one embodiment, the genetic modification step(s) is carried out by a method selected from the group comprising, but not limited to, transfection, transduction or gene editing.

Examples of methods of gene editing that may be used in the present invention include, but are not limited to, methods based on engineered nucleases, methods based on recombinant Adeno-Associated Virus (or AAV), methods based on Transposons (e.g., Sleeping Beauty transposon system), methods based on homologous recombination, conditional targeting using site-specific recombinases (e.g., Cre-LoxP and Flp-FRT systems), and Multiplex Automated Genomic Engineering (MAGE).

Non-limiting examples of engineered nucleases include, but are not limited to, clustered regularly interspaced short palindromic repeats (CRISPR) transcription-activator like effector nuclease (TALEN), zinc finger endonuclease (ZFN), meganuclease (mn, also known as homing endonuclease), or megaTAL (combining a TAL effector with a mn cleavage domain).

In one embodiment, the method for obtaining immune cells of the invention comprises:
an isolation step of immune cells from a PBMC population (e.g., recovered by leukapheresis)
a transduction or transfection step with a vector comprising a nucleic acid sequence encoding a CAR as described hereinabove,
optionally an expansion step,
optionally a washing step and,
optionally a freezing step.

The present invention further relates to an immune cell expressing a CAR as described herein, and to a population of such immune cells.

In some embodiments, a nucleic acid encoding a CAR of the present invention is introduced into an immune cell, thereby generating an engineered cell expressing the CAR on the cell surface. Thus, present invention also relates to nucleic acid encoding a CAR of the present invention.

In some embodiments, an immune cell of the invention is a mammalian immune cell, e.g., a human immune cell, an immune cell from a farm animal (e.g., a cow, pig, or horse), or an immune cell from a pet (e.g., a cat or a dog).

In some embodiments, the immune cell is selected from the group consisting of lymphocytes, myeloid-derived cells, and any combination thereof. In certain embodiments, the immune cell is a lymphocyte, e.g., selected from the group consisting of T cells, B cells, natural killer (NK) cells, and any combination thereof. In particular embodiments, the immune cell is a T cell, which in certain embodiments is selected from the group consisting of CD4⁺ T cells, CD8⁺ T cells, γδ T cells, double negative (DN) T cells, and any combination thereof. In certain embodiments, the immune cell is a CD4⁺ T cell, such as, for example, a T helper cell, a regulatory T cell, an effector T cell, and any combination thereof. In some embodiments, the immune cell is a CD8⁺ T cell, such as, for example, a cytotoxic CD8⁺ T cell or a CD8⁺ regulatory T cell. In some embodiments, the immune cell is a γδ T cell. In some embodiments the immune cell is a T cell engineered to express a defined Gamma delta TCR (TEGγδ) cells. In some embodiments, the immune cell is a DN T cell. In some embodiments, the immune cell is a NK cell.

In some embodiments, the immune cell is a regulatory immune cell, such as, for example, any regulatory immune cell suitable for use in cellular therapy. In certain embodiments, the regulatory immune cell is selected from the group consisting of a regulatory T cell, a CD4⁺ regulatory T cell, a CD8⁺ regulatory T cell, a regulatory γδ T cell, a regulatory DN T cell, a regulatory B cell, a regulatory NK cell, a regulatory macrophage, a regulatory dendritic cell, and any combination thereof.

In some embodiments, the regulatory immune cell is a regulatory T cell (Treg), in particular, a thymus derived Treg or an adaptive or induced Treg. In certain embodiments, the immune cell is a CD4⁺ regulatory T cell (Treg). In certain embodiments, the Treg is a thymus derived Treg or an adaptive or induced Treg. In certain embodiments, the Treg is a CD4⁺FoxP3⁺ regulatory T cell or a CD4⁺FoxP3⁻ regulatory T cell (Tr1 cell). In particular embodiments, the immune cell is a CD4⁺FoxP3⁺ regulatory T cell.

In some embodiments, the immune cell is a CD8⁺ regulatory T cell. Examples of CD8⁺regulatory T cells include, but are not limited to, a CD8⁺ CD28⁻ regulatory T cell, a CD8⁺ CD103⁺ regulatory T cell, a CD8⁺FoxP3⁺ regulatory T cell, a CD8⁺ CD122⁺regulatory T cell, and any combination thereof.

In some embodiments, the regulatory immune cell is a regulatory γδ T cell.

In some embodiments, the regulatory immune cell is a regulatory DN T cell.

In some embodiments, the regulatory immune cell is a regulatory NK cell.

In some embodiments, the immune cell is an effector immune cell, such as, for example, any effector immune cell suitable for use in cellular therapy. In certain embodiments, the effector immune cell is selected from the group consisting of an effector T cell, a CD4⁺effector T cell, a CD8⁺effector T cell, an effector γδ T cell, an effector DN T cell, an effector NK cell, and any combination thereof.

In some embodiments, the immune cell is an effector T cell. In certain embodiments, the effector immune cell is a CD4⁺effector T cell. Examples of CD4⁺effector T cells include, but are not limited to, Th1 cells, Th2 cells, Th9 cells, Th17 cells, Th22 cells, CD4⁺ T follicular helper (Tfh) cells, and any combination thereof. In some embodiments, the effector immune cell is a CD8⁺effector T cell. Examples of CD8⁺effector T cells include, but are not limited to, a CD8⁺ CD45RO⁺CCR7⁻ CD62L⁻ effector T cell, a CD8⁺ CD45RA⁺CCR7⁻ CD62L⁻ effector T cell, and any combination thereof.

In one embodiment, the immune cell is an effector γδ T cell.

In one embodiment, the immune cell is an effector DN T cell.

In some embodiments, the immune cell is an effector NK cell.

In some embodiments, the immune cell is selected from the group consisting of T cells, natural killer (NK) cells, γδ T cells, double negative (DN) cells, regulatory immune cells, regulatory T cells, effector immune cells, effector T cells, and any combination thereof.

In some embodiments, a nucleic acid encoding a CAR of the present invention is introduced into a non-Treg lymphoid cell that is differentiated into a Treg cell after genome editing. The edited non-Treg cells may be differentiated into Treg cells before engrafting into a patient as described above. Alternatively, the edited non-Treg cells may be induced to differentiate into Treg cells after engrafting into a patient.

In some embodiments, the expression level of molecules is determined by flow cytometry, immunofluorescence or image analysis, for example high content analysis. In certain embodiments, the expression level of molecules is determined by flow cytometry.

In particular embodiments, before conducting flow cytometry analysis, cells are fixed and permeabilized, thereby allowing detection of intracellular proteins.

In some embodiments, determining the expression level of a molecule in a cell population comprises determining the percentage of cells of the cell population expressing the molecule (i.e., cells “+” for the molecule). In certain embodiments, said percentage of cells expressing the molecule is measured by FACS.

The terms “expressing,” “positive,” or “+” and “not expressing,” “negative,” or -” are well known in the art and refer to the expression level of the cell marker of interest, in that the expression level of the cell marker corresponding to “+” is high or intermediate (also referred to as “+/−”), and the expression level of the cell marker corresponding to “−” is null.

The term “low” or “lo” or “lo/−” is well known in the art and refers to the expression level of the cell marker of interest, in that the expression level of the cell marker is low in comparison with the expression level of that cell marker in the population of cells being analyzed as a whole. More particularly, the term “lo” refers to a distinct population of cells that express the cell marker at a lower level than one or more other distinct populations of cells.

The term “high” or “hi” or “bright” is well known in the art and refers to the expression level of the cell marker of interest, in that the expression level of the cell marker is high in comparison with the expression level of that cell marker in the population of cells being analyzed as a whole.

Generally, cells in the top 2, 3, 4, or 5% of staining intensity are designated “hi,” with those falling in the top half of the population categorized as being “+”. Those cells falling below 50%, of fluorescence intensity are designated as “lo” cells and below 5% as “−” cells.

The expression level of the cell marker of interest is determined by comparing the Median Fluorescence Intensity or Mean Fluorescence Intensity (MFI) of the cells from the cell population stained with fluorescently labeled antibody specific for this marker to the fluorescence intensity (FI) of cells from the same cell population stained with fluorescently labeled antibody with an irrelevant specificity but with the same isotype, the same fluorescent probe and originated from the same species (referred to as isotype control). The cells from the population stained with fluorescently labeled antibody specific for this marker and that show equivalent MFI or a lower MFI than the cells stained with the isotype control are considered as not expressing this marker and are designated (−) or negative. The cells from the population stained with fluorescently labeled antibody specific for this marker and that show a MFI value superior to the cells stained with the isotype control are considered as expressing this marker and are designated (+) or positive.

The invention also relates to an isolated and/or substantially purified population of immune cells as defined herein.

Thus, the invention provides an isolated and/or substantially purified population of immune cells, wherein the cells of the population comprise a CAR as described herein.

As used herein, an “isolated population” refers to a cell population that is removed from its natural environment (such as the peripheral blood) and that is isolated, purified or separated, and is at least about 75% free, 80% free, 85% free, and in certain embodiments about 90%, 95%, 96%, 97%, 98%, 99% free, from other cells with which it is naturally present, but which lack the cell surface markers based on which the cells were isolated.

The present invention further relates to an enriched population of immune cells as defined herein.

In some embodiments, the isolated, purified and/or enriched immune cell population of the invention has been frozen and thawed.

In some embodiments, regulatory immune cells of the invention may be selected form the group consisting of CD4⁺ CD25⁺FoxP3⁺ Treg, Tr1 cells, TGF-β secreting Th3 cells, regulatory NK T cells, regulatory γδ T cells, regulatory CD8⁺ T cells, and double negative regulatory T cells.

In some embodiments, the immune cells are autologous cells.

In some embodiments, the immune cells are heterologous cells.

In some embodiments, the immune cells are allogenic cells.

In some embodiments, an immune cell population of the invention expresses at its cell surface a CAR of the invention (herein referred to as “first receptor”), and another receptor (herein referred to as “second receptor”) that binds to another, distinct ligand than human CD45RC. In certain embodiments, the second receptor comprises an extracellular ligand binding domain, optionally a hinge domain, at least one transmembrane domain, and at least one intracellular signaling domain, e.g., as described herein.

In some embodiments, the second receptor is endogenous (such as, for example, the endogenous TCR). In some embodiments, the second receptor is exogenous, and its expression is induced in the immune cell population of the invention by transformation or transduction of a nucleic acid encoding it Said exogenous receptor may be an exogenous TCR or a CAR. Therefore, in some embodiments, the immune cell population of the invention expresses two CARs, wherein the first one recognizes human CD45RC and the second one recognizes a distinct ligand. In some embodiments, the immune cell population of the invention expresses two CARs, wherein the first one recognizes a first epitope on human CD45RC, and the second one recognizes a second, distinct epitope on human CD45RC. In some embodiments, the immune cell population of the invention expresses two CARs, wherein the first one recognizes human CD45RC, and the second one recognizes a second, distinct antigen (such as, for example, an antigen variant of human CD45RC).

In some embodiments, at least one of the CAR of the invention and the second receptor (e.g., a second CAR) is inducible, i.e., its expression on the cell surface may be induced.

In some embodiments, the expression of at least one of the CAR of the invention and the second receptor (e.g., second CAR) is induced by the activation of the other receptor. In certain embodiments, the expression of the CAR of the invention is induced by the activation of the second receptor. In certain embodiments, the expression of the second receptor is induced by the activation of the CAR of the invention. Inducible CARs have been described in the art, such as, for example, by Roybal et al (Cell, 2006).

In one embodiment, the CAR of the invention comprises a first intracellular signaling domain, and the second receptor comprises a distinct second intracellular signaling domain. In a first embodiment, the CAR of the invention comprises a T cell primary signaling domain (such as, for example, CD3 zeta), and the second receptor comprises a costimulatory signaling domain (such as, for example, of 4-1BB, CD28 or a combination of costimulatory signaling domain of 4-1BB and CD28). In a second embodiment, the CAR of the invention comprises a costimulatory signaling domain (such as, for example, of 4-1BB, CD28 or a combination of costimulatory signaling domain of 4-1BB and CD28), and the second receptor comprises a T cell primary signaling domain (such as, for example, CD3 zeta).

Consequently, according to these embodiments, the complete activation of the immune cell population of the invention requires both the binding of the CAR of the invention to human CD45RC, and the binding of the second receptor to the ligand to which it is directed.

In one embodiment, the ligand recognized by the second receptor is expressed or present at the diseased tissue or organ, or at the site of the autoimmune response. Consequently, suppressive activity for cells expressing human CD45RC will be induced only at the diseased tissue or organ or at the site of the autoimmune response, when said ligand will be present and recognized by the second receptor on the cells of immune cell population.

In one embodiment, the chimeric receptor of the invention further comprises an extracellular ligand binding domain recognizing a ligand distinct from human CD45RC recognized by the chimeric receptor. In one embodiment, said ligand binding domain is an antibody or an antigen binding fragment thereof.

In one embodiment, the chimeric receptor of the invention comprises an extracellular ligand binding domain comprising a human CD45RC binding domain and another ligand binding domain recognizing a ligand distinct from said human CD45RC. In one embodiment, said ligand binding domain is a bifunctional antibody recognizing both the human CD45RC and the distinct ligand.

A further object of the present invention is a composition comprising the isolated T-cell population expressing the CAR, binding to hCD45RC according to the present invention.

A further object of the present invention is a composition comprising at least one nucleic acid encoding the isolated T-cell population expressing the CAR binding to hCD45RC according to the present invention.

A further object of the present invention is a composition comprising at least one expression vector comprising at least one nucleic acid encoding the isolated T-cell population expressing the CAR binding to hCD45RC according to the present invention.

A further object of the present invention is a pharmaceutical composition comprising the isolated T-cell population expressing the CAR, binding to hCD45RC according to the present invention; and at least one pharmaceutically acceptable excipient.

A further object of the present invention is a pharmaceutical composition comprising at least one nucleic acid encoding the isolated T-cell population expressing the CAR binding to hCD45RC according to the present invention; and at least one pharmaceutically acceptable excipient.

A further object of the present invention is a pharmaceutical composition comprising at least one expression vector comprising at least one nucleic acid encoding the isolated T-cell population expressing the CAR binding to hCD45RC according to the present invention; and at least one pharmaceutically acceptable excipient or vehicle.

The term “pharmaceutically acceptable excipient” includes any and all solvents, dispersion media, coatings, antibacterial and antifungal agents, isotonic and absorption delaying agents and the like. Said excipient does not produce an adverse, allergic or other untoward reaction when administered to an animal, preferably a human. For human administration, preparations should meet sterility, pyrogenicity, and general safety and purity standards as required by regulatory offices, such as, for example, FDA Office or EMA.

Pharmaceutically acceptable excipients that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene- block polymers, polyethylene glycol and wool fat.

In one embodiment, the pharmaceutical compositions according to the present invention comprise vehicles which are pharmaceutically acceptable for a formulation capable of being injected to a subject. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

A further object of the present invention is a medicament comprising the isolated T-cell population expressing the CAR, binding to hCD45RC according to the present invention. A further object of the present invention is a medicament comprising at least one nucleic acid encoding the isolated T-cell population expressing the CAR binding to hCD45RC according to the present invention.

A further object of the present invention is a medicament comprising at least one expression vector comprising at least one nucleic acid encoding the isolated T-cell population expressing the CAR binding to hCD45RC according to the present invention.

The present invention further relates to methods of inducing immune tolerance in a subject in need thereof, by administering the isolated T-cell population expressing the CAR, composition, or pharmaceutical composition according to the present invention.

It also relates to the T-cell population expressing the CAR, composition, or pharmaceutical composition according to the present invention, for use in inducing immune tolerance in a subject in need thereof.

The term “immune tolerance”, as used herein, relates to a state of unresponsiveness of the immune system to specific substances or tissues that have the capacity to elicit an immune response while preserving immune response against other substances or tissues.

The term “immune response”, as used herein, includes T cell-mediated and/or B cell-mediated immune responses. Exemplary immune responses include, but are not limited to, T cell responses (e.g., cytokine production and cellular cytotoxicity), but also immune responses that are indirectly affected by T cell activation (e.g., macrophages). Immune cells involved in the immune response include lymphocytes (such as B cells and T cells, including CD4⁺, CD8⁺, Th1 and Th2 cells), antigen presenting cells (e.g., professional antigen presenting cells such as dendritic cells), natural killer cells, myeloid cells (such as macrophages, eosinophils, mast cells, basophils, and granulocytes).

The present invention further relates to methods of depleting CD45RC^{high} cells in a subject in need thereof, by administering the isolated T-cell population expressing the CAR, composition, pharmaceutical composition according to the present invention. It also relates to the isolated T-cell population expressing the CAR, composition, pharmaceutical composition according to the present invention, for use in depleting CD45RC^{high} cells in a subject in need thereof.

The relative level of expression of hCD45RC is measured using cytometry. Three types of cells can be distinguished: cells presenting a high, intermediary or negative level of hCD45RC expression.

In one embodiment, the isolated T-cell population expressing the CAR, composition, pharmaceutical composition according to the present invention depletes CD45RC^{high}T cells.

“CD45R^{high} Cell Antigen” or “CD45RC^{high} Cell Surface Marker”

As used herein, the terms “CD45RC^{high} cell antigen” or “CD45RC^{high} cell surface marker” refer to an antigen (or epitope) of sequence SEQ ID NO: 23, which is expressed or displayed at the surface of a CD45RC^{high} cells (including T cells, B cells and natural killer (NK) cells) which can be targeted with an anti-CD45RC agent which binds thereto (such as an antibody or an aptamer). Exemplary CD45RC^{high} T cell surface markers include but are not limited to the CD45RC as previously described or other antigens that characterize said population of T cells. The CD45RC^{high} T cells surface marker of particular interest is preferentially expressed on CD45RC^{high} T cells compared to other non-CD45RC^{high}T cells of a mammal.

Then, after raising antibodies directed against the CD45RC cell surface marker as above described, the skilled man in the art can easily select those that act on CD45RC^{high} cells, and that can be used to deplete CD45RC^{high} cells via antibody-dependent cell mediated cytotoxicity (ADCC), complement dependent cytotoxicity (CDC), or induction of CD45RC^{high} but not CD45RC^{low/-} cell death (e.g., via apoptosis) after direct binding of the antibody (Picarda et al., 2017. JCI Insight. 2(3):e90088).

“CD45RC^{high} T cells” are T cells that express the CD45RC marker at a high level, as defined above. It is readily understood by the one skilled in the art that the isolated T-cell population expressing the CAR, composition, pharmaceutical composition according to the present invention that depletes CD45RC^{high} T cells may also be able to deplete other types of CD45RC^{high} cells, such as CD45RC^{high} NK cells or CD45RC^{high} B cells.

As used herein, the terms “deplete” or “depleting”, with respect to cells expressing CD45RC, refer to a measurable decrease in the number of cells in the subject. The reduction can be at least about 10%, e.g., at least about 20%, 30%, 40%, 50%, 60%, 70%, 80%, 90%, 95%, 96%, 97%, 98%, 99%, or more. In some embodiments, the terms refer to a decrease in the number of CD45RC^{high} cells in a subject or in a sample to an amount below detectable limits. According to the present invention, the isolated T-cell population expressing the CAR, composition, pharmaceutical composition specifically mediates depletion of the effector cells strongly expressing CD45RC, in particular those designed as CD45RC^{high} Teff.

In particular, said isolated T-cell population expressing the CAR, composition, pharmaceutical composition according to the present invention depletes CD45RC^{high} T cells by binding to hCD45RC and transducing pro-apoptotic signals and/or by activating antibody-dependent cell mediated cytotoxicity (ADCC), complement dependent cytotoxicity (CDC) or antibody dependent phagocytosis.

In some embodiments, the isolated T-cell population expressing the CAR, composition, pharmaceutical composition according to the present invention mediates complement dependent cytotoxicity.

In a particular embodiment, the isolated CAR according to the present invention may be conjugated to a cytotoxic agent or a growth inhibitory agent.

The present invention further relates to methods of expanding and/or potentiating regulatory T cells in a subject in need thereof, by administering the isolated T-cell population expressing the CAR, composition, pharmaceutical composition according to the present invention. It also relates to the isolated T-cell population expressing the CAR, composition, pharmaceutical composition according to the present invention, for use in expanding and/or potentiating regulatory T cells in a subject in need thereof.

As used herein, the term “expand” refers to the process of converting and/or amplifying a given population of cells (e.g., immune cells such as Tregs). Expansion of a population of cells can occur in vivo, in vitro or ex vivo.

As used herein, the term “potentiate” refers to the process of increasing the function of a given population of cells (e.g., increasing the suppressive capacity of Tregs cells). Potentiation of a population of cells can occur in vivo, in vitro or ex vivo.

“Regulatory T cells” or “Tregs” are T cells that suppress an abnormal or excessive immune response and play a role in immune tolerance. Tregs are typically “forkhead box P3 (Foxp3⁺) regulatory T cells” and/or “CD45RC^{low/-} cells”.

As used herein, the terms “forkhead box P3 (Foxp3⁺) regulatory T cells” and “CD45RC^{low/-} cells” refer to 0.1-10% of CD4⁺ and/or CD8⁺ T cells in humans and rodents whose characteristic marker is the transcription factor Foxp3.

In one embodiment, the methods and uses are for expanding and/or potentiating Foxp3⁺ and/or CD45RC^{low/-} Tregs.

In one embodiment, CD45RC^{low/-} Tregs are expanded by stimulation. In one embodiment, CD45RC^{low/-} Tregs are expanded by stimulation in the presence of IL-2 and IL-15. In one embodiment, CD45RC^{low/-} Tregs are expanded by stimulation with anti-CD3/anti-CD28 antibodies and/or allogeneic antigen-presenting cells (APCs) and/or specific antigens.

Additionally or alternatively, the invention relates to in vitro or ex vivo methods of purifying CD45RC^{low/-} Tregs.

In one embodiment, CD45RC^{low/-} Tregs are CD8⁺/CD4⁺ T cells. In one embodiment, CD45RC^{low/-} Tregs are CD8⁺/CD4⁻ T cells. In one embodiment, CD45RC^{low/-} Tregs are CD8⁻/CD4⁺ T cells.

In one embodiment, purified CD45RC^{low/-} Tregs can be further expanded and/or potentiated prior to, concomitantly with or after administration to a subject in need thereof.

The present invention further relates to methods of preventing and/or reducing transplant rejections, by administering to a subject in need thereof the isolated T-cell population expressing the CAR, composition, pharmaceutical composition according to the present invention. It also relates to the isolated T-cell population expressing the CAR, composition, pharmaceutical composition according to the present invention, for use in preventing and/or reducing transplant rejections in a subject in need thereof.

The terms “preventing transplant rejections” and “reducing transplant rejections” are meant to encompass prevention or inhibition of immune transplant rejection, as well as delaying the onset or the progression of immune transplant rejection. The terms are also meant to encompass prolonging survival of a transplant in a subject, or reversing failure of a transplant in a subject. Further, the terms are meant to encompass ameliorating a symptom of an immune transplant rejection, including, for example, ameliorating an immunological complication associated with immune rejection, such as, e.g., interstitial fibrosis, chronic graft arteriosclerosis, or vasculitis.

The term “transplantation” and variations thereof refer to the insertion of a transplant (also called graft) into a recipient, whether the transplantation is syngeneic (where the donor and recipient are genetically identical), allogeneic (where the donor and recipient are of different genetic origins but of the same species), or xenogeneic (where the donor and recipient are from different species). Thus, in a typical scenario, the host is human and the graft is an isograft, derived from a human of the same or different genetic origins. In another scenario, the graft is derived from a species different from that into which it is transplanted, including animals from phylogenically widely separated species, for example, a baboon heart being transplanted into a human host.

The term “transplant rejection”, as used herein, encompasses both acute and chronic transplant rejection.

“Acute rejection” is the rejection by the immune system of a tissue transplant-recipient when the transplanted tissue is immunologically foreign. Acute rejection is characterized by infiltration of the transplant tissue by immune cells of the recipient, which carry out their effector function and destroy the transplant tissue. The onset of acute rejection is rapid and generally occurs in humans within a few weeks after transplant surgery. Generally, acute rejection can be inhibited or suppressed with immunosuppressive drugs such as rapamycin, cyclosporin, anti-CD40L monoclonal antibody and the like.

“Chronic rejection” generally occurs in humans within several months to years after engraftment, even in the presence of successful immunosuppression of acute rejection. Fibrosis is a common factor in chronic rejection of all types of organ transplants.

In one embodiment, the transplant rejection is an allogeneic transplant rejection. Accordingly, in one embodiment, the donor of the transplant is a human. The donor of the transplant can be a living donor or a deceased donor, namely a cadaveric donor.

In one embodiment, the transplant is an organ, a tissue or cells.

As used herein, the term “organ” refers to a solid vascularized organ that performs a specific function or group of functions within an organism. The term organ includes, but is not limited to, heart, lung, kidney, liver, pancreas, skin, uterus, bone, cartilage, small or large bowel, bladder, brain, breast, blood vessels, esophagus, fallopian tube, gallbladder, ovaries, pancreas, prostate, placenta, spinal cord, limb including upper and lower, spleen, stomach, testes, thymus, thyroid, trachea, ureter, urethra, uterus.

As used herein, the term “tissue” refers to any type of tissue in human or animals, and includes, but is not limited to, vascular tissue, skin tissue, hepatic tissue, pancreatic tissue, neural tissue, urogenital tissue, gastrointestinal tissue, skeletal tissue including bone and cartilage, adipose tissue, connective tissue including tendons and ligaments, amniotic tissue, chorionic tissue, dura, pericardia, muscle tissue, glandular tissue, facial tissue, ophthalmic tissue.

As used herein, the term “cells” refers to a composition enriched for cells of interest, preferably a composition comprising at least 30%, preferably at least 50%, even more preferably at least 65% of said cells.

In one embodiment, the “cells” are selected from the group comprising or consisting of: multipotent hematopoietic stem cells derived from bone marrow, peripheral blood, or umbilical cord blood; or pluripotent (i.e., embryonic stem cells [ES] or induced pluripotent stem cells [iPS]) or multipotent stem cell-derived differentiated cells of different cell lineages, including, but not limited to, cardiomyocytes, β-pancreatic cells, hepatocytes, neurons and the like.

In one embodiment where the transplantation is an allogeneic hematopoietic stem cell transplantation (HSCT), the “cells” are selected from the group comprising or consisting of: multipotent hematopoietic stem cells, usually derived from bone marrow, peripheral blood, or umbilical cord blood.

“HSCT” or “hematopoietic stem cell transplantation” is a transplantation therapy which can be curative for patients affected with leukemia and lymphomas (including, without limitation, acute myeloid leukemia (AML), acute lymphoid leukemia (ALL), chronic myeloid leukemia (CML), myelodysplasia syndrome (MDS), myeloproliferative syndrome, Hodgkin lymphomas, non-Hodgkin lymphomas, chronic lymphatic leukemia (CLL) and multiple myeloma). However, an important limitation of allogeneic HSCT is the development of graft-versus-host-disease (GVHD), which occurs in a severe form in about 30-50% of humans who receive this therapy.

Therefore, in one embodiment, the isolated T-cell population expressing the CAR, composition, pharmaceutical composition according to the present invention is used to prevent and/or reduce GVHD.

In a further embodiment, the isolated T-cell population expressing the CAR, composition, pharmaceutical composition according to the present invention may be used in combination with multipotent hematopoietic stem cells to prevent and/or treat leukemia and/or lymphomas (including, without limitation, acute myeloid leukemia (AML), acute lymphoid leukemia (ALL), chronic myeloid leukemia (CML), myelodysplasia syndrome (MDS), myeloproliferative syndrome, Hodgkin lymphomas, non-Hodgkin lymphomas, chronic lymphatic leukemia (CLL) and multiple myeloma).

Additionally or alternatively, the isolated T-cell population expressing the CAR, composition, pharmaceutical composition according to the present invention may be used may be used for graft engineering.

In one embodiment, the transplant to be grafted is treated with the isolated T-cell population expressing the CAR, composition, pharmaceutical composition according to the present invention prior to transplantation, to deplete CD45RC^{high} cells.

In a preferred embodiment, the transplant is bone marrow, and is treated with the isolated T-cell population expressing the CAR, composition, pharmaceutical composition according to the present invention prior to transplantation to deplete CD45RC^{high} T cells. In one embodiment, the bone marrow comprises CD34⁺ cells containing CD45RC^{high}T cells and CD45RC^{low/-} T cells.

The present invention further relates to methods of preventing, reducing and/or treating hCD45RC^{high}-related diseases, disorders or conditions, by administering to a subject in need thereof the isolated T-cell population expressing the CAR, composition, pharmaceutical composition according to the present invention. It also relates to the isolated T-cell population expressing the CAR, composition, pharmaceutical composition according to the present invention, for use in preventing and/or treating hCD45RC^{high}-related diseases, disorders or conditions.

As used herein, the term “hCD45RC^{high} diseases, disorders or conditions” refers to diseases, disorders or conditions caused by or potentialized by an increased proportion of cells expressing hCD45RC cells in a subject and/or by an increased level of expression of hCD45RC in cells of the subject.

By “increased proportion of CD45RC^{high} cells in a subject” is meant an increase of about 5%, preferably about 10%, 15%, 20%, 25%, 30%, 35%, 40%, 45%, 50%, 60%, 70%, 80%, 90%, 100% or more of the number of cells expressing CD45RC (i.e., CD45RC^{high} cells) in a given subject as compared to a reference, such as, e.g., the number of CD45RC^{high} cells in a substantially healthy subject.

By “increased level of expression of hCD45RC in cells of the subject” is meant an increase of about 5%, preferably about 10%, 15%, 20%, 25%, 30%, 35%, 40%, 45%, 50%, 60%, 70%, 80%, 90%, 100% or more of the expression level of hCD45RC, whether at the mRNA level or at the protein level, in cells of a given subject as compared to a reference, such as, e.g., the expression level of hCD45RC in cells of a substantially healthy subject.

In one embodiment, hCD45RC^{high}-related diseases, disorders or conditions are selected from the group comprising or consisting of autoimmune diseases, undesired immune responses, monogenic diseases and lymphoma or cancer.

In one embodiment, hCD45RC^{high}-related diseases, disorders or conditions are selected from the group comprising or consisting of autoimmune diseases, undesired immune responses and monogenic diseases.

As used herein, the term “autoimmune disease” refers to a disease in which the immune system produces an immune response (e.g., a B cell or a T cell response) against an antigen that is part of the normal host (that is an auto-antigen), with consequent injury to tissues. In an autoimmune disease, the immune system of the host fails to recognize a particular antigen as “self” and an immune reaction is mounted against the host's tissues expressing the antigen.

Exemplary autoimmune diseases contemplated in the present invention include, but are not limited to, rheumatoid arthritis, juvenile oligoarthritis, collagen-induced arthritis, adjuvant-induced arthritis, Sjogren's syndrome, multiple sclerosis, experimental autoimmune encephalomyelitis, inflammatory bowel disease (including Crohn's disease and ulcerative colitis), autoimmune gastric atrophy, pemphigus vulgaris, psoriasis, vitiligo, type 1 diabetes, non-obese diabetes, myasthenia gravis, Grave's disease, Hashimoto's thyroiditis, sclerosing cholangitis, sclerosing sialadenitis, systemic lupus erythematosis, autoimmune thrombocytopenia purpura, Goodpasture's syndrome, Addison's disease, systemic sclerosis, polymyositis, dermatomyositis, acquired hemophilia, thrombotic thrombocytopenic purpura, uveitis, IgG4-associated autoimmune diseases (such as, e.g., diseases listed in Table 1 of Kleger et al., 2015. Dtsch Arztebl Int. 112(8):128-135, which Table is incorporated by reference) and the like.

In a preferred embodiment, the autoimmune disease is systemic lupus erythematosis.

In a preferred embodiment, the autoimmune disease is inflammatory bowel disease, including Crohn's disease and ulcerative colitis. In a preferred embodiment, the autoimmune disease is Crohn's disease. In a preferred embodiment, the autoimmune disease is ulcerative colitis.

As used herein, the term “undesired immune response” refers to any unwanted immune reaction, preferably any unwanted immune reaction directed to (i) proteins expressed in the course of gene therapy, (ii) vectors (such as, e.g., viral vectors) used in the course of gene therapy and/or (iii) therapeutic proteins. Such proteins include for example factor VIII (hemophilia A) and other coagulation factors, enzyme replacement therapies, monoclonal antibodies (e.g., natalizumab, rituximab, infliximab), polyclonal antibodies, enzymes and cytokines (e.g., IFNβ). The term “undesired immune response” also refers to allergies and allergic reactions.

In one embodiment, the isolated T-cell population expressing the CAR, composition, pharmaceutical composition according to the present invention may be administered to a subject in order to suppress an immune response, especially to prevent immune reactions to specific proteins when their expression is restored by gene therapy in those subjects with corresponding genetic deficiencies. Thus, the isolated T-cell population expressing the CAR, composition, pharmaceutical composition according to the present invention may be used to prevent immune reactivity towards proteins normally absent in the subject due to mutations, while their reconstitution is achieved by gene therapy. Moreover, protein therapy is an area of medical innovation that is becoming more widespread, and involves the application of proteins, such as enzymes, antibodies or cytokines, directly to subjects as therapeutic products. One of the major hurdles in delivery of such medicaments involves the immune responses directed against the therapeutic protein themselves. Administration of protein-based therapeutics is often accompanied by administration of immune suppressants, which are used in order to facilitate a longer lifetime of the protein and therefore increased uptake of the protein into the cells and tissues of the organism. General immune suppressants can however be disadvantageous due to the unspecific nature of the immune suppression that is carried out, resulting in unwanted side effects in the patient. Therefore, this approach can be applied to suppress an immune response against therapeutic proteins and peptides, such as therapeutic antibodies, cytokines, enzymes or any other protein administered to a patient.

In one embodiment, the isolated T-cell population expressing the CAR, composition, pharmaceutical composition according to the present invention may be administered to a subject in order to suppress an immune response, especially to prevent immune reactions to vectors used in gene therapy, in particular viral vectors used in gene therapy. Such viral vectors include, e.g., adeno-associated virus (AAV) vectors, adenoviral (Ad) vectors, lentiviral vectors and the like. For a review, see Nayak & Herzog, 2010. Gene Ther. 17(3):295-304.

As used herein, the term “allergy” or “allergies” refers to an improper reaction of the immune system. Allergic reactions occur to normally harmless environmental substances known as allergens; these reactions are acquired, predictable and rapid. Strictly, allergy is one of four forms of hypersensitivity and is called type I (or immediate) hypersensitivity. It is characterized by excessive activation of certain white blood cells called mast cells and basophils by a type of antibody known as IgE, resulting in an extreme inflammatory response. Common allergic reactions include eczema, hives, hay fever, asthma, food allergies, and reactions to the venom of stinging insects such as wasps and bees.

The term “monogenic diseases”, as used herein, refers to diseases resulting from a mutation in a single gene selected among the following genes:
(i) genes which are not associated with immune function but whose deficiency is associated with inflammation and/or immune reactions, such as genes deficient in the following diseases: Duchenne muscular dystrophy (DMD), cystic fibrosis, lysosomal diseases and al-anti-trypsin deficiency; and
(ii) genes involved in the immune system and whose deficiency generates inflammation and/or autoimmune reactions, such as genes deficient in the following diseases: T cell primary immunodeficiency such as IPEX (immunodysregulation polyendocrinopathy enteropathy X-linked syndrome), APECED (autoimmune polyendocrinopathy-candidiasis-ectodermal dystrophy), B cell primary immunodeficiencies, Muckle-Wells syndrome, mixed autoinflammatory and autoimmune syndrome, NLRP12-associated hereditary periodic fever syndrome, and tumor necrosis factor receptor 1 associated periodic syndrome.

In a preferred embodiment, the autoimmune disease is APECED (autoimmune polyendocrinopathy-candidiasis-ectodermal dystrophy).

In a preferred embodiment, the autoimmune disease is Duchenne muscular dystrophy (DMD).

The term “lymphoma or cancer”, as used herein, encompass lymphoma or cancer which are associated with CD45RC^{high} cells. Exemplary lymphoma or cancer associated with CD45RC^{high} cells include, but are not limited to, acute myeloid leukemia (AML), acute lymphoid leukemia (ALL), chronic myeloid leukemia (CML), myelodysplasia syndrome (MDS)/myeloproliferative syndrome, lymphomas (such as, e.g., Hodgkin and non-Hodgkin lymphomas), chronic lymphatic leukemia (CLL) and multiple myeloma.

The present invention relates therefore to a method of depleting CD45RC^{high} cells in a subject in need thereof, thereby expending and/or potentiating regulatory T cells, preferably Foxp3⁺ and/or CD45RC^{low} Tregs, thereby preventing and/or reducing transplant rejections; or preventing, reducing and/or treating hCD45RC^{high}-related diseases, disorders or conditions, by administering the T-cell population expressing the CAR, composition, pharmaceutical composition according to the present invention.

In a preferred embodiment, the hCD45RC^{high}-related disease, disorder or condition is systemic lupus erythematosis, inflammatory bowel disease (including Crohn's disease and ulcerative colitis), APECED (autoimmune polyendocrinopathy-candidiasis-ectodermal dystrophy), or Duchenne muscular dystrophy (DMD).

In a preferred embodiment, the hCD45RC^{high}-related disease, disorder or condition is systemic lupus erythematosis, inflammatory bowel disease (including Crohn's disease and ulcerative colitis), or APECED (autoimmune polyendocrinopathy-candidiasis-ectodermal dystrophy).

It also relates to the T-cell population expressing the CAR, composition, pharmaceutical composition according to the present invention for use in depleting CD45RC^{high} cells in a subject in need thereof, thereby expending and/or potentiating regulatory T cells, preferably Foxp3⁺ and/or CD45RC^{low} Tregs, thereby preventing and/or reducing transplant rejections; or preventing, reducing and/or treating hCD45RC^{high}-related diseases, disorders or conditions.

The CAR-engineered immune cells of the present invention may be administered either alone or as a pharmaceutical composition described herein (e.g., in combination with diluents and/or with other components, including, without limitation, IL-2 or other cytokines or cell populations).

The pharmaceutical compositions of the present invention may be administered to a subject in any suitable manner, including by aerosol inhalation, injection, ingestion, transfusion, implantation or transplantation. In some embodiments, the pharmaceutical compositions described herein may be administered to a subject by parenteral administration. In certain embodiments, the pharmaceutical compositions described herein may be administered to a subject subcutaneously, intradermally, intratumorally, intranodally, intramedullary, intramuscularly, intrasternally, by intravenous (i.v.) injection, by infusion techniques or intraperitoneally. In particular embodiments, the CAR-modified immune cell compositions of the present invention may be administered to a subject by intradermal or subcutaneous injection. In some embodiments, the CAR-modified immune cell compositions of the present invention may be administered by i.v. injection. In some embodiments, the compositions of CAR-modified immune cells may be injected directly into a lymph node, site of infection, site of inflammation or site of tissue or organ rejection. In some embodiments, the compositions of CAR-modified immune cells may be injected directly into the site of the autoimmune and/or inflammatory disease.

In some embodiments, the subject is administered (or is to be administered) with autologous cells.

In some embodiments, the subject is administered (or is to be administered) with allogenic cells.

In some embodiments, the subject may be a mammal. In particular embodiments, the subject may be a human.

Pharmaceutical compositions of the present invention may be administered in a manner appropriate to the disease to be prevented or treated. The quantity and frequency of administration will be determined by such factors as the condition of the subject and the type and severity of the subject's disease, although appropriate dosages may be determined by clinical trials.

When an “effective amount” or “therapeutic amount” is indicated, the precise amount of the compositions of the present invention to be administered may be determined with consideration of individual differences in age, weight, antibody titer, and condition of the subject. It can generally be stated that a pharmaceutical composition comprising the CAR-engineered immune cells as described herein may be administered at a dosage of at least 1×10², 1×10³, 1×10⁴, 1×10⁵, 1×10⁶, 1×10⁷, 1×10⁸ or 1×10⁹ cells/kg body weight or 1×10⁵ to 100×10⁵ cells/kg body weight, including all integer values within those ranges. CAR-engineered immune cell compositions may also be administered multiple times at any of these dosages or any combination thereof. The CAR-engineered immune cells can be administered by using infusion techniques that are commonly known in immunotherapy. The optimal dosage and treatment regimen for a particular subject can readily be determined by monitoring the subject for signs of disease and adjusting the treatment accordingly.

In one embodiment, the isolated immune cell population expressing the CAR, composition or pharmaceutical composition according to the present invention is to be administered before, concomitantly with or after a therapeutic drug.

In some embodiments, the CAR-engineered immune cells of the present invention may be administered to a subject in conjunction with (e.g., before, simultaneously or following) any number of relevant treatment modalities, including but not limited to treatment with agents such as immunosuppressants, cytotoxins, chemotherapeutic agents, cytokines, immune stimulators, lytic peptides and radioisotopes.

It will be understood by the one skilled in the art that the co-administration of the isolated T-cell population expressing the CAR, composition, pharmaceutical composition according to the present invention with a particular therapeutic drug, which may be chosen among those recited herein but without being limited thereto, will depend on the disease or condition to be prevented and/or treated.

Examples of immunosuppressants include, without limitation, mTOR inhibitors such as, e.g., sirolimus, everolimus, ridaforolimus, temsirolimus, umirolimus and zotarolimus; IL-1 receptor antagonists such as, e.g., anakinra; antimetabolites such as, e.g., azathioprine, leflunomide, methotrexate, mycophenolic acid and teriflunomide; IMiDs such as, e.g., apremilast, lenalidomide, pomalidomide and thalidomide; and antibodies such as, e.g., eculizumab, adalimumab, afelimomab, certolizumab pegol, golimumab, infliximab, nerelimomab, mepolizumab, omalizumab, faralimomab, elsilimomab, lebrikizumab, ustekinumab, secukinumab, muromonab-CD3, otelixizumab, teplizumab, visilizumab, clenoliximab, keliximab, zanolimumab, efalizumab, erlizumab, obinutuzumab, rituximab, ocrelizumab, pascolizumab, gomiliximab, lumiliximab, teneliximab, toralizumab, aselizumab, galiximab, gavilimomab, ruplizumab, belimumab, blisibimod, ipilimumab, tremelimumab, bertilimumab, lerdelimumab, metelimumab, natalizumab, tocilizumab, odulimomab, basiliximab, daclizumab, inolimomab, zolimomab aritox, atorolimumab, cedelizumab, fontolizumab, maslimomab, morolimumab, pexelizumab, reslizumab, rovelizumab, siplizumab, talizumab, telimomab aritox, vapaliximab, vepalimomab, abatacept, belatacept, etanercept, pegsunercept, aflibercept, alefacept and rilonacept. Examples of cytotoxins include, without limitation, radionuclides (e.g., ³⁵S, ¹⁴C, ³²P¹²⁵I, ¹³¹I ⁹⁰Y⁸⁹Zr, ²⁰¹Tl, ¹⁸⁶Re, ¹⁸⁸Re, ⁵⁷Cu, ²¹³Bi, and ²¹¹At), conjugated radionuclides, and chemotherapeutic agents. Further examples of cytotoxins include, but are not limited to, antimetabolites (e.g., 5-fluorouricil (5-FU), methotrexate (MTX), fludarabine, etc.), anti-microtubule agents (e.g., vincristine, vinblastine, colchicine, taxanes (such as paclitaxel and docetaxel), etc.), alkylating agents (e.g., cyclophasphamide, melphalan, bischloroethylnitrosurea (BCNU), etc.), platinum agents (e.g., cisplatin (also termed cDDP), carboplatin, oxaliplatin, JM-216, CI-973, etc.), anthracyclines (e.g., doxorubicin, daunorubicin, etc.), antibiotic agents (e.g., mitomycin-C), topoisomerase inhibitors (e.g., etoposide, tenoposide, and camptothecins), or other cytotoxic agents such as ricin, diptheria toxin (DT), Pseudomonas exotoxin (PE) A, PE40, abrin, saporin, pokeweed viral protein, ethidium bromide, glucocorticoid, anthrax toxin and others.

Examples of chemotherapeutic agents include, without limitation, platinum coordination compounds (such as, e.g., cisplatin, carboplatin or oxalyplatin); taxane compounds (such as, e.g., paclitaxel or docetaxel); topoisomerase I inhibitors (such as, e.g., irinotecan or topotecan); topoisomerase II inhibitors (such as, e.g., etoposide or teniposide); vinca alkaloids (such as, e.g., vinblastine, vincristine or vinorelbine); anti-tumor nucleoside derivatives (such as, e.g., 5-fluorouracil, gemcitabine or capecitabine); alkylating agents (such as, e.g., nitrogen mustard or nitrosourea, cyclophosphamide, chlorambucil, carmustine or lomustine; anti-tumor anthracycline derivatives (such as, e.g., daunorubicin, doxorubicin, idarubicin or mitoxantrone); anti-HER2 antibodies (such as, e.g., trastuzumab); estrogen receptor antagonists or selective estrogen receptor modulators (such as, e.g., tamoxifen, toremifene, droloxifene, faslodex or raloxifene); aromatase inhibitors (such as, e.g., exemestane, anastrozole, letrazole or vorozole); differentiating agents (such as, e.g., retinoids, vitamin D and retinoic acid metabolism blocking agents [RAMBA] such as accutane); DNA methyl transferase inhibitors (such as, e.g., azacytidine); kinase inhibitors (such as, e.g., flavoperidol, imatinib mesylate or gefitinib); farnesyltransferase inhibitors; and HDAC inhibitors.

Examples of cytokines include, without limitation, chemokines (such as, e.g., CCL1, CCL2/MCP1, CCL3/MIP1α, CCL4/MIP1β, CCL5/RANTES, CCL6, CCL7, CCL8, CCL9, CCL11, CCL12, CCL13, CCL14, CCL15, CCL16, CCL17, CCL18/PARC/DCCK1/AMAC1/MIP4, CCL19, CCL20, CCL21, CCL22, CCL23, CCL24, CCL25, CCL26, CCL27, CCL28, CXCL1/KC, CXCL2, CXCL3, CXCL4, CXCL5, CXCL6, CXCL7, CXCL8/IL8, CXCL9, CXCL10, CXCL11, CXCL12, CXCL13, CXCL14, CXCL15, CXCL16, CXCL17, CX3CL1, XCL1 and XCL2), tumor necrosis factors (such as, e.g., TNFA, Lymphotoxin, TNFSF4, TNFSF5/CD40LG, TNFSF6, TNFSF7, TNFSF8, TNFSF9, TNFSF10, TNFSF11, TNFSF13, TNFSF13B and EDA) and interleukins (such as, e.g., IL-1a, IL-10, IL-1Ra, IL-2, IL-3, IL-4, IL-5, IL-6, IL-7, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36u, IL-360, IL-36γ, IL-36Ra, IL-37, IL-38, IFNα, IFNβ, IFNκ, IFNω and GM-CSF).

Examples of immune stimulators include, without limitation, filgrastim, pegfilgrastim, lenograstim, molgramostim, sargramostim, ancestim, albinterferon, interferon alfa, peginterferon alfa, interferon beta, peginterferon beta, interferon gamma, aldesleukin, oprelvekin, growth hormone, immunocyanin, pegademase, prolactin, tasonermin, histamine dihydrochloride, poly ICLC, vitamin D, lentinan, plerixafor, roquinimex, mifamurtide, glatiramer acetate, thymopentin, thymosin al, thymulin, polyinosinic:polycytidylic acid, pidotimod, Bacillus Calmette-Gudrin vaccine, melanoma vaccine and sipuleucel-T vaccine.

Examples of lytic peptides include, without limitation, toxins (such as, e.g., Diptheria toxin or Pseudomonas exotoxin).

Examples of radioisotopes include, without limitation, the radionuclides of technetium (e.g., Tc-99 and Tc-97), potassium (e.g., K-40), rubidium (e.g., Rb-82), iodine (e.g., I-123, I-124, I-125, I-129, I-131), cesium (e.g., Cs-135, Cs-137), cobalt (e.g., Co-60), palladium (e.g., Pd-103, Pd-107), cadmium (e.g., Cd-113), strontium (e.g., Sr-89, Sr-90), europium (e.g., Eu-55), tin (e.g., Sn-121, Sn-126), phosphorus (e.g., P-32, P-33), thallium (e.g., T1-201), indium (e.g., In-111), gallium (e.g., Ga-67, Ga-68), yttrium (e.g., Y-90), iridium (e.g., Ir-192), bismuth (e.g., Bi-213), radium (e.g., Ra-223, Ra-225), and ruthenium (e.g., Ru-106).

The CAR-engineered immune cells of the present invention may be administered to the subject before, after, or concomitant with the immunosuppressant agent.

The CAR-engineered immune cells of the present invention and/or the immunosuppressant agent(s) may be administered to the subject after transplantation. Alternatively, or in addition, the CAR-engineered immune cells of the present invention and/or the immunosuppressant agent(s) may be administered to the subject before transplantation. In some embodiments, the CAR-engineered immune cells of the present invention and/or the immunosuppressant agent may be administered to the subject during transplantation surgery.

In some embodiments, the administration of CAR-engineered immune cells to the subject is carried out once immunosuppressive therapy has been initiated.

In some embodiments, the method is carried out more than once, e.g., to monitor the transplant recipient over time, and, if applicable, in different immunosuppressive therapy regimes.

In some embodiments, immunosuppressive therapy is reduced if the transplant recipient is predicted to be tolerant of the transplant. In some embodiments, no immunosuppressive therapy is prescribed, e.g., immunosuppressive therapy is ceased, if the transplant recipient is predicted to be tolerant of the transplant.

The CAR-engineered immune cells of the present invention may be administered following a diagnosis of transplant organ or tissue rejection followed by doses of both the CAR-engineered immune cells of the invention and immunosuppressant agent(s) until symptoms of organ or tissue rejection subside.

In a further embodiment, the CAR-engineered immune cell compositions of the present invention may be administered to a subject in conjunction with (e.g., before, simultaneously with, or following) bone marrow transplantation.

In some embodiments, the CAR-engineered immune cells of the present invention may be administered following B cell ablative therapy such as agents that react with CD20, e.g., rituximab. For example, in some embodiments, subjects may undergo standard treatment with high dose chemotherapy followed by peripheral blood stem cell transplantation. In certain embodiments, following the transplant, subjects may receive an infusion of the expanded CAR-engineered immune cells of the present invention. In certain embodiments, expanded CAR-engineered immune cells may be administered before or following surgery.

In some embodiments, the subject (e.g., human) receives an initial administration of an immune cell or population of the invention, and one or more subsequent administrations, wherein the one or more subsequent administrations are administered less than 15 days, e.g., 14, 13, 12, 11, 10, 9, 8, 7, 6, 5, 4, 3, or 2 days after the previous administration.

In some embodiments, a therapeutically effective amount of immune cells of the invention is administered or is to be administered to the subject.

In some embodiments, the number of immune cells of the immune cell population of the invention administered to the subject is at least of 10², 10³, 10⁴, 10⁵, 10⁶, 10⁷, 108 or 10⁹ cells.

In some embodiments, the number of immune cells of the immune cell population of the invention administered to the subject ranges from about 10² to about 10⁹, from about 10³ to about 10⁸, from about 104 to about 107, or from about 105 to about 10⁶ cells.

In some embodiments, the number of immune cells of the immune cell population of the invention administered to the subject ranges from about 10² to about 10⁹, from about 10² to 10⁸, from about 10² to 10⁷, from about 10² to 10⁶, from about 10² to 10⁵, from about 10² to 10⁴, or from about 10² to 10³ cells. In some embodiments, the number of immune cells of the immune population of the invention administrated to the subject is about 10², about 10³, about 10⁴, about 10⁵, about 10⁶, about 10⁷, about 10⁸, or about 10⁹ cells.

In some embodiments, the number of immune cells of the immune cell population of the invention administered to the subject is at least 10², 10³, 10⁴, 10⁵, 10⁶, 10⁷, 108 or 10⁹ cells/kg body weight.

In some embodiments, the number of immune cells of the immune cell population of the invention administered to the subject ranges from about 10² to 10⁹ cells/kg body weight or 10³ to 10⁸ cells/kg body weight, including all integer values within those ranges.

In some embodiments, the subject receives more than one administration of the immune cell population of the invention per week, e.g., 2, 3, or 4 administrations of an immune cell population of the invention administered per week to the subject.

In some embodiments, the immune cell population is administered to the subject in need thereof in combination with an active agent. According to some embodiments, the immune cell population is administered before, at the same time as, or after the administration of an active agent.

In some embodiments, it may be desirable to administer activated immune cells of the invention to a subject and then subsequently redraw blood (or have an apheresis performed), activate immune cells therefrom according to the present invention, and reinfuse the subject with these activated and expanded immune cells. This process can be carried out multiple times every few weeks. In certain embodiments, immune cells can be activated from blood draws of from 10 cc to 400 cc. In certain embodiments, immune cells are activated from blood draws of 20 cc, 30 cc, 40 cc, 50 cc, 60 cc, 70 cc, 80 cc, 90 cc, or 100 cc. Not to be bound by theory, using this multiple blood draw/multiple reinfusion protocol may serve to select out certain populations of immune cells.

It is understood that the CARs, cell populations, and compositions described herein may be used in a method of treatment as described herein, may be for use as a medicament as described herein, may be for use in a treatment as described herein, and/or may be for use in the manufacture of a medicament for a treatment as described herein.

As apparent from the specification, the CAR of the invention, or the antibody or an antigen-binding fragment comprised in said CAR, or the immune cell population engineered to express said CAR, presents numerous advantages.

In one embodiment, the CAR of the invention, or the antibody or an antigen-binding fragment comprised in said CAR, or the immune cell population engineered to express said CAR, binds to human CD45RC with an equilibrium dissociation constant (K_{d}) of about 5×10⁻⁷ M or less, preferably of about 2.5×10⁻⁷ M or less, about 1×10⁻⁷ M or less, about 7.5×10⁻⁸ M or less, about 5×10⁻⁸ M or less, about 1×10⁻⁸ M or less.

In one embodiment, the CAR of the invention, or the antibody or an antigen-binding fragment comprised in said CAR, or the immune cell population engineered to express said CAR, has cytotoxic activity against CD45RC^{high} cells.

In one embodiment, upon binding to its target antigen, the CAR of the invention induces CAR-transduced cell activation.

In one embodiment, upon binding to its target antigen, the CAR of the invention induces CAR-transduced T cell activation. In one embodiment, upon binding to its target antigen, the CAR of the invention induces CAR-transduced Treg activation. In one embodiment, upon binding to its target antigen, the CAR of the invention induces CAR-transduced CD4⁺ Tregs activation. In another embodiment, upon binding to its target antigen, the CAR of the invention induces CAR-transduced CD8⁺ Tregs activation.

In one embodiment, the immune cell population engineered to express said CAR of the invention has pro-apoptotic activity against target cells (for instance CD45RC⁺ T cells).

In one embodiment, the immune cell population engineered to express said CAR of the invention is a population of CD4⁺ Tregs, and said CAR-transduced CD4⁺ Tregs have pro-apoptotic activity against target cells (for instance CD45RC⁺ T cells). In one embodiment, the immune cell population engineered to express said CAR of the invention is a population of CD8⁺ Tregs, and said CAR-transduced CD8⁺ Tregs have pro-apoptotic activity against target cells (for instance CD45RC⁺ T cells).

In one embodiment, the CAR of the invention, or the antibody or an antigen-binding fragment comprised in said CAR, or the immune cell population engineered to express said CAR, decreases or abrogates graft rejection (e.g., skin graft rejection) and GVHD.

### BRIEF DESCRIPTION OF THE DRAWINGS

FIG. 1 A1(1)-B(2) shows the expression level of CD45RC (as detected by ABIS-45RC or by the commercially available MT2 antibody) on different leukocyte types in human blood. Staining with ABIS-45RC or MT2 was realized on different cell types in total blood (EDTA) from healthy volunteers. Red blood cells were then lysed (Versalyse, Beckman Coulter) before cytometer analysis (Navios, Beckman Coulter). Cells were first gated on morphology, doublet cells and lived cells. (A(1) through A(3)) Representative dot plot analysis of CD45RC expression detected by ABIS-45RC or MT2 from one out of three healthy volunteers analyzed on different leukocyte types. ABIS-45RC is shown on left panels and MT2 on right panels. x-axis shows the fluorescence intensity of cell lineage markers labelling for each type of leukocytes as indicated; y-axis represents the fluorescence intensity of anti-CD45RC antibody labelling. The horizontal lines define cells with high, intermediate/low and negative levels of CD45RC expression as indicated in the upper left dot plot and numbers represent the percentage of cells in each category. (B(1) and B(2)) Mean expression+/− SEM of CD45RC^{high}, CD45RC^{low} and CD45RC⁻ on different leukocyte types of three donors labelled with ABIS-45RC (B(1)) or MT2 (B(2)).

FIG. 2 shows that both ABIS-45RC and the commercial anti-CD45RC MT2 antibody compete for the same epitope. PBMCs were isolated from blood of healthy volunteers and T cells were labeled with an anti-CD3 labeled mAb, with chimeric ABIS-45RC (at the indicated concentrations) and anti-CD45RC (mouse clone MT2)-FITC labeled at 1.33 mg/mL. ABIS-45RC reactivity was revealed using a biotin donkey anti-human IgG+Strepta PerCP-Cy 5.5 secondary antibody. Numbers in the windows of the dot plots of the upper row represent the percentage of cells that were co-labeled by both antibodies.

FIG. 3 A-F shows that cytotoxicity induced by ABIS-45RC is higher compared to commercial anti-CD45RC MT2. PBMCs from healthy volunteers (n=3) were incubated at 37° C. with medium, isotype negative control (2.5 or 10 mg/mL), ABIS-CD45RC (2.5 or 10 mg/mL) or dexamethasone (10 mg/mL) as a positive control for the indicated time points and then cells were labeled with an anti-CD3-FITC mouse antibody and apoptotic cells by labeling with Annexin-V-PE. (A-E) Graphs indicate % of Annexin V⁺ cells in indicated cell populations. (F) Representative dot plots of Annexin V⁺ (early apoptotic) and DAPI⁺ (late apoptotic) cells. Numbers indicate the percentage of cells in each category.

FIG. 4 A-C shows the use of ABIS-45RC to treat GVHD in immune humanized NSG immunodeficient mice. (A) Experimental procedure showing that peripheral blood mononuclear cells (PBMCs) from healthy donor volunteers were infused intravenously (iv) (day 0) into previously (day −1) sublethaly (2 Gy) irradiated NSG immunodeficient mice. ABIS-45RC was administered intraperitoneally (ip) with the indicated protocol between day 0 to 20. Isotype control were human IgG (IVIg preparation) and was administered using the same protocol as ABIS-45RC. (B-C) Survival curves for NSG mice and statistics were analyzed using a Kaplan-Meier analysis (* p<0.01, ** p<0.001).

FIG. 5 A-J is a combination of flow cytometry dot plots, showing the reactivity against human T cells of humanized ABIS-45RC antibodies variants and of the murine ABIS-45RC antibody, at two concentrations (2 μg/mL on the left panel, 1 μg/mL on the right panel). (A) humanized ABIS-45RC variant A; (B) humanized ABIS-45RC variant B; (C) humanized ABIS-45RC variant C; (D) humanized ABIS-45RC variant D; (E) humanized ABIS-45RC variant E; (F) humanized ABIS-45RC variant F; (G) humanized ABIS-45RC variant G; (H) humanized ABIS-45RC variant H; (I) humanized ABIS-45RC variant I; (J) murine ABIS-45RC.

FIG. 6 shows two dot plots of flow cytometry showing that both ABIS-45RC (left panel) and engineered Asn/Phe ABIS-45RC (right panel) have an equivalent pattern of reactivity against human T cells.

FIG. 7 A-C shows the expression level of CD45RC on CD3⁺ leukocyte in human blood from three healthy volunteers. Cells were first gated on morphology, doublet cells and lived cells. (A) representative dot plot analysis of CD45RC expression detected by murine ABIS-45RC from one out of three healthy volunteers analyzed; (B) representative dot plot analysis of CD45RC expression detected by humanized ABIS-45RC variant A1 from one out of three healthy volunteers analyzed; (C) representative dot plot analysis of CD45RC expression detected by humanized ABIS-45RC variant A3 from one out of three healthy volunteers analyzed. x-axis shows the FSC; y-axis represents the fluorescence intensity of anti-CD45RC antibody labelling. The squares define cells with high, intermediate/low and negative levels of CD45RC expression as indicated and numbers represent the percentage of cells in each category.

FIG. 8 A-B shows that apoptosis induced by ABIS-45RC or by the humanized variants A1 and A3 is comparable. PBMCs from healthy volunteers were incubated at 37° C. with an isotype negative control (10 μg/mL), with murine ABIS-CD45RC (10 μg/mL), with the humanized variant A1 (10 μg/mL) or with the humanized variant A3 (10 μg/mL) for the indicated time points, and cells were then labeled with anti-CD3 and anti-CD45RA antibodies and apoptotic cells by labeling with Annexin-V-PE. The graphs indicate fold apoptosis in CD3⁺ CD45RA^{hi} cells (A) and in CD3⁻ cells (B), compared to the isotype control condition.

FIG. 9 shows the skin graft survival of treated humanized mice with anti-human CD45RC treatment. NSG mice transferred with total human PBMCs to induce human skin rejection were treated with murine ABIS-45RC or humanized variant A1, together with rapamycin (Rapa). Results are expressed in skin graft survival score.

FIG. 10 depicts a schematic view of a CAR structure and of an exemplary structure of a CD45RC-CAR. The CAR comprises an extracellular domain (e.g. a scFv CD45RC), optionally a hinge domain (e.g. derived from CD8α), a transmembrane (TM) domain (e.g. derived from CD8), a costimulatory intracellular domain (e.g. derived from CD28) and a primary signaling domain (e.g. derived from CD3ζ). In the same plasmid, GFP coding sequences may optionally be present immediately 3′ of the CD45RC-CAR sequences separated from them by a T2A self-splicing sequence (not drawn). Thus, GFP may be used as a surrogate marker of CAR-CD45RC expression.

FIG. 11 shows that HEK (human embryonic kidney 293 cells) cells transfected with the plasmid encoding CD45RC-CAR and GFP express GFP 3 days after transfection (left panel). As a positive control of transfection, HEK cells were transfected with a plasmid encoding for GFP only (right panel).

FIG. 12 shows that Jurkat cells can be transduced with the CD45RC-CAR and GFP encoding lentiviral vector after 6 days of culture in comparison with non-transduced Jurkat cells.

FIG. 13 shows that Jurkat cells expressed at cell surface the transduced CD45RC-CAR (as shown by the protein L staining) and that expression of the CAR correlate with expression of the GFP.

FIG. 14 shows that Jurkat cells transduced with the CD45RC-CAR lentiviral vector can induce apoptosis in human T cells. Human T cells were cultured in different cell ratio conditions with Jurkat cells transduced with CD45RC-CAR or Ctrl-CAR (a control CAR having different antigenic specificity). Apoptosis was evaluated by flow cytometry after 18h of culture. The anti-CD45RC monoclonal antibody (chimeric human IgG1) used to generate the CAR was used as a positive control and a human non-reactive IgG1 as an isotype control, Ctrl-CAR Jurkat expressing a CAR recognizing a target non-expressed in this assay and non-transduced Jurkat cells were used as a negative control.

FIG. 15 shows that Jurkat cells transduced with the CD45RC-CAR lentiviral vector can be activated after a contact with human T cells. Human T cells were cultured in different cell ratio conditions with Jurkat cells transduced with CD45RC-CAR (sorted or not based on the expression of GFP) or Ctrl-CAR. The mean fluorescence intensity (MFI) of CD69, a marker of T cell activation, was evaluated by flow cytometry after 18h of culture. Ctrl-CAR Jurkat sorted or not and non-transduced Jurkat were used as a negative control.

FIG. 16 depicts CD45RC-CAR binding to the CD45RC target. HEK 293T were non-transduced or transduced with CD45RC-CAR lentiviral vector and cultured for 5 days. Cells were then incubated with biotinylated CD45R-ABC protein and then stained with streptavidin-APC-Cy7 and analyzed by flow cytometry.

FIG. 17 A-B shows the expansion of CD4⁺Tregs transduced with CD45RC-CAR lentiviral vectors. CD4⁺Tregs were activated between day 0 and day 1 and then transduced twice with CD45RC-CAR on day 1 and 2. GFP+ cells were sorted on day 7. CD45RC-CAR CD4⁺Tregs were analyzed for GFP expression after 7 or 14 days of expansion: (A) Representative histograms and dot plot of GFP expression in CD45RC-CAR transduced CD4⁺Tregs at day 7 and 14; (B) Percentage of cells expressing the CD45RC-CAR at day 7 and 14.

FIG. 18 shows that CD45RC-CAR CD4⁺Tregs are specifically activated through the CAR. CD45RC-CAR CD4⁺Tregs and Ctrl-CAR CD4⁺Tregs expanded for 14 days were cultured for 24h with coated CD45R-ABC protein, in the presence of brefeldin A for the 4 last hours, and then analyzed by flow cytometry for activation markers. MFI of each marker is expressed as a ratio to unstimulated cells.

FIG. 19 A-C shows that CD45RC-CAR CD4⁺Tregs induce cell death of CD45RC^{high} T cells through apoptosis. Apoptosis was analyzed on PBMC incubated 4h with different ratios of allogenic CD4⁺ Tregs transduced with CD45RC-CAR (average of 60% GFP+ transduced cells), Ctrl-CAR (>90% LNGFR+ transduced cells) or non-transduced CD4⁺Tregs, as well as with anti-CD45RC mAb (ABIS-45RC at 10 μg/mL) followed by staining with annexin V and DAPI. Results are expressed as relative proportion of annexin V⁺ cells among (A) T cells, (B) CD45RC^{low/neg} T cells, (C) CD45RC^{high} T cells. The grey diamond-shaped isolated point represents apoptosis in the presence of the anti-CD45RC mAb.

### EXAMPLES

The present invention is further illustrated by the following examples.

Throughout the examples, the following nomenclature applies:

“ABIS-45RC”: the murine anti-hCD45RC antibody of the invention, comprising:
a heavy chain variable region with SEQ ID NO: 61;
a heavy chain constant region with SEQ ID NO: 93;
a light chain variable region with SEQ ID NO: 81; and
a light chain constant region with SEQ ID NO: 94.

“Anti-45RC Variant A”: a humanized variant of ABIS-45RC, comprising:
a heavy chain variable region with SEQ ID NO: 62;
a heavy chain constant region with SEQ ID NO: 91;
a light chain variable region with SEQ ID NO: 82; and
a light chain constant region with SEQ ID NO: 92.

“Anti-45RC Variant B”: a humanized variant of ABIS-45RC, comprising:
a heavy chain variable region with SEQ ID NO: 62;
a heavy chain constant region with SEQ ID NO: 91;
a light chain variable region with SEQ ID NO: 83; and
a light chain constant region with SEQ ID NO: 92.

“Anti-45RC Variant C”: a humanized variant of ABIS-45RC, comprising:
a heavy chain variable region with SEQ ID NO: 62;
a heavy chain constant region with SEQ ID NO: 91;
a light chain variable region with SEQ ID NO: 84; and
a light chain constant region with SEQ ID NO: 92.

“Anti-45RC Variant D”: a humanized variant of ABIS-45RC, comprising:
a heavy chain variable region with SEQ ID NO: 63;
a heavy chain constant region with SEQ ID NO: 91;
a light chain variable region with SEQ ID NO: 82; and
a light chain constant region with SEQ ID NO: 92.

“Anti-45RC Variant E”: a humanized variant of ABIS-45RC, comprising:
a heavy chain variable region with SEQ ID NO: 63;
a heavy chain constant region with SEQ ID NO: 91;
a light chain variable region with SEQ ID NO: 83; and
a light chain constant region with SEQ ID NO: 92.

“Anti-45RC Variant F”: a humanized variant of ABIS-45RC, comprising:
a heavy chain variable region with SEQ ID NO: 63;
a heavy chain constant region with SEQ ID NO: 91;
a light chain variable region with SEQ ID NO: 84; and
a light chain constant region with SEQ ID NO: 92.

“Anti-45RC Variant G”: a humanized variant of ABIS-45RC, comprising:
a heavy chain variable region with SEQ ID NO: 64;
a heavy chain constant region with SEQ ID NO: 91;
a light chain variable region with SEQ ID NO: 83; and
a light chain constant region with SEQ ID NO: 92.

“Anti-45RC Variant H”: a humanized variant of ABIS-45RC, comprising:
a heavy chain variable region with SEQ ID NO: 64;
a heavy chain constant region with SEQ ID NO: 91;
a light chain variable region with SEQ ID NO: 84; and
a light chain constant region with SEQ ID NO: 92.

“Anti-45RC Variant I”: a humanized variant of ABIS-45RC, comprising:
a heavy chain variable region with SEQ ID NO: 64;
a heavy chain constant region with SEQ ID NO: 91;
a light chain variable region with SEQ ID NO: 82; and
a light chain constant region with SEQ ID NO: 92.

“Anti-45RC Variant A1”: a humanized variant of ABIS-45RC, comprising:
a heavy chain variable region with SEQ ID NO: 101;
a heavy chain constant region with SEQ ID NO: 91;
a light chain variable region with SEQ ID NO: 85; and
a light chain constant region with SEQ ID NO: 92.

“Anti-45RC Variant A2”: a humanized variant of ABIS-45RC, comprising:
a heavy chain variable region with SEQ ID NO: 101;
a heavy chain constant region with SEQ ID NO: 91;
a light chain variable region with SEQ ID NO: 103; and
a light chain constant region with SEQ ID NO: 92.

“Anti-45RC Variant A3”: a humanized variant of ABIS-45RC, comprising:
a heavy chain variable region with SEQ ID NO: 65;
a heavy chain constant region with SEQ ID NO: 91;
a light chain variable region with SEQ ID NO: 85; and
a light chain constant region with SEQ ID NO: 92.

“Anti-45RC Variant A4”: a humanized variant of ABIS-45RC, comprising:
a heavy chain variable region with SEQ ID NO: 65;
a heavy chain constant region with SEQ ID NO: 91;
a light chain variable region with SEQ ID NO: 103; and
a light chain constant region with SEQ ID NO: 92.

“Anti-45RC Variant A5”: a humanized variant of ABIS-45RC, comprising:
a heavy chain variable region with SEQ ID NO: 62;
a heavy chain constant region with SEQ ID NO: 91;
a light chain variable region with SEQ ID NO: 85; and
a light chain constant region with SEQ ID NO: 92.

“Anti-45RC Variant A6”: a humanized variant of ABIS-45RC, comprising:
a heavy chain variable region with SEQ ID NO: 101;
a heavy chain constant region with SEQ ID NO: 91;
a light chain variable region with SEQ ID NO: 82; and
a light chain constant region with SEQ ID NO: 92.

“Anti-45RC Variant A7”: a humanized variant of ABIS-45RC, comprising:
a heavy chain variable region with SEQ ID NO: 121;
a heavy chain constant region with SEQ ID NO: 91;
a light chain variable region with SEQ ID NO: 85; and
a light chain constant region with SEQ ID NO: 92.

“Anti-45RC Variant A8”: a humanized variant of ABIS-45RC, comprising:
a heavy chain variable region with SEQ ID NO: 122;
a heavy chain constant region with SEQ ID NO: 91;
a light chain variable region with SEQ ID NO: 85; and
a light chain constant region with SEQ ID NO: 92.

“Anti-45RC Variant A9”: a humanized variant of ABIS-45RC, comprising:
a heavy chain variable region with SEQ ID NO: 123;
a heavy chain constant region with SEQ ID NO: 91;
a light chain variable region with SEQ ID NO: 85; and
a light chain constant region with SEQ ID NO: 92.

“Anti-45RC Variant A10”: a humanized variant of ABIS-45RC, comprising:
a heavy chain variable region with SEQ ID NO: 124;
a heavy chain constant region with SEQ ID NO: 91;
a light chain variable region with SEQ ID NO: 85; and
a light chain constant region with SEQ ID NO: 92.

“Anti-45RC Variant D1”: a humanized variant of ABIS-45RC, comprising:
a heavy chain variable region with SEQ ID NO: 63;
a heavy chain constant region with SEQ ID NO: 91;
a light chain variable region with SEQ ID NO: 85; and
a light chain constant region with SEQ ID NO: 92.

“Anti-45RC Variant I1”: a humanized variant of ABIS-45RC, comprising:
a heavy chain variable region with SEQ ID NO: 67;
a heavy chain constant region with SEQ ID NO: 91;
a light chain variable region with SEQ ID NO: 85; and
a light chain constant region with SEQ ID NO: 92.

“Anti-45RC Variant I2”: a humanized variant of ABIS-45RC, comprising:
a heavy chain variable region with SEQ ID NO: 67;
a heavy chain constant region with SEQ ID NO: 91;
a light chain variable region with SEQ ID NO: 103; and
a light chain constant region with SEQ ID NO: 92.

“MT2”: a murine anti-hCD45RC antibody commercially available at OriGene, under Ref. AM39022PU-N.

“Engineered Asn/Phe ABIS-45RC”: the murine ABIS-45RC, chimerized by engineering its LCVR by insertion of one residue in the CDR1 and substitution of one residue in the FR3 (as described in Example 8). Engineered Asn/Phe ABIS-45RC comprises:
a heavy chain variable region with SEQ ID NO: 61;
a heavy chain constant region with SEQ ID NO: 93;
a light chain variable region with SEQ ID NO: 71 with X₁₂ being Asn (N); and
a light chain constant region with SEQ ID NO: 94.

“Engineered Ser/Phe ABIS-45RC”: the murine ABIS-45RC, chimerized by engineering its LCVR by insertion of one residue in the CDR1 and substitution of one residue in the FR3 (as described in Example 8). Engineered Ser/Phe ABIS-45RC comprises:
a heavy chain variable region with SEQ ID NO: 61;
a heavy chain constant region with SEQ ID NO: 93;
a light chain variable region with SEQ ID NO: 71 with X₁₂ being Ser (S); and
a light chain constant region with SEQ ID NO: 94.

“Engineered Gly/Phe ABIS-45RC”: the murine ABIS-45RC, chimerized by engineering its LCVR by insertion of one residue in the CDR1 and substitution of one residue in the FR3 (as described in Example 8). Engineered Gly/Phe ABIS-45RC comprises:
a heavy chain variable region with SEQ ID NO: 61;
a heavy chain constant region with SEQ ID NO: 93;
a light chain variable region with SEQ ID NO: 71 with X₁₂ being Gly (G); and
a light chain constant region with SEQ ID NO: 94.

“Chimeric N50A ABIS-45RC”: a chimeric variant of the murine ABIS-45RC, comprising the murine ABIS-45RC heavy chain and a humanized light chain. Chimeric N50A ABIS-45RC comprises:
a heavy chain variable region with SEQ ID NO: 61;
a heavy chain constant region with SEQ ID NO: 93;
a light chain variable region with SEQ ID NO: 113; and
a light chain constant region with SEQ ID NO: 92.

“Chimeric S52A ABIS-45RC”: a chimeric variant of the murine ABIS-45RC, comprising the murine ABIS-45RC heavy chain and a humanized light chain. Chimeric S52A ABIS-45RC comprises:
a heavy chain variable region with SEQ ID NO: 61;
a heavy chain constant region with SEQ ID NO: 93;
a light chain variable region with SEQ ID NO: 126; and
a light chain constant region with SEQ ID NO: 92.

“Chimeric N50X ABIS-45RC”: a chimeric variant of the murine ABIS-45RC, comprising the murine ABIS-45RC heavy chain and a humanized light chain. Chimeric N50X ABIS-45RC comprises:
a heavy chain variable region with SEQ ID NO: 61;
a heavy chain constant region with SEQ ID NO: 93;
a light chain variable region with SEQ ID NO: 129, with X₁₃ being any amino acid but Ala (A) or Asn (N); and
a light chain constant region with SEQ ID NO: 92.

### Example 1

Reactivity of ABIS-45RC

Material and Methods

PBMC Staining and Data Acquisition

50 μL or 100 μL of fresh EDTA whole blood were stained with combinations of appropriate monoclonal antibodies (Abs) followed by erythrocyte lysis (versalyse, Beckman Coulter). After washing, cells were analyzed on a Navios flow cytometer and data analyzed using Kaluza software (Beckman Coulter, Marseille, France) and FlowJo Software (Tree Star Inc).

Antibodies and Flow Cytometry

TABLE 6
Antibodies used for flow cytometry analysis. For each antibody in the left
column, the clone used is given in the right column.

| Antibody (specificity) | Clone |
|---|---|
| CD117 | 104D2D1 |
| CD11c | BU15 |
| CD123 | 9F5 |
| CD127 | R34.34 |
| CD14 | RMO52 |
| CD16 | 3G8 |
| CD161 | 191B8 |
| CD19 | SJ25C1 |
| CD25 | 2A3 |
| CD3 | SK7 |
| CD3 | UCHT1 |
| CD336 | Z231 |
| CD4 | 13B8.2 |
| CD45 | J.33 |
| CD56 | N901 |
| CD56 | NCAM162 |
| CD8 | B9.11 |
| CD8 | SK1 |
| Cocktail ILCs | — |
| CRTH2 | BM16 |
| HLADR | L243 |
| Lineage1 DCs | — |
| TCRab | IP26A |
| TCRgd | IMMU510 |
| Va24 | 6B11 |
| Va7, 2 | REA179 |
| CD45RC | MT2 |
| CD45RC | ABIS |
| Streptavidine | Alexa fluor 405 |

Results

As a first screening, ABIS-45RC did not react against CD45RC− cells sorted using the commercial anti-CD45RC mAb MT2 clone suggesting that ABIS-45RC could recognize CD45RC (data not shown).

To further characterize ABIS-45RC, the inventors analyzed its reactivity with human PBMCs.

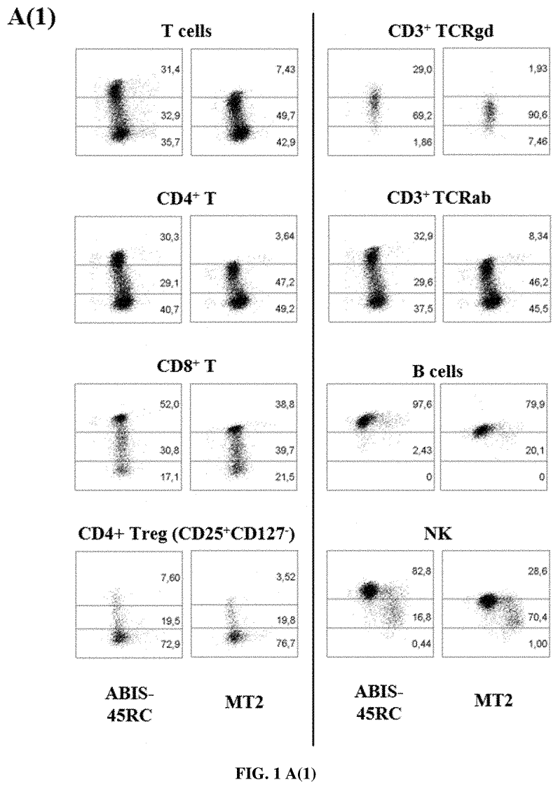

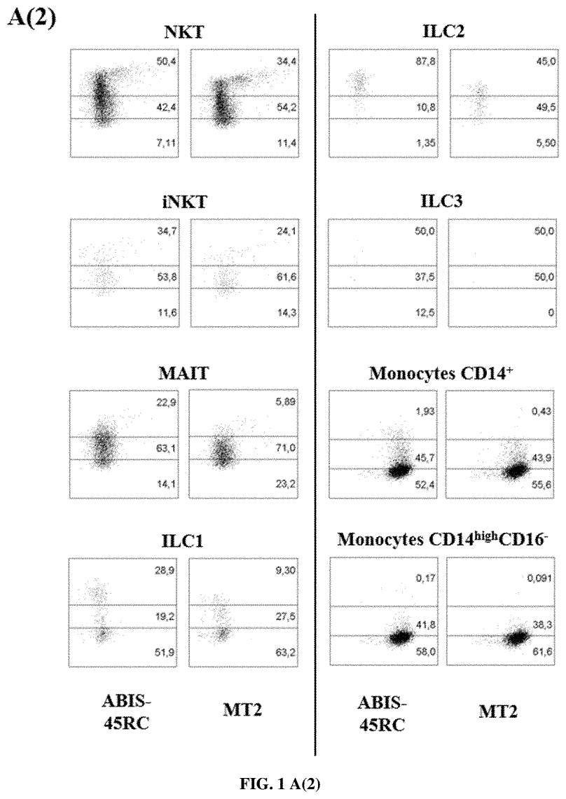

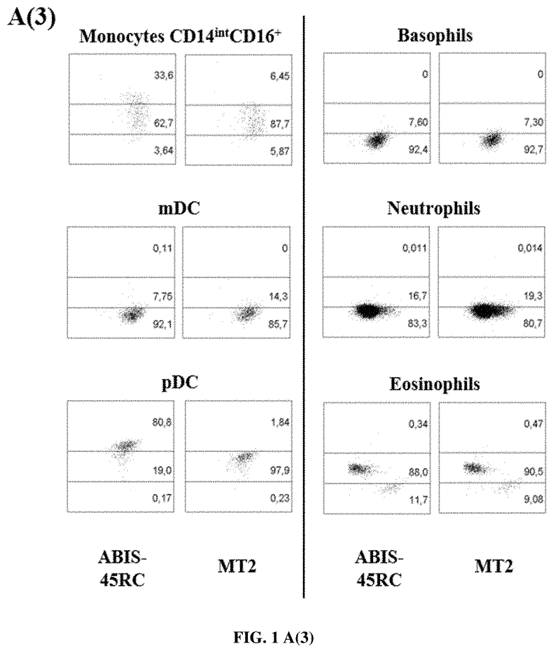

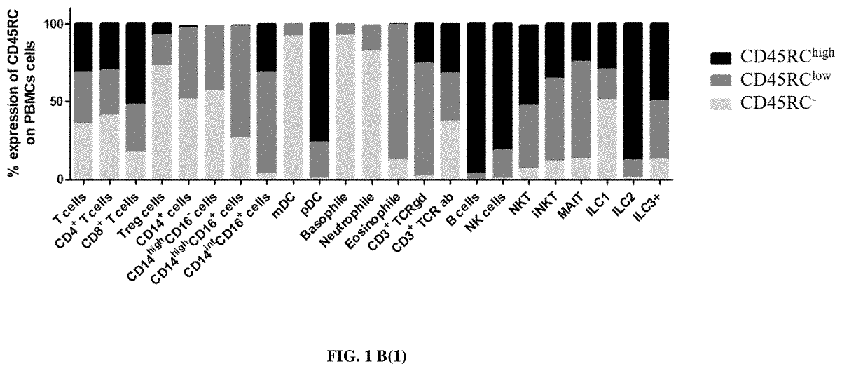

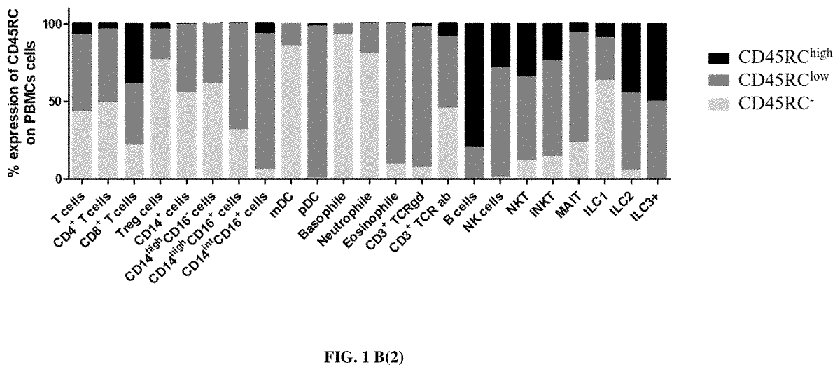

As shown in FIGS. 1A and B, a fraction of T CD4⁺ and T CD8⁺ cells is ABIS-45RC^{high} whereas the rest is ABIS-45RC^{low} or ABIS-45RC⁻. Most B and NK cells as well as pDCs were ABIS-45RC^{high} Most of NKT, iNKT MAIT, ILC2, ILC3 and of CD14^{int},CD16⁺ monocytes were ABIS-45RC^{high} or ABIS-45RC^{low}. CD14^{high}CD16⁻ monocytes, mDC, basophils and neutrophils were predominantly ABIS-45RC⁻. CD4⁺ Tregs and CD8⁺ Foxp3⁺ Tregs were largely ABIS-45RC^{low/-}.

The analysis of the major PBMCs populations showed that ABIS-45RC had a pattern of reactivity comparable to the commercial anti-CD45RC mouse MT2 antibody (FIG. 1 and Picarda et al., 2017. JCI Insight. 2(3):e90088).

### Example 2

Comparison of ABIS-45RC and the Commercial Anti-CD45RC Antibody “MT2”

Material and Methods

PBMC Isolation

Blood healthy volunteers is collected and peripheral blood mononuclear cells (PBMC) were isolated by Ficoll gradient centrifugation, which enables removal of unwanted fractions of blood products such as granulocytes, platelets and reaming red blood cell contaminants.

Antibodies and Flow Cytometry

Human PBMC were labeled with the ABIS-45RC antibody (at the indicated concentrations), an anti-CD3 antibody and an anti-CD45RC (mouse clone MT2, Biolegend)-FITC labeled at 1.33 mg/mL. The ABIS-45RC reactivity was revealed using a biotin donkey anti-human IgG⁺ Streptavidin PerCP-Cy 5.5 secondary antibody.

A Canto II cytometer (BD Biosciences) was used to measure fluorescence intensity and data were analyzed using the FLOWJO software (Tree Star Inc.). Cells were first gated by their morphology and dead cells were excluded by selecting DAPI-negative cells.

Cytotoxicity Analysis

Human PBMCs were incubated with medium at 37° C., isotype control antibody (Ms IgG1, clone 107.3, 10 μg/ml), ABIS-45RC or anti-CD45RC (mouse clone MT2) at 2.5 or 10 μg/ml for 10 minutes to 18 hours. Then, cells were stained with anti-CD3 (clone SK7, BD Biosciences), annexin-V, and DAPI. Percentage of apoptosis was obtained by gating on Annexin V⁺ and DAPI⁺ cells among T or non-T cells by flow cytometry.

Results

Both ABIS-45RC and Commercial Anti-CD45RC MT2 Antibodies Compete for the Same Epitope

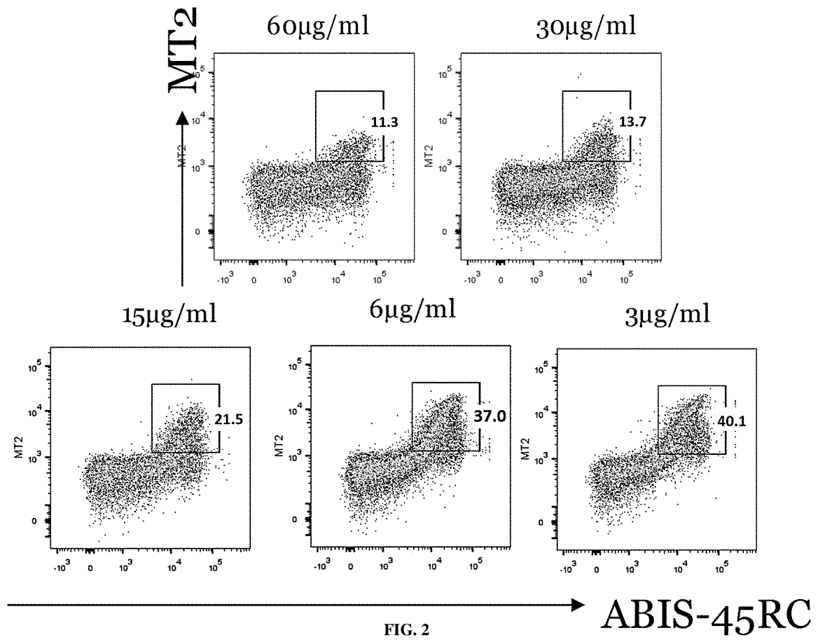

A shown in FIG. 2, co-labelling with the commercial anti-CD45RC MT2 clone showed that both antibodies competed and thus recognized the same or close epitope of human CD45RC.

Cytotoxicity Induced by ABIS-45RC is Higher Compared to Commercial Anti-CD45RC

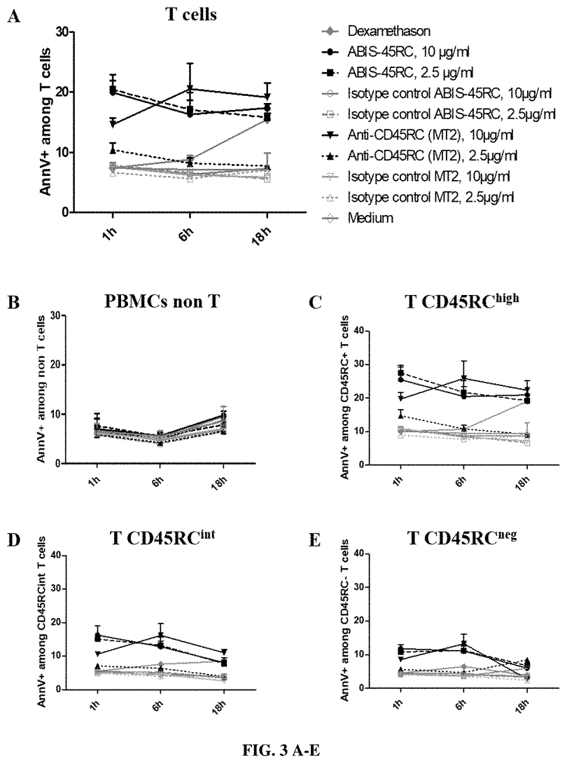

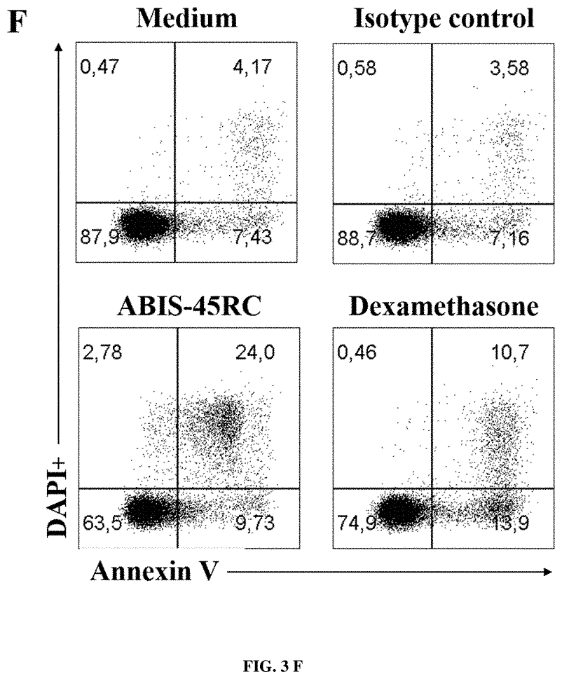

As shown in FIG. 3, ABIS-45RC was cytotoxic to T cells but not non-T cells.

Moreover, the T cells cytotoxicity was directly correlated to the level of CD45RC expression and importantly, ABIS-45RC performed better at 2.5 μg/mL as compared to the MT2 clone at 10 μg/mL.

### Example 3

Affinity of ABIS-45RC

Material and Methods

Briefly, 1×10⁷ CD45RC^{high} PBMCs or CHO cells expressing CD45RC after plasmid transfection were solubilized using the Mem-PER membrane isolation kit (Thermo-fisher). ABIS-45RC was immobilized on a biochip CM5 and cell membranes were incubated at 25° C. to measure affinity constants using single cycle kinetics and calibration free concentration analysis on a BIAcore 3000 and a BIAcore T200.

Results

Measurement of the affinity of CD45RC antibody was assessed by surface Plasmon Resonance (SPR), a technology for characterizing antibody-antigen interactions, and revealed an affinity (K_{D}) of 5×10⁻⁸ M, with a Kₒₙ of 2.91×10⁵ M⁻¹·sec⁻¹ and a K_{off} of 1.44×10⁻² sec⁻¹.

### Example 4

Treatment of Graft-Versus-Host-Disease (GVHD) with ABIS-45RC

Material and Methods

PBMC Isolation

Blood was collected at the Établissement Français du Sang (Nantes, France) from healthy individuals. Written informed consent was provided according to institutional guidelines. PBMC were isolated by Ficoll-Paque density-gradient centrifugation (Eurobio, Courtaboeuf, France). Remaining red cells and platelets were eliminated with a hypotonic solution and centrifugation.

Animals

8- to 12-week-old NOD/SCID/IL2Rγ^{/-} (NSG) mice were bred in our own animal facilities in SPF conditions (accreditation number C44-278).

GVHD Model

Adult NSG immunodeficient mice were whole-body sublethaly irradiated (irradiation dose of 2 Gy at day −1) to induce lesions in tissues that will favor the development of GVHD. The following day (day 0), 1.5×10⁷ PBMCs (including CD45RC^{high} and CD45RC^{low/-} T cells) from healthy volunteers were injected intravenously in these mice. Human PBMCs, and in particular T cells, react against and attack mouse tissues inducing lesions. These T cells and the lesions observed in liver, intestine, lungs and skin mimic the GVHD observed following bone marrow transplantation in humans or other GVHD experimental systems using rodents as donors and recipients. In particular, these tissue lesions typically induce a body weight loss that begins—depending on the number of PBMCs injected and in our experimental system—around day 13 after injection of the PBMCs. Body weight loss is monitored daily and animals are sacrificed when it drops to 20% of the original body weight to avoid unnecessary suffering.

Treatment

NSG mice were treated intraperitoneally with purified ABIS-45RC, with MT2 anti-CD45RC antibody or with an irrelevant control (an IVIg preparation used clinically comprising human purified IgG, and containing predominantly IgG1 antibodies) at 0.8 mg/kg from day 0 and every 2.5 days during 20 days.

NSG mice treated with ABIS-45RC or control antibodies also received intraperitoneally rapamycin from day 0 to day 10 at a suboptimal dose of 0.4 mg/day.

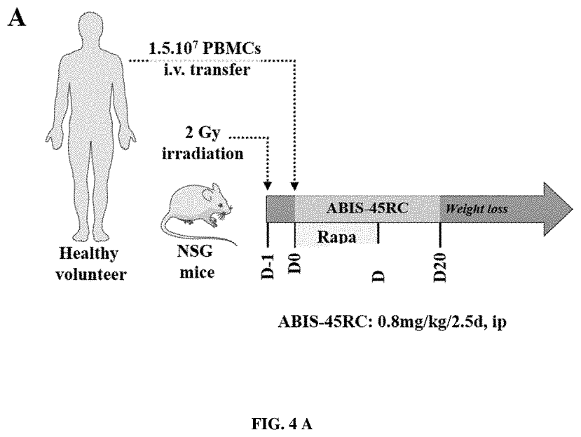

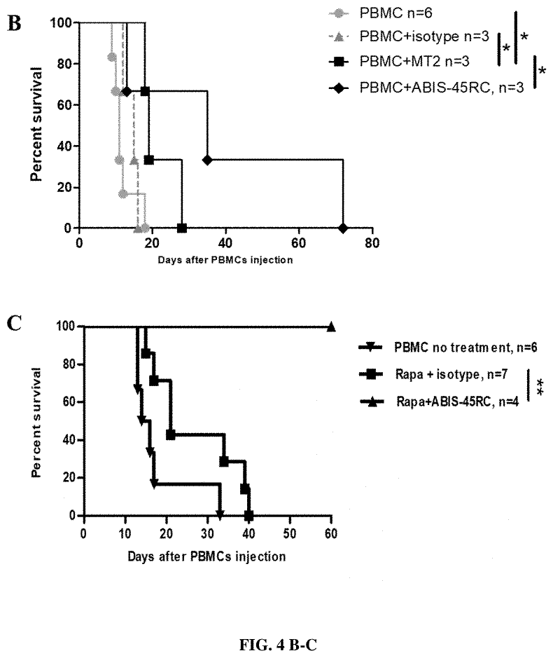

The experimental procedure is summarized in FIG. 4A.

Results

Treatment with PBMCs only induced weight loss, initiated around day 14, and, as shown in FIG. 4, death of all mice by day 33 (median survival: 11 days (FIG. 4B) to 15 days (FIG. 4C) days).

Treatment with control antibody and rapamycin only prolonged survival without reaching statistical significance (median survival: 21 days (FIG. 4C)).

While treatment with MT2 significantly prolonged survival of the mice (median survival: 19 days (FIG. 4B)), treatment with ABIS-45RC significantly increased this prolongation of survival of the mice up to 72 days (median survival: 35 days (FIG. 4B)).

Finally, combinatorial administration of ABIS-45RC with rapamycin completely prevented death as a consequence of GVHD (100% survival, FIG. 4C).

### Example 5

Humanization of ABIS-45RC

The design for the humanization of ABIS-45RC by grafting of the CDRs into human germline antibody sequences was undertaken. ABIS-45RC was humanized by grafting the three CDRs from the LCVR (with SEQ ID NOs: 15, 16 and 17) into a human germline LCVR that was as homologous as possible to ABIS-45RC's LCVR. Similarly, the three CDRs from the HCVR (with SEQ ID NOs: 1, 4 and 3) were grafted into a human germline HCVR that was as homologous as possible to ABIS-45RC's HCVR.

In addition, a few amino acid residues in the framework regions (FR) of the selected human germline variable regions were changed to the amino acid residues that were present in the murine variable regions (so called back-mutations). Based upon information on the structure of immunoglobulin variable regions, and with the guidance of an homology molecular model of the Fv of ABIS-45RC, these few residues in the FRs were identified as having key roles in either maintaining the CDRs in the right conformation or in HCVR/LCVR packing, and thus they were retained in a first humanized version (version A) or substituted with their human germline counterparts, if possible, in subsequent humanized version (versions B and C). Under guidance of the homology molecular model, in versions B and C, when judged possible, the CDR residues were also substituted for their human germline counterparts.

Homology Model Building

A model of ABIS-45RC was constructed according to established protocols (Ramos, 2012. Methods Mol Biol. 907:39-55).

Light Chain

In this section, unless specified otherwise, amino acid numbering is based on SEQ ID NO: 81.

The LCVR's framework residues were used to search the sequences of solved antibody structures via protein BLAST. The top hits were Protein Data Bank (PDB) ID: 4NCC (2.50 Å resolution) having 83 of 89 FR residues identical, and 85 out of 89 FR residues similar, to those of ABIS-45RC's LCVR, and PDB ID: 1QOK (2.40 Å resolution) having 83 of 87 framework residues identical, and 84 out of 87 similar, to those of ABIS-45RC's LCVR.

The sequences of these two structures both differed from that of ABIS-45RC's LCVR (with SEQ ID NO: 81) with the substitutions T9A, T39P, R44K, N49S and P54A. In addition, PDB ID: 4NCC differed in sequence from ABIS-45RC's LCVR with the substitutions L95F, A99G and L105I.

A comparison of the two structures showed high homology. However, the carbon chains adopted slightly different conformations in the regions A13-E17 and E104-K106.

Based upon the results of the subsequent CDR searches, and the presence of the two N-terminal residues, the LCVR of the structure of PDB ID: 4NCC was selected as the LCVR framework template, and the rotameric conformation of L105I was selected (in PyMol) with reference to PDB ID: 1QOK.

Subsequently, the sequences of ABIS-45RC LCVR's CDR1, CDR2 and CDR3, with the addition of two residues on each end, were used to search the sequences of solved antibody structures via protein BLAST.

For CDR1, the top hits consisted of a cluster of sequences having 9 out of 9 identical residues. Amongst these were PDB ID: 4NCC and PDB ID: 1QOK. Thus, the PDB ID: 4NCC structure was adopted as the template for CDR1.

For CDR2, the top hits consisted of a cluster of sequences having 6 out of 7 identical residues. Amongst these were again PDB ID: 4NCC and PDB ID: 1QOK. The PDB ID: 4NCC structure was therefore also adopted as the template for CDR2.

For CDR3, the top hit, containing no gaps, was PDB ID: 1QOK, having 13 out of 13 identical residues. PDB ID: 4NCC was however a close second, having 12 out of 13 identical residues. A comparison of the two structures showed essentially identical conformations, excepting for the L95F substitution. Thus, the PDB ID: 4NCC structure was adopted as the template for CDR3, and the rotameric conformation of L95F was selected (in PyMol) with reference to PDB ID: 1QOK.

It was thus not necessary to fit any CDR templates to the LCVR framework template because PDB ID: 4NCC was selected as the primary template for all of the LCVR's CDRs.

Finally, the LCVR partial model was manually subjected to mutagenesis at 8 positions (in PyMol), with selection of optimal rotamers, in order to match the ABIS-45RC LCVR sequence.

Heavy Chain

In this section, unless specified otherwise, amino acid numbering is based on SEQ ID NO: 61.

Next, the HCVR's framework residues were used to search the sequences of solved antibody structures via protein BLAST. The top hit was PDB ID: 30PZ (3.40 Å resolution), having 84 out of 90 framework residues identical, and 85 out of 90 similar, to those of ABIS-45RC's HCVR.

Since PDB ID: 30PZ was missing the N terminal residue, and was resolved with fairly poor resolution, additional hits with the highest identity/similarity scores were also surveyed. The top amongst these were PDB ID: 4CAD (2.50 Å resolution), having 78 out of 91 framework residues identical, and 87 out of 91 similar, to those of ABIS-45RC's HCVR; and PDB ID: 1RUR (1.50 Å resolution), having 75 out of 91 framework residues identical, and 87 out of 91 similar, to those of ABIS-45RC's HCVR.

A comparison of the PDB ID: 30PZ and PDB ID: 4CAD structures showed high homology with alternative residue rotamers being the principal differences.

A comparison of the PDB ID: 30PZ and PDB ID: 1RUR structures similarly showed high homology; however, there was a significant conformational change in the V_{H}-FR2 loop L45-G₄₉ relative to the PDB ID: 30PZ and PDB ID: 4CAD structures.

Further, based upon sequence, ABIS-45RC's HCVR and PDB ID: 4CAD were predicted to exhibit the Honegger Type III (Honegger & Pluckthun, 2001. J Mol Biol. 309(3):687-99) conformation of the N-terminal strand 5-12 because of the presence of a glutamine in position 6. However, PDB ID: 1RUR was predicted to exhibit the Honegger Type I conformation, due to the presence of a glutamic acid in position 6. Nevertheless, the three structures exhibited the identical conformation of the 5-12 strand. Also, the sequences of ABIS-45RC's HCVR, PDB ID: 4CAD and PDB ID: 1RUR were predicted to adopt the K-form (kinked base conformation) defined by the revised Shirai's rules for HCVR's CDR3 (Kuroda et al., 2008. Proteins. 73(3):608-20).

Based upon the results of the subsequent CDR searches, higher overall sequence similarity, structural concordance with PDB ID: 30PZ, and higher experimental resolution, the HCVR of the structure PDB ID: 1RUR was selected as the HCVR framework template; however, the 45-49 loop of PDB ID: 4CAD (having the same conformation as that of PDB ID: 30PZ) was substituted for that of PDB ID: 1RUR in the HCVR template using two residues N and C-terminal overhangs on the 45-49 ends to anchor the loop template fragment to the framework template.

Subsequently, the sequences of HCVR's CDR1, CDR2 and CDR3, with the addition of two residues on each end, were used to search the sequences of solved antibody structures via protein BLAST.

For CDR1, there was a cluster of sequence hits having 9 out of 12 identical residues. Amongst these was PDB ID: 1RUR. Thus, the PDB ID: 1RUR structure was selected as the template for CDR1.

For CDR2, the top hit was PDB ID: 3NTC (1.55 Å resolution) having 8 out of 12 residues identical, and 9 out of 12 similar, to those of ABIS-45RC's HCVR. However, PDB ID: 1RUR was a close second having 7 of 12 residues identical, and 9 out of 12 similar, to those of ABIS-45RC's HCVR. Comparison of the two structures showed essentially identical conformations, and the higher identity of PDB ID: 3NTC was due to 2 C terminal residues added to the CDR2 sequence for purposes of BLAST search. Thus, the PDB ID: 1RUR structure was preferred as the template for CDR2.

For CDR3, the top two hits, containing no gaps, were PDB ID: 1NGY (2.20 Å resolution) and PDB ID: 1NGZ (1.60 Å resolution), both having 8 out of 11 residues identical, and 9 out of 11 similar, to those of ABIS-45RC's HCVR. A comparison of the two structures showed a significantly different mainchain conformation. Without willing to be bound to a theory, the Inventors hypothesized that this difference might be due to the residue in position 101. In PDB ID: 1NGY, a larger methionine cannot adopt the orientation of the smaller serine of PDB ID: 1NGZ, which directs its sidechain into the core of the protein. Since the desired substitution to match the ABIS-45RC's HCVR sequence is F101, the PDB ID: 1NGY structure was adopted as the template for CDR3. Next, in order to complete the HCVR partial model, the CDR3 template was grafted onto the modified PDB ID: 1RUR HCVR template using the two residues overhang on its ends to anchor the CDR template fragment to the framework template (in PyMol).

Finally, the HCVR partial model was manually subjected to mutagenesis at 23 positions (in PyMol), with selection of optimal rotamers, in order to match the ABIS-45RC's HCVR sequence.

Final Model Assembly

Subsequently, the best tertiary arrangement of the HCVR and LCVR partial models were selected to assemble the final model. The HCVR and LCVR template sequences were submitted to the Packing Angle Prediction Server (PAPS) (Abhinandan & Martin, 2010. Protein Eng Des Sel. 23(9):689-97) to find a predicted best-fit tertiary arrangement. The PAPS server predicted that the solved antibody structure PDB ID: 1MNU, with a relative packing angle of −45.6°, would provide the best tertiary arrangement of HCVR and LCVR. Thus, the final model was assembled by fitting the backbone coordinates of the conserved anchor segments of the HCVR and LCVR partial models to PDB ID: 1MNU (in PyMol).

Lastly, the coordinates of this final model were subjected to a round of energy minimization employing GROMACS (Van der Spoel et al., 2005. J Comput Chem. 26(16):1701-18) with the GROMOS96 force-field (Scott et al., 1999. J Phys Chem A. 103(19):3596-3607).

Human Germlines

For the design of CDR-grafted versions of ABIS-45RC's HCVR and LCVR, two times three human germlines were selected:
IGHV1-2*01, IGHV5-51*01 and IGHV3-11*05 for the HCVR; and
IGKV1-9*01, IGKV6-21*02 and IGKV3-11*01 for the LCVR.

Design of humanized HCVR and LCVR The humanized versions A for both HCVR and LCVR are conservative versions that explicitly minimize and/or avoid alteration of CDR residues. These versions are thus expected to give a similar or better binding and/or potency activity as a chimeric antibody (ABIS-45RC's HCVR [SEQ ID NO: 61] and LCVR [SEQ ID NO: 81] fused to human constant regions [SEQ ID NOs: 91 and 92]).

The humanized versions B for both HCVR and LCVR are designed to reach a percentage of sequence identity with the closest human germline of at least 85%. This can be achieved by germlining (i.e., substituting the mouse residue with the corresponding human germline residue) FR and/or CDR amino acid residues. 85% is the cut-off percentage identity necessary to get the substem -zu- for “humanized”, denomination according to the 2014 World Health Organization (WHO) guidance on antibody International Nonproprietary Names (INN).

The humanized versions C for both HCVR and LCVR are designed to reach the highest degree of humanness (i.e., the highest degree of sequence identity with the corresponding human germline). Following inspection of the homology molecular model, a number of residues have been identified as candidates for germlining. Therefore, all the residues that could reasonably be germlined have been taken into consideration.

HCVR, Using IGHV1-2*01

To design humanized HCVR version A from IGHV1-2*01, the murine CDRs (with SEQ ID NOs: 1, 4 and 3) were grafted into IGHV1-2*01 and 4 residues in FR2 and FR3 were back-mutated to the parental murine residues, to maintain the full activity of the antibody. These residues are 148, L70, A72 and V97 in SEQ ID NO: 61. The resulting HCVR is as set forth in SEQ ID NO: 62 and shares 81.6% sequence identity with IGHV1-2*01 human germline.

To design humanized HCVR version B from IGHV1-2*01, in addition to version A, amino acid residues in the CDR2 were further germlined (i.e., substituted by the corresponding IGHV1-2*01 human germline residues). These residues are D56G, A58T, S60Y, N61A and K65Q in SEQ ID NO: 61. The resulting HCVR is as set forth in SEQ ID NO: 65 and shares 86.7% sequence identity with IGHV1-2*01 human germline.

To design humanized HCVR version C from IGHV1-2*01, in addition to version B, 2 amino acid residues in the CDR2 were further germlined. These residues are D50R and E62Q in SEQ ID NO: 61. The resulting HCVR is as set forth in SEQ ID NO: 68 and shares 88.8% sequence identity with IGHV1-2*01 human germline.

Various other humanized versions of the HCVR from IGHV1-2*01 were further designed, starting from version B. Indeed, to get a well humanized monoclonal antibody, 85% is supposed to be sufficient (version B from IGHV1-2*01 shares 86.7% sequence identity with IGHV1-2*01 human germline). In order to reduce the risk of introducing mutations, versions D, E, F, G and H were thus designed to reach 85% and no more.

The resulting HCVR versions D, E, F, G and H from IGHV1-2*01 are as set forth in SEQ ID NOs: 101, 121, 122, 123 and 124, respectively, and all share 85.7% sequence identity with IGHV1-2*01 human germline.

HCVR, Using IGHV5-51*01

To design humanized HCVR version A from IGHV5-51*01, the murine CDRs (with SEQ ID NOs: 1, 4 and 3) were grafted into IGHV5-51*01 and 6 residues in FR1, FR2 and FR3 were back-mutated to the parental murine residues, to maintain the full activity of the antibody. These residues are A24, T28, 148, L70, L83 and V97 in SEQ ID NO: 61. The resulting HCVR is as set forth in SEQ ID NO: 63 and shares 79.6% sequence identity with IGHV5-51*01 human germline.

To design humanized HCVR version B from IGHV5-51*01, in addition to version A, 1 amino acid residue in the FR1 and 6 amino acid residues in the CDR2 were further germlined. These residues are A24G, D56S, A58T, S60Y, N61S, K63S and K65Q in SEQ ID NO: 61. The resulting HCVR is as set forth in SEQ ID NO: 66 and shares 86.7% sequence identity with IGHV5-51*01 human germline.

To design humanized HCVR version C from IGHV5-51*01, in addition to version B, 1 amino acid residue in the FR1 and 2 amino acid residues in the CDR2 were further germlined. These residues are T28S, D50I and E62P in SEQ ID NO: 61. The resulting HCVR is as set forth in SEQ ID NO: 69 and shares 89.8% sequence identity with IGHV5-51*01 human germline.

HCVR, Using IGHV3-11*05

To design humanized HCVR version A from IGHV3-11*05, the murine CDRs (with SEQ ID NOs: 1, 4 and 3) were grafted into IGHV3-11*05 and 9 residues in FR1, FR2 and FR3 were back-mutated to the parental murine residues, to maintain the full activity of the antibody. These residues are Y27, T30, 148, G49, L70, A72, T74, A79 and V97 in SEQ ID NO: 61. The resulting HCVR is as set forth in SEQ ID NO: 64 and shares 76.5% sequence identity with IGHV3-11*05 human germline.

To design humanized HCVR version B from IGHV3-11*05, in addition to version A, 2 amino acid residues in the FR1, 6 amino acid residues in the CDR2 and 1 amino acid residue in the FR3 were further germlined. These residues are Y27F, T30S, D56S, A58T, S60Y, N61A, E62D, K63S and A79L in SEQ ID NO: 61. The resulting HCVR is as set forth in SEQ ID NO: 67 and shares 89.8% sequence identity with IGHV3-11*05 human germline.

To design humanized HCVR version C from IGHV3-11*05, in addition to version B, 1 amino acid residue in the FR2 and 1 amino acid residue in the CDR2 were further germlined. These residues are I48V and P53S in SEQ ID NO: 61. The resulting HCVR is as set forth in SEQ ID NO: 70 and shares 87.8% sequence identity with IGHV3-11*05 human germline.

LCVR, Using IGKV1-9*01

To design humanized LCVR version A from IGKV1-9*01, the murine CDRs (with SEQ ID NOs: 15, 16 and 17 with X₁₂ being absent) were grafted into IGKV1-9*01 and 3 residues in FR2 and FR3 were back-mutated to the parental murine residues, to maintain the full activity of the antibody. These residues are F35, W46 and Y70 in SEQ ID NO: 81. The resulting LCVR is as set forth in SEQ ID NO: 82 and shares 83.2% sequence identity with IGKV1-9*01 human germline.

To design humanized LCVR version B from IGKV1-9*01, in addition to version A, 1 amino acid residue in the CDR1 and 1 amino acid residue in the FR2 were further germlined. These residues are S24R and F35Y in SEQ ID NO: 81. The resulting LCVR is as set forth in SEQ ID NO: 85 and shares 85.3% sequence identity with IGKV1-9*01 human germline.

To design humanized LCVR version C from IGKV1-9*01, in addition to version B, 2 amino acid residues in the CDR2 and 1 amino acid residue in the FR3 were further germlined. These residues are N49A, P54Q and Y70F in SEQ ID NO: 81. The resulting LCVR is as set forth in SEQ ID NO: 88 and shares 88.4% sequence identity with IGKV1-9*01 human germline.

A LCVR version D from IGKV1-9*01 was further designed, in order to introduce an extra residue (Ser; S) in the CDR1 as found in the human germline IGKV1-9*01. The introduction of the extra residue in the CDR1 loop has shown that the binding activity was conserved (data not shown). The introduction of a serine residue in the CDR1 of version B brings the sequence identity to 86.3%, so in order to reduce the risk of introducing mutations, version D was designed where Kabat residue L36 was reverted to the original mouse residue Phe (F). The resulting LCVR is as set forth in SEQ ID NO: 103.

LCVR, Using IGKV6-21*02

To design humanized LCVR version A from IGKV6-21*02, the murine CDRs (with SEQ ID NOs: 15, 16 and 17 with X₁₂ being absent) were grafted into IGKV6-21*02 and 4 residues in FR2 and FR3 were back-mutated to the parental murine residues, to maintain the full activity of the antibody. These residues are F35, W46, Y48 and Y70 in SEQ ID NO: 81. The resulting LCVR is as set forth in SEQ ID NO: 83 and shares 81.1% sequence identity with IGKV6-21*02 human germline.

To design humanized LCVR version B from IGKV6-21*02, in addition to version A, 1 amino acid residue in the CDR1, 1 amino acid residue in the FR2, 1 amino acid residue in the CDR2 and 1 amino acid residue in the FR3 were further germlined. These residues are S24R, F35Y, L53S and Y70F in SEQ ID NO: 81. The resulting LCVR is as set forth in SEQ ID NO: 86 and shares 85.3% sequence identity with IGKV6-21*02 human germline.

To design humanized LCVR version C from IGKV6-21*02, in addition to version B, 1 amino acid residue in the CDR3 was further germlined. This residue is Q88H in SEQ ID NO: 81. The resulting LCVR is as set forth in SEQ ID NO: 89 and shares 86.3% sequence identity with IGKV6-21*02 human germline.

LCVR, Using IGKV3-11*01

To design humanized LCVR version A from IGKV3-11*01, the murine CDRs (with SEQ ID NOs: 15,16 and 17 with X₁₂ being absent) were grafted into IGKV3-11*01 and 3 residues in FR2 and FR3 were back-mutated to the parental murine residues, to maintain the full activity of the antibody. These residues are F35, W46 and Y70 in SEQ ID NO: 81. The resulting LCVR is as set forth in SEQ ID NO: 84 and shares 84.2% sequence identity with IGKV3-11*01 human germline.

To design humanized LCVR version B from IGKV3-11*01, in addition to version A, 1 amino acid residue in the CDR1 was further germlined. This residue is S24R in SEQ ID NO: 81. The resulting LCVR is as set forth in SEQ ID NO: 87 and shares 85.3% sequence identity with IGKV3-11*01 human germline.

To design humanized LCVR version C from IGKV3-11*01, in addition to version B, 1 amino acid residue in the FR2, 3 amino acid residues in the CDR2 and 1 amino acid residue in the FR3 were further germlined. These residues are F35Y, L53R, P54A, S55 and Y70F in SEQ ID NO: 81. The resulting LCVR is as set forth in SEQ ID NO: 90 and shares 90.5% sequence identity with IGKV3-11*01 human germline.

### Example 6

Production, Purification and Characterization of Humanized Anti-45RC Antibodies

Analytical size exclusion chromatography (SEC-HPLC) and differential scanning calorimetry (DSC) were used to compare the profile and the thermal stability, respectively, of 9 humanized anti-45RC variants A to I. These variants correspond to antibody comprising the “versions A” HCVR and LCVR described in Example 5. Analytical size exclusion chromatography (SEC-HPLC) and differential scanning calorimetry (DSC) were also used to compare the profile and the thermal stability, respectively, of 4 other humanized anti-45RC variants A1, A2, I1 and I2.

Material and Methods

SEC-HPLC

A Shimadzu Prominence HPLC system was used, with a Superdex 200 Increase 5/150 GL column (GE Healthcare). The column was previously calibrated in the same buffer and conditions used during sample analysis (using the Molecular Weight SEC Calibration kits from GE Healthcare, in PBS 1×, at 0.25 mL/min, with the column oven set to 30° C.).

All samples were centrifuged (20.000 g, 5 minutes, 4° C.) and had their protein content quantitated by Nanodrop ND-1000 spectrophotometer with IgG analysis program, prior to SEC analysis.

The isocratic program was set to inject about 15 μg of each sample, at 0.25 m/min during 18 minutes. After SEC analysis, 280 nm chromatogram was extracted from the raw data, and analyzed by peak integration.

DSC

A MicrocalTM VP-Capillary DSC system was used to perform differential scanning calorimetry experiments.

Samples in 1×PBS buffer were centrifuged (20.000 g, 5 minutes, 4° C.), and had their protein content quantitated Nanodrop ND-1000 spectrophotometer with IgG analysis program, prior to DSC analysis. Samples were then diluted in PBS to a final concentration of 1 mg/mL.

The pre-equilibration time was 3 minutes and the thermograms that followed were acquired between 20 and 110° C. with a scanning rate of 60° C./hour, a filtering period of seconds and medium feedback.

Prior to sample analysis, 5 buffer/buffer scans were measured to stabilize the instrument, and a buffer/buffer scan was performed between each protein/buffer scan.

The data was fitted to a non-2-state unfolding model, with the pre- and post-transition adjusted baseline subtracted. The calorimetric enthalpy (ΔH) is determined as the area under the peak of the transition, whereas the van′t Hoff enthalpy (ΔHv) is determined from the model used.

Results

SEC-HPLC

A summary of the SEC parameters is given in Table 7 below.

TABLE 7
SEC parameters of the humanized ABIS-45RC variants
A-I, A1, A2; I1 and I2.

| ABIS-45RC | | | | Area | Calculated |
|---|---|---|---|---|---|
| variant | Peak # | RT | Area | % | MW |
| A | 1 | 4.939 | 373336 | 10.62 | 510 |
| | 2 | 5.212 | 790998 | 22.50 | 404 |
| | 3 | 6.099 | 2351531 | 66.88 | 190 |
| B | 1 | 4.671 | 1305266 | 26.47 | 640 |
| | 2 | 5.013 | 1451918 | 29.44 | 478 |
| | 3 | 5.899 | 2174527 | 44.10 | 225 |
| C | 1 | 4.896 | 405089 | 14.04 | 529 |
| | 2 | 5.148 | 527782 | 18.29 | 426 |
| | 3 | 5.980 | 1788411 | 61.97 | 210 |
| | 4 | 7.708 | 164621 | 5.70 | 48 |
| D | 1 | 4.939 | 126757 | 5.38 | 510 |
| | 2 | 5.268 | 430061 | 18.25 | 385 |
| | 3 | 6.137 | 1799214 | 76.37 | 184 |
| E | 1 | 4.738 | 607732 | 18.87 | 605 |
| | 2 | 5.039 | 686150 | 21.31 | 468 |
| | 3 | 5.925 | 1925975 | 59.82 | 220 |
| F | 1 | 4.811 | 791680 | 13.93 | 568 |
| | 2 | 5.124 | 1306598 | 22.99 | 435 |
| | 3 | 5.977 | 3585434 | 63.08 | 211 |
| G | 1 | 4.768 | 185266 | 3.54 | 589 |
| | 2 | 5.092 | 889883 | 16.99 | 447 |
| | 3 | 5.893 | 4159958 | 79.46 | 226 |
| H | 1 | 4.896 | 105313 | 1.90 | 529 |
| | 2 | 5.158 | 506367 | 9.11 | 423 |
| | 3 | 5.962 | 4941812 | 88.99 | 213 |
| I | 1 | 5.142 | 123472 | 3.16 | 429 |
| | 2 | 6.081 | 3783288 | 96.84 | 193 |
| A1 | 1 | 5.074 | 129770 | 2.65 | 478 |
| | 2 | 6.098 | 4775239 | 97.35 | 189 |
| A2 | 1 | 5.074 | 129770 | 2.65 | 478 |
| | 2 | 6.098 | 4775239 | 97.35 | 189 |
| I1 | 1 | 4.885 | 131671 | 2.5 | 567 |
| | 2 | 5.146 | 355754 | 6.78 | 447 |
| | 3 | 6.142 | 4760685 | 90.7 | 181 |
| I2 | 1 | 4.864 | 32276 | 0.73 | 578 |
| | 2 | 5.150 | 168687 | 3.79 | 446 |
| | 3 | 6.181 | 4246352 | 95.48 | 175 |

RT: retention time (in minutes)
MW: molecular weight (in kDa)

Table 7 above shows in bold the peaks corresponding to the anti-CD45RC antibodies (peak 3 for each of variants A to H, I1 and I2; and peak 2 for each of variants I, A1 and A2), with RT and calculated MW expected for a monomeric, non-precipitated and non-dissociated antibody.

DSC

A summary of the DSC parameters is given in Table 8 below.

TABLE 8
DSC parameters of the humanized ABIS-45RC variants A-I, A1, A2;
I1 and I2. Denaturation of the antibody happens in two steps, hence two
melting temperatures are given, one of each step.

| ABIS-45RC variant | Conc. | T_{1/2} | ΔH | Tₒₙₛₑₜ | Tₘ₁ | Tₘ₂ |
|---|---|---|---|---|---|---|
| A | 0.0035 | 7.09 | 763 | 55.59 | 66.44 | 80.64 |
| B | 0.0056 | 7.09 | 776 | 55.96 | 64.73 | 81.01 |
| C | 0.0031 | 7.93 | 596 | 55.95 | 63.05 | 79.74 |
| D | 0.0023 | 5.83 | 863 | 60.09 | 70.91 | 80.10 |
| E | 0.0042 | 5.83 | 762 | 57.09 | 71.28 | 80.05 |
| F | 0.0099 | 5.41 | 867 | 58.63 | 71.15 | 80.34 |
| G | 0.0087 | 5.01 | 824 | 60.76 | 69.93 | 80.79 |
| H | 0.0093 | 4.59 | 877 | 59.79 | 68.14 | 80.24 |
| I | 0.0055 | 5.42 | 809 | 62.07 | 70.83 | 81.27 |
| A1 | 0.0049 | 3.32 | 969 | 61.09 | 68.17 | 81.91 |
| A2 | 0.0070 | 5.83 | 1150 | 60.21 | 66.92 | 81.94 |
| I1 | 0.0067 | 3.75 | 1080 | 63.08 | 73.08 | 81.84 |
| I2 | 0.0055 | 4.16 | 1030 | 63.68 | 72.43 | 82.02 |

Conc.: concentration, in mM.
T_{1/2}: width of transition at half height of the peak, in ° C.
ΔH: calorimetric enthalpy of unfolding, in cal/M.
Tₒₙₛₑₜ: temperature at which the unfolding transition begins, in ° C.
Tₘ₁: denaturing/melting temperature of the first step, in ° C.
Tₘ₂: denaturing/melting temperature of the second step, in ° C.

### Example 7

Reactivity of Humanized ABIS-45RC Variants A-I

Material and Methods

PBMC Isolation

Blood healthy volunteers is collected and peripheral blood mononuclear cells (PBMC) were isolated by Ficoll gradient centrifugation, which enables removal of unwanted fractions of blood products such as granulocytes, platelets and reaming red blood cell contaminants.

Antibodies and Flow Cytometry

Human PBMC were labeled with the murine ABIS-45RC antibody or each of the humanized ABIS-45RC antibodies variants A-I (at 2 μg/mL and 1 μg/mL); and an anti-CD3 antibody. The murine and humanized ABIS-45RC antibodies reactivity was revealed using a biotin donkey anti-human IgG⁺ Streptavidin PerCP-Cy 5.5 secondary antibody.

A Canto II cytometer (BD Biosciences) was used to measure fluorescence intensity and data were analyzed using the FLOWJO software (Tree Star Inc.). Cells were first gated by their morphology and dead cells were excluded by selecting DAPI-negative cells.

Results

Both ABIS-45RC and Commercial Anti-CD45RC MT2 Antibodies Compete for the Same Epitope

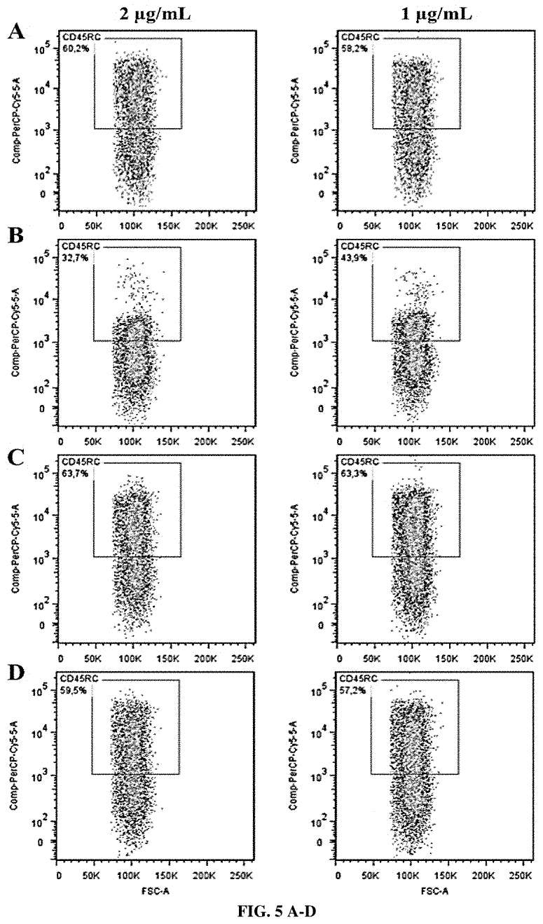

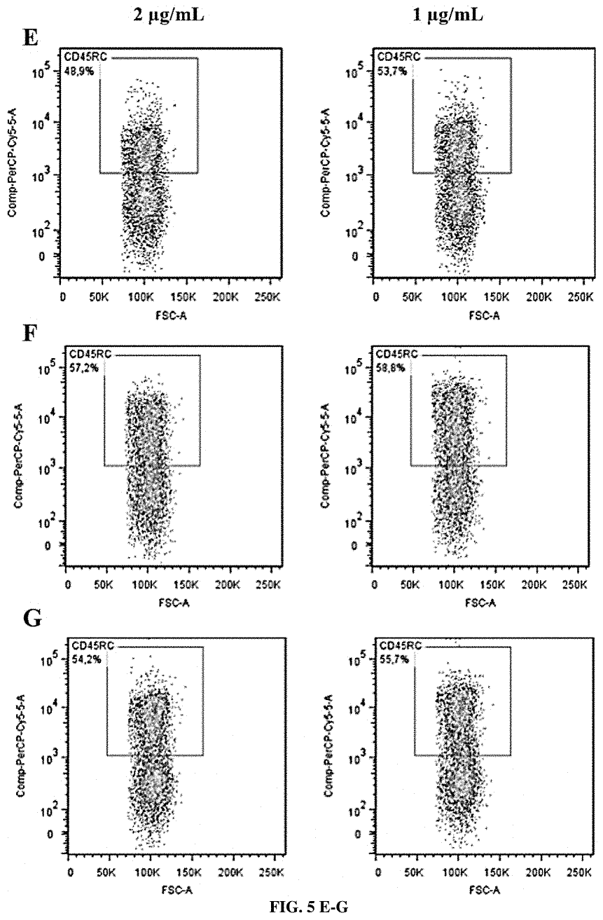

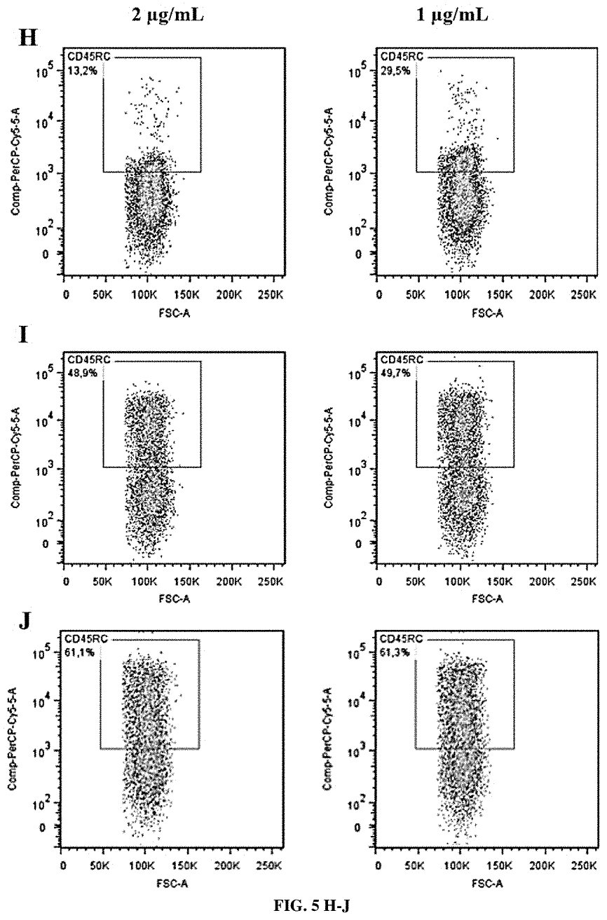

Labelling with either of the humanized ABIS-45RC antibodies (variant A, FIG. 5A; variant B, FIG. 5B; variant C, FIG. 5C; variant D, FIG. 5D; variant E, FIG. 5E; variant F, FIG. 5F; variant G, FIG. 5G; variant H, FIG. 5H; variant I, FIG. 5I) or the murine ABIS-45RC (FIG. 5J) shows that the antibodies recognized human CD45RC in a similar manner.

### Example 8

Engineered Antibodies

The CDR1 of ABIS-45RC's LCVR has a canonical structure unique to mouse antibodies, with a length of 10 amino acid residues (SEQ ID NO: 15 with X₁₂ being absent, i.e., SASSSVSYMH).

For the design of humanized versions A, B and C of the LCVR described in Example 5, this 10-amino-acid-residue CDR1 was grafted into the human germlines, with backmutation and/or germlining but no addition or deletion of any residue. However, in human germlines, the LCVR's CDR1 has a minimum length of 11 amino acid residues.

Therefore, to increase the humanness of the humanized antibodies, the Inventors have sought to engineer ABIS-45RC V_{L}-CDR1 “SASSSVSYMH” to extend it by one extra residue. One candidate position is position 8 in SEQ ID NO: 15 (designated as X₁₂), i.e., between S30 and Y31 in SEQ ID NO: 81. In all of the candidate germlines for the humanization design, this position is occupied with Asn (N), Ser (S) or Gly (G), while in the murine germline, this position is empty.

In order to investigate the structural relevance and stability of such insertion, ABIS-45RC V_{L}-CDR1 was expanded by insertion of an asparagine, i.e., SEQ ID NO: 15 with X₁₂ being Asn (N), i.e., SASSSVSNYMH. A search of the sequences of solved antibody structures via protein BLAST was then conducted. The top hit was the LCVR CDR1 of the structure PDB ID: 5CMA. Subsequently, this structural segment was grafted onto the ABIS-45RC model using the two-residue overhang on its ends to anchor the CDR template fragment to the model. It was observed that, in order to accommodate the additional residue, there was a conformational change that shifted the neighboring residue, thereby presenting a slight steric clash with Y70. However, in all of the human germlines, this residue is a more accommodating phenylalanine.

Engineered Mouse Antibody

Based on the above, the Inventors have engineered the ABIS-RC45 antibody, by inserting an asparagine residue (Asn, N) in the VL-CDR1, and further mutating Y70 of SEQ ID NO: 81 into a phenylalanine (Phe, F). The resulting “engineered Asn/Phe ABIS-RC45” LCVR is set forth in SEQ ID NO: 71, with X₁₂ being Asn (N).

Two other mouse antibodies have also been produced on the same basis, by inserting a serine residue (Ser, S) or a glycine residue (Gly, G) in the V_{L}-CDR1, and further mutating Y70 of SEQ ID NO: 81 into a phenylalanine (Phe, F). The two resulting “engineered Ser/Phe ABIS-RC45” and “engineered Gly/Phe ABIS-RC45” LCVR are set forth in SEQ ID NO: 71, with X₁₂ being Ser (S) or Gly (G), respectively.

Engineered Humanized Antibodies

Based on the above, engineered humanized LCVR versions A, B and C (as described in Example 5) can be further designed, as set forth in SEQ ID NOs: 72-80 where X₁₂ is Asn (N), Ser (S) or Gly (G) and the residue in position 70 is a Phe (F).

### Example 9

Reactivity of Engineered Asn/Phe ABIS-45RC

Material and Methods

Blood from healthy volunteers was collected and peripheral blood mononuclear cells (PBMC) were isolated by Ficoll gradient centrifugation, which enables removal of unwanted fractions of blood products such as granulocytes, platelets and reaming red blood cell contaminants.

Human PBMCs were labeled with ABIS-45RC or with engineered Asn/Phe ABIS-45RC and an anti-CD3 antibody. The reactivity was revealed using a biotin donkey anti-human IgG⁺ Streptavidin PerCP-Cy 5.5 secondary antibody.

A Canto II cytometer (BD Biosciences) was used to measure fluorescence intensity and data were analyzed using the FLOWJO software (Tree Star Inc.). Cells were first gated by their morphology and dead cells were excluded by selecting DAPI-negative cells.

Results

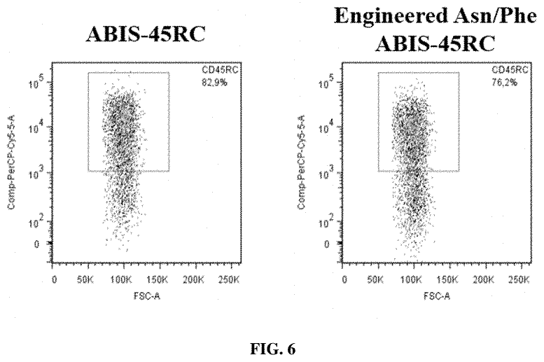

As shown in FIG. 6, labelling with either ABIS-45RC (left panel) or the engineered Asn/Phe ABIS-45RC (right panel) shows that both antibodies recognized human CD45RC in a similar manner.

### Example 10

Reactivity of Humanized ABIS-45RC

Material and Methods

Blood from healthy volunteers was collected and peripheral blood mononuclear cells (PBMC) were isolated by Ficoll gradient centrifugation, which enables removal of unwanted fractions of blood products such as granulocytes, platelets and reaming red blood cell contaminants.

Human PBMCs were labeled with ABIS-45RC or with humanized ABIS-45RC at 20, 5, 1.25 or 0.3 μg/mL and an anti-CD3 antibody. The reactivity was revealed using a biotin donkey anti-human IgG⁺ Streptavidin PercpCy 5.5 secondary antibody.

A Canto II cytometer (BD Biosciences) was used to measure fluorescence intensity and data were analyzed using the FLOWJO software (Tree Star Inc.). Cells were first gated by their morphology and dead cells were excluded by selecting DAPI-negative cells.

Results

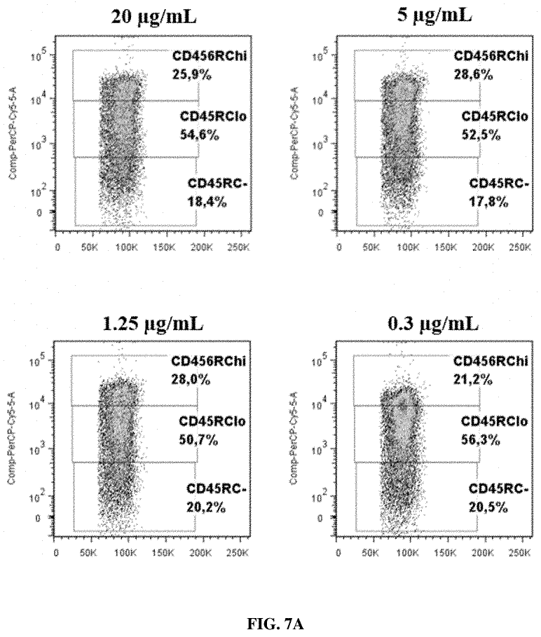

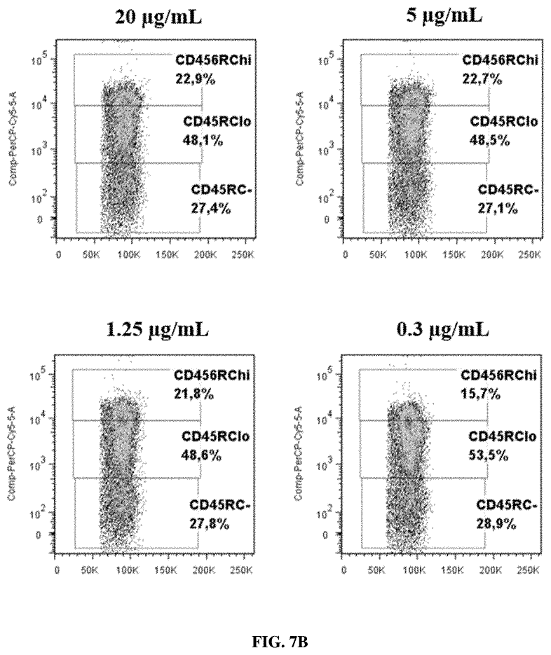

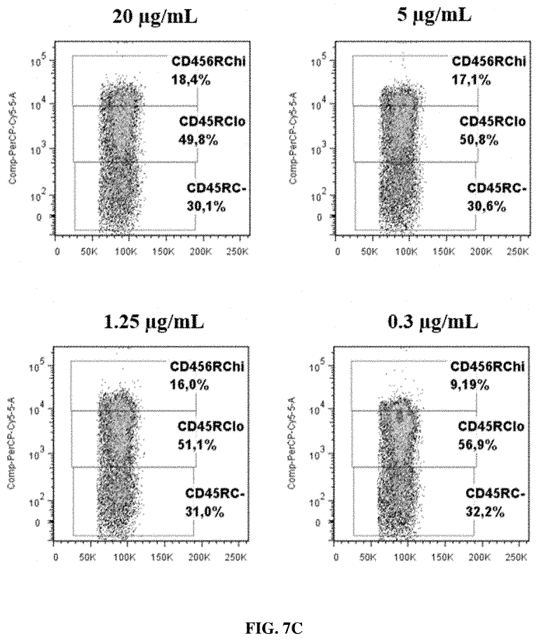

Labelling with either murine ABIS-45RC (FIG. 7A) or the humanized ABIS-45RC variant A1 (FIG. 7B) or variant A3 (FIG. 7C), at various concentrations, shows that both antibodies recognized human CD45RC in a similar manner.

### Example 11

Cell Death Induction by Humanized ABIS-45RC Variants

Material and Methods

Human PBMCs were incubated with medium, isotype control Ab or anti-CD45RC variants (10 μg/mL) for 6 hours. Then, cells were stained with anti-CD3 and anti-CD45RA, annexin V and DAPI. Percentage of total apoptosis was obtained by gating on DAPI⁺ annexin V⁺+DAPI⁻ Annexin V⁺ cells among T or non-T cells by flow cytometry.

Results

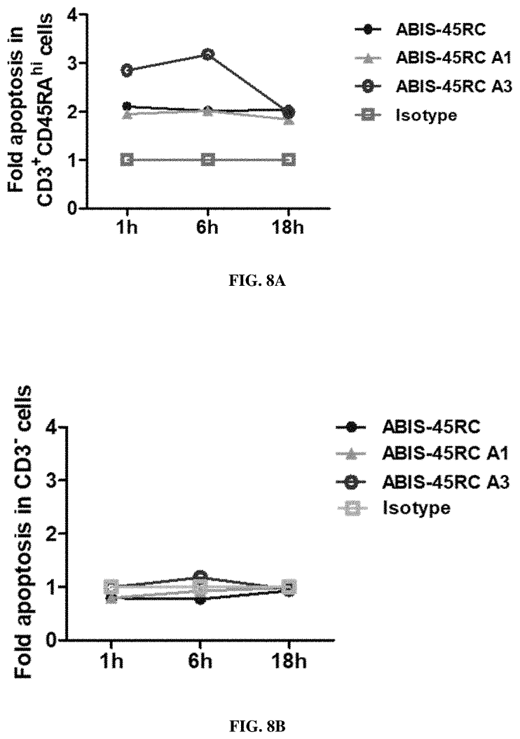

ABIS-45RC or the humanized variants A1 or A3 efficiently induced cell death of CD3⁺ cells (FIG. 8A) but not CD3⁻ cells (FIG. 8B).

### Example 12

Treatment of Human Skin Rejection with ABIS-45RC and Humanized Variant A1

Material and Methods

PBMC Isolation

Blood was collected at the Établissement Français du Sang (Nantes, France) from healthy individuals. Written informed consent was provided according to institutional guidelines. PBMC were isolated by Ficoll-Paque density-gradient centrifugation (Eurobio, Courtaboeuf, France). Remaining red cells and platelets were eliminated with a hypotonic solution and centrifugation.

Animals

8- to 12-week-old NOD/SCID/IL2Rγ^{/-} (NSG) mice were bred in our own animal facilities in SPF conditions (accreditation number C44-278).

Human Skin Transplantation Model

Human skins were obtained from healthy volunteers from abdominoplasty surgery and transplantation was performed as previously described (Bézie et al., 2018. Front Immunol. 8:2014). One month later, 5×10⁶ PBMCs from allogeneic healthy volunteers were intravenously injected with or without antibodies.

Graft rejection was scored from 0 to 5 based on dryness (score 1), rigidity (score 2), scab (score 3), partial loss (score 4) and complete loss of the skin (score 5) by macroscopic observation.

Human PBMCs engraftment was monitored in blood by flow cytometry.

Treatment

NSG mice were treated intraperitoneally with purified ABIS-45RC or humanized variant A1 antibodies at 0.8 mg/kg from day 0 and every 2.5 days during 20 days, together with intraperitoneal administration of rapamycin from day 0 to day 10 at a suboptimal dose of 0.4 mg/day.

Results

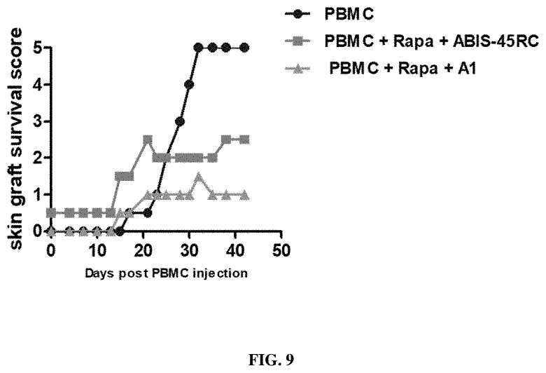

Treatment with PBMCs only induced weight loss, initiated around day 14, and, as shown in FIG. 9, death of all mice by day 33.

The inventors previously showed that treatment with rapamycin only did not prolonged survival (median survival: 21 days (Bézie et al., 2018. Front Immunol. 8:2014)). Here, treatment with ABIS-45RC or the humanized variant A1 completely abrogated the skin graft rejection.

### Example 13

Cell Engineering with the CD45RC-CAR

Material and Methods

Lentiviral Vectors

A self-inactivating second-generation lentiviral vector encoding for CAR-CD45R was generated. In this vector, the EF1alpha promoter controls the following sequences in the indicated order: a signal peptide, the variable regions of the heavy and light chains of the anti-CD45RC mAb (ABIS-45RC) fused by a linker, the transmembrane region of CD8-CD28 costimulatory signals region, the CD3zeta transducing signals region, P2 Å self-splicing sequences, GFP. The lentiviral vector was pseudotyped with VSV-G. Ctrl-CAR, used as a control, encodes the variable heavy and light chains of a monoclonal antibody having an antigenic specificity other than CD45RC (as well as the transmembrane region of CD8⁻ CD28 co-stimulatory signals region, and the CD3zeta transducing signals) under the control of the EF1alpha promoter and LNGFR under the control of a CMV promoter. This Ctrl-CAR lentiviral vector was also VSV-G pseudotyped.

Cell Transduction

HEK (293) or Jurkat cells were harvested and counted at day 1, then plated in 6 wells plate at 500 000 cells/well in 2 mL of DMEM 10% FBS, 10 μg/mL penicillin-streptomycin, 2 nM L-Glutamin. At day 2, the medium was removed by pipetting and 2 mL of fresh medium (pre-warmed at 37° C.) is added. In parallel, 500p L of the following preparation was added per well:
2.5 μg of DNA is diluted in 250 μL of Optimem (Gibco, life technology) during 5 min at room temperature (RT)
10 μL of lipofectamine TM 2000 (Life technology, Invitrogen) is dilute in 250 μL of Optimem during 5 min at RT
DNA and lipofectamine are mixed 20 min at RT.

GFP Detection

A Canto II cytometer (BD Biosciences) was used to measure fluorescence intensity and data were analyzed using the FLOWJO software (Tree Star Inc.). Cells were gated by their morphology and then dead cells were excluded by selecting DAPI⁻ negative cells. GFP was analyzed by flow cytometry after staining of the cells with Protein L (Genscript).

Results

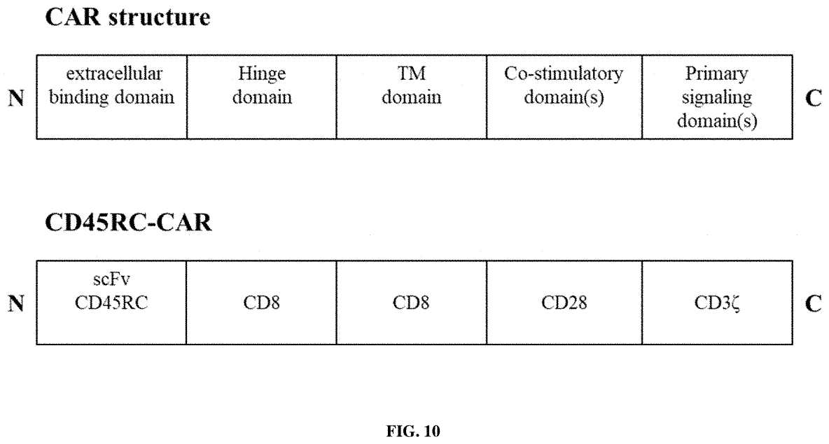

CAR were designed to have at least one extracellular domain, optionally a hinge domain, a transmembrane domain, at least one co-stimulatory domain and at least one primary signaling domain (FIG. 10). One CAR construct of the invention is a CAR harboring an scFv CD45RC, a CD8α hinge domain and transmembrane domain, a CD28 co-stimulatory domain and a CD3ζ primary signaling domain. Optionally, GFP coding sequences may be present immediately 3′ of the CD45RC-CAR sequences separated from them by a T2 Å self-splicing sequence (not drawn). In this case, GFP may be used as a surrogate marker of CAR-CD45RC expression.

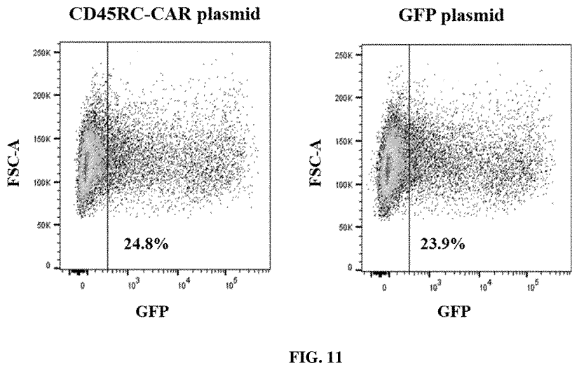

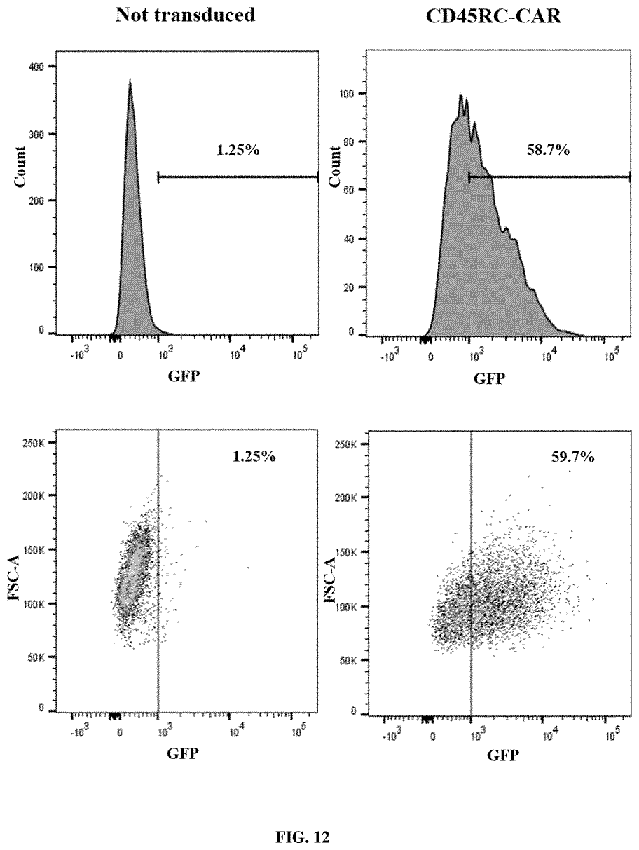

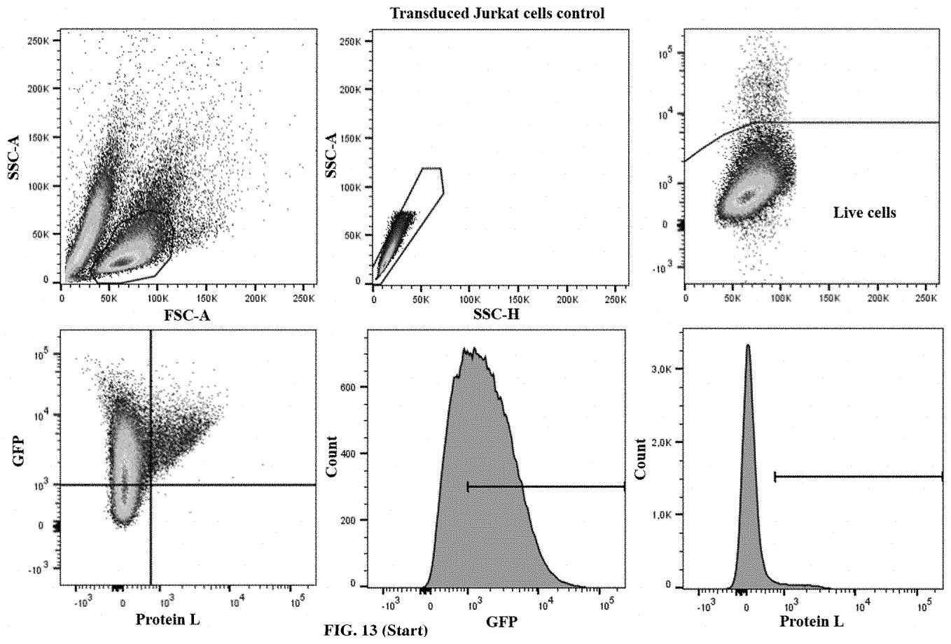

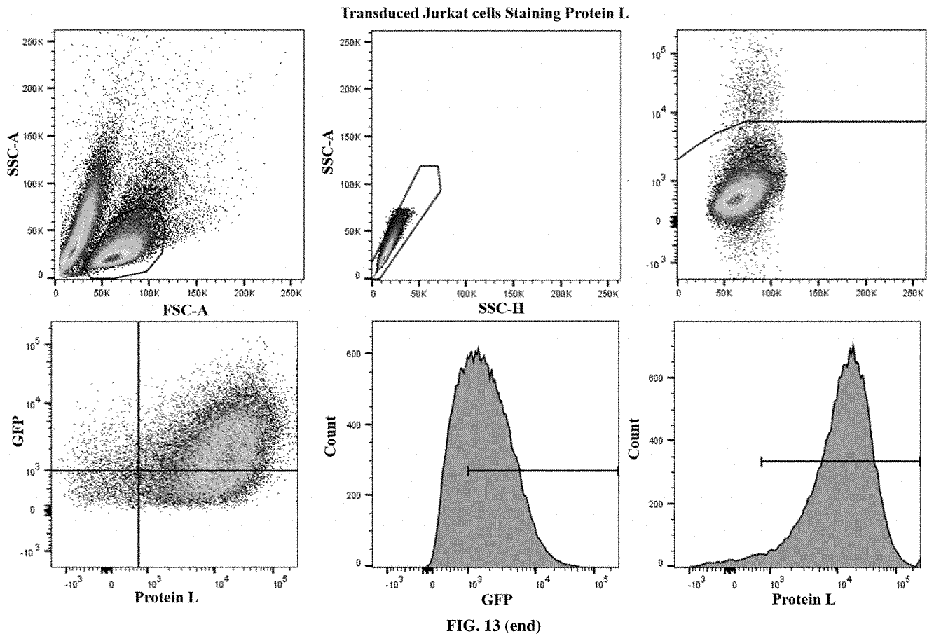

The inventors showed that HEK cells are able to be transfected with the CD45RC-CAR as compared to a GFP mock plasmid as shown by the GFP staining (FIG. 11). In addition, this CAR construction can also be transduced with lentiviral vectors in a human immortalized T-cell cell line (Jurkat). Indeed, the level of GFP, showing the presence of the CAR is of 58.7% in Jurkat cells transduced with the CD45RC CAR and only of 1.25% in Jurkat cells non transduced (FIG. 12). In particular, the surface expression of the CAR in Jurkat cells was confirmed by Protein L staining and demonstrated a good correlation with GFP staining, demonstrating altogether that the lentivirus was functional (FIG. 13).

### Example 14

CD45RC-CAR Induction of Target T Cell Apoptosis

Material and Methods

Total T cells were negatively sorted using magnetic sorting (Miltenyi Biotech) from human PBMC. Cells were labelled with CPD 670 and plated in 96 wells bottom V plates (20,000 cells per well in complete medium RPMI (10% SVF, amino acid, penicillin/streptomycin, glutamine, sodium pyruvate, HEPES). 100,000 Jurkat cells were plated in 96-well bottom plate in complete DMEM medium and transduced with 10 uL of the CD45RC-CAR or Ctrl-CAR control lentiviral vectors described in Example 13, incubated 2 days at 370° C. Jurkat cells were then counted and added to T cells at different ratios of T:Jurkat cells in complete RPMI medium (ratio 1:0-1:1-1:5-1:10). The cells were incubated 18h at 37° C. After incubation, the cells were marked with annexin V in annexin buffer for 20 min. The DAPI was added in the annexin buffer and cells were directly analyzed in a Canto II cytometer (BD Biosciences).

Results

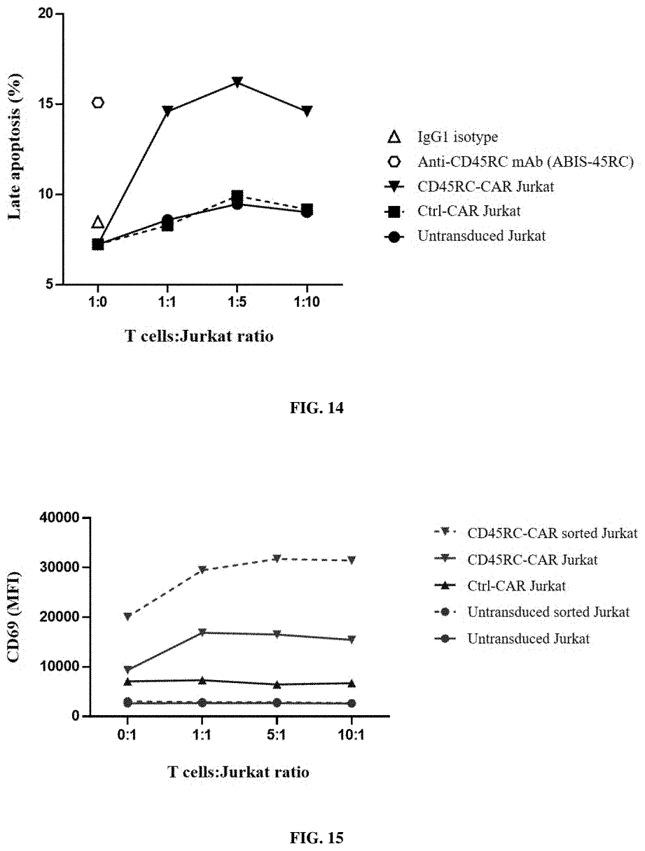

Human T cells were cultured during 18 hours in presence of Jurkat cells previously transduced with a CD45RC-CAR of a Ctrl-CAR and then apoptosis was evaluated by flow cytometry. In presence of Jurkat cells harboring the CD45RC-CAR, T cells were 15% apoptotic and only 7-8% in Ctrl-CAR or untransduced cells (FIG. 14). The level of apoptosis observed was equivalent to the level obtained with an anti-CD45RC antibody. Thus, Jurkat cells expressing the CD45RC-CAR can induce apoptosis in human T cells.

### Example 15

CD45RC-CAR Induction of T Cell Activation

Material and Methods

Total T cells were negatively sorted using magnetic sorting (Miltenyi Biotech) from human PBMC. Cells were labelled with CPD 670 and plated in 96 wells bottom V plates (20,000 cells per well in complete medium RPMI (10% SVF, amino acid, penicillin/streptomycin, glutamine, sodium pyruvate, HEPES). 100,000 Jurkat cells were plated in 96-well bottom plate in complete DMEM medium and transduced with 10 uL of the CD45RC-CAR or Ctrl-CAR control lentiviral vectors described in Example 13, incubated 2 days at 37° C. Jurkat cells were then counted and added to T cells at different ratios of T: Jurkat cells in complete RPMI medium (ratio 1:0-1:1-1:5-1:10). The cells were incubated 18h at 37° C. After incubation, the cells were marked with an anti-CD69 mAb for 20 min. The DAPI was added and the cells were directly analyzed in a Canto II cytometer (BD Biosciences).

Results

Human T cells were cultured during 18 hours in presence of Jurkat cells transduced with a CD45RC-CAR or a Ctrl-CAR and then T cell activation was measured by flow cytometry using the CD69 activation marker.

The inventors observed that the CD45RC-CAR induce a T cells activation (as shown by CD69 expression) compared to Ctrl-CAR and untransduced Jurkat cells. Interestingly, when the cells are sorted, meaning that the CD45RC-CAR is expressed by all cells, the activation is even better compared to non-sorted CD45RC-CAR Jurkat cells (FIG. 15). Altogether, this shown that the CAR is functional.

### Example 16

CD45RC-CAR Induction of CD4⁺ Treg Activation and of Target T Cell Apoptosis

Material and Methods

CD4⁺ Treg Lentiviral Transduction and Expansion Protocol

At day 0, CD4⁺ CD127^{1ow} CD25⁺ CD45RC⁻ Tregs were sorted on a FACSAria™ and seeded at 10⁵ cells in 100 μL medium per in 96-well flat bottom plates previously coated with 1 μg/mL anti-CD3 monoclonal antibodies (clone OKT3). Medium used at day 0 was RPMI 1640 supplemented with penicillin, streptomycin, sodium pyruvate, HEPES buffer, amino acids, glutamine, 5% CTS serum, anti-CD28 mAbs (clone CD28.2 at 1 μg/mL) and 1000 U/mL IL-2. At day 1 and day 2, cells were transduced with 10 μL of the lentiviral vectors described in Example 13, which code for anti-CD45RC chimeric antigen receptor (CD45RC-CAR) and GFP, or for a control CAR having a different antigenic specificity (Ctrl-CAR) and LNGFR. At day 3, medium was added to reach a 10% CTS serum final concentration. At day 7, cells were harvested and sorted on CAR expression based on GFP or LNGFR and then newly stimulated with anti-CD3 and anti-CD28 mAbs for a second round of 7 days of expansion. Cytokines were freshly added in culture medium every 2 days, and fresh medium was added when required.

CD45RC-CAR Detection

HEK 293T cells were also transduced with the lentivirus encoding for CD45RC-CAR once at day 1. CD4⁺ Tregs were transduced with lentivirus as explained above. HEK 293T and CD4⁺ Tregs were analyzed 5 days after the last transduction. Cells were treated with human Fc block (BD Biosciences) and then incubated with 2 μg/mL of CD45R-ABC protein (R&D) biotinylated in house diluted in PBS BSA 1% for 1 h at 4° C. CD45R-ABC-biotin protein was revealed with streptavidin-APC-Cy7 at 4 μg/mL. DAPI was added to exclude dead cells and the cells were analyzed in a FACSCanto™.

CAR-Mediated Activation Assay

A total of 10⁵ CD45RC-CAR CD4⁺ Tregs or Ctrl-CAR CD4⁺ Tregs were plated in 96-wells U-bottom plates previously coated with CD45R-ABC protein (1 μg/mL in PBS, 1h30 at 37° C.) in complete medium RPMI (10% CTS serum, amino acid, penicillin/streptomycin, glutamine, sodium pyruvate, HEPES). The cells were incubated 24h at 37° C. and brefeldin A was added the 4 last hours of culture. After incubation, the cells were marked with a viability dye and stained extracellularly with anti-CD69, anti-CD25, anti-CD71 mAbs for 30 min. Cells were fixed and permeabilized and stained intracellularly with anti-CTLA-4 mAb for 1 h. Cells were analyzed in the FACSCanto™.

Apoptosis Assay

Human PBMC were labelled with CPD670 and plated in 96 wells V-bottom plates (50,000 cells per well) in complete medium RPMI (10% human AB serum, amino acid, penicillin/streptomycin, glutamine, sodium pyruvate, HEPES). CD4⁺ Tregs non-transduced or transduced with CD45RC-CAR or Ctrl-CAR were counted after 15 days of expansion and added to allogenic PBMC at different ratios of PBMC:Tregs in complete RPMI medium (ratio 1:0-1:1-1:2-1:5). The cells were incubated 4h at 37° C. After incubation, cells were stained with anti-CD3, anti-CD19, anti-CD14, anti-CD56 and anti-CD45RA mAbs and then cells were marked with annexin V in annexin buffer for 20 min. DAPI was added in the annexin buffer and the cells were rapidly analyzed in the FACSCanto™. Anti-CD45RA mAb was used as surrogate markers for identification of CD45RC^{high} cells in each cell subset. As a control of apoptosis, ABIS-45RC was incubated with the PBMCs at 10 μg/mL. The percentage of total apoptotic cells was calculated by the sum of annexin⁺ DAPI⁺ cells and annexin⁺ DAPI⁻ cells.

Results

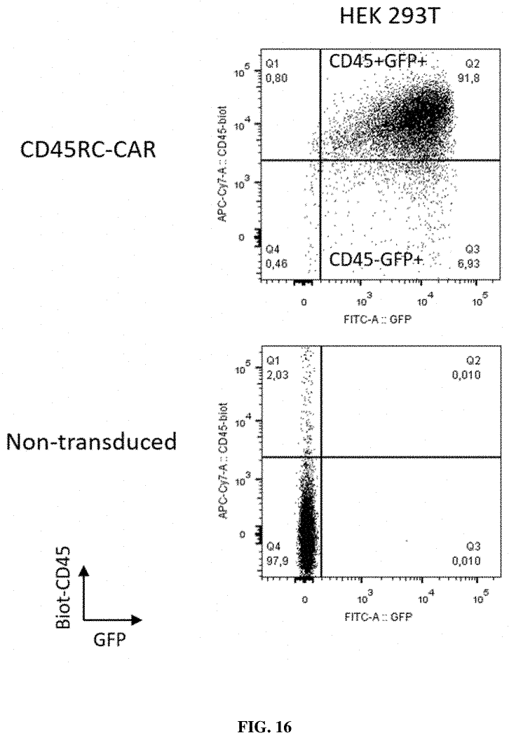

Human HEK 293 were transduced with a lentiviral vector encoding for CD45RC-CAR and GFP and analyzed by cytofluorimetry (FIG. 16). CD45RC-CAR detection using a CD45R-ABC protein labeled with biotin and revealed with streptavidin-APC-Cy7 showed a clear positive signal and co-expression with GFP as compared to non-transduced cells, demonstrating that the CD45RC-CAR is expressed and functional in its specificity to CD45RC.

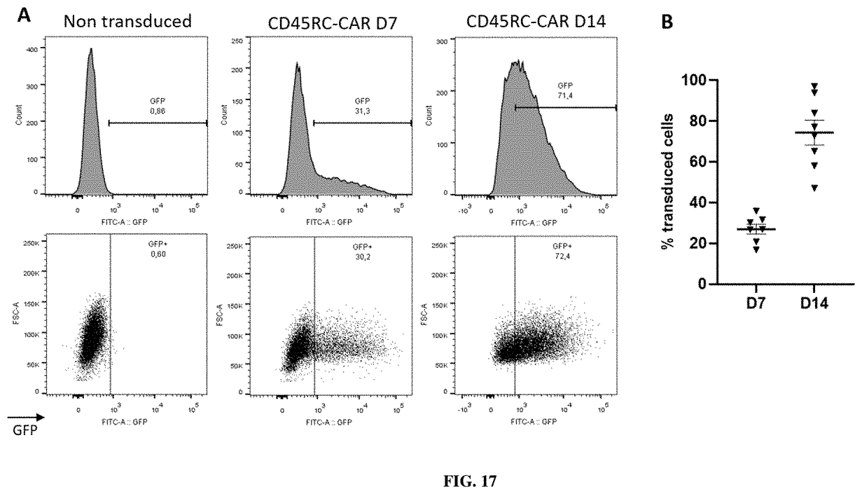

Transduction of CD4⁺ Tregs with CD45RC-CAR and cell sorting selection using GFP at day 7 followed by in vitro expansion during 14 days allowed obtention of a population of 74.38%+/−17.3% of transduced cells (FIG. 17), demonstrating that CD4⁺ Tregs can be transduced and that CD45RC-CAR CD4⁺ Tregs can be expanded.

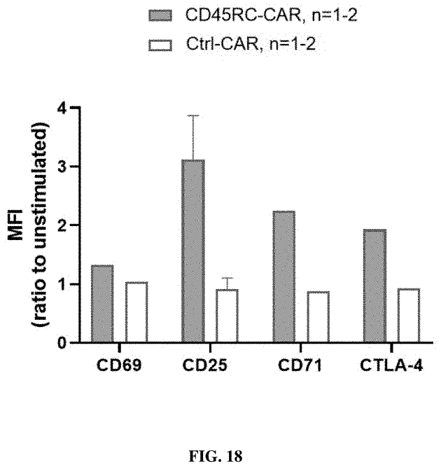

The inventors then analyzed the activation of CD45RC-CAR-transduced and expanded CD4⁺ Tregs by incubating the cells with CD45R-ABC protein and analyzing activation markers by cytofluorimetry (FIG. 18). They observed in CD4⁺ Tregs transduced by CD45RC-CAR, but not by Ctrl-CAR, increased protein expression of CD69, CD25, CD71 and CTLA-4 upon incubation with CD45R-ABC protein for 24h, demonstrating that the CAR is signaling and directly activating the Tregs.

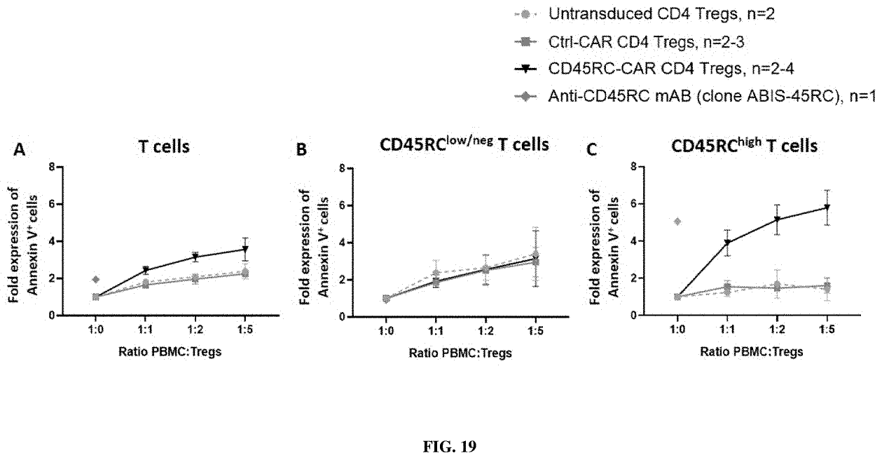

Apoptosis on different cell populations of PBMC by allogenic CAR-transduced or not transduced CD4⁺ Tregs after 14 days of expansion was analyzed and compared to the apoptosis obtained with the anti-CD45RC mAb used to generate the CD45RC-CAR (FIG. 19). The inventors observed that CD45RC-CAR-transduced CD4⁺ Tregs induced apoptosis of CD45RC^{high} T cells equivalent to the anti-CD45RC mAb, in contrast to Ctrl-CAR and non-transduced CD4⁺ Tregs that did not induce apoptosis. This apoptotic effect of CD45RC-CAR CD4⁺ Tregs was dose dependent, increasing with higher ratios of PBMCs:CD4⁺ Tregs.

Altogether, these results showed that the CD45RC-CAR is functional since it induced activation of CD45RC-CAR-transduced CD4⁺ Tregs. Moreover, CD45RC-CAR-transduced CD4⁺ Tregs induced apoptosis of CD45RC^{high} T cells.

1.-34. (canceled)

35. A chimeric antigen receptor (CAR) specific for human CD45RC, wherein said CAR comprises:
(a) at least one extracellular binding domain, wherein said binding domain binds to said human CD45RC,
(b) optionally at least one extracellular hinge domain,
(c) at least one transmembrane domain, and
(d) at least one intracellular signaling domain, wherein the intracellular domain comprises at least one T cell primary signaling domain and optionally at least one T cell costimulatory signaling domain.

36. The CAR according to claim 35, wherein the extracellular binding domain comprises at least one antigen-binding fragment that binds to human CD45RC comprising:
(a) a HCVR which comprises the following three CDRs:
(i) V_{H}-CDR1 of sequence SEQ ID NO: 1;
(ii) V_{H}-CDR2 with a sequence selected from the group comprising sequences SEQ ID NOs: 4, 5, 6, 8, 100, 116, 117, 118 and 119; and
(iii) V_{H}-CDR3 of sequence SEQ ID NO: 3; and
(b) a LCVR which comprises the following three CDRs:
(i) V_{L}-CDR1 with a sequence selected from the group comprising sequences SEQ ID NO: 15 (SASSSVS-X₁₂-YMH) and 18 (RASSSVS-X₁₂-YMH), wherein Xu is absent or is selected from Asn (N), Ser (S) and Gly (G);
(ii) VL-CDR2 with a sequence selected from the group comprising sequences SEQ ID NO: 16, 111, and 120; and
(iii) V_{L}-CDR3 of sequence SEQ ID NO: 17.

37. The CAR according to claim 35, wherein the extracellular binding domain comprises at least one antigen-binding fragment that binds to human CD45RC comprising:
(a) a HCVR which comprises the following three CDRs:
(i) V_{H}-CDR1 of sequence SEQ ID NO: 1;
(ii) V_{H}-CDR2 with a sequence selected from the group comprising sequences SEQ ID NOs: 4 and 5; and
(iii) V_{H}-CDR3 of sequence SEQ ID NO: 3; and
(b) a LCVR which comprises the following three CDRs:
(i) V_{L}-CDR1 of sequence SEQ ID NO: 15, wherein X₁₂ is absent;
(ii) V_{L}-CDR2 of sequence SEQ ID NO: 16; and
(iii) V_{L}-CDR3 of sequence SEQ ID NO: 17.

38. The CAR according to claim 35, wherein the extracellular binding domain comprises at least one antigen-binding fragment that binds to human CD45RC comprising:
(a) a HCVR which comprises the following three CDRs:
(i) V_{H}t-CDR1 of sequence SEQ ID NO: 1;
(ii) V_{H}-CDR2 of sequence 4; and
(iii) V_{H}-CDR3 of sequence SEQ ID NO: 3; and
(b) a LCVR which comprises the following three CDRs:
(i) V_{L}-CDR1 of sequence SEQ ID NO: 15, wherein X₁₂ is absent;
(ii) V_{L}-CDR2 of sequence SEQ ID NO: 16, and
(iii) V_{L}-CDR3 of sequence SEQ ID NO: 17.

39. The CAR according to claim 35, wherein the extracellular binding domain comprises at least one antigen-binding fragment that binds to human CD45RC comprising:
(a) a HCVR which comprises the following three CDRs:
(i) V_{H}t-CDR1 of sequence SEQ ID NO: 1;
(ii) V_{H}-CDR2 with a sequence selected from the group comprising sequences SEQ ID NOs: 4, 6, and 100; and
(iii) V_{H}-CDR3 of sequence SEQ ID NO: 3; and
(b) a LCVR which comprises the following three CDRs:
(i) V_{L}-CDR1 with a sequence selected from the group comprising sequences SEQ ID NOs: 15 and 18, wherein X₁₂ is absent:
(ii) V_{L}-CDR2 with a sequence selected from the group comprising sequences SEQ ID NO: 16, 111, and 120; and
(iii) V_{L}-CDR3 of sequence SEQ ID NO: 17.

40. The CAR according to claim 35, wherein the extracellular binding domain comprises at least one antigen-binding fragment that binds to human CD45RC comprising:
1) a HCVR of sequence SEQ ID NO: 61 and a LCVR of sequence SEQ ID NO: 81;
2) a HCVR of sequence SEQ ID NO: 62 and a LCVR of sequence SEQ ID NO: 82;
3) a HCVR of sequence SEQ ID NO: 62 and a LCVR of sequence SEQ ID NO: 83;
4) a HCVR of sequence SEQ ID NO: 62 and a LCVR of sequence SEQ ID NO: 84:
5) a HCVR of sequence SEQ ID NO: 63 and a LCVR of sequence SEQ ID NO: 82;
6) a HCVR of sequence SEQ ID NO: 63 and a LCVR of sequence SEQ ID NO: 83;
7) a HCVR of sequence SEQ ID NO: 63 and a LCVR of sequence SEQ ID NO: 84:
8) a HCVR of sequence SEQ ID NO: 64 and a LCVR of sequence SEQ ID NO: 82;
9) a HCVR of sequence SEQ ID NO: 64 and a LCVR of sequence SEQ ID NO: 83;
10) a HCVR of sequence SEQ ID NO: 64 and a LCVR of sequence SEQ ID NO: 84;
11) a HCVR of sequence SEQ ID NO: 101 and a LCVR of sequence SEQ ID NO: 85;
12) a HCVR of sequence SEQ ID NO: 101 and a LCVR of sequence SEQ ID NO: 103;
13) a HCVR of sequence SEQ ID NO: 65 and a LCVR of sequence SEQ ID NO: 85;
14) a HCVR of sequence SEQ ID NO: 65 and a LCVR of sequence SEQ ID NO: 103;
15) a HCVR of sequence SEQ ID NO: 62 and a LCVR of sequence SEQ ID NO: 85;
16) a HCVR of sequence SEQ ID NO: 101 and a LCVR of sequence SEQ ID NO: 82;
17) a HCVR of sequence SEQ ID NO: 121 and a LCVR of sequence SEQ ID NO: 85;
18) a HCVR of sequence SEQ ID NO: 122 and a LCVR of sequence SEQ ID NO: 85;
19) a HCVR of sequence SEQ ID NO: 123 and a LCVR of sequence SEQ ID NO: 85;
20) a HCVR of sequence SEQ ID NO: 124 and a LCVR of sequence SEQ ID NO: 85;
21) a HCVR of sequence SEQ ID NO: 63 and a LCVR of sequence SEQ ID NO: 85;
22) a HCVR of sequence SEQ ID NO: 67 and a LCVR of sequence SEQ ID NO: 85;
23) a HCVR of sequence SEQ ID NO: 67 and a LCVR of sequence SEQ ID NO: 103;
24) a HCVR of sequence SEQ ID NO: 61 and a LCVR of sequence SEQ ID NO: 113;
25) a HCVR of sequence SEQ ID NO: 61 and a LCVR of sequence SEQ ID NO: 126; or
26) a HCVR and a LCVR comprising a sequence of the non-CDR regions sharing at least 70% of identity with the sequence of the non-CDR regions of the HCVR and LCVR according to 1) to 23).

41. The CAR according to claim 35, wherein the extracellular binding domain comprises at least one antigen-binding fragment that binds to human CD45RC comprising:
(a) a HCVR which comprises the following three CDRs:
(i) V_{H}-CDR1 of sequence SEQ ID NO: 1;
(ii) VH-CDR2 with a sequence selected from the group comprising sequences SEQ ID NOs: 4, 5, 6, 8, 100, 116, 117, 118 and 119; and
(iii) V_{H}-CDR3 of sequence SEQ ID NO: 3; and
(b) a LCVR which comprises the following three CDRs:
(i) V_{L}-CDR1 with a sequence selected from the group comprising sequences SEQ ID NOs: 15 and 18, wherein X₁₂ in SEQ ID NOs: 15 and 18 is selected from Asn (N), Ser (S) and Gly (G);
(ii) V_{L}-CDR2 of sequence SEQ ID NO: 16; and
(iii) V_{L}-CDR3 of sequence SEQ ID NO: 17.

42. The CAR according to claim 41, wherein the amino acid residue at Kabat position L71 of the LCVR is Phe (F).

43. The CAR according to claim 35, comprising:
(i) an anti-human CD45RC scFv,
(ii) a hinge domain derived from CD8α,
(iii) a human CD8α transmembrane domain, and
(iv) an intracellular signaling domain comprising a human CD28 signaling domain and a human CD3 zeta signaling domain.

44. The CAR according to claim 43, comprising:
(i) an anti-human CD45RC scFv comprising a HCVR having the sequence of SEQ ID NO: 61 and a LCVR having the sequence of SEQ ID NO: 81, linked by a linker having the sequence of SEQ ID NO: 134,
(ii) a hinge domain derived from CD8α having the sequence of SEQ ID NO: 145,
(iii) a human CD8α transmembrane domain having the sequence of SEQ ID NO: 153, and
(iv) an intracellular signaling domain comprising a human CD28 signaling domain having the sequence of SEQ ID NO: 167 and a human CD3 zeta signaling domain having the sequence of SEQ ID NO: 157.

45. A nucleic acid encoding the CAR according to claim 35.

46. An expression vector comprising the nucleic acid according to claim 45.

47. An immune cell population, engineered to express at the cell surface a CAR according to claim 35.

48. The immune cell population according to claim 47, wherein said immune cell population is a regulatory T cell population.

49. The immune cell population according to claim 48, wherein said regulatory T cell population is selected from the group consisting of CD4⁺ CD25⁺ Foxp3⁺ Treg, Tr1 cells, TGF-β secreting Th3 cells, regulatory NKT cells, regulatory γδ T cells, regulatory CD8⁺ T cells, and double negative regulatory T cells.

50. A composition comprising at least one immune cell population engineered to express at the cell surface a CAR according to claim 35.

51. The composition according to claim 50, wherein said composition is a pharmaceutical composition further comprising at least one pharmaceutically acceptable excipient or carrier.

52. A method of treating a subject in need thereof comprising administering to said subject a therapeutically effective amount of the immune cell population according to claim 35, or a composition comprising at least one of said immune cell population.

53. A method of inducing immune tolerance, or of preventing or reducing transplant rejection, or of preventing or treating graft-versus-host disease (GVHD), or of preventing, reducing and/or treating a CD45RChigh-related condition selected from the group consisting of an autoimmune disease, an undesired immune response, a monogenic disease, lymphoma and cancer, in a subject in need thereof, said method comprising administering to said subject a therapeutically effective amount of the immune cell population according to claim 35, or a composition comprising at least one of said immune cell population.


SEQUENCE LISTING (173 sequences)

SEQ ID NO: 1 - protein, 5 aa, Artificial Sequence
  VH-CDR1
NYYIG

SEQ ID NO: 2 - protein, 17 aa, Artificial Sequence
  SITE 1: Xaa is selected from Asp (D), Ile (I) and Arg (R)
  VH-CDR2
  SITE 4: Xaa is selected from Pro (P) and Ser (S)
  SITE 7: Xaa is selected from Asp (D), Ser (S) and Gly (G)
  SITE 9: Xaa is selected from Ala (A) and Thr (T)
  SITE 11: Xaa is selected from Ser (S) and Tyr (Y)
  SITE 12: Xaa is selected from Asn (N), Ala (A) and Ser (S)
  SITE 13: Xaa is selected from Glu (E), Asp (D), Pro (P) and Gln (Q)
  SITE 14: Xaa is selected from Lys (K) and Ser (S)
  SITE 15: Xaa is selected from Phe (F) and Val (V)
  SITE 16: Xaa is selected from Lys (K) and Gln (Q)
XIFXGGXYXNXXXXXXG

SEQ ID NO: 3 - protein, 5 aa, Artificial Sequence
  VH-CDR3
RNFDY

SEQ ID NO: 4 - protein, 17 aa, Artificial Sequence
  VH-CDR2
DIFPGGDYANSNEKFKG

SEQ ID NO: 5 - protein, 17 aa, Artificial Sequence
  VH-CDR2
DIFPGGDYANSNEKVKG

SEQ ID NO: 6 - protein, 17 aa, Artificial Sequence
  VH-CDR2
DIFPGGGYTNYAEKFQG

SEQ ID NO: 7 - protein, 17 aa, Artificial Sequence
  VH-CDR2
DIFPGGSYTNYSESFQG

SEQ ID NO: 8 - protein, 17 aa, Artificial Sequence
  VH-CDR2
DIFPGGSYTNYADSVKG

SEQ ID NO: 9 - protein, 17 aa, Artificial Sequence
  VH-CDR2
RIFPGGGYTNYAQKFQG

SEQ ID NO: 10 - protein, 17 aa, Artificial Sequence
  VH-CDR2
IIFPGGSYTNYSPSFQG

SEQ ID NO: 11 - protein, 17 aa, Artificial Sequence
  VH-CDR2
DIFSGGSYTNYADSVKG

SEQ ID NO: 12 - protein, 11 aa, Artificial Sequence
  SITE 1: Xaa is selected from Ser (S) and Arg (R)
  VL-CDR1
  SITE 8: Xaa is absent, or is selected from Asn (N), Ser (S) and Gly (G)
XASSSVSXYMH

SEQ ID NO: 13 - protein, 7 aa, Artificial Sequence
  SITE 1: Xaa is selected from Asn (N) and Ala (A); or Xaa is any amino acid but Ala (A) or Asn (N)
  VL-CDR2
  SITE 5: Xaa is selected from Leu (L), Ser (S) and Arg (R)
  SITE 6: Xaa is selected from Pro (P), Ala (A) and Gln (Q)
  SITE 7: Xaa is selected from Ser (S) and Thr (T)
XTSNXXX

SEQ ID NO: 14 - protein, 10 aa, Artificial Sequence
  SITE 1: Xaa is selected from Gln (Q) and His (H)
  VL-CDR3
XQRSSYPLTF

SEQ ID NO: 15 - protein, 11 aa, Artificial Sequence
  VL-CDR1
  SITE 8: Xaa is absent or is selected from Asn (N), Ser (S) and Gly (G)
SASSSVSXYMH

SEQ ID NO: 16 - protein, 7 aa, Artificial Sequence
  VL-CDR2
NTSNLPS

SEQ ID NO: 17 - protein, 10 aa, Artificial Sequence
  VL-CDR3
QQRSSYPLTF

SEQ ID NO: 18 - protein, 11 aa, Artificial Sequence
  VL-CDR1
  SITE 8: Xaa is absent or is selected from Asn (N), Ser (S) and Gly (G)
RASSSVSXYMH

SEQ ID NO: 19 - protein, 7 aa, Artificial Sequence
  VL-CDR2
NTSNSPS

SEQ ID NO: 20 - protein, 7 aa, Artificial Sequence
  VL-CDR2
ATSNLQS

SEQ ID NO: 21 - protein, 10 aa, Artificial Sequence
  VL-CDR3
HQRSSYPLTF

SEQ ID NO: 22 - protein, 7 aa, Artificial Sequence
  VL-CDR2
NTSNRAT

SEQ ID NO: 23 - protein, 48 aa, Homo sapiens
  C determinant of hCD45RC
DVPGERSTASTFPTDPVSPLTTTLSLAHHSSAALPARTSNTTITANTS

SEQ ID NO: 24 - DNA, 144 bp, Homo sapiens
  Exon 6 of hCD45RC
gatgtcccaggagagaggagtacagccagcacctttcctacagacccagtttccccattgacaaccaccctcagccttgcacaccacagctctgctgccttacctgcacgcacctccaacaccaccatcacagcgaacacctca

SEQ ID NO: 25 - protein, 30 aa, Artificial Sequence
  VH-FR1
QVQLQQSGAELVRPGTSVKMSCKAAGYTFT

SEQ ID NO: 26 - protein, 14 aa, Artificial Sequence
  VH-FR2
WVKQRPGHGLEWIG

SEQ ID NO: 27 - protein, 32 aa, Artificial Sequence
  VH-FR3
KATLTADTSSSTAYMQLSSLTSEDSAIYYCVR

SEQ ID NO: 28 - protein, 11 aa, Artificial Sequence
  VH-FR4
WGQGTTLTVSS

SEQ ID NO: 29 - protein, 30 aa, Artificial Sequence
  VH-FR1
QVQLVQSGAEVKKPGASVKVSCKASGYTFT

SEQ ID NO: 30 - protein, 14 aa, Artificial Sequence
  VH-FR2
WVRQAPGQGLEWIG

SEQ ID NO: 31 - protein, 32 aa, Artificial Sequence
  VH-FR3
RVTLTADTSISTAYMELSRLRSDDTVVYYCVR

SEQ ID NO: 32 - protein, 11 aa, Artificial Sequence
  VH-FR4
WGQGTLVTVSS

SEQ ID NO: 33 - protein, 30 aa, Artificial Sequence
  VH-FR1
EVQLVQSGAEVKKPGESLKISCKASGYTFT

SEQ ID NO: 34 - protein, 14 aa, Artificial Sequence
  VH-FR2
WVRQMPGKGLEWIG

SEQ ID NO: 35 - protein, 32 aa, Artificial Sequence
  VH-FR3
QVTLSADKSISTAYLQLSSLKASDTAMYYCVR

SEQ ID NO: 36 - protein, 30 aa, Artificial Sequence
  VH-FR1
QVQLVESGGGLVKPGGSLRLSCAASGYTFT

SEQ ID NO: 37 - protein, 14 aa, Artificial Sequence
  VH-FR2
WIRQAPGKGLEWIG

SEQ ID NO: 38 - protein, 32 aa, Artificial Sequence
  VH-FR3
RFTLSADTAKNSAYLQMNSLRAEDTAVYYCVR

SEQ ID NO: 39 - protein, 30 aa, Artificial Sequence
  VH-FR1
EVQLVQSGAEVKKPGESLKISCKGSGYTFT

SEQ ID NO: 40 - protein, 30 aa, Artificial Sequence
  VH-FR1
QVQLVESGGGLVKPGGSLRLSCAASGFTFS

SEQ ID NO: 41 - protein, 32 aa, Artificial Sequence
  VH-FR3
RFTLSADTAKNSLYLQMNSLRAEDTAVYYCVR

SEQ ID NO: 42 - protein, 30 aa, Artificial Sequence
  VH-FR1
EVQLVQSGAEVKKPGESLKISCKGSGYSFT

SEQ ID NO: 43 - protein, 14 aa, Artificial Sequence
  VH-FR2
WIRQAPGKGLEWVG

SEQ ID NO: 44 - protein, 23 aa, Artificial Sequence
  VL-FR1
QIVLTQSPTIMSASPGEKVTITC

SEQ ID NO: 45 - protein, 15 aa, Artificial Sequence
  VL-FR2
WFQQKTGTSPRLWIY

SEQ ID NO: 46 - protein, 32 aa, Artificial Sequence
  VL-FR3
  SITE 15: Xaa is selected from Tyr (Y) and Phe (F)
GVPARFSGSGSGTSXSLTISRMEAEDAATYYC

SEQ ID NO: 47 - protein, 9 aa, Artificial Sequence
  VL-FR4
GAGTKLELK

SEQ ID NO: 48 - protein, 23 aa, Artificial Sequence
  VL-FR1
DIQLTQSPSFLSASVGDRVTITC

SEQ ID NO: 49 - protein, 15 aa, Artificial Sequence
  VL-FR2
WFQQKPGKAPKLWIY

SEQ ID NO: 50 - protein, 32 aa, Artificial Sequence
  VL-FR3
  SITE 15: Xaa is selected from Tyr (Y) and Phe (F)
GVPSRFSGSGSGTEXTLTISSLQPEDFATYYC

SEQ ID NO: 51 - protein, 9 aa, Artificial Sequence
  VL-FR4
GGGTKVEIK

SEQ ID NO: 52 - protein, 23 aa, Artificial Sequence
  VL-FR1
EIVLTQSPDFQSVTPKEKVTITC

SEQ ID NO: 53 - protein, 15 aa, Artificial Sequence
  VL-FR2
WFQQKPDQSPKLWIY

SEQ ID NO: 54 - protein, 32 aa, Artificial Sequence
  VL-FR3
  SITE 15: Xaa is selected from Tyr (Y) and Phe (F)
GVPSRFSGSGSGTDXTLTINSLEAEDAATYYC

SEQ ID NO: 55 - protein, 23 aa, Artificial Sequence
  VL-FR1
EIVLTQSPATLSLSPGERATLSC

SEQ ID NO: 56 - protein, 15 aa, Artificial Sequence
  VL-FR2
WFQQKPGQAPRLWIY

SEQ ID NO: 57 - protein, 32 aa, Artificial Sequence
  VL-FR3
  SITE 15: Xaa is selected from Tyr (Y) and Phe (F)
GIPARFSGSGSGTDXTLTISSLEPEDFAVYYC

SEQ ID NO: 58 - protein, 15 aa, Artificial Sequence
  VL-FR2
WYQQKPGKAPKLWIY

SEQ ID NO: 59 - protein, 15 aa, Artificial Sequence
  VL-FR2
WYQQKPDQSPKLWIY

SEQ ID NO: 60 - protein, 15 aa, Artificial Sequence
  VL-FR2
WYQQKPGQAPRLWIY

SEQ ID NO: 61 - protein, 114 aa, Artificial Sequence
  Murine HCVR
QVQLQQSGAELVRPGTSVKMSCKAAGYTFTNYYIGWVKQRPGHGLEWIGDIFPGGDYANSNEKFKGKATLTADTSSSTAYMQLSSLTSEDSAIYYCVRRNFDYWGQGTTLTVSS

SEQ ID NO: 62 - protein, 114 aa, Artificial Sequence
  Humanized HCVR
QVQLVQSGAEVKKPGASVKVSCKASGYTFTNYYIGWVRQAPGQGLEWIGDIFPGGDYANSNEKFKGRVTLTADTSISTAYMELSRLRSDDTVVYYCVRRNFDYWGQGTLVTVSS

SEQ ID NO: 63 - protein, 114 aa, Artificial Sequence
  Humanized HCVR
EVQLVQSGAEVKKPGESLKISCKASGYTFTNYYIGWVRQMPGKGLEWIGDIFPGGDYANSNEKFKGQVTLSADKSISTAYLQLSSLKASDTAMYYCVRRNFDYWGQGTLVTVSS

SEQ ID NO: 64 - protein, 114 aa, Artificial Sequence
  Humanized HCVR
QVQLVESGGGLVKPGGSLRLSCAASGYTFTNYYIGWIRQAPGKGLEWIGDIFPGGDYANSNEKVKGRFTLSADTAKNSAYLQMNSLRAEDTAVYYCVRRNFDYWGQGTLVTVSS

SEQ ID NO: 65 - protein, 114 aa, Artificial Sequence
  Humanized HCVR
QVQLVQSGAEVKKPGASVKVSCKASGYTFTNYYIGWVRQAPGQGLEWIGDIFPGGGYTNYAEKFQGRVTLTADTSISTAYMELSRLRSDDTVVYYCVRRNFDYWGQGTLVTVSS

SEQ ID NO: 66 - protein, 114 aa, Artificial Sequence
  Humanized HCVR
EVQLVQSGAEVKKPGESLKISCKGSGYTFTNYYIGWVRQMPGKGLEWIGDIFPGGSYTNYSESFQGQVTLSADKSISTAYLQLSSLKASDTAMYYCVRRNFDYWGQGTLVTVSS

SEQ ID NO: 67 - protein, 114 aa, Artificial Sequence
  Humanized HCVR
QVQLVESGGGLVKPGGSLRLSCAASGFTFSNYYIGWIRQAPGKGLEWIGDIFPGGSYTNYADSVKGRFTLSADTAKNSLYLQMNSLRAEDTAVYYCVRRNFDYWGQGTLVTVSS

SEQ ID NO: 68 - protein, 114 aa, Artificial Sequence
  Humanized HCVR
QVQLVQSGAEVKKPGASVKVSCKASGYTFTNYYIGWVRQAPGQGLEWIGRIFPGGGYTNYAQKFQGRVTLTADTSISTAYMELSRLRSDDTVVYYCVRRNFDYWGQGTLVTVSS

SEQ ID NO: 69 - protein, 114 aa, Artificial Sequence
  Humanized HCVR
EVQLVQSGAEVKKPGESLKISCKGSGYSFTNYYIGWVRQMPGKGLEWIGIIFPGGSYTNYSPSFQGQVTLSADKSISTAYLQLSSLKASDTAMYYCVRRNFDYWGQGTLVTVSS

SEQ ID NO: 70 - protein, 114 aa, Artificial Sequence
  Humanized HCVR
QVQLVESGGGLVKPGGSLRLSCAASGFTFSNYYIGWIRQAPGKGLEWVGDIFSGGSYTNYADSVKGRFTLSADTAKNSLYLQMNSLRAEDTAVYYCVRRNFDYWGQGTLVTVSS

SEQ ID NO: 71 - protein, 107 aa, Artificial Sequence
  Murine LCVR
  SITE 31: Xaa is selected from Asn (N), Ser (S) and Gly (G)
QIVLTQSPTIMSASPGEKVTITCSASSSVSXYMHWFQQKTGTSPRLWIYNTSNLPSGVPARFSGSGSGTSFSLTISRMEAEDAATYYCQQRSSYPLTFGAGTKLELK

SEQ ID NO: 72 - protein, 107 aa, Artificial Sequence
  Humanized LCVR
  SITE 31: Xaa is selected from Asn (N), Ser (S) and Gly (G)
DIQLTQSPSFLSASVGDRVTITCSASSSVSXYMHWFQQKPGKAPKLWIYNTSNLPSGVPSRFSGSGSGTEFTLTISSLQPEDFATYYCQQRSSYPLTFGGGTKVEIK

SEQ ID NO: 73 - protein, 107 aa, Artificial Sequence
  Humanized LCVR
  SITE 31: Xaa is selected from Asn (N), Ser (S) and Gly (G)
EIVLTQSPDFQSVTPKEKVTITCSASSSVSXYMHWFQQKPDQSPKLWIYNTSNLPSGVPSRFSGSGSGTDFTLTINSLEAEDAATYYCQQRSSYPLTFGGGTKVEIK

SEQ ID NO: 74 - protein, 107 aa, Artificial Sequence
  Humanized LCVR
  SITE 31: Xaa is selected from Asn (N), Ser (S) and Gly (G)
EIVLTQSPATLSLSPGERATLSCSASSSVSXYMHWFQQKPGQAPRLWIYNTSNLPSGIPARFSGSGSGTDFTLTISSLEPEDFAVYYCQQRSSYPLTFGGGTKVEIK

SEQ ID NO: 75 - protein, 107 aa, Artificial Sequence
  Humanized LCVR
  SITE 31: Xaa is selected from Asn (N), Ser (S) and Gly (G)
DIQLTQSPSFLSASVGDRVTITCRASSSVSXYMHWYQQKPGKAPKLWIYNTSNLPSGVPSRFSGSGSGTEFTLTISSLQPEDFATYYCQQRSSYPLTFGGGTKVEIK

SEQ ID NO: 76 - protein, 107 aa, Artificial Sequence
  Humanized LCVR
  SITE 31: Xaa is selected from Asn (N), Ser (S) and Gly (G)
EIVLTQSPDFQSVTPKEKVTITCRASSSVSXYMHWYQQKPDQSPKLWIYNTSNSPSGVPSRFSGSGSGTDFTLTINSLEAEDAATYYCQQRSSYPLTFGGGTKVEIK

SEQ ID NO: 77 - protein, 107 aa, Artificial Sequence
  Humanized LCVR
  SITE 31: Xaa is selected from Asn (N), Ser (S) and Gly (G)
EIVLTQSPATLSLSPGERATLSCRASSSVSXYMHWFQQKPGQAPRLWIYNTSNLPSGIPARFSGSGSGTDFTLTISSLEPEDFAVYYCQQRSSYPLTFGGGTKVEIK

SEQ ID NO: 78 - protein, 107 aa, Artificial Sequence
  Humanized LCVR
  SITE 31: Xaa is selected from Asn (N), Ser (S) and Gly (G)
DIQLTQSPSFLSASVGDRVTITCRASSSVSXYMHWYQQKPGKAPKLWIYATSNLQSGVPSRFSGSGSGTEFTLTISSLQPEDFATYYCQQRSSYPLTFGGGTKVEIK

SEQ ID NO: 79 - protein, 107 aa, Artificial Sequence
  Humanized LCVR
  SITE 31: Xaa is selected from Asn (N), Ser (S) and Gly (G)
EIVLTQSPDFQSVTPKEKVTITCRASSSVSXYMHWYQQKPDQSPKLWIYNTSNSPSGVPSRFSGSGSGTDFTLTINSLEAEDAATYYCHQRSSYPLTFGGGTKVEIK

SEQ ID NO: 80 - protein, 107 aa, Artificial Sequence
  Humanized LCVR
  SITE 31: Xaa is selected from Asn (N), Ser (S) and Gly (G)
EIVLTQSPATLSLSPGERATLSCRASSSVSXYMHWYQQKPGQAPRLWIYNTSNRATGIPARFSGSGSGTDFTLTISSLEPEDFAVYYCQQRSSYPLTFGGGTKVEIK

SEQ ID NO: 81 - protein, 106 aa, Artificial Sequence
  Murine LCVR
QIVLTQSPTIMSASPGEKVTITCSASSSVSYMHWFQQKTGTSPRLWIYNTSNLPSGVPARFSGSGSGTSYSLTISRMEAEDAATYYCQQRSSYPLTFGAGTKLELK

SEQ ID NO: 82 - protein, 106 aa, Artificial Sequence
  Humanized LCVR
DIQLTQSPSFLSASVGDRVTITCSASSSVSYMHWFQQKPGKAPKLWIYNTSNLPSGVPSRFSGSGSGTEYTLTISSLQPEDFATYYCQQRSSYPLTFGGGTKVEIK

SEQ ID NO: 83 - protein, 106 aa, Artificial Sequence
  Humanized LCVR
EIVLTQSPDFQSVTPKEKVTITCSASSSVSYMHWFQQKPDQSPKLWIYNTSNLPSGVPSRFSGSGSGTDYTLTINSLEAEDAATYYCQQRSSYPLTFGGGTKVEIK

SEQ ID NO: 84 - protein, 106 aa, Artificial Sequence
  Humanized LCVR
EIVLTQSPATLSLSPGERATLSCSASSSVSYMHWFQQKPGQAPRLWIYNTSNLPSGIPARFSGSGSGTDYTLTISSLEPEDFAVYYCQQRSSYPLTFGGGTKVEIK

SEQ ID NO: 85 - protein, 106 aa, Artificial Sequence
  Humanized LCVR
DIQLTQSPSFLSASVGDRVTITCRASSSVSYMHWYQQKPGKAPKLWIYNTSNLPSGVPSRFSGSGSGTEYTLTISSLQPEDFATYYCQQRSSYPLTFGGGTKVEIK

SEQ ID NO: 86 - protein, 106 aa, Artificial Sequence
  Humanized LCVR
EIVLTQSPDFQSVTPKEKVTITCRASSSVSYMHWYQQKPDQSPKLWIYNTSNSPSGVPSRFSGSGSGTDFTLTINSLEAEDAATYYCQQRSSYPLTFGGGTKVEIK

SEQ ID NO: 87 - protein, 106 aa, Artificial Sequence
  Humanized LCVR
EIVLTQSPATLSLSPGERATLSCRASSSVSYMHWFQQKPGQAPRLWIYNTSNLPSGIPARFSGSGSGTDYTLTISSLEPEDFAVYYCQQRSSYPLTFGGGTKVEIK

SEQ ID NO: 88 - protein, 106 aa, Artificial Sequence
  Humanized LCVR
DIQLTQSPSFLSASVGDRVTITCRASSSVSYMHWYQQKPGKAPKLWIYATSNLQSGVPSRFSGSGSGTEFTLTISSLQPEDFATYYCQQRSSYPLTFGGGTKVEIK

SEQ ID NO: 89 - protein, 106 aa, Artificial Sequence
  Humanized LCVR
EIVLTQSPDFQSVTPKEKVTITCRASSSVSYMHWYQQKPDQSPKLWIYNTSNSPSGVPSRFSGSGSGTDFTLTINSLEAEDAATYYCHQRSSYPLTFGGGTKVEIK

SEQ ID NO: 90 - protein, 106 aa, Artificial Sequence
  Humanized LCVR
EIVLTQSPATLSLSPGERATLSCRASSSVSYMHWYQQKPGQAPRLWIYNTSNRATGIPARFSGSGSGTDFTLTISSLEPEDFAVYYCQQRSSYPLTFGGGTKVEIK

SEQ ID NO: 91 - protein, 330 aa, Artificial Sequence
  Human HCCR
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 107 aa, Artificial Sequence [sequence omitted]

SEQ ID NO: 93 - protein, 323 aa, Artificial Sequence
  Murine HCCR
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPG

SEQ ID NO: 94 - protein, 107 aa, Artificial Sequence
  Murine LCCR
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 95 - DNA, 342 bp, Artificial Sequence
  Murine HCVR
caggtccagctgcaacagtctggcgctgagctggttaggcctgggacttcagtgaagatgtcctgcaaggccgctggatacaccttcactaactactacataggttgggtaaagcagaggcctggacatggccttgagtggatcggagatattttccctggaggtgactatgccaacagcaatgagaagttcaagggcaaagccacactgactgcagacacatcctccagcacagcctacatgcagctcagcagcctgacatctgaggactctgccatctattactgtgtgagaaggaactttgactactggggccaaggcaccactctcacagtgtcctca

SEQ ID NO: 96 - DNA, 318 bp, Artificial Sequence
  Murine LCVR
caaattgttctcacccagtctccaacaatcatgtctgcatctccaggggagaaggtgaccataacctgcagtgccagctcaagtgtaagttacatgcactggttccagcagaagacaggcacttctcccagactctggatttataacacatccaacctgccttctggagtccccgctcgcttcagtggcagtggatctgggacctcttactctctcacaatcagccgaatggaggctgaagatgctgccacttattactgccagcaaaggagtagttacccactcacgttcggtgctgggaccaagctggagctgaaa

SEQ ID NO: 97-98 - skipped (2 entries)

SEQ ID NO: 99 - protein, 1306 aa, Homo sapiens
  hCD45
  ref <308>: DATABASE ACCESSION NUMBER: UniProt Accession P08575-3
  ref <309>: DATABASE ENTRY DATE: 2018 03 28
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 100 - protein, 17 aa, Artificial Sequence
  VH-CDR2
DIFPGGDYTNYAEKFQG

SEQ ID NO: 101 - protein, 114 aa, Artificial Sequence
  Humanized HCVR
QVQLVQSGAEVKKPGASVKVSCKASGYTFTNYYIGWVRQAPGQGLEWIGDIFPGGDYTNYAEKFQGRVTLTADTSISTAYMELSRLRSDDTVVYYCVRRNFDYWGQGTLVTVSS

SEQ ID NO: 102 - protein, 107 aa, Artificial Sequence
  Humanized LCVR
  SITE 31: Xaa is selected from Asn (N), Ser (S) and Gly (G)
DIQLTQSPSFLSASVGDRVTITCRASSSVSXYMHWFQQKPGKAPKLWIYNTSNLPSGVPSRFSGSGSGTEFTLTISSLQPEDFATYYCQQRSSYPLTFGGGTKVEIK

SEQ ID NO: 103 - protein, 107 aa, Artificial Sequence
  Humanized LCVR
DIQLTQSPSFLSASVGDRVTITCRASSSVSSYMHWFQQKPGKAPKLWIYNTSNLPSGVPSRFSGSGSGTEFTLTISSLQPEDFATYYCQQRSSYPLTFGGGTKVEIK

SEQ ID NO: 104 - protein, 1193 aa, Homo sapiens
  hCD45RC
  ref <308>: DATABASE ACCESSION NUMBER: UniProt Accession P08575-10
  ref <309>: DATABASE ENTRY DATE: 2018 03 28
MTMYLWLKLLAFGFAFLDTEVFVTGQSPTPSPTDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 105 - protein, 66 aa, Homo sapiens
  A determinant of hCD45
GLTTAKMPSVPLSSDPLPTHTTAFSPASTFERENDFSETTTSLSPDNTSTQVSPDSLDNASAFNTT

SEQ ID NO: 106 - protein, 47 aa, Homo sapiens
  B determinant of hCD45
GVSSVQTPHLPTHADSQTPSAGTDTQTFSGSAANAKLNPTPGSNAIS

SEQ ID NO: 107 - protein, 1211 aa, Homo sapiens
  hCD45RA
  ref <308>: DATABASE ACCESSION NUMBER: UniProt Accession P08575-8
  ref <309>: DATABASE ENTRY DATE: 2018 03 28
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 108 - protein, 1192 aa, Homo sapiens
  hCD45RB
  ref <308>: DATABASE ACCESSION NUMBER: UniProt Accession P08575-9
  ref <309>: DATABASE ENTRY DATE: 2018 03 28
MTMYLWLKLLAFGFAFLDTEVFVTGQSPTPSPTGVSSVQTPHLPTHADSQTPSAGTDTQTFSGSAANAKLNPTPGSNAISDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 109 - protein, 1258 aa, Homo sapiens
  hCD45RAB
  ref <308>: DATABASE ACCESSION NUMBER: UniProt Accession P08575-5
  ref <309>: DATABASE ENTRY DATE: 2018 03 28
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 110 - protein, 1145 aa, Homo sapiens
  hCD45R0
  ref <308>: DATABASE ACCESSION NUMBER: UniProt Accession P08575-4
  ref <309>: DATABASE ENTRY DATE: 2018 03 28
MTMYLWLKLLAFGFAFLDTEVFVTGQSPTPSPTDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 111 - protein, 7 aa, Artificial Sequence
  VL-CDR2
ATSNLPS

SEQ ID NO: 112 - protein, 107 aa, Artificial Sequence
  Humanized LCVR
  SITE 31: Xaa is selected from Asn (N), Ser (S) and Gly (G)
DIQLTQSPSFLSASVGDRVTITCRASSSVSXYMHWYQQKPGKAPKLWIYATSNLPSGVPSRFSGSGSGTEFTLTISSLQPEDFATYYCQQRSSYPLTFGGGTKVEIK

SEQ ID NO: 113 - protein, 106 aa, Artificial Sequence
  Humanized LCVR
DIQLTQSPSFLSASVGDRVTITCRASSSVSYMHWYQQKPGKAPKLWIYATSNLPSGVPSRFSGSGSGTEYTLTISSLQPEDFATYYCQQRSSYPLTFGGGTKVEIK

SEQ ID NO: 114-115 - skipped (2 entries)

SEQ ID NO: 116 - protein, 17 aa, Artificial Sequence
  VH-CDR2
DIFPGGGYANYAEKFQG

SEQ ID NO: 117 - protein, 17 aa, Artificial Sequence
  VH-CDR2
DIFPGGGYTNYAEKFKG

SEQ ID NO: 118 - protein, 17 aa, Artificial Sequence
  VH-CDR2
DIFPGGGYTNYNEKFQG

SEQ ID NO: 119 - protein, 17 aa, Artificial Sequence
  VH-CDR2
DIFPGGGYTNSAEKFQG

SEQ ID NO: 120 - protein, 7 aa, Artificial Sequence
  VL-CDR2
NTANLPS

SEQ ID NO: 121 - protein, 114 aa, Artificial Sequence
  Humanized HCVR
QVQLVQSGAEVKKPGASVKVSCKASGYTFTNYYIGWVRQAPGQGLEWIGDIFPGGGYANYAEKFQGRVTLTADTSISTAYMELSRLRSDDTVVYYCVRRNFDYWGQGTLVTVSS

SEQ ID NO: 122 - protein, 114 aa, Artificial Sequence
  Humanized HCVR
QVQLVQSGAEVKKPGASVKVSCKASGYTFTNYYIGWVRQAPGQGLEWIGDIFPGGGYTNYAEKFKGRVTLTADTSISTAYMELSRLRSDDTVVYYCVRRNFDYWGQGTLVTVSS

SEQ ID NO: 123 - protein, 114 aa, Artificial Sequence
  Humanized HCVR
QVQLVQSGAEVKKPGASVKVSCKASGYTFTNYYIGWVRQAPGQGLEWIGDIFPGGGYTNYNEKFQGRVTLTADTSISTAYMELSRLRSDDTVVYYCVRRNFDYWGQGTLVTVSS

SEQ ID NO: 124 - protein, 114 aa, Artificial Sequence
  Humanized HCVR
QVQLVQSGAEVKKPGASVKVSCKASGYTFTNYYIGWVRQAPGQGLEWIGDIFPGGGYTNSAEKFQGRVTLTADTSISTAYMELSRLRSDDTVVYYCVRRNFDYWGQGTLVTVSS

SEQ ID NO: 125 - protein, 107 aa, Artificial Sequence
  Humanized LCVR
  SITE 31: Xaa is selected from Asn (N), Ser (S) and Gly (G)
QIVLTQSPTIMSASPGEKVTITCSASSSVSXYMHWFQQKTGTSPRLWIYNTANLPSGVPARFSGSGSGTSFSLTISRMEAEDAATYYCQQRSSYPLTFGAGTKLELK

SEQ ID NO: 126 - protein, 106 aa, Artificial Sequence
  Humanized LCVR
QIVLTQSPTIMSASPGEKVTITCSASSSVSYMHWFQQKTGTSPRLWIYNTANLPSGVPARFSGSGSGTSYSLTISRMEAEDAATYYCQQRSSYPLTFGAGTKLELK

SEQ ID NO: 127 - protein, 7 aa, Artificial Sequence
  SITE 1: Xaa is any amino acid but Ala (A) or Asn (N)
  VL-CDR2
XTSNLPS

SEQ ID NO: 128 - protein, 107 aa, Artificial Sequence
  Humanized LCVR
  SITE 31: Xaa is selected from Asn (N), Ser (S) and Gly (G)
  SITE 50: Xaa is any amino acid but Ala (A) or Asn (N)
DIQLTQSPSFLSASVGDRVTITCRASSSVSXYMHWYQQKPGKAPKLWIYXTSNLPSGVPSRFSGSGSGTEFTLTISSLQPEDFATYYCQQRSSYPLTFGGGTKVEIK

SEQ ID NO: 129 - protein, 106 aa, Artificial Sequence
  Humanized LCVR
  SITE 49: Xaa is any amino acid but Ala (A) or Asn (N)
DIQLTQSPSFLSASVGDRVTITCRASSSVSYMHWYQQKPGKAPKLWIYXTSNLPSGVPSRFSGSGSGTEYTLTISSLQPEDFATYYCQQRSSYPLTFGGGTKVEIK

SEQ ID NO: 130 - protein, 4 aa, Artificial Sequence
  linker Gly3 Ser
GGGS

SEQ ID NO: 131 - protein, 16 aa, Artificial Sequence
  linker (Gly3 Ser)4
GGGSGGGSGGGSGGGS

SEQ ID NO: 132 - protein, 5 aa, Artificial Sequence
  linker Gly4 Ser
GGGGS

SEQ ID NO: 133 - protein, 10 aa, Artificial Sequence
  linker (Gly4 Ser)2
GGGGSGGGGS

SEQ ID NO: 134 - protein, 15 aa, Artificial Sequence
  linker (Gly4 Ser)3
GGGGSGGGGSGGGGS

SEQ ID NO: 135 - protein, 20 aa, Artificial Sequence
  linker (Gly4 Ser)4
GGGGSGGGGSGGGGSGGGGS

SEQ ID NO: 136 - DNA, 45 bp, Artificial Sequence
  (G4S)3 linker
ggaggtggaggctctggcggtggaggaagtggtgggggaggctct

SEQ ID NO: 137 - DNA, 45 bp, Artificial Sequence
  (G4S)3 linker
ggtggcggtggctcgggcggtggtgggtcgggtggcggcggatct

SEQ ID NO: 138 - DNA, 705 bp, Artificial Sequence
  scFv CD45RC-CAR
aaggtccagctgcaacagtctggcgctgagctggttaggcctgggacttcagtgaagatgtcctgcaaggccgctggatacaccttcactaactactacataggttgggtaaagcagaggcctggacatggccttgagtggatcggagatattttccctggaggtgactatgccaacagcaatgagaagttcaagggcaaagccacactgactgcagacacatcctccagcacagcctacatgcagctcagcagcctgacatctgaggactctgccatctattactgtgtgagaaggaactttgactactggggccaaggcaccactctcacagtgtcctcaggtggcggtggctcgggcggtggtgggtcgggtggcggcggatctcaaattgttctcacccagtctccaacaatcatgtctgcatctccaggggagaaggtgaccataacctgcagtgccagctcaagtgtaagttacatgcactggttccagcagaagacaggcacttctcccagactctggatttataacacatccaacctgccttctggagtccccgctcgcttcagtggcagtggatctgggacctcttactctctcacaatcagccgaatggaggctgaagatgctgccacttattactgccagcaaaggagtagttacccactcacgttcggtgctgggaccaagctggagctgaaa

SEQ ID NO: 139 - protein, 16 aa, Artificial Sequence
  hinge
AGSSSSGGSTTGGSTT

SEQ ID NO: 140 - protein, 16 aa, Artificial Sequence
  hinge
GTTAASGSSGGSSSGA

SEQ ID NO: 141 - protein, 16 aa, Artificial Sequence
  hinge
SSATATAGTGSSTGST

SEQ ID NO: 142 - protein, 16 aa, Artificial Sequence
  hinge
TSGSTGTAASSTSTST

SEQ ID NO: 143 - protein, 30 aa, Artificial Sequence
  hinge
GGTGGCGGAGGTTCTGGAGGTGGAGGTTCC

SEQ ID NO: 144 - protein, 7 aa, Artificial Sequence
  hinge KIR2DS2
KIRRDSS

SEQ ID NO: 145 - protein, 45 aa, Artificial Sequence
  CD8 hinge
TTTPAPRPPTPAPTIASQPLSLRPEACRPAAGGAVHTRGLDFACD

SEQ ID NO: 146 - DNA, 135 bp, Artificial Sequence
  CD8 hinge
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 147 - protein, 230 aa, Artificial Sequence
  IgG4 hinge
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKM

SEQ ID NO: 148 - DNA, 690 bp, Artificial Sequence
  IgG4 hinge
gagagcaagtacggccctccctgccccccttgccctgcccccgagttcctgggcggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttcaatagcacctaccgggtggtgtccgtgctgaccgtgctgcaccaggactggctgaacggcaaggaatacaagtgtaaggtgtccaacaagggcctgcccagcagcatcgagaaaaccatcagcaaggccaagggccagcctcgggagccccaggtgtacaccctgccccctagccaagaggagatgaccaagaaccaggtgtccctgacctgcctggtgaagggcttctaccccagcgacatcgccgtggagtgggagagcaacggccagcccgagaacaactacaagaccaccccccctgtgctggacagcgacggcagcttcttcctgtacagccggctgaccgtggacaagagccggtggcaggagggcaacgtctttagctgctccgtgatgcacgaggccctgcacaaccactacacccagaagagcctgagcctgtccctgggcaagatg

SEQ ID NO: 149 - protein, 282 aa, Artificial Sequence
  IgD hinge
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 150 - DNA, 847 bp, Artificial Sequence
  IgD hinge
aggtggcccgaaagtcccaaggcccaggcatctagtgttcctactgcacagccccaggcagaaggcagcctagccaaagctactactgcacctgccactacgcgcaatactggccgtggcggggaggagaagaaaaaggagaaagagaaagaagaacaggaagagagggagaccaagacccctgaatgtccatcccatacccagccgctgggcgtctatctcttgactcccgcagtacaggacttgtggcttagagataaggccacctttacatgtttcgtcgtgggctctgacctgaaggatgcccatttgacttgggaggttgccggaaaggtacccacagggggggttgaggaagggttgctggagcgccattccaatggctctcagagccagcactcaagactcacccttccgagatccctgtggaacgccgggacctctgtcacatgtactctaaatcatcctagcctgcccccacagcgtctgatggcccttagagagccagccgcccaggcaccagttaagcttagcctgaatctgctcgccagtagtgatcccccagaggccgccagctggctcttatgcgaagtgtccggctttagcccgcccaacatcttgctcatgtggctggaggaccagcgagaagtgaacaccagcggcttcgctccagcccggcccccaccccagccgggttctaccacattctgggcctggagtgtcttaagggtcccagcaccacctagcccccagccagccacatacacctgtgttgtgtcccatgaagatagcaggaccctgctaaatgcttctaggagtctggaggtttcctacgtgactgaccatt

SEQ ID NO: 151 - protein, 39 aa, Artificial Sequence
  CD28 hinge
IEVMYPPPYLDNEKSNGTIIHVKGKHLCPSPLFPGPSKP

SEQ ID NO: 152 - DNA, 117 bp, Artificial Sequence
  CD28 hinge
attgaagttatgtatcctcctccttacctagacaatgagaagagcaatggaaccattatccatgtgaaagggaaacacctttgtccaagtcccctatttcccggaccttctaagccc

SEQ ID NO: 153 - protein, 24 aa, Artificial Sequence
  CD8 transmembrane
IYIWAPLAGTCGVLLLSLVITLYC

SEQ ID NO: 154 - DNA, 72 bp, Artificial Sequence
  CD8 transmembrane
atctacatctgggcgcccttggccgggacttgtggggtccttctcctgtcactggttatcaccctttactgc

SEQ ID NO: 155 - protein, 27 aa, Artificial Sequence
  CD28 transmembrane
FWVLVVVGGVLACYSLLVTVAFIIFWV

SEQ ID NO: 156 - DNA, 81 bp, Artificial Sequence
  CD28 transmembrane
ttttgggtgctggtggtggttggtggagtcctggcttgctatagcttgctagtaacagtggcctttattattttctgggtg

SEQ ID NO: 157 - protein, 112 aa, Artificial Sequence
  CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 158-161 - skipped (4 entries)

SEQ ID NO: 162 - DNA, 336 bp, Artificial Sequence
  CD3 zeta
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 163 - protein, 42 aa, Artificial Sequence
  4-1BB intracellular
KRGRKKLLYIFKQPFMRPVQTTQEEDGCSCRFPEEEEGGCEL

SEQ ID NO: 164 - DNA, 126 bp, Artificial Sequence
  4-1BB intracellular
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 165-166 - skipped (2 entries)

SEQ ID NO: 167 - protein, 41 aa, Artificial Sequence
  CD28 intracellular
RSKRSRLLHSDYMNMTPRRPGPTRKHYQPYAPPRDFAAYRS

SEQ ID NO: 168 - DNA, 123 bp, Artificial Sequence
  CD28 intracellular
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 169 - protein, 222 aa, Artificial Sequence
  CD8-CD28-CD3zeta
TTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 170 - DNA, 666 bp, Artificial Sequence
  CD8-CD28-CD3zeta
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 171 - protein, 457 aa, Artificial Sequence
  CD45RC-CD8-CD28-CD3zeta
KVQLQQSGAELVRPGTSVKMSCKAAGYTFTNYYIGWVKQRPGHGLEWIGDIFPGGDYANSNEKFKGKATLTADTSSSTAYMQLSSLTSEDSAIYYCVRRNFDYWGQGTTLTVSSGGGGSGGGGSGGGGSQIVLTQSPTIMSASPGEKVTITCSASSSVSYMHWFQQKTGTSPRLWIYNTSNLPSGVPARFSGSGSGTSYSLTISRMEAEDAATYYCQQRSSYPLTFGAGTKLELKTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 172 - DNA, 1371 bp, Artificial Sequence
  CD45RC-CD8-CD28-CD3zeta
aaggtccagctgcaacagtctggcgctgagctggttaggcctgggacttcagtgaagatgtcctgcaaggccgctggatacaccttcactaactactacataggttgggtaaagcagaggcctggacatggccttgagtggatcggagatattttccctggaggtgactatgccaacagcaatgagaagttcaagggcaaagccacactgactgcagacacatcctccagcacagcctacatgcagctcagcagcctgacatctgaggactctgccatctattactgtgtgagaaggaactttgactactggggccaaggcaccactctcacagtgtcctcaggtggcggtggctcgggcggtggtgggtcgggtggcggcggatctcaaattgttctcacccagtctccaacaatcatgtctgcatctccaggggagaaggtgaccataacctgcagtgccagctcaagtgtaagttacatgcactggttccagcagaagacaggcacttctcccagactctggatttataacacatccaacctgccttctggagtccccgctcgcttcagtggcagtggatctgggacctcttactctctcacaatcagccgaatggaggctgaagatgctgccacttattactgccagcaaaggagtagttacccactcacgttcggtgctgggaccaagctggagctgaaaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 173 - protein, 235 aa, Artificial Sequence
  ScFv CD45RC-CAR
KVQLQQSGAELVRPGTSVKMSCKAAGYTFTNYYIGWVKQRPGHGLEWIGDIFPGGDYANSNEKFKGKATLTADTSSSTAYMQLSSLTSEDSAIYYCVRRNFDYWGQGTTLTVSSGGGGSGGGGSGGGGSQIVLTQSPTIMSASPGEKVTITCSASSSVSYMHWFQQKTGTSPRLWIYNTSNLPSGVPARFSGSGSGTSYSLTISRMEAEDAATYYCQQRSSYPLTFGAGTKLELK